(19) United States
(12) Patent Application Publication
(10) Pub. No.: US 2023/0357242 A1
(43) Pub. Date: Nov. 9, 2023

(54) COMPOUND AS AKT KINASE INHIBITOR

(71) Applicant: CHIA TAI TIANQING PHARMACEUTICAL GROUP CO., LTD., Lianyungang, Jiangsu (CN)

(72) Inventors: Yinsheng ZHANG, Nanjing City, Jiangsu (CN); Yong GAO, Nanjing City, Jiangsu (CN); Wei SHI, Nanjing City, Jiangsu (CN); Damin ZHAO, Nanjing City, Jiangsu (CN); Yuan YIN, Nanjing City, Jiangsu (CN)

(21) Appl. No.: 18/246,251

(22) PCT Filed: Sep. 30, 2021
(86) PCT No.: PCT/CN2021/122040

§ 371 Date: Mar. 22, 2023

(30) Foreign Application Priority Data
Sep. 30, 2020 (CN) 202011057860.0
Mar. 10, 2021 (CN) 202110261605.6

(51) Int. Cl. C07D 471/04; C07D 519/00

(52) U.S. Cl. CPC C07D 471/04; C07D 519/00

(57) ABSTRACT

Provided is a compound as an Akt kinase inhibitor. The present invention specifically relates to a compound of formula (I) or a pharmaceutically acceptable salt thereof, a preparation method therefor, a pharmaceutical composition containing the compound, and the use thereof in the preparation of a drug for treating Akt kinase-related diseases.

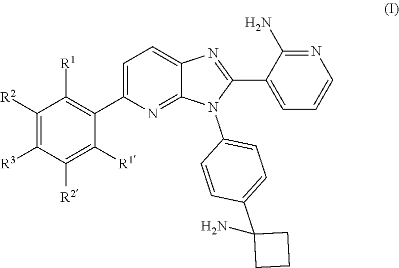

### CROSS-REFERENCE TO RELATED APPLICATIONS

The present application is a National Stage Application of International Application No. PCT/CN2021/122040, filed Sep. 30, 2021, which claims priority to and benefit of the Chinese Patent Application No. 202011057860.0 filed with China National Intellectual Property Administration on Sep. 30, 2020 and the Chinese Patent Application No. 202110261605.6 filed with China National Intellectual Property Administration on Mar. 10, 2021, the disclosure of each of which is incorporated herein by reference in its entirety.

### TECHNICAL FIELD

The present application belongs to the field of pharmaceutical chemistry, provides a compound as an Akt kinase inhibitor or a pharmaceutically acceptable salt thereof, a preparation method therefor and a pharmaceutical composition containing the compound, and relates to use thereof in preparing a drug for treating an Akt kinase-related disease of a patient in need thereof, such as treating cancer.

### BACKGROUND

Akt, also known as Protein Kinase B (PKB) or Rac, is a serine/threonine kinase of the AGC family with high homology to Protein Kinase A (PKA) and Protein Kinase C (PKC). Akt mainly has three domains: a PH domain (which has an affinity for PIP3 and is therefore critical for binding to cell membranes), a catalytic domain and a regulatory domain. Studies have shown that Akt in humans comprises three subtypes: Akt1, Akt2 and Akt3, each of which has unique function and expression pattern. Akt1, Akt2 and Akt3 are key mediators of PI3K/AKT/mTOR signaling pathway, and can promote various physiological processes such as proliferation, migration, anti-apoptotic survival and protein synthesis. Akt1 has wide expression in tissues and is mainly involved in the survival pathway and growth control of cells; Akt2 is mainly expressed in muscle and adipocytes and involved in insulin-mediated glycometabolism and the like; Akt3 is mainly expressed in testis and brain and plays an important role in maintaining normal brain volume.

Akt is one of the most frequently activated protein kinases in human cancers. Over-activation of Akt may induce cell growth, resulting in cell proliferation and helping to resist apoptosis. In cancer, Akt activity is often elevated due to oncogenic growth factors, angiogenic factors, cytokines and genetic alterations, including mutations and/or amplification of the Akt1, Akt2 and Akt3 genes. Akt signaling can be regulated from various levels, for example several molecules upstream thereof can regulate the dephosphorylation of PIP3 that can be dephosphorylated to PIP2 by PTEN and SHIP1. Akt can also be dephosphorylated by PPA2 and PHLPP, resulting in inactivation. In addition, with phosphorylation of NFkB and IRS-1, positive and negative feedback regulation and control can be performed on Akt. Studies have shown that Akt is overexpressed in various human tumors, and the abnormal function of the Akt is closely related to the occurrence, development and tolerance of chemotherapy and radiotherapy of the tumors.

According to different binding sites, Akt inhibitors may be classified into PH domain inhibitors, ATP competitive inhibitors, allosteric inhibitors, biologics and the like. Compared with Capivasertib (AZD5363) that is developed by AstraZeneca and is a pan-Akt inhibitor, ATP competitive inhibitors and allosteric inhibitors (e.g., MK-2206) do not show monotherapy activity in many clinical trials. ARQ 092 and ARQ 751 are highly selective allosteric inhibitors, both of which show better activity in early studies.

Akt has become an anti-tumor target with a great development prospect, and an Akt inhibitor with high efficiency (activity), novelty (structure) and high selectivity (safety) is an important strategy for solving the unmet medical requirements at present.

### BRIEF SUMMARY

In one aspect, the present application provides a compound of formula (I) or a pharmaceutically acceptable salt thereof,

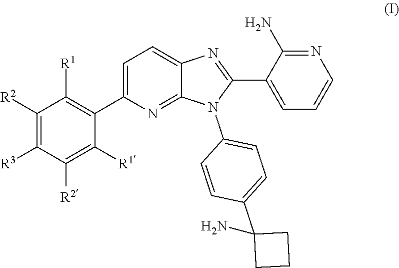

wherein,
R¹ and R^{1′} are each independently selected from the group consisting of hydrogen and halogen;
R², R^{2′} and R³ are each independently selected from the group consisting of hydrogen, amino, amino-C₁-C₆ alkyl-, 7-9 membered spiro-heterocyclyl, 10 membered spiro-heterocyclyl whose ring atoms consist of a nitrogen atom and a carbon atom, 7-10 membered fused-heterocyclyl, 7-10 membered bridged heterocyclyl, 5-6 membered heterocyclyl whose ring atoms consist of a nitrogen atom and a carbon atom, 5-6 membered heterocyclyl whose ring atoms contain a silicon atom or a phosphorus atom,

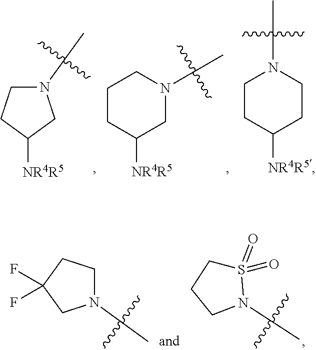

and wherein the amino or amino-C₁-C₆ alkyl- is substituted with one or more R²¹, and optionally substituted with one or more R^{21′}, wherein the 5-6 membered heterocyclyl whose ring atoms consist of a nitrogen atom and a carbon atom is substituted with one or more R²², and optionally substituted with one or more R^{22′}, wherein the 7-9 membered spiro-heterocyclyl, 10 membered spiro-heterocyclyl whose ring atoms consist of a nitrogen atom and a carbon atom, 7-10 membered heterocycloalkenyl or 5-6 membered heterocyclyl whose ring atoms contain a silicon atom or a phosphorus atom is optionally substituted with one or more R²³, wherein the 7-10 membered bridged heterocyclyl is substituted with one or more R²⁴,
or, R³ is connected to R² or R^{2′}, and together with the carbon atoms connected thereto, forms morpholinyl optionally substituted with one or more R²⁵,
and R², R^{2′} and R³ are not hydrogen at the same time;
R²¹ is selected from the group consisting of C₂-C₆ alkenyl-C(O)—, C₃-C₁₂ cycloalkyl, 4-12 membered heterocyclyl or C₁-C₆ alkylacylamino-C₁-C₆ alkyl-, wherein the C₃-C₁₂ cycloalkyl, 4-12 membered heterocyclyl or C₁-C₆ alkylacylamino-C₁-C₆ alkyl- is optionally substituted with one or more of the following groups selected from the group consisting of: hydroxy, C₁-C₆ alkyl and C₁-C₆ alkylacyl; R²² is selected from the group consisting of C₂-C₆ alkynyl-C(O)—, 4-5 membered heterocyclyl or 6 membered heterocyclyl whose ring atoms consist of a nitrogen atom and a sulfur atom, wherein the 4-5 membered heterocyclyl or 6 membered heterocyclyl whose ring atoms consist of a nitrogen atom and a sulfur atom is optionally substituted with one or more of the following groups selected from the group consisting of: [symbol] , halogen, C₁-C₃ alkyl and C₁-C₆ alkylacyl;
R^{21′} and R^{22′} are each independently selected from the group consisting of deuterium and C₁-C₆ alkyl;
R²³ and R²⁴ are each independently selected from the group consisting of [symbol] , C₁-C₆ alkyl, C₁-C₆ alkylsulfonyl, C₁-C₆ alkylacyl and C₁-C₆ alkylacyl-N(C₁-C₆ alkyl)-; [symbol]
R²⁵ is selected from C₁-C₆ alkylacyl;
R⁴ is selected from the group consisting of C₁-C₆ alkylacyl and C₁-C₆ alkylsulfonyl;
R⁵ is selected from C₁-C₆ alkyl;
R^{5′} is selected from the group consisting of —CD₃ and —CH₃, and when R^{5′} is —CH₃, R¹ or R^{1′} is halogen.

In another aspect, the present application further provides a pharmaceutical composition comprising the compound or the pharmaceutically acceptable salt thereof of the present application described above.

In another aspect, the present application further provides a method for treating a disease mediated by Akt kinase in a mammal, comprising administering to a mammal in need of such treatment a therapeutically effective amount of the compound, the pharmaceutically acceptable salt thereof or the pharmaceutical composition thereof described above.

In another aspect, the present application further provides use of the compound, the pharmaceutically acceptable salt thereof or the pharmaceutical composition thereof described above in preparing a medicament for treating a disease mediated by Akt kinase.

In another aspect, the present application further provides use of the compound, the pharmaceutically acceptable salt thereof or the pharmaceutical composition thereof described above in treating a disease mediated by Akt kinase.

In another aspect, the present application further provides the compound, the pharmaceutically acceptable salt thereof or the pharmaceutical composition thereof of the present application described above for use in treating a disease mediated by Akt kinase.

### SUMMARY

In one aspect, the present application provides a compound of formula (I) or a pharmaceutically acceptable salt thereof,

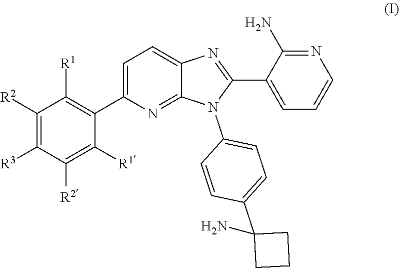

wherein,
R¹ and R^{1′} are each independently selected from the group consisting of hydrogen and halogen;
R², R^{2′} and R³ are each independently selected from the group consisting of hydrogen, amino, amino-C₁-C₆ alkyl-, 7-9 membered spiro-heterocyclyl, 10 membered spiro-heterocyclyl whose ring atoms consist of a nitrogen atom and a carbon atom, 7-10 membered fused-heterocyclyl, 7-10 membered bridged heterocyclyl, 5-6 membered heterocyclyl whose ring atoms consist of a nitrogen atom and a carbon atom, 5-6 membered heterocyclyl whose ring atoms contain a silicon atom or a phosphorus atom,

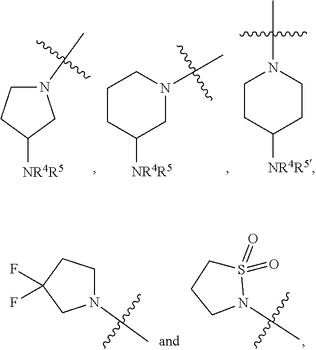

wherein the amino or amino-C₁-C₆ alkyl- is substituted with one or more R²¹, and optionally substituted with one or more R^{21′}, wherein the 5-6 membered heterocyclyl whose ring atoms consist of a nitrogen atom and a carbon atom is substituted with one or more R²², and optionally substituted with one or more R^{22′}, wherein the 7-9 membered spiro-heterocyclyl, 10 membered spiro-heterocyclyl whose ring atoms consist of a nitrogen atom and a carbon atom, 7-10 membered heterocycloalkenyl or 5-6 membered heterocyclyl whose ring atoms contain a silicon atom or a phosphorus atom is optionally substituted with one or more R²³, wherein the 7-10 membered bridged heterocyclyl is substituted with one or more R²⁴,
or, R³ is connected to R² or R^{2′}, and together with the carbon atoms connected thereto, forms morpholinyl optionally substituted with one or more R²,
and R², R^{2′} and R³ are not hydrogen at the same time;
R²¹ is selected from the group consisting of C₂-C₆ alkenyl-C(O)—, C₃-C₁₂ cycloalkyl, 4-12 membered heterocyclyl or C₁-C₆ alkylacylamino-C₁-C₆ alkyl-, wherein the C₃-C₁₂ cycloalkyl, 4-12 membered heterocyclyl or C₁-C₆ alkylacylamino-C₁-C₆ alkyl- is optionally substituted with one or more of the following groups selected from the group consisting of: hydroxy, C₁-C₆ alkyl and C₁-C₆ alkylacyl;
R²² is selected from the group consisting of C₂-C₆ alkynyl-C(O)—, 4-5 membered heterocyclyl or 6 membered heterocyclyl whose ring atoms consist of a nitrogen atom and a sulfur atom, wherein the 4-5 membered heterocyclyl or 6 membered heterocyclyl whose ring atoms consist of a nitrogen atom and a sulfur atom is optionally substituted with one or more of the following groups selected from the group consisting of: [symbol] , halogen, C₁-C₃ alkyl and C₁-C₆ alkylacyl;
R^{21′} and R^{22′} are each independently selected from the group consisting of deuterium and C₁-C₆ alkyl;
R²³ and R²⁴ are each independently selected from the group consisting of, C₁-C₆ alkyl, C₁-C₆ alkylsulfonyl, C₁-C₆ alkylacyl and C₁-C₆ alkylacyl-N(C₁-C₆ alkyl)-;
R²⁵ is selected from C₁-C₆ alkylacyl;
R⁴ is selected from the group consisting of C₁-C₆ alkylacyl and C₁-C₆ alkylsulfonyl;
R⁵ is selected from C₁-C₆ alkyl;
R^{5′} is selected from the group consisting of —CD₃ and —CH₃, and when R^{5′} is —CH₃, R¹ or R^{1′} is halogen.

In some embodiments, the present application provides a compound of formula (I) or a pharmaceutically acceptable salt thereof, wherein,
R¹ and R^{1′} are each independently selected from the group consisting of hydrogen and halogen;
R², R^{2′} and R³ are each independently selected from the group consisting of hydrogen, amino, amino-C₁-C₆ alkyl-, 7-9 membered spiro-heterocyclyl, 7-10 membered fused-heterocyclyl, 7-10 membered bridged heterocyclyl, 5-6 membered heterocyclyl whose ring atoms consist of a nitrogen atom and a carbon atom, 5-6 membered heterocyclyl whose ring atoms contain a silicon atom or a phosphorus atom,

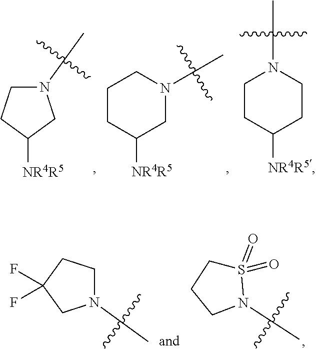

wherein the amino or amino-C₁-C₆ alkyl- is substituted with one or more R²¹, and optionally substituted with one or more R^{21′}, wherein the 5-6 membered heterocyclyl whose ring atoms consist of a nitrogen atom and a carbon atom is substituted with one or more R²², and optionally substituted with one or more R^{22′}, wherein the 7-9 membered spiro-heterocyclyl, 7-10 membered heterocycloalkenyl or 5-6 membered heterocyclyl whose ring atoms contain a silicon atom or a phosphorus atom is optionally substituted with one or more R²³, wherein the 7-10 membered bridged heterocyclyl is substituted with one or more R²⁴ or, R³, together with R² or R^{2′} and the carbon atoms connected thereto, forms morpholinyl optionally substituted with one or more R²,
and R², R^{2′} and R³ are not hydrogen at the same time;
R²¹ is selected from the group consisting of C₂-C₆ alkenyl-C(O)—, C₃-C₁₂ cycloalkyl, 4-12 membered heterocyclyl or C₁-C₆ alkylacylamino-C₁-C₆ alkyl-, wherein the C₃-C₁₂ cycloalkyl, 4-12 membered heterocyclyl or C₁-C₆ alkylacylamino-C₁-C₆ alkyl- is optionally substituted with one or more of the following groups selected from the group consisting of: hydroxy, C₁-C₆ alkyl and C₁-C₆ alkylacyl;
R²² is selected from the group consisting of C₂-C₆ alkynyl-C(O)— and 4-5 membered heterocyclyl, wherein the 4-5 membered heterocyclyl is optionally substituted with one or more [symbol] ;
R^{21′} and R^{22′} are each independently selected from C₁-C₆ alkyl;
R²³ and R²⁴ are each independently selected from the group consisting of, C₁-C₆ alkyl, C₁-C₆ alkylsulfonyl, C₁-C₆ alkylacyl and C₁-C₆ alkylacyl-N(C₁-C₆ alkyl)-;
R²⁵ is selected from C₁-C₆ alkylacyl;
R⁴ is selected from the group consisting of C₁-C₆ alkylacyl and C₁-C₆ alkylsulfonyl;
R⁵ is selected from C₁-C₆ alkyl;
R^{5′} is selected from the group consisting of —CD₃ and —CH₃, and when R^{5′} is —CH₃, R¹ or R^{1′} is halogen.

In some embodiments, the present application provides a compound of formula (I) or a pharmaceutically acceptable salt thereof, wherein,
R¹ and R^{1′} are each independently selected from the group consisting of hydrogen and halogen;
R² and R^{2′} are each independently selected from the group consisting of hydrogen, amino, amino-C₁-C₆ alkyl-, 7-9 membered spiro-heterocyclyl, 10 membered spiro-heterocyclyl whose ring atoms consist of a nitrogen atom and a carbon atom, 7-10 membered fused-heterocyclyl, 7-10 membered bridged heterocyclyl, 5-6 membered heterocyclyl whose ring atoms consist of a nitrogen atom and a carbon atom, 5-6 membered heterocyclyl whose ring atoms contain a silicon atom or a phosphorus atom,

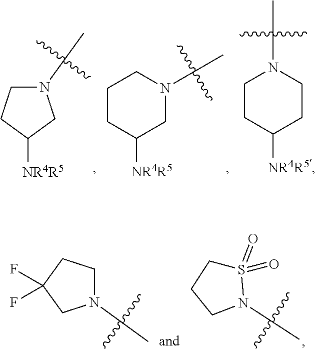

wherein the amino or amino-C₁-C₆ alkyl- is substituted with one or more R²¹, and optionally substituted with one or more R^{21′}, wherein the 5-6 membered heterocyclyl whose ring atoms consist of a nitrogen atom and a carbon atom is substituted with one or more R²², and optionally substituted with one or more R^{22′}, wherein the 7-9 membered spiro-heterocyclyl, 10 membered spiro-heterocyclyl whose ring atoms consist of a nitrogen atom and a carbon atom, 7-10 membered heterocycloalkenyl or 5-6 membered heterocyclyl whose ring atoms contain a silicon atom or a phosphorus atom is optionally substituted with one or more R²³, wherein the 7-10 membered bridged heterocyclyl is substituted with one or more R²⁴,
R³ is hydrogen;
or, R³ is connected to R² or R^{2′}, and together with the carbon atoms connected thereto, forms morpholinyl optionally substituted with one or more R²,
and R² and R^{2′} are not hydrogen at the same time;
R²¹ is selected from the group consisting of C₂-C₆ alkenyl-C(O)—, C₃-C₁₂ cycloalkyl, 4-12 membered heterocyclyl or C₁-C₆ alkylacylamino-C₁-C₆ alkyl-, wherein the C₃-C₁₂ cycloalkyl, 4-12 membered heterocyclyl or C₁-C₆ alkylacylamino-C₁-C₆ alkyl- is optionally substituted with one or more of the following groups selected from the group consisting of: hydroxy, C₁-C₆ alkyl and C₁-C₆ alkylacyl;
R²² is selected from the group consisting of C₂-C₆ alkynyl-C(O)—, 4-5 membered heterocyclyl or 6 membered heterocyclyl whose ring atoms consist of a nitrogen atom and a sulfur atom, wherein the 4-5 membered heterocyclyl or 6 membered heterocyclyl whose ring atoms consist of a nitrogen atom and a sulfur atom is optionally substituted with one or more of the following groups selected from the group consisting of: [symbol] , halogen, C₁-C₃ alkyl and C₁-C₆ alkylacyl;
R^{21′} and R^{22′} are each independently selected from the group consisting of deuterium and C₁-C₆ alkyl;
R²³ and R²⁴ are each independently selected from the group consisting of [symbol] , C₁-C₆ alkyl, C₁-C₆ alkylsulfonyl, C₁-C₆ alkylacyl and C₁-C₆ alkylacyl-N(C₁-C₆ alkyl)-;
R²⁵ is selected from C₁-C₆ alkylacyl;
R⁴ is selected from the group consisting of C₁-C₆ alkylacyl and C₁-C₆ alkylsulfonyl;
R⁵ is selected from C₁-C₆ alkyl;
R^{5′} is selected from the group consisting of —CD₃ and —CH₃, and when R^{5′} is —CH₃, R¹ or R^{1′} is halogen.

In some embodiments, the present application provides a compound of formula (I) or a pharmaceutically acceptable salt thereof, wherein,
R¹ and R^{1′} are each independently selected from the group consisting of hydrogen and halogen; one of R² and R^{2′} is hydrogen and the other is selected from the group consisting of amino, amino-C₁-C₆ alkyl-, 7-9 membered spiro-heterocyclyl, 10 membered spiro-heterocyclyl whose ring atoms consist of a nitrogen atom and a carbon atom, 7-10 membered fused-heterocyclyl, 7-10 membered bridged heterocyclyl, 5-6 membered heterocyclyl whose ring atoms consist of a nitrogen atom and a carbon atom, 5-6 membered heterocyclyl whose ring atoms contain a silicon atom or a phosphorus atom,

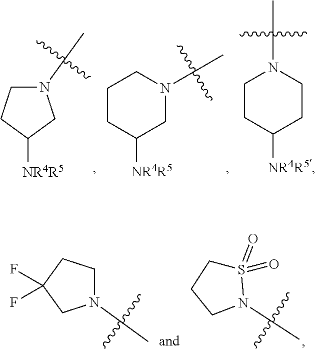

wherein the amino or amino-C₁-C₆ alkyl- is substituted with one or more R²¹, and optionally substituted with one or more R^{21′}, wherein the 5-6 membered heterocyclyl whose ring atoms consist of a nitrogen atom and a carbon atom is substituted with one or more R²², and optionally substituted with one or more R^{22′}, wherein the 7-9 membered spiro-heterocyclyl, 10 membered spiro-heterocyclyl whose ring atoms consist of a nitrogen atom and a carbon atom, 7-10 membered heterocycloalkenyl or 5-6 membered heterocyclyl whose ring atoms contain a silicon atom or a phosphorus atom is optionally substituted with one or more R²³, wherein the 7-10 membered bridged heterocyclyl is substituted with one or more R²⁴,
R³ is hydrogen;
or, R³ is connected to R² or R^{2′}, and together with the carbon atoms connected thereto, forms morpholinyl optionally substituted with one or more R²⁵;
R²¹ is selected from the group consisting of C₂-C₆ alkenyl-C(O)—, C₃-C₁₂ cycloalkyl, 4-12 membered heterocyclyl or C₁-C₆ alkylacylamino-C₁-C₆ alkyl-, wherein the C₃-C₁₂ cycloalkyl, 4-12 membered heterocyclyl or C₁-C₆ alkylacylamino-C₁-C₆ alkyl- is optionally substituted with one or more of the following groups selected from the group consisting of: hydroxy, C₁-C₆ alkyl and C₁-C₆ alkylacyl;
R²² is selected from the group consisting of C₂-C₆ alkynyl-C(O)—, 4-5 membered heterocyclyl or 6 membered heterocyclyl whose ring atoms consist of a nitrogen atom and a sulfur atom, wherein the 4-5 membered heterocyclyl or 6 membered heterocyclyl whose ring atoms consist of a nitrogen atom and a sulfur atom is optionally substituted with one or more of the following groups selected from the group consisting of: [symbol] , halogen, C₁-C₃ alkyl and C₁-C₆ alkylacyl;
R^{21′} and R^{22′} are each independently selected from the group consisting of deuterium and C₁-C₆ alkyl;
R²³ and R²⁴ are each independently selected from the group consisting of, C₁-C₆ alkyl, C₁-C₆ alkylsulfonyl, C₁-C₆ alkylacyl and C₁-C₆ alkylacyl-N(C₁-C₆ alkyl)-;
R²⁵ is selected from C₁-C₆ alkylacyl;
R⁴ is selected from the group consisting of C₁-C₆ alkylacyl and C₁-C₆ alkylsulfonyl;
R⁵ is selected from C₁-C₆ alkyl;
R^{5′} is selected from the group consisting of —CD₃ and —CH₃, and when R^{5′} is —CH₃, R¹ or R^{1′} is halogen.

In some embodiments, the 7-9 membered spiro-heterocyclyl, 7-10 membered fused-heterocyclyl and 7-10 membered bridged heterocyclyl each independently contain 1-3 heteroatoms selected from the group consisting of N, O and S.

In some embodiments, the 7-9 membered spiro-heterocyclyl, 7-10 membered fused-heterocyclyl and 7-10 membered bridged heterocyclyl each independently contain 1 or 2 heteroatoms selected from the group consisting of N, O and S.

In some embodiments, the 10 membered spiro-heterocyclyl whose ring atoms consist of a nitrogen atom and a carbon atom contains 1, 2, or 3 nitrogen atoms.

In some embodiments, the 5-6 membered heterocyclyl whose ring atoms consist of a nitrogen atom and a carbon atom contains 1, 2, or 3 nitrogen atoms.

In some embodiments, the 5-6 membered heterocyclyl whose ring atoms contain a silicon atom or a phosphorus atom contains 1 silicon atom or 1 phosphorus atom, and 1 or 2 heteroatoms selected from the group consisting of N, O and S.

In some embodiments, the 4-5 membered heterocyclyl and 4-12 membered heterocyclyl each independently contain 1-3 heteroatoms selected from the group consisting of N, O and S.

In some embodiments, R¹ and R^{1′} are each independently selected from the group consisting of hydrogen and fluorine.

In some embodiments, R¹ and R^{1′} are both selected from hydrogen.

In some embodiments, R¹ is selected from hydrogen, and R^{1′} is selected from halogen; or R¹ is selected from halogen, and R^{1′} is selected from hydrogen.

In some embodiments, R¹ is selected from hydrogen, and R^{1′} is selected from fluorine; or R¹ is selected from fluorine, and R^{1′} is selected from hydrogen.

In some embodiments, R², R^{2′} and R³ are each independently selected from the group consisting of hydrogen, amino, amino-C₁-C₆ alkyl-, 7-9 membered spiro-heterocyclyl, 7-10 membered fused-heterocyclyl, 7-10 membered bridged heterocyclyl, 5-6 membered heterocyclyl whose ring atoms consist of a nitrogen atom and a carbon atom, 5-6 membered heterocyclyl whose ring atoms contain a silicon atom or a phosphorus atom,

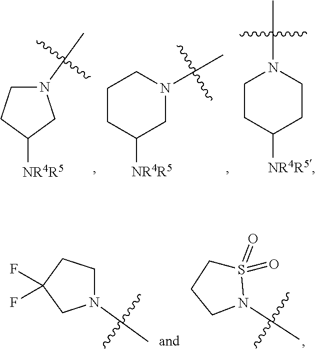

wherein the amino or amino-C₁-C₆ alkyl- is substituted with one or more R²¹, and optionally substituted with one or more R^{21′}, wherein the 5-6 membered heterocyclyl whose ring atoms consist of a nitrogen atom and a carbon atom is substituted with one or more R²², and optionally substituted with one or more R^{22′}, wherein the 7-9 membered spiro-heterocyclyl, 7-10 membered heterocycloalkenyl or 5-6 membered heterocyclyl whose ring atoms contain a silicon atom or a phosphorus atom is optionally substituted with one or more R²³, wherein the 7-10 membered bridged heterocyclyl is substituted with one or more R²⁴
or, R³ is connected to R² or R^{2′}, and together with the carbon atoms connected thereto, forms morpholinyl optionally substituted with one or more R²,
and R², R^{2′} and R³ are not hydrogen at the same time.

In some embodiments, R², R^{2′} and R³ are each independently selected from the group consisting of hydrogen, amino, amino-C₁-C₄ alkyl-, 7, 8 or 9 membered spiro-heterocyclyl, 10 membered spiro-heterocyclyl whose ring atoms consist of a nitrogen atom and a carbon atom, 7, 8 or 9 membered fused-heterocyclyl, 7-8 membered bridged heterocyclyl, 5-6 membered monoheterocyclyl whose ring atoms consist of a nitrogen atom and a carbon atom, 5-6 membered monoheterocyclyl whose ring atoms consist of a silicon atom or a phosphorus atom,

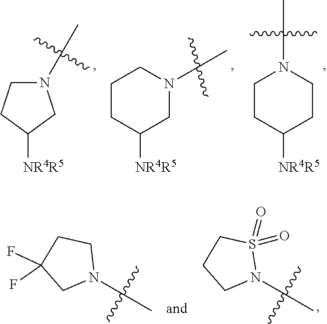

wherein the amino or amino-C₁-C₄ alkyl- is substituted with one or more R²¹, and optionally substituted with one or more R^{21′}, wherein the 5-6 membered monoheterocyclyl whose ring atoms consist of a nitrogen atom and a carbon atom is substituted with one or more R²², and optionally substituted with one or more R^{22′}, wherein the 7, 8 or 9 membered spiro-heterocyclyl, 10 membered spiro-heterocyclyl whose ring atoms consist of a nitrogen atom and a carbon atom, 7, 8 or 9 membered fused-heterocyclyl, or 5-6 membered monoheterocyclyl whose ring atoms contain a silicon atom or a phosphorus atom is optionally substituted with one or more R²³, wherein the 7-8 membered bridged heterocyclyl is substituted with one or more R²⁴
or, R³ is connected to R² or R^{2′}, and together with the carbon atoms connected thereto, forms morpholinyl optionally substituted with one or more R²,
and R², R^{2′} and R³ are not hydrogen at the same time.

In some embodiments, R², R^{2′} and R³ are each independently selected from the group consisting of hydrogen, amino, amino-C₁-C₄ alkyl-, 7, 8 or 9 membered spiro-heterocycloalkyl, 10 membered spiro-heterocycloalkyl whose ring atoms consist of a nitrogen atom and a carbon atom, 7, 8 or 9 membered fused-heterocycloalkyl, 7-8 membered bridged heterocycloalkyl, 5-6 membered monoheterocycloalkyl whose ring atoms consist of a nitrogen atom and a carbon atom, 5-6 membered monoheterocycloalkyl whose ring atoms contain a silicon atom or a phosphorus atom,

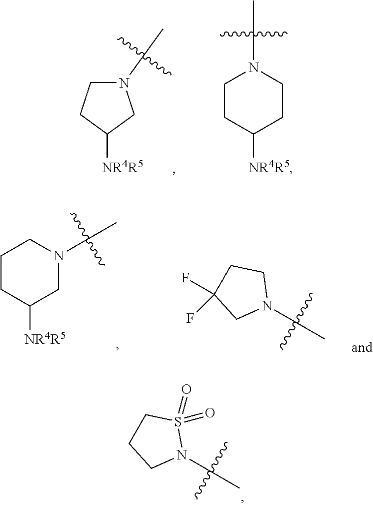

wherein the amino or amino-C₁-C₄ alkyl- is substituted with one or more R²¹, and optionally substituted with one or more R^{21′}, wherein the 5-6 membered monoheterocycloalkyl whose ring atoms consist of a nitrogen atom and a carbon atom is substituted with one or more R²², and optionally substituted with one or more R^{22′}, wherein the 7, 8 or 9 membered spiro-heterocycloalkyl, 10 membered spiro-heterocycloalkyl whose ring atoms consist of a nitrogen atom and a carbon atom, 7, 8 or 9 membered fused-heterocycloalkyl, or 5-6 membered monoheterocycloalkyl whose ring atoms contain a silicon atom or a phosphorus atom is optionally substituted by one or more R²³, wherein the 7-8 membered bridged heterocycloalkyl is substituted by one or more R²⁴
or, R³ is connected to R² or R^{2′}, and together with the carbon atoms connected thereto, forms morpholinyl optionally substituted with one or more R²,
and R², R^{2′} and R³ are not hydrogen at the same time.

In some embodiments, R², R^{2′} and R³ are each independently selected from the group consisting of hydrogen, amino, amino-C₁-C₄ alkyl-, 7, 8 or 9 membered spiro-heterocycloalkyl, 10 membered spiro-heterocycloalkyl whose ring atoms consist of a nitrogen atom and a carbon atom, 5 membered fused 4 membered heterocycloalkyl, 5 membered fused 5 membered heterocycloalkyl, 5 membered fused 6 membered heterocycloalkyl, 7-8 membered bridged heterocycloalkyl, 5-6 membered monoheterocycloalkyl whose ring atoms consist of a nitrogen atom and a carbon atom, 6 membered monoheterocycloalkyl whose ring atoms contain a silicon atom or a phosphorus atom,

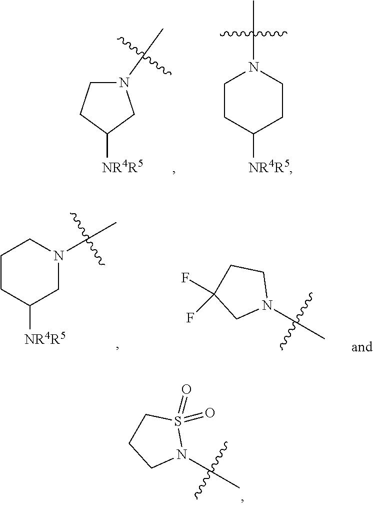

wherein the amino or amino-C₁-C₄ alkyl- is substituted with one or more R²¹, and optionally substituted with one or more R^{21′}, wherein the 5 membered or 6 membered monoheterocycloalkyl whose ring atoms consist of a nitrogen atom and a carbon atom is substituted with one or more R²², and optionally substituted with one or more R^{22′} wherein the 7, 8 or 9 membered spiro-heterocycloalkyl, 10 membered spiro-heterocycloalkyl whose ring atoms consist of a nitrogen atom and a carbon atom, 5 membered fused 4 membered heterocycloalkyl, 5 membered fused 5 membered heterocycloalkyl, 5 membered fused 6 membered heterocycloalkyl, or 6 membered monoheterocycloalkyl whose ring atoms contain a silicon atom or a phosphorus atom is optionally substituted with one or more R²³, wherein the 7-8 membered bridged heterocycloalkyl is substituted with one or more R²⁴
or, R³ is connected to R² or R^{2′}, and together with the carbon atoms connected thereto, forms morpholinyl optionally substituted with one or more R²,
and R², R^{2′} and R³ are not hydrogen at the same time.

In some embodiments, R², R^{2′} and R³ are each independently selected from the group consisting of hydrogen, amino, aminomethyl,

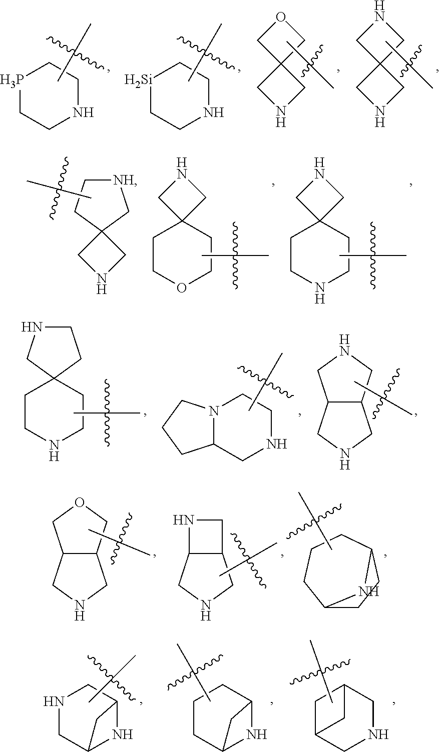

pyrrolidinyl, piperidinyl, piperazinyl,

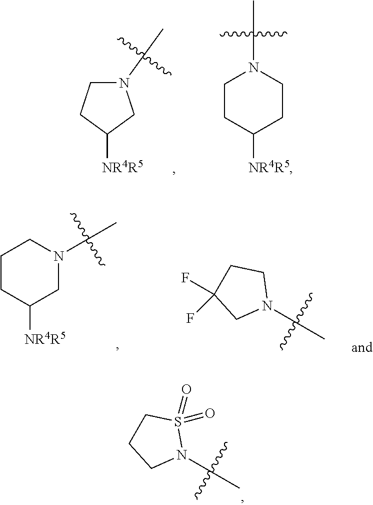

wherein the amino or aminomethyl is substituted with one or more R²¹, and optionally substituted with one or more R^{21′}, wherein the pyrrolidinyl, piperidinyl or piperazinyl is substituted with one or more R²², and optionally substituted with one or more R^{22′}, wherein the

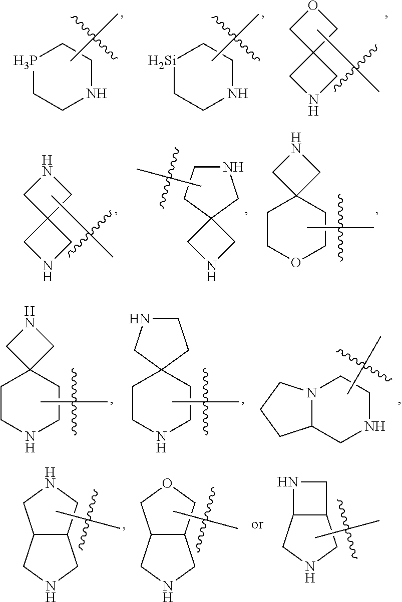

is substituted with one or more R²³, wherein the

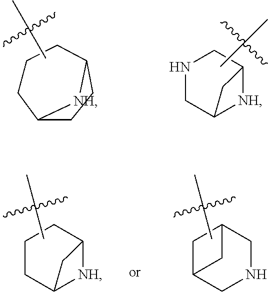

is substituted with one or more R²⁴
or, R³ is connected to R² or R^{2′}, and together with the carbon atoms connected thereto, forms a

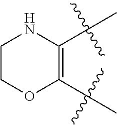 [SMILES: *C1=C(*)OCCN1]

group optionally substituted with one or more R²⁵,
and R², R^{2′} and R³ are not hydrogen at the same time.

In some embodiments, R², R^{2′} and R³ are each independently selected from the group consisting of hydrogen, amino, aminomethyl,

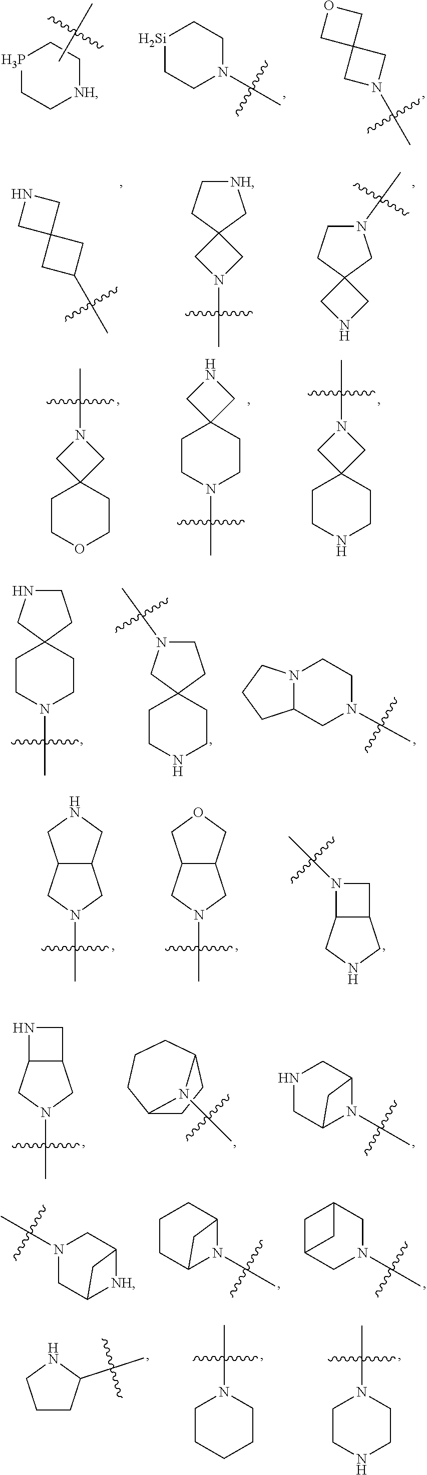

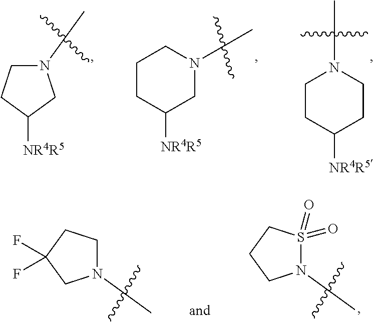

wherein the amino or aminomethyl is substituted with one or more R²¹, and optionally substituted with one or more R^{21′}, wherein the

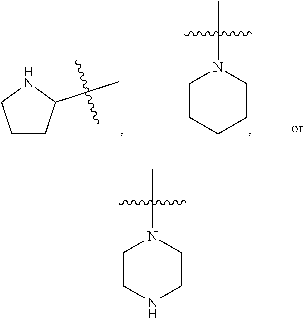 [SMILES: *C1CCCN1 ; *N1CCCCC1 ; *N1CCNCC1]

is substituted with one or more R²², and optionally substituted with one or more R^{22′}, wherein the

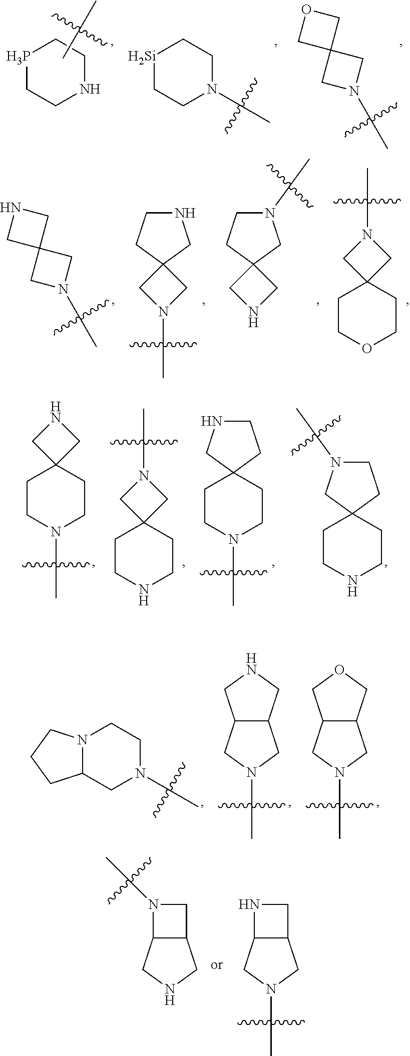

is substituted

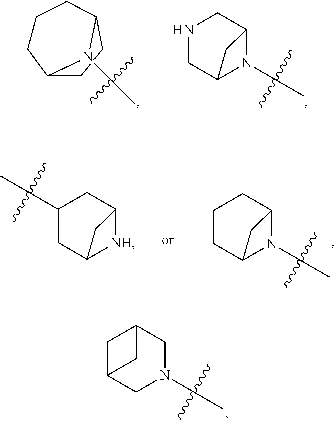 [SMILES: *N1C2CCCC1CC2 ; *N1C2CNCC1C2 ; *C1CC2CC(C1)N2 ; *N1C2CCCC1C2 ; *N1CC2CC(C2)C1]

with one or more R²⁴,
or, R³ is connected to R² or R^{2′}, and together with the carbon atoms connected thereto, forms a

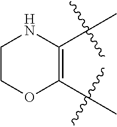 [SMILES: *C1=C(*)OCCN1]

group optionally substituted with one or more R²⁵,
and R², R^{2′} and R³ are not hydrogen at the same time.

In some embodiments, R², R^{2′} and R³ are each independently selected from the group consisting of hydrogen, amino aminomethyl

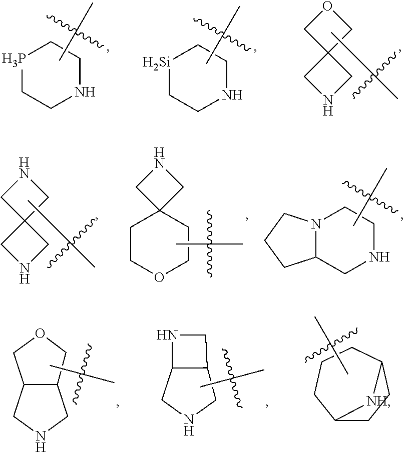

pyrrolidinyl, piperidinyl, piperazinyl,

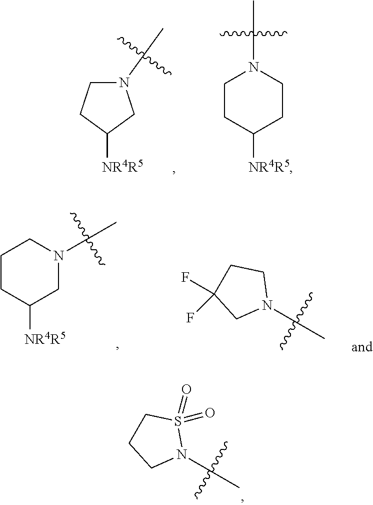

wherein the amino and aminomethyl are substituted with one or more R and optionally substituted with one or more R^{21′}, wherein the pyrrolidinyl, piperidinyl or piperazinyl is substituted with one or more R²², and optionally substituted with one or more R^{22′}, wherein the

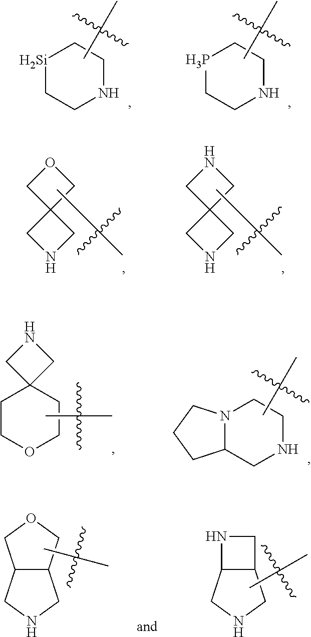

are optionally substituted with one or more R²³, wherein the

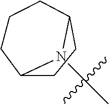 [SMILES: *N1C2CCCC1CC2]

is substituted with one or more R²⁴,
or, R³ together with R² or R^{2′} and the carbon atoms connected thereto, forms a

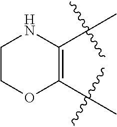 [SMILES: *C1=C(*)OCCN1]

group optionally substituted with one or more R²,
and R², R^{2′} and R³ are not hydrogen at the same time.

In some embodiments, R², R^{2′} and R³ are each independently selected from the group consisting of hydrogen, amino, aminomethyl

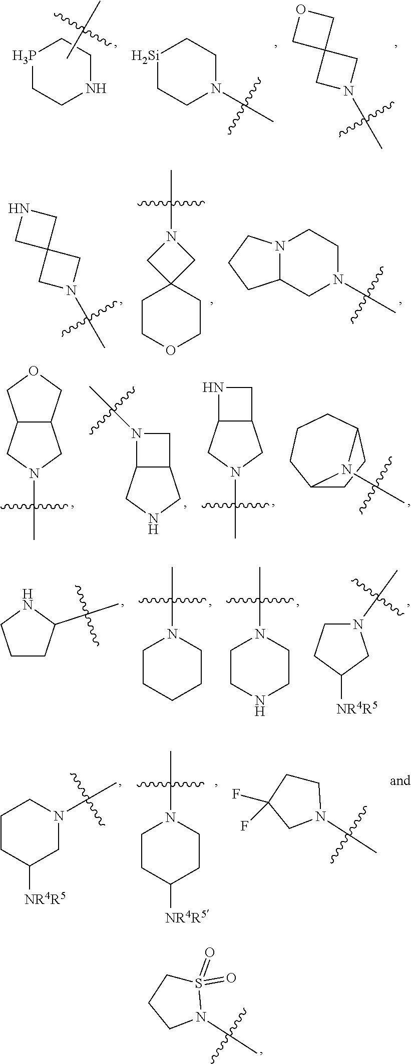

wherein the amino and aminomethyl are substituted with one or more R²¹, and optionally substituted with one or more R^{21′}, wherein the

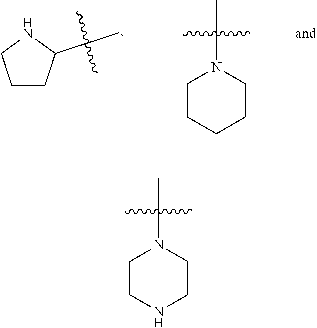 [SMILES: *C1CCCN1 ; *N1CCCCC1 ; *N1CCNCC1]

are substituted with one or more R²² and optionally substituted with one or more R^{22′}, wherein the

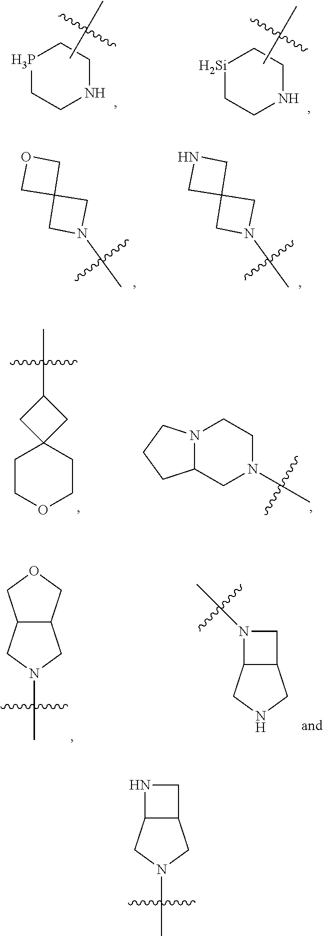

are optionally substituted with one or more R²³, wherein the

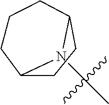 [SMILES: *N1C2CCCC1CC2]

is substituted with one or more R²⁴,
or, R³ together with R² or R^{2′} and the carbon atoms connected thereto, forms a

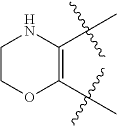 [SMILES: *C1=C(*)OCCN1]

group optionally substituted with one or more R²,
and R², R^{2′} and R³ are not hydrogen at the same time.

In some embodiments, one of R² and R^{2′} is hydrogen and the other is selected from the group consisting of amino, amino-C₁-C₆ alkyl-, 7-9 membered spiro-heterocyclyl, 7-10 membered fused-heterocyclyl, 7-10 membered bridged heterocyclyl, 5-6 membered heterocyclyl whose ring atoms consist of a nitrogen atom and a carbon atom, 5-6 membered heterocyclyl whose ring atoms contain a silicon atom or a phosphorus atom,

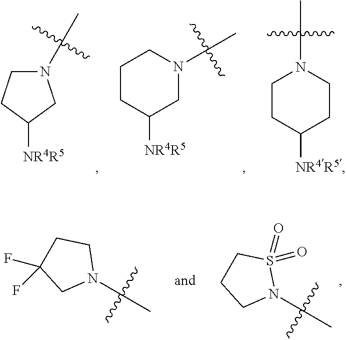

wherein the amino or amino-C₁-C₆ alkyl- is substituted with one or more R²¹, and optionally substituted with one or more R^{21′}, wherein the 5-6 membered heterocyclyl whose ring atoms consist of a nitrogen atom and a carbon atom is substituted with one or more R²², and optionally substituted with one or more R^{22′}, wherein the 7-9 membered spiro-heterocyclyl, 7-10 membered heterocycloalkenyl or 5-6 membered heterocyclyl whose ring atoms contain a silicon atom or a phosphorus atom is optionally substituted with one or more R²³, wherein the 7-10 membered bridged heterocyclyl is substituted with one or more R²⁴
and R³ is hydrogen.

In some embodiments, one of R² and R^{2′} is hydrogen and the other is selected from the group consisting of amino, amino-C₁-C₄ alkyl-, 7, 8 or 9 membered spiro-heterocyclyl, 10 membered spiro-heterocyclyl whose ring atoms consist of a nitrogen atom and a carbon atom, 7, 8 or 9 membered fused-heterocyclyl, 7-8 membered bridged heterocyclyl, 5-6 membered monoheterocyclyl whose ring atoms consist of a nitrogen atom and a carbon atom, 5-6 membered monoheterocyclyl whose ring atoms consist of a silicon atom or a phosphorus atom,

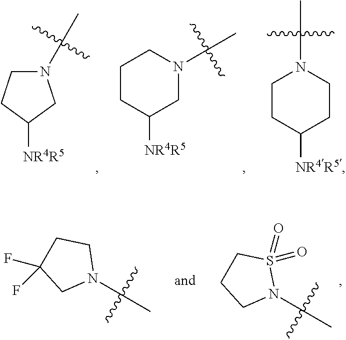

wherein the amino or amino-C₁-C₄ alkyl- is substituted with one or more R²¹, and optionally substituted with one or more R^{21′}, wherein the 5-6 membered monoheterocyclyl whose ring atoms consist of a nitrogen atom and a carbon atom is substituted with one or more R²², and optionally substituted with one or more R^{22′}, wherein the 7, 8 or 9 membered spiro-heterocyclyl, 10 membered spiro-heterocyclyl whose ring atoms consist of a nitrogen atom and a carbon atom, 7, 8 or 9 membered fused-heterocyclyl, or 5-6 membered monoheterocyclyl whose ring atoms contain a silicon atom or a phosphorus atom is optionally substituted with one or more R²³, wherein the 7-8 membered bridged heterocyclyl is substituted with one or more R²⁴
and R³ is hydrogen.

In some embodiments, one of R² and R^{2′} is hydrogen and the other is selected from the group consisting of amino, amino-C₁-C₄ alkyl-, 7, 8 or 9 membered spiro-heterocycloalkyl, 10 membered spiro-heterocycloalkyl whose ring atoms consist of a nitrogen atom and a carbon atom, 7, 8 or 9 membered fused-heterocycloalkyl, 7-8 membered bridged heterocycloalkyl, 5-6 membered monoheterocycloalkyl whose ring atoms consist of a nitrogen atom and a carbon atom, 5-6 membered monoheterocycloalkyl whose ring atoms contain a silicon atom or a phosphorus atom,

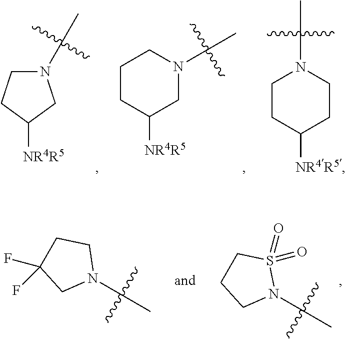

wherein the amino or amino-C₁-C₄ alkyl- is substituted with one or more R²¹, and optionally substituted with one or more R^{21′}, wherein the 5-6 membered monoheterocycloalkyl whose ring atoms consist of a nitrogen atom and a carbon atom is substituted with one or more R²², and optionally substituted with one or more R^{22′}, wherein the 7, 8 or 9 membered spiro-heterocycloalkyl, 10 membered spiro-heterocycloalkyl whose ring atoms consist of a nitrogen atom and a carbon atom, 7, 8 or 9 membered fused-heterocycloalkyl, or 5-6 membered monoheterocycloalkyl whose ring atoms contain a silicon atom or a phosphorus atom is optionally substituted by one or more R²³, wherein the 7-8 membered bridged heterocycloalkyl is substituted by one or more R²⁴
and R³ is hydrogen.

In some embodiments, one of R² and R^{2′} is hydrogen and the other is selected from the group consisting of amino, amino-C₁-C₄ alkyl-, 7, 8 or 9 membered spiro-heterocycloalkyl, 10 membered spiro-heterocycloalkyl whose ring atoms consist of a nitrogen atom and a carbon atom, 5 membered fused 4 membered heterocycloalkyl, 5 membered fused 5 membered heterocycloalkyl, 5 membered fused 6 membered heterocycloalkyl, 7-8 membered bridged heterocycloalkyl, 5-6 membered monoheterocycloalkyl whose ring atoms consist of a nitrogen atom and a carbon atom, 6 membered monoheterocycloalkyl whose ring atoms contain a silicon atom or a phosphorus atom,

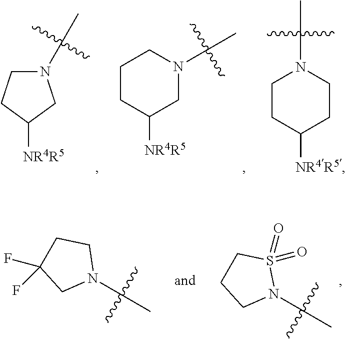

wherein the amino or amino-C₁-C₄ alkyl- is substituted with one or more R²¹, and optionally substituted with one or more R^{21′}, wherein the 5 membered or 6 membered monoheterocycloalkyl whose ring atoms consist of a nitrogen atom and a carbon atom is substituted with one or more R²², and optionally substituted with one or more R^{22′} wherein the 7, 8 or 9 membered spiro-heterocycloalkyl, 10 membered spiro-heterocycloalkyl whose ring atoms consist of a nitrogen atom and a carbon atom, 5 membered fused 4 membered heterocycloalkyl, 5 membered fused 5 membered heterocycloalkyl, 5 membered fused 6 membered heterocycloalkyl, or 6 membered monoheterocycloalkyl whose ring atoms contain a silicon atom or a phosphorus atom is optionally substituted with one or more R²³, wherein the 7-8 membered bridged heterocycloalkyl is substituted with one or more R²⁴
and R³ is hydrogen.

In some embodiments, one of R² and R^{2′} is hydrogen and the other is selected from the group consisting of amino, aminomethyl,

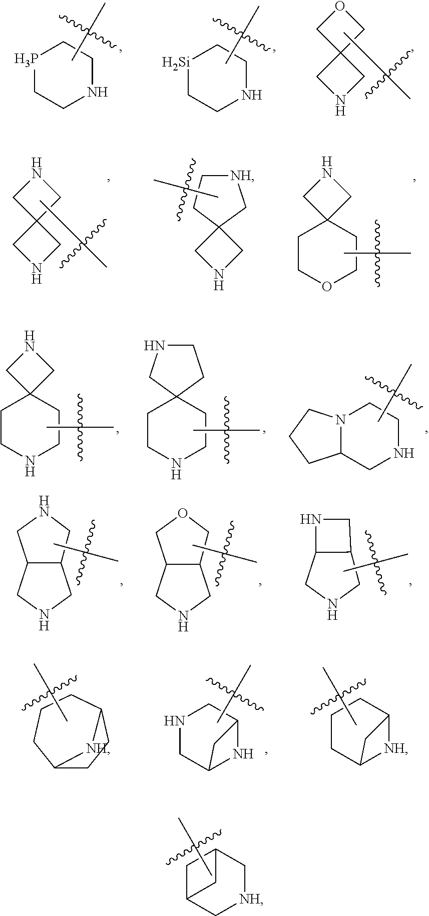

pyrrolidinyl, piperidinyl, piperazinyl,

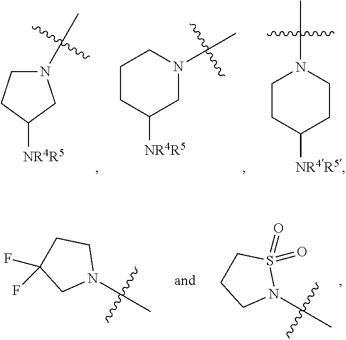

wherein the amino or aminomethyl is substituted with one or more R²¹, and optionally substituted with one or more R^{21′}, wherein the pyrrolidinyl, piperidinyl or piperazinyl is substituted with one or more R²², and optionally substituted with one or more R^{22′}, wherein the

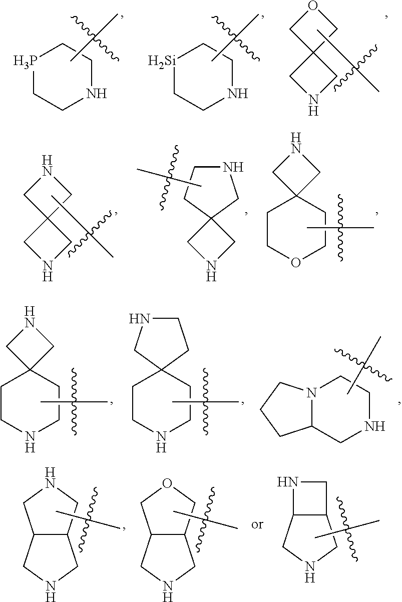

is substituted with one or more R²³, wherein the

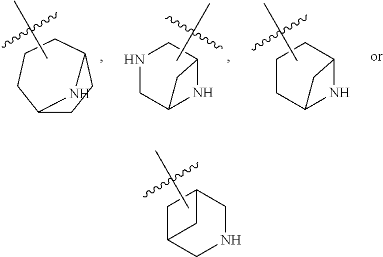

is substituted with one or more R²⁴
and R³ is hydrogen;
or, R³ is connected to R² or R^{2′}, and together with the carbon atoms connected thereto, forms a

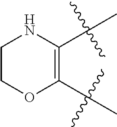 [SMILES: *C1=C(*)OCCN1]

group optionally substituted with one or more R²⁵.

In some embodiments, one of R² and R^{2′} is hydrogen and the other is selected from the group consisting of amino, aminomethyl,

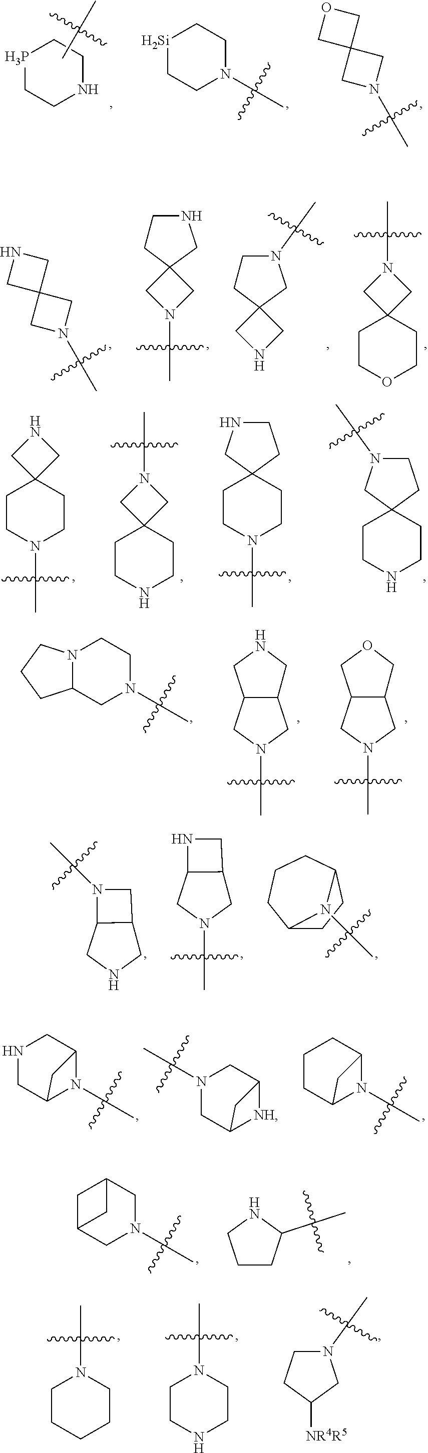

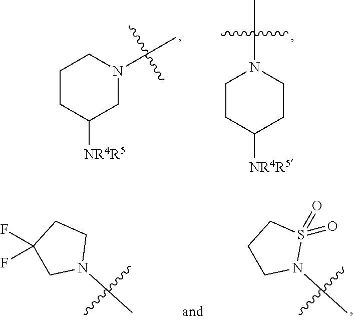

wherein the amino or aminomethyl is substituted with one or more R²¹, and optionally substituted with one or more R²¹, wherein the

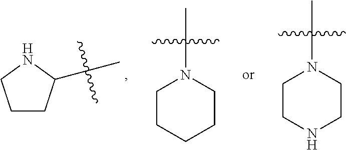 [SMILES: *C1CCCN1 ; *N1CCCCC1 ; *N1CCNCC1]

is substituted with one or more R²², and optionally substituted with one or more R²², wherein the

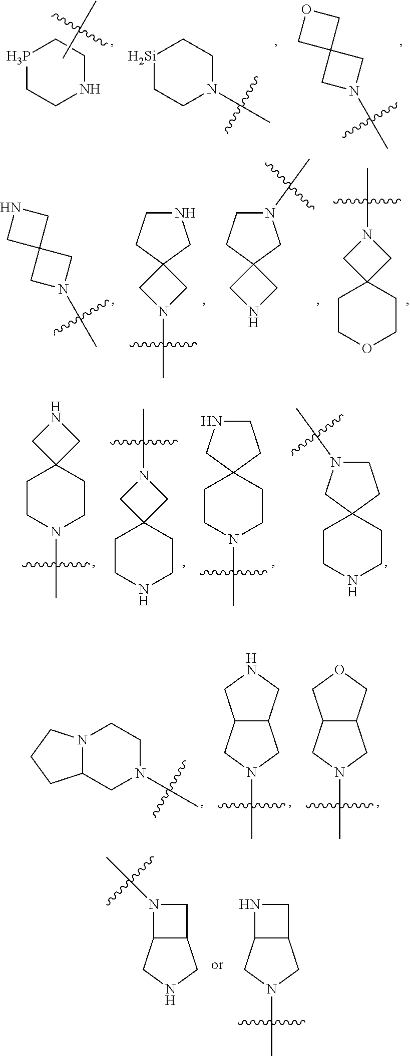

is substituted with one or more R²³, wherein the

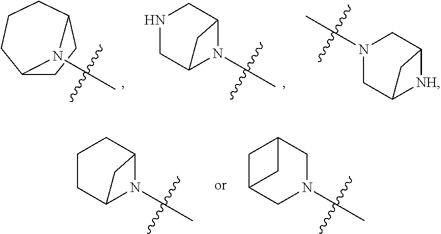 [SMILES: *N1C2CCCC1CC2 ; *N1C2CNCC1C2 ; *N1CC2CC(C1)N2 ; *N1C2CCCC1C2 ; *N1CC2CC(C2)C1]

is substituted with one or more R²⁴
and R³ is hydrogen;
or, R³ is connected to R² or R^{2′}, and together with the carbon atoms connected thereto, forms a

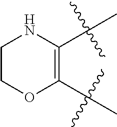 [SMILES: *C1=C(*)OCCN1]

group optionally substituted with one or more R²⁵.

In some embodiments, R³ is connected to R² or R^{2′}, such that the structural unit

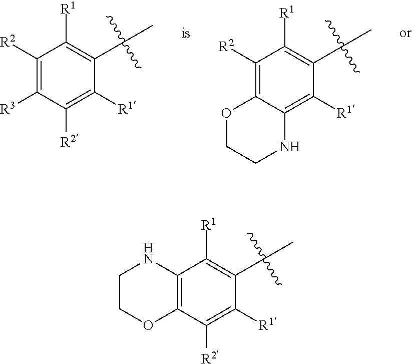

optionally substituted with one or more R²⁵.

In some embodiments, R²¹ is selected from the group consisting of C₂-C₄ alkenyl-C(O)—, C₅-C₁₀ cycloalkyl, 4-6 membered heterocyclyl or C₁-C₄ alkylacylamino-C₁-C₄ alkyl-, wherein the C₅-C₁₀ cycloalkyl, 4-6 membered heterocyclyl or C₁-C₄ alkylacylamino-C₁-C₄ alkyl- is optionally substituted with one or more of the following groups selected from the group consisting of: hydroxy, C₁-C₆ alkyl and C₁-C₄ alkylacyl.

In some embodiments, R²¹ is selected from the group consisting of C₂-C₃ alkenyl-C(O)—, C₇-C₁₀ bridged cycloalkyl, 4-6 membered monoheterocycloalkyl and C₁-C₃ alkylacylamino-C₁-C₃ alkyl-, wherein the C₇-C₁₀ bridged cycloalkyl, 4-6 membered monoheterocycloalkyl or C₁-C₃ alkylacylamino-C₁-C₃ alkyl- is optionally substituted with one or more of the following groups selected from the group consisting of: hydroxy, C₁-C₄ alkyl and C₁-C₄ alkylacyl.

In some embodiments, R²¹ is selected from the group consisting of C₂-C₃ alkenyl-C(O)—, C₁₀ bridged cycloalkyl, 5 membered or 6 membered monoheterocycloalkyl, or C₁-C₃ alkylacylamino-C₁-C₃ alkyl-, wherein the C₁₀ bridged cycloalkyl, 5 membered or 6 membered monoheterocycloalkyl, or C₁-C₃ alkylacylamino-C₁-C₃ alkyl- is optionally substituted with one or more of the following groups selected from the group consisting of: hydroxy, C₁-C₄ alkyl and C₁-C₄ alkylacyl.

In some embodiments, R²¹ is selected from the group consisting of H₂C═CHC(O)—,

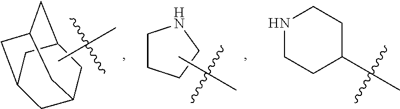

and CH₃C(O)N(CH₃)—CH₃CH₂—, wherein the

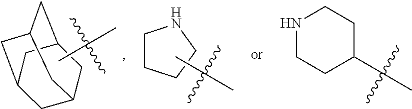

is optionally substituted with one or more of the following groups selected from the group consisting of: hydroxy and C₁-C₄ alkylacyl.

In some embodiments, R²¹ is selected from the group consisting of H₂C═CHC(O)—,

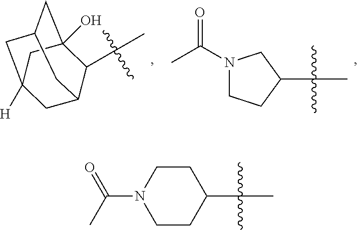

and CH₃C(O)N(CH₃)—CH₃CH₂—.

In some embodiments, R²¹ is selected from the group consisting of H₂C═CHC(O)—,

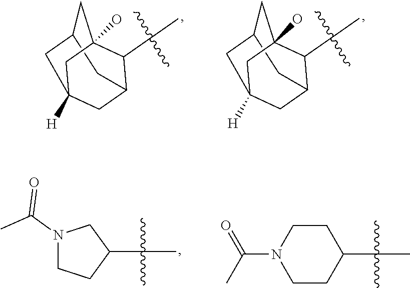

and CH₃C(O)N(CH₃)—CH₃CH₂—.

In some embodiments, one of R² and R^{2′} is hydrogen and the other is amino, wherein the amino is substituted with one R²¹, R²¹ being selected from the group consisting of C₂-C₃ alkenyl-C(O), C₁₀ bridged cycloalkyl, 5 membered or 6 membered monoheterocycloalkyl, and C₁-C₃ alkylacylamino-C₁-C₃ alkyl-, wherein the C₁₀ bridged cycloalkyl, 5 membered or 6 membered monoheterocycloalkyl, or C₁-C₃ alkylacylamino-C₁-C₃ alkyl- is optionally substituted with one or more of the following groups selected from the group consisting of: hydroxy, C₁-C₄ alkyl and C₁-C₄ alkylacyl; and/or R³ is hydrogen.

In some embodiments, one of R² and R^{2′} is hydrogen and the other is amino, wherein the amino is substituted with one R²¹, R²¹ being selected from the group consisting of H₂C═CHC(O)—,

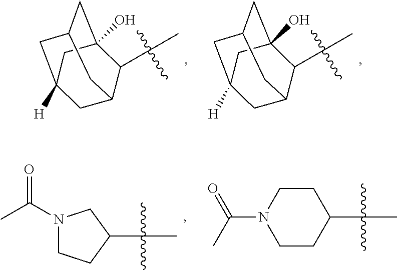

and CH₃C(O)N(CH₃)—CH₃CH₂—; and/or R³ is hydrogen.

In some embodiments, one of R² and R^{2′} is hydrogen and the other is amino-C₁-C₆ alkyl-, wherein the amino-C₁-C₆ alkyl- is substituted with C₂-C₃ alkenyl-C(O)—; and/or R³ is hydrogen.

In some embodiments, R²² is selected from the group consisting of C₂-C₆ alkynyl-C(O)— and 4-5 membered heterocyclyl, wherein the 4-5 membered heterocyclyl is optionally substituted with one or more [symbol] .

In some embodiments, R²² is selected from the group consisting of C₂-C₃ alkynyl-C(O)—, 4 membered or 5 membered heterocyclyl and 6 membered heterocyclyl whose ring atoms consist of a nitrogen atom and a sulfur atom, wherein the 4 membered or 5 membered heterocyclyl, or 6 membered heterocyclyl whose ring atoms consist of a nitrogen atom and a sulfur atom is optionally substituted with one or more of the following groups selected from the group consisting of: [symbol] , halogen, C₁-C₃ alkyl and C₁-C₆ alkylacyl.

In some embodiments, R²² is selected from the group consisting of C₂-C₃ alkynyl-C(O)— and 4 membered or 5 membered heterocyclyl, wherein the 4 membered or 5 membered heterocyclyl is optionally substituted with one or more [symbol] .

In some embodiments, R²² is selected from the group consisting of C₂-C₃ alkynyl-C(O)—, 4 membered or 5 membered heterocycloalkyl and

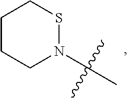 [SMILES: *N1CCCCS1]

wherein the 4 membered or 5 membered heterocycloalkyl or

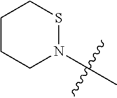 [SMILES: *N1CCCCS1]

is optionally substituted with one or more of the following groups selected from the group consisting of: [symbol] , halogen, C₁-C₃ alkyl and C₁-C₆ alkylacyl.

In some embodiments, R²² is selected from the group consisting of C₂-C₃ alkynyl-C(O)— and 4 membered or 5 membered heterocycloalkyl, wherein the 4 membered or 5 membered heterocycloalkyl is optionally substituted with one or more [symbol] .

In some embodiments, R²² is selected from the group consisting of H₃CC≡CC(O)—,

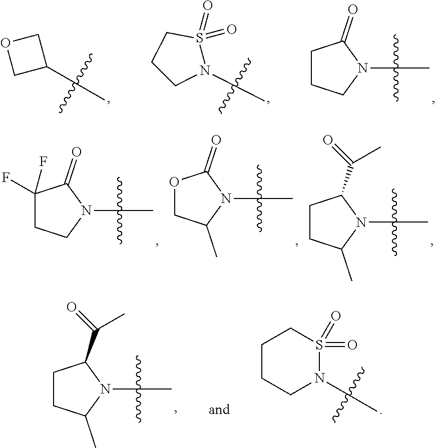

In some embodiments, R²² is selected from the group consisting of H₃CC≡CC(O)—,

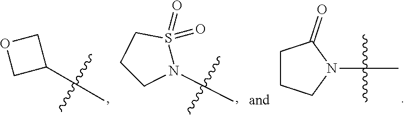 [SMILES: *C1COC1 ; *N1CCCS1(=O)=O ; *N1CCCC1=O]

In some embodiments, R^{21′} and R^{22′} are each independently selected from the group consisting of deuterium and C₁-C₄ alkyl.

In some embodiments, R^{21′} and R^{22′} are each independently selected from the group consisting of deuterium and methyl.

In some embodiments, R^{21′} and R^{22′} are each independently selected from C₁-C₄ alkyl.

In some embodiments, R^{21′} and R^{22′} are each independently selected from methyl.

In some embodiments, R²³ is selected from the group consisting of [symbol] , C₁-C₄ alkyl, C₁-C₄ alkylsulfonyl and C₁-C₄ alkylacyl.

In some embodiments, R²³ is selected from the group consisting of [symbol] , CH₃—, CH₃S(O)₂— and CH₃C(O)—.

In some embodiments, R²⁴ is selected from C₁-C₄ alkylacyl-N(C₁-C₄ alkyl)-.

In some embodiments, R²⁴ is selected from CH₃C(O)N(CH₃)—.

In some embodiments, R²⁵ is selected from C₁-C₄ alkylacyl.

In some embodiments, R²⁵ is selected from CH₃C(O)—.

In some embodiments, R⁴ is selected from the group consisting of C₁-C₄ alkylacyl and C₁-C₄ alkylsulfonyl.

In some embodiments, R⁴ is selected from the group consisting of CH₃C(O)— and CH₃S(O)₂—.

In some embodiments, R⁵ is selected from C₁-C₄ alkyl.

In some embodiments, R⁵ is selected from methyl.

In some embodiments, R⁴ is selected from CH₃C(O)—; R⁵ is selected from methyl.

In some embodiments, R⁴ is selected from CH₃S(O)₂—; R⁵ is selected from methyl.

In some embodiments, R^{5′} is selected from —CD₃ and —CH₃, and when R^{5′} is —CH₃, R¹ or R^{1′} is fluorine.

In some embodiments, one of R² and R^{2′} is hydrogen and the other is selected from 7-9 membered spiro-heterocyclyl, wherein the 7-9 membered spiro-heterocyclyl is optionally substituted with 1 or 2 R²³, R²³ being selected from the group consisting of [symbol] , C₁-C₄ alkyl, C₁-C₄ alkylsulfonyl and C₁-C₄ alkylacyl; and/or R³ is hydrogen.

In some embodiments, one of R² and R^{2′} is hydrogen and the other is selected from 7-9 membered spiro-heterocyclyl, wherein the 7-9 membered spiro-heterocyclyl is optionally substituted with 1 or 2 R²³, R²³ being selected from the group consisting of [symbol] , CH₃—, CH₃S(O)₂— and CH₃C(O)—; and/or
R³ is hydrogen.

In some embodiments, the 7-9 membered spiro-heterocyclyl is a 7, 8, or 9 membered monospiro heterocyclic ring containing 1 or 2 heteroatoms selected from the group consisting of N, O and S. In some embodiments, the 7-9 membered spiro-heterocyclyl is a 7, 8, or 9 membered monospiro heterocyclic ring containing 1 or 2 heteroatoms selected from the group consisting of N and O.

In some embodiments, one of R² and R^{2′} is hydrogen and the other is 10 membered spiro-heterocyclyl whose ring atoms consist of a nitrogen atom and a carbon atom, wherein the 10 membered spiro-heterocyclyl is optionally substituted with 1 or 2 R²³, R²³ being selected from the group consisting of [symbol] , C₁-C₄ alkyl, C₁-C₄ alkylsulfonyl and C₁-C₄ alkylacyl; and/or R³ is hydrogen.

In some embodiments, one of R² and R^{2′} is hydrogen and the other is 10 membered spiro-heterocyclyl whose ring atoms consist of a nitrogen atom and a carbon atom, wherein the 10 membered spiro-heterocyclyl is optionally substituted with 1 or 2 R²³, R²³ being selected from the group consisting of and C₁-C₄ alkyl; and/or R³ is hydrogen.

In some embodiments, the 10 membered spiro-heterocyclyl contains 1 or 2 heteroatoms selected from N.

In some embodiments, one of R² and R^{2′} is hydrogen and the other is selected from 7-10 membered fused-heterocyclyl, wherein the 7-10 membered fused-heterocyclyl is optionally substituted with 1 or 2 R²³, R²³ being selected from the group consisting of [symbol] , C₁-C₄ alkyl, C₁-C₄ alkylsulfonyl and C₁-C₄ alkylacyl; and/or R³ is hydrogen.

In some embodiments, one of R² and R^{2′} is hydrogen and the other is selected from 7-9 membered fused-heterocyclyl, wherein the 7-9 membered fused-heterocyclyl is optionally substituted with 1 [symbol] and C₁-C₄ alkylacyl; and/or R³ is hydrogen.

In some embodiments, the 7-9 membered fused-heterocyclyl contains 1 or 2 heteroatoms selected from the group consisting of N, O and S. In some embodiments, the 7-9 membered fused-heterocyclyl contains 1 or 2 heteroatoms selected from the group consisting of N and O.

In some embodiments, one of R² and R^{2′} is hydrogen and the other is selected from 5-6 membered heterocyclyl whose ring atoms consist of a nitrogen atom and a carbon atom, wherein the 5-6 membered heterocyclyl is substituted with one R²² and optionally substituted with one R^{22′}; R²² is selected from the group consisting of C₂-C₃ alkynyl-C(O)—, 4 membered or 5 membered heterocyclyl or 6 membered heterocyclyl whose ring atoms consist of a nitrogen atom and a sulfur atom, wherein the 4 membered or 5 membered heterocyclyl or 6 membered heterocyclyl whose ring atoms consist of a nitrogen atom and a sulfur atom is optionally substituted with 1, 2 or 3 groups selected from the group consisting of [symbol] , halogen, C₁-C₃ alkyl and C₁-C₃ alkylacyl; R^{22′} is selected from the group consisting of deuterium and C₁-C₃ alkyl; and/or R³ is hydrogen.

In some embodiments, the 5-6 membered heterocyclyl whose ring atoms consist of a nitrogen atom and a carbon atom contains 1 or 2 nitrogen atoms.

In some embodiments, one of R² and R^{2′} is hydrogen and the other is selected from 7-10 membered bridged heterocyclyl, wherein the 7-10 membered bridged heterocyclyl is substituted with 1 or 2 R²⁴, R²⁴ being selected from C₁-C₄ alkylacyl-N(C₁-C₄ alkyl)-; and/or R³ is hydrogen.

In some embodiments, one of R² and R^{2′} is hydrogen and the other is selected from 7-10 membered bridged heterocyclyl, wherein the 7-10 membered bridged heterocyclyl is substituted with 1 CH₃C(O)N(CH₃)—; and/or R³ is hydrogen.

In some embodiments, the 7-10 membered bridged heterocyclyl contains 1 or 2 heteroatoms selected from the group consisting of N, O and S. In some embodiments, the 7-10 membered bridged heterocyclyl contains 1 or 2 heteroatoms selected from the group consisting of N and O.

In some embodiments, one of R² and R^{2′} is hydrogen and the other is selected from 5-6 membered heterocyclyl whose ring atoms contain a silicon atom and a phosphorus atom, wherein the 5-6 membered heterocyclyl is optionally substituted with 1 or 2 R²³, R²³ being selected from the group consisting of [symbol] and C₁-C₄ alkyl; and/or R³ is hydrogen.

In some embodiments, the 5-6 membered heterocyclyl whose ring atoms contain a silicon atom or a phosphorus atom contains 1 silicon atom or 1 phosphorus atom, and further contains 1 heteroatom selected from the group consisting of N, O and S. In some embodiments, the 5-6 membered heterocyclyl whose ring atoms contain a silicon atom or a phosphorus atom contains 1 silicon atom or 1 phosphorus atom, and further contains 1 N atom.

In some embodiments, R², R^{2′} and R³ are each independently selected from the group consisting of hydrogen,

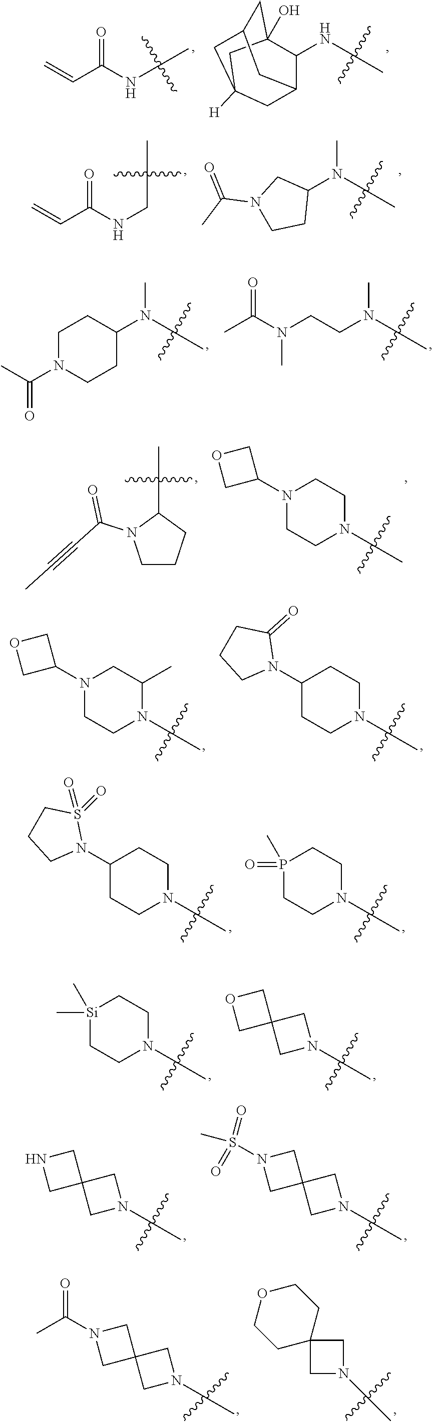

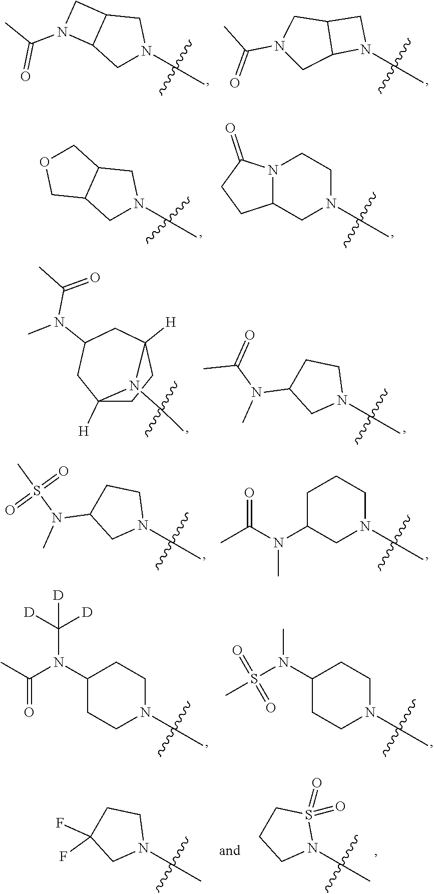

or, R³ is connected to R² or R^{2′}, and together with the carbon atoms connected thereto, forms a

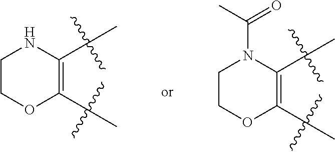 [SMILES: *C1=C(*)OCCN1 ; *C1=C(*)N(C(C)=O)CCO1]

group, and R², R² and R³ are not hydrogen at the same time.

In some embodiments, one of R² and R^{2′} is hydrogen and the other is selected from

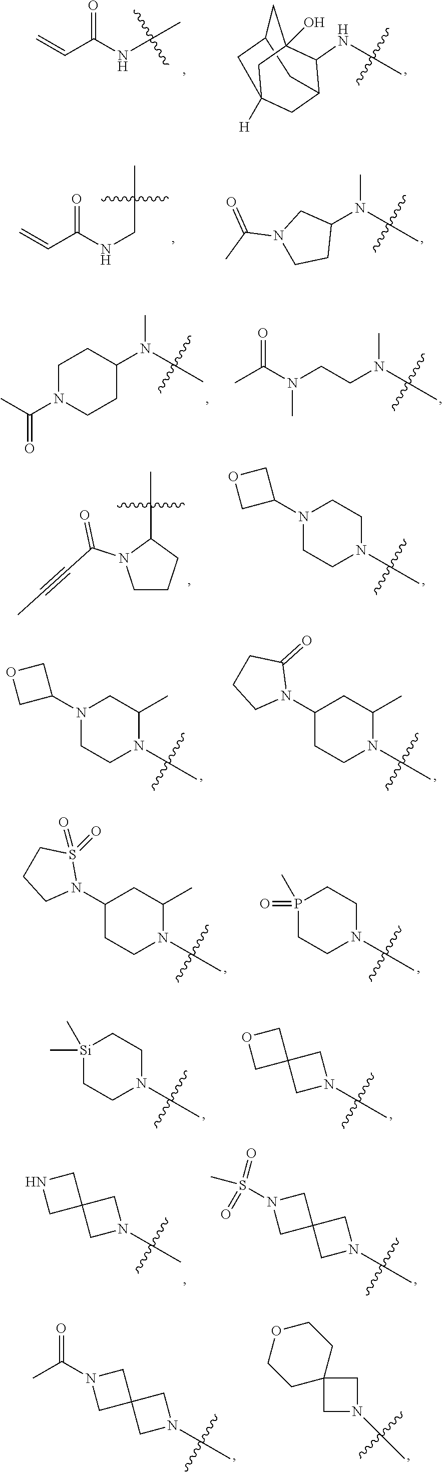

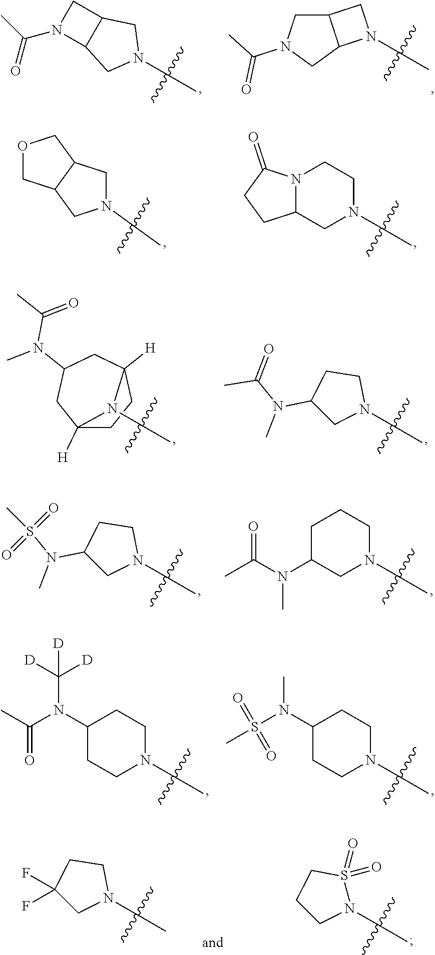

and R³ is hydrogen.

In some embodiments, R², R^{2′} and R³ are each independently selected from the group consisting of hydrogen,

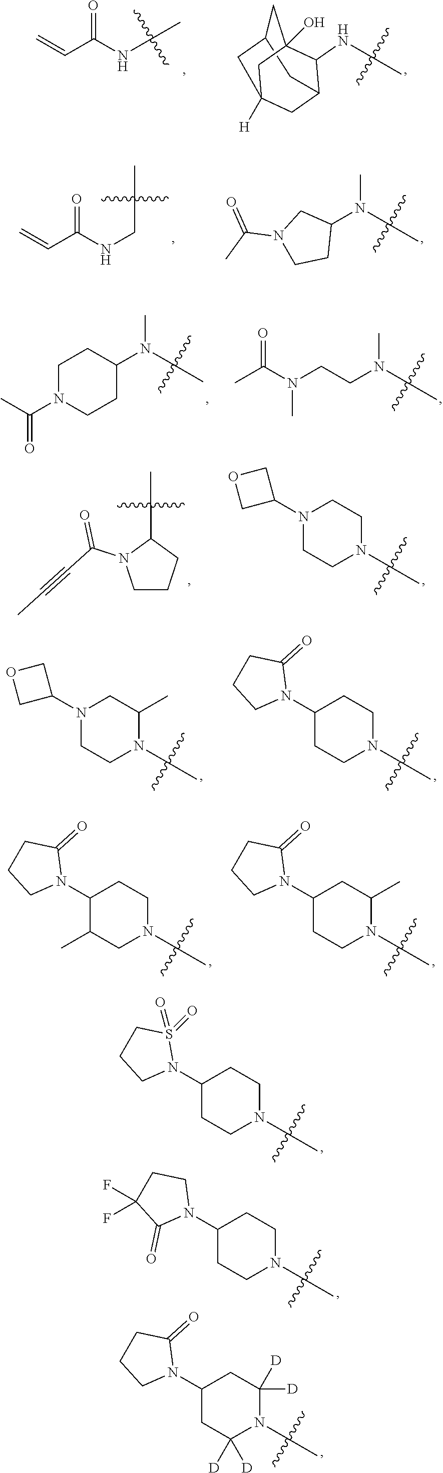

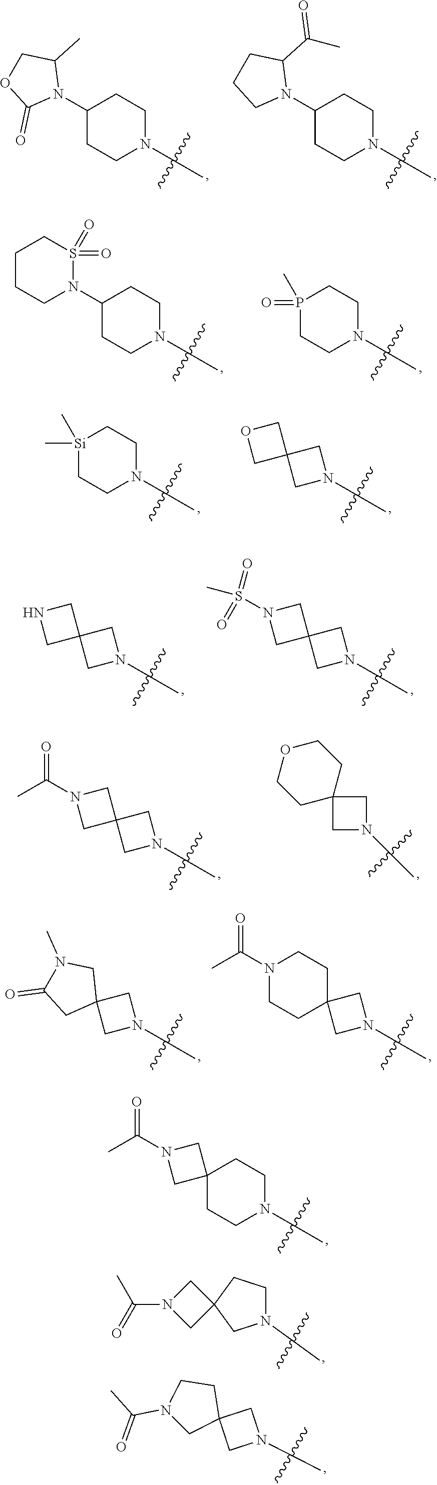

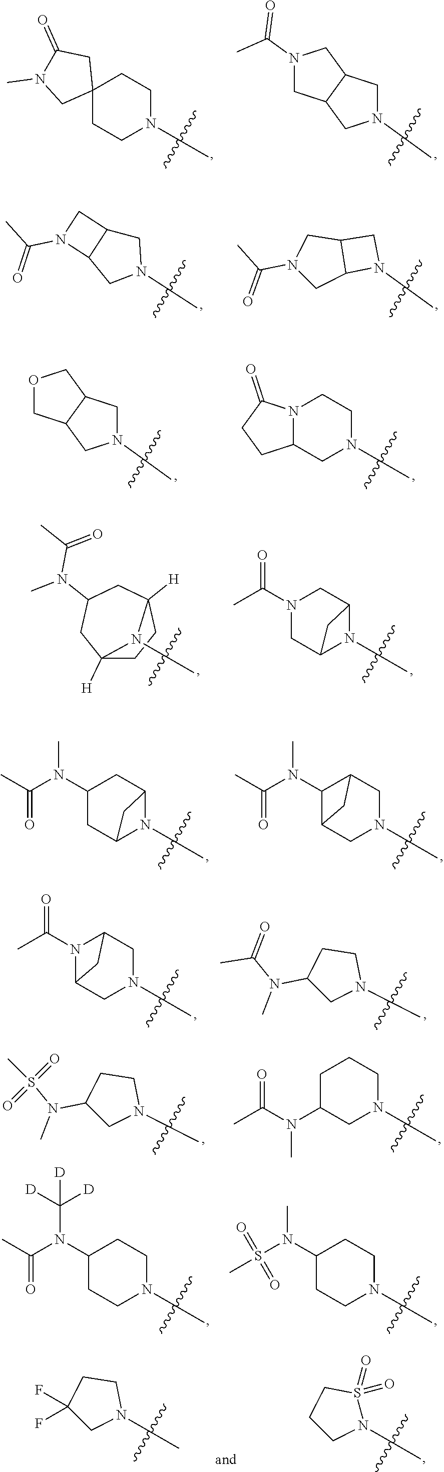

or, R³ is connected to R² or R^{2′}, and together with the carbon atoms connected thereto, forms a

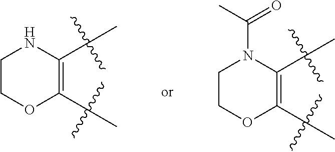 [SMILES: *C1=C(*)OCCN1 ; *C1=C(*)N(C(C)=O)CCO1]

group, and R², R^{2′}, and R³ are not hydrogen at the same time.

In some embodiments, one of R² and R^{2′} is hydrogen and the other is selected from the group consisting of

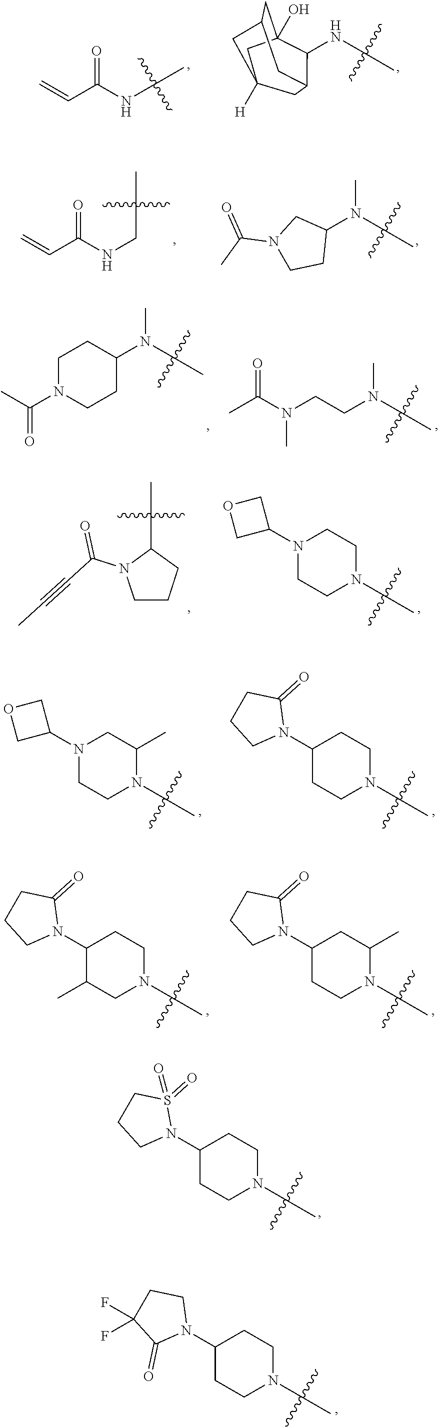

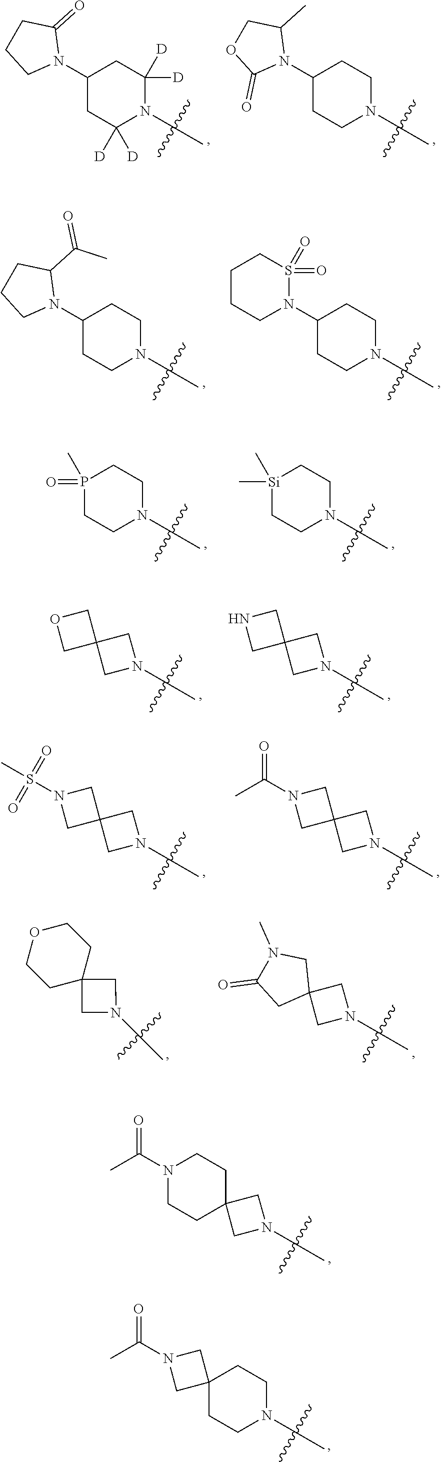

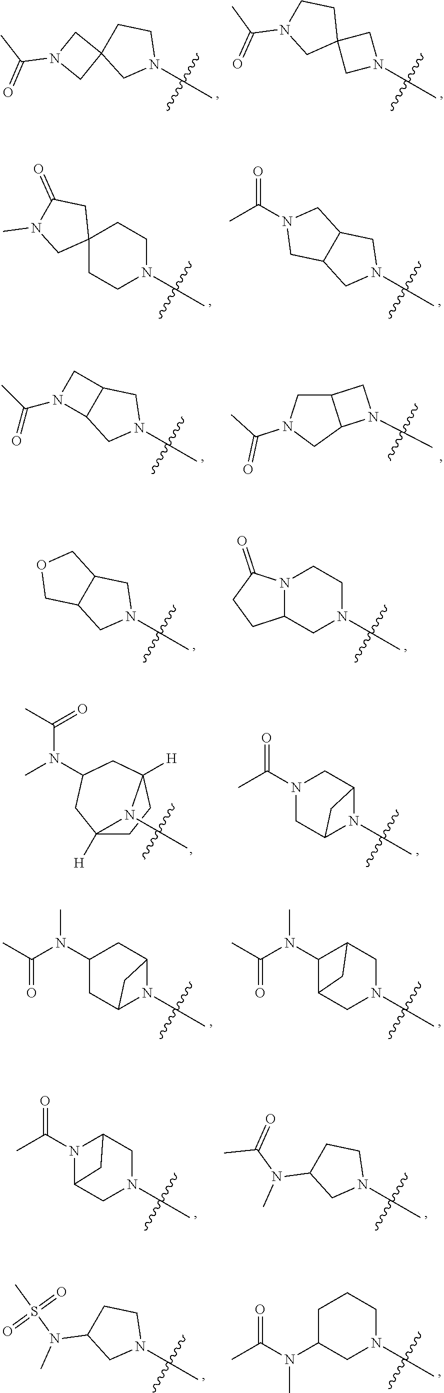

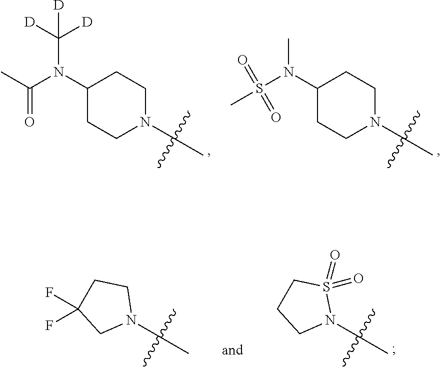 [SMILES: *N1CCC(N(C(C)=O)C([2H])([2H])[2H])CC1 ; *N1CCC(N(C)S(C)(=O)=O)CC1 ; *N1CCC(F)(F)C1 ; *N1CCCS1(=O)=O]

and R³ is hydrogen.

In some embodiments, R², R^{2′} and R³ are each independently selected from the group consisting of hydrogen,

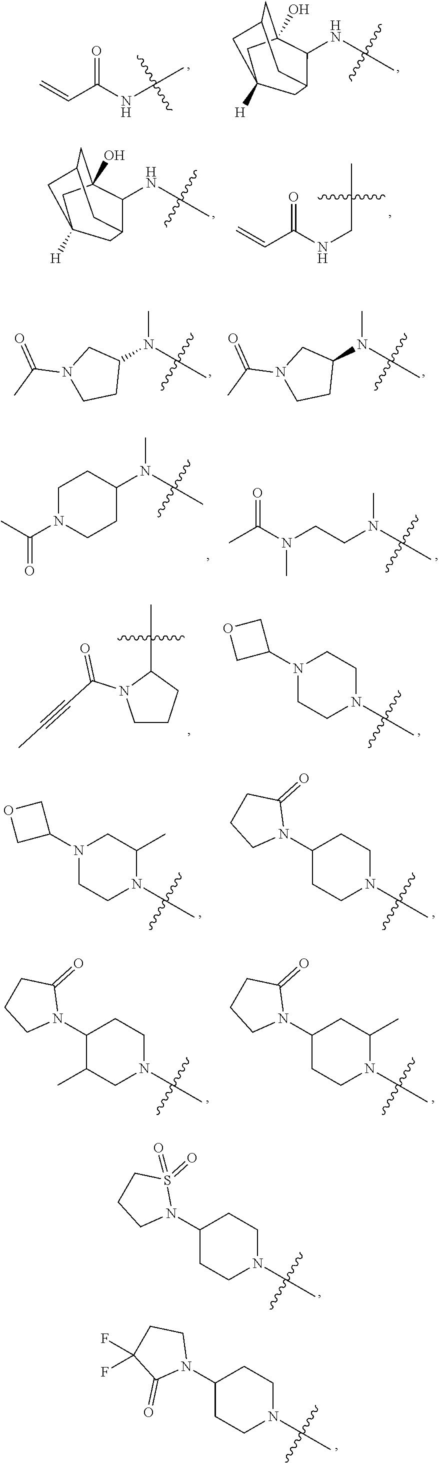

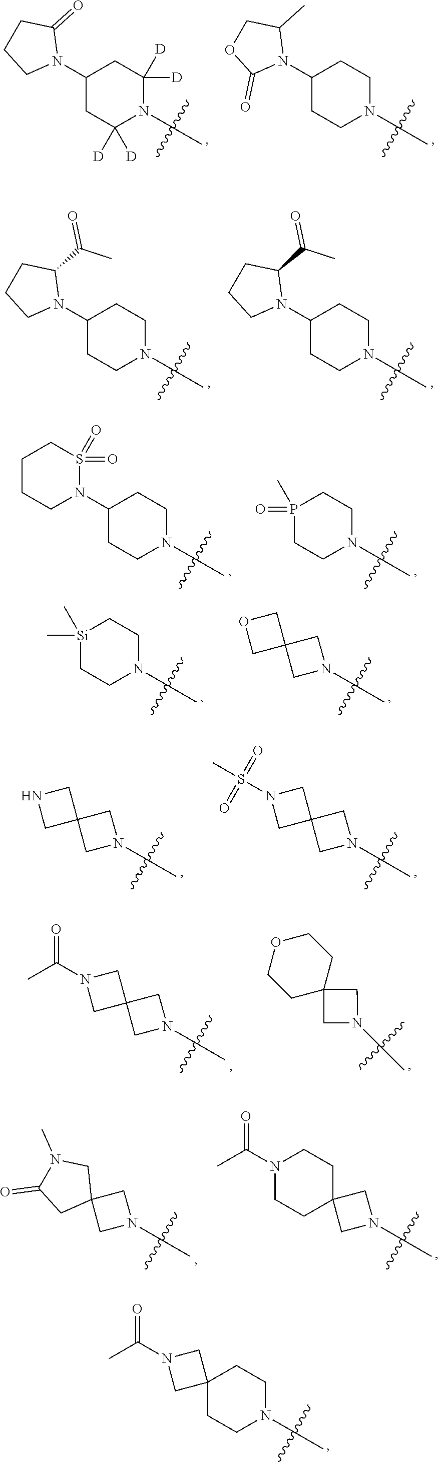

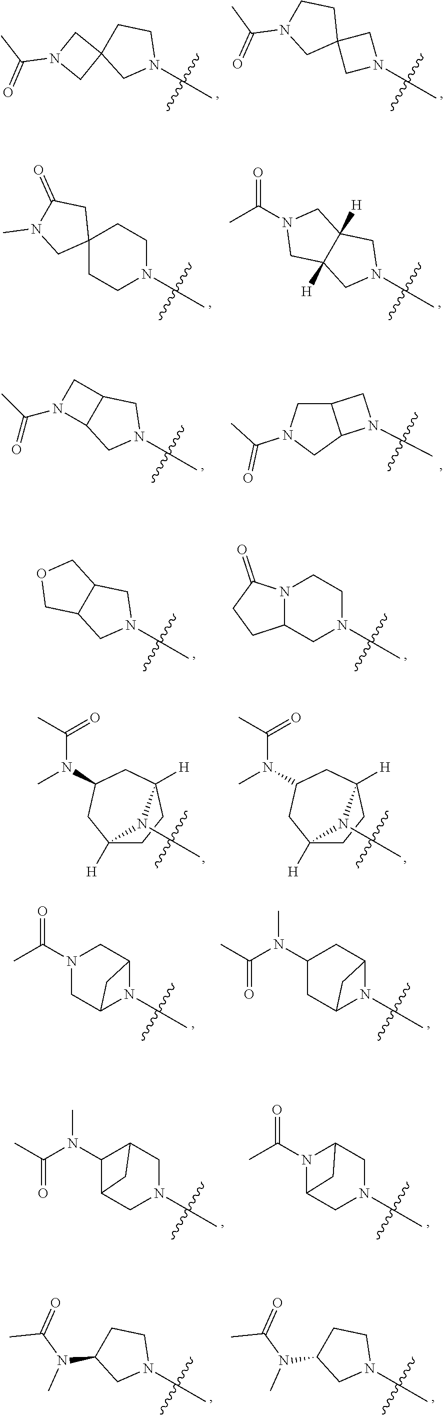

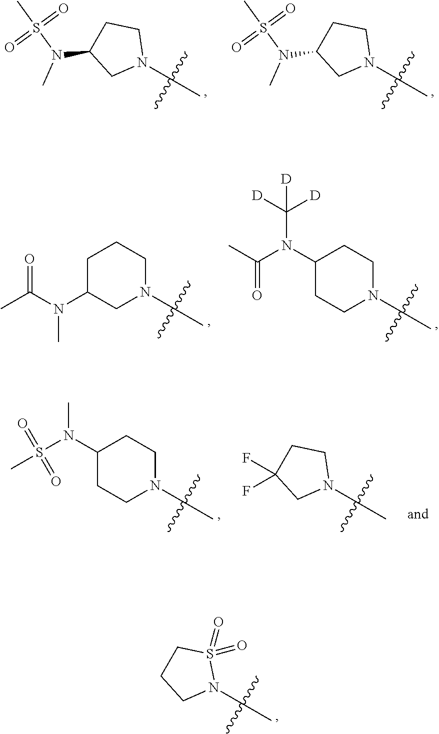

or R³ is connected to R² or R^{2′} such that the structural unit

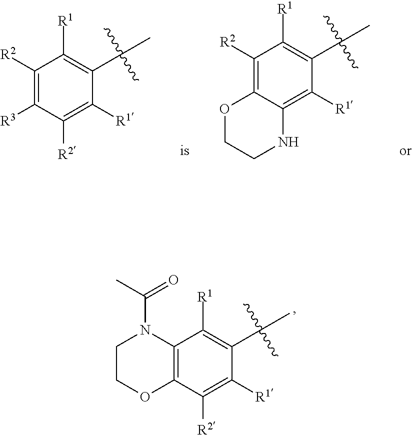

and R², R^{2′} and R³ are not hydrogen at the same time.

In some embodiments, one of R² and R^{2′} is hydrogen and the other is selected from the group consisting of

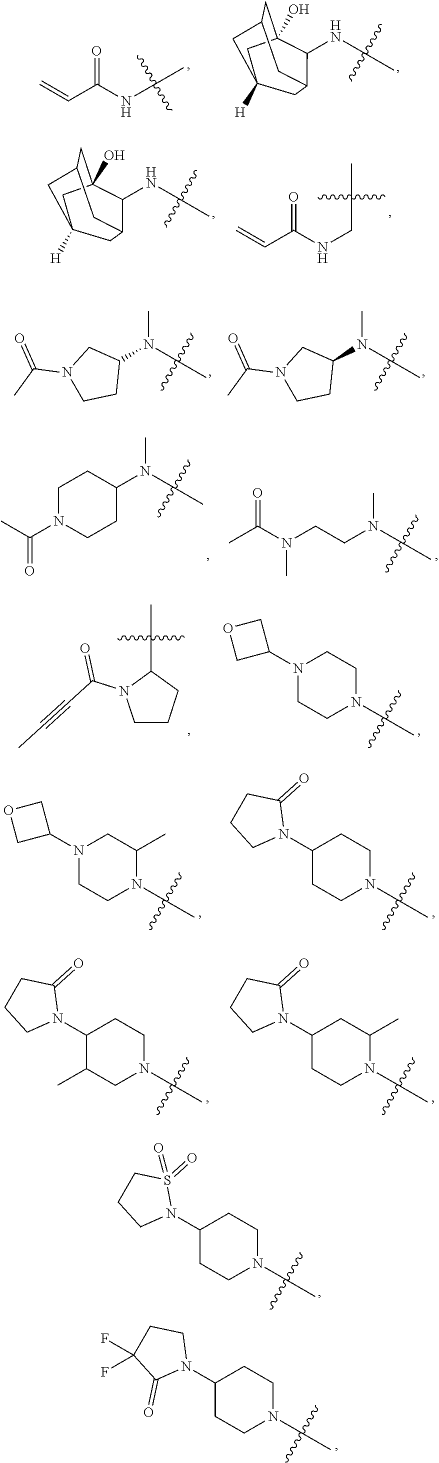

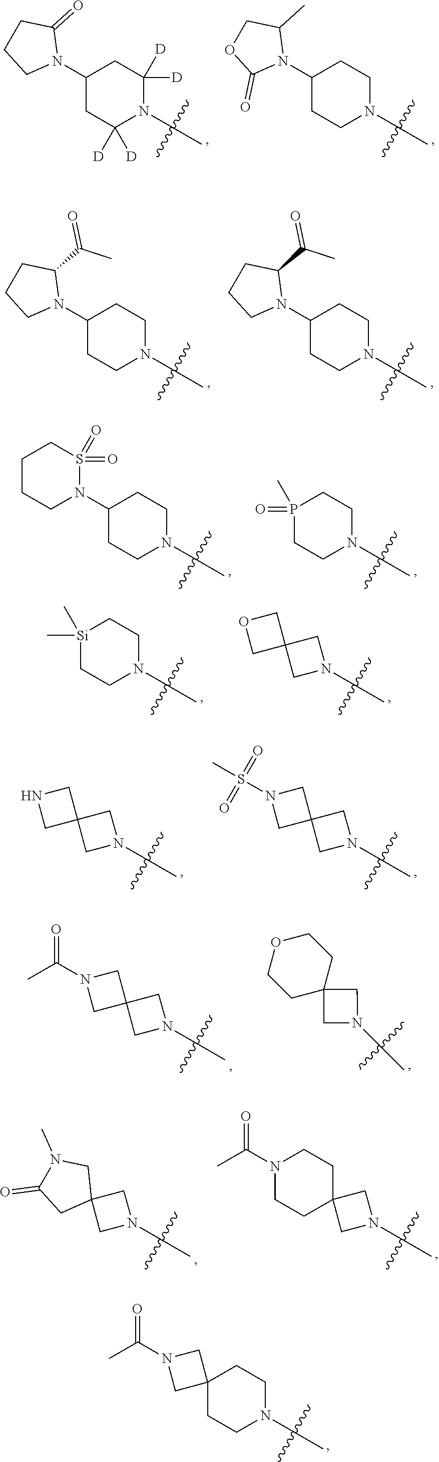

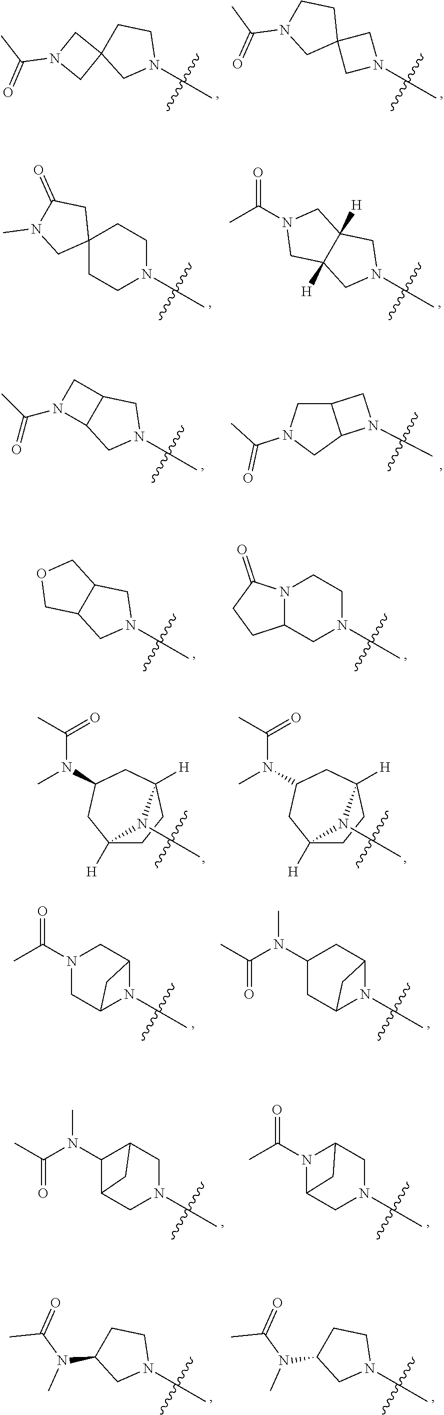

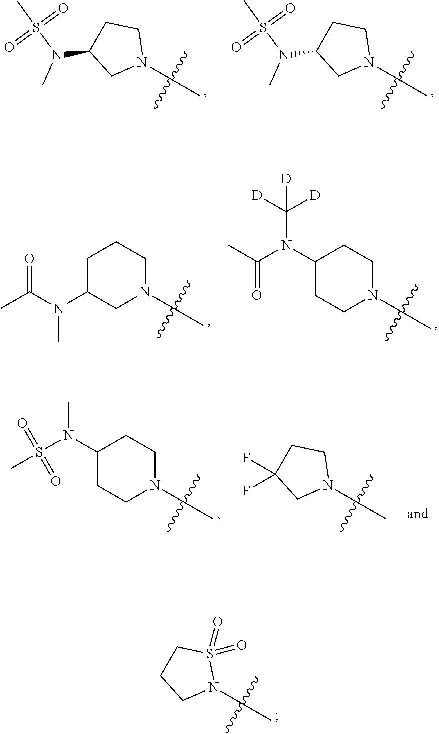

and R³ is hydrogen. In some embodiments, when R², R^{2′} or R³ are each independently selected from the group consisting of hydrogen and

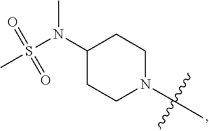 [SMILES: *N1CCC(N(C)S(C)(=O)=O)CC1]

R¹ or R^{1′} is fluorine.

In some embodiments, 2 of R², R^{2′} or R³ are hydrogen.

In some embodiments, R² and R^{2′} are hydrogen, and R³ is not hydrogen.

In some embodiments, R² is not hydrogen, and R^{2′} and R³ are hydrogen.

In some embodiments, R^{2′} is not hydrogen, and R² and R³ are hydrogen.

In some embodiments, the present application encompasses the variables defined above and embodiments thereof, as well as any combination thereof.

In another aspect, the present application provides a compound of the following formulas or a pharmaceutically acceptable salt thereof,

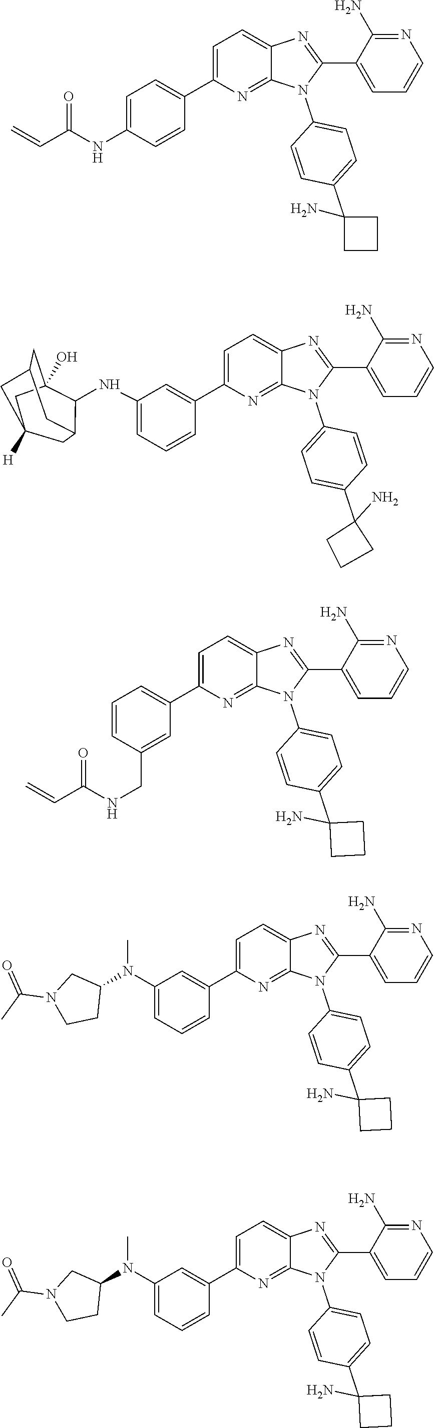

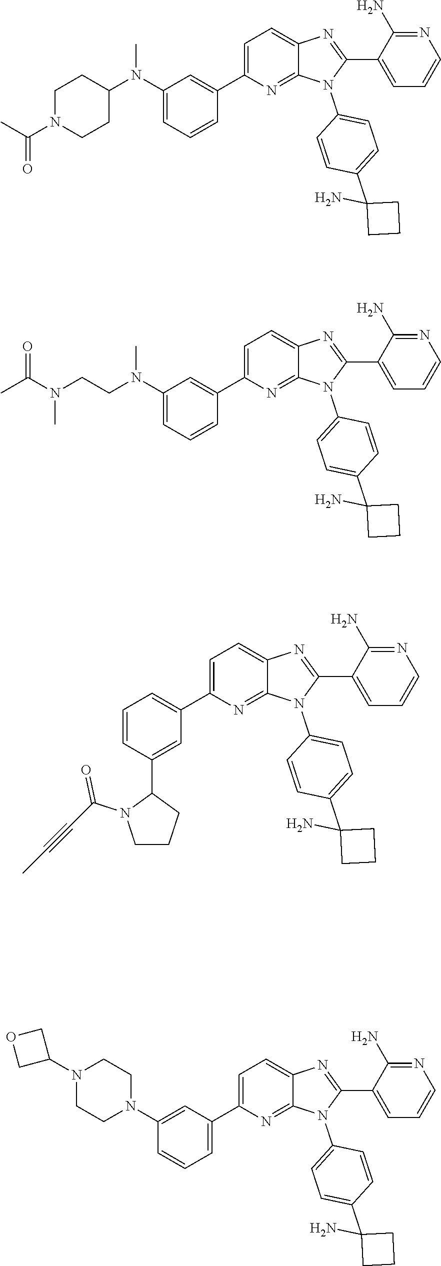 [SMILES: CC(=O)N1CCC(N(C)c2cccc(-c3ccc4nc(-c5cccnc5N)n(-c5ccc(C6(N)CCC6)cc5)c4n3)c2)CC1 ; CC(=O)N(C)CCN(C)c1cccc(-c2ccc3nc(-c4cccnc4N)n(-c4ccc(C5(N)CCC5)cc4)c3n2)c1 ; CC#CC(=O)N1CCCC1c1cccc(-c2ccc3nc(-c4cccnc4N)n(-c4ccc(C5(N)CCC5)cc4)c3n2)c1 ; Nc1ncccc1-c1nc2ccc(-c3cccc(N4CCN(C5COC5)CC4)c3)nc2n1-c1ccc(C2(N)CCC2)cc1]

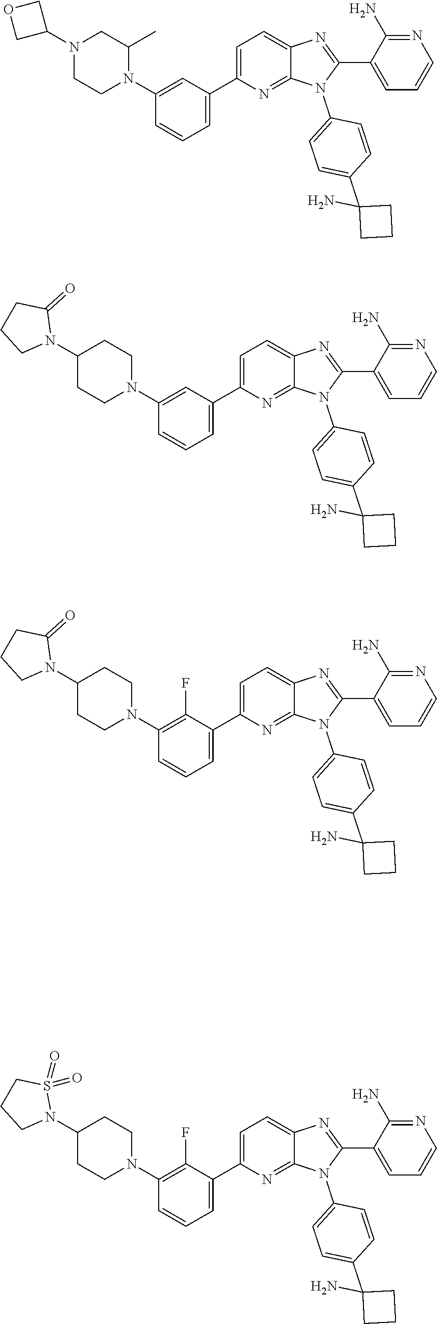 [SMILES: CC1CN(C2COC2)CCN1c1cccc(-c2ccc3nc(-c4cccnc4N)n(-c4ccc(C5(N)CCC5)cc4)c3n2)c1 ; Nc1ncccc1-c1nc2ccc(-c3cccc(N4CCC(N5CCCC5=O)CC4)c3)nc2n1-c1ccc(C2(N)CCC2)cc1 ; Nc1ncccc1-c1nc2ccc(-c3cccc(N4CCC(N5CCCC5=O)CC4)c3F)nc2n1-c1ccc(C2(N)CCC2)cc1 ; Nc1ncccc1-c1nc2ccc(-c3cccc(N4CCC(N5CCCS5(=O)=O)CC4)c3F)nc2n1-c1ccc(C2(N)CCC2)cc1]

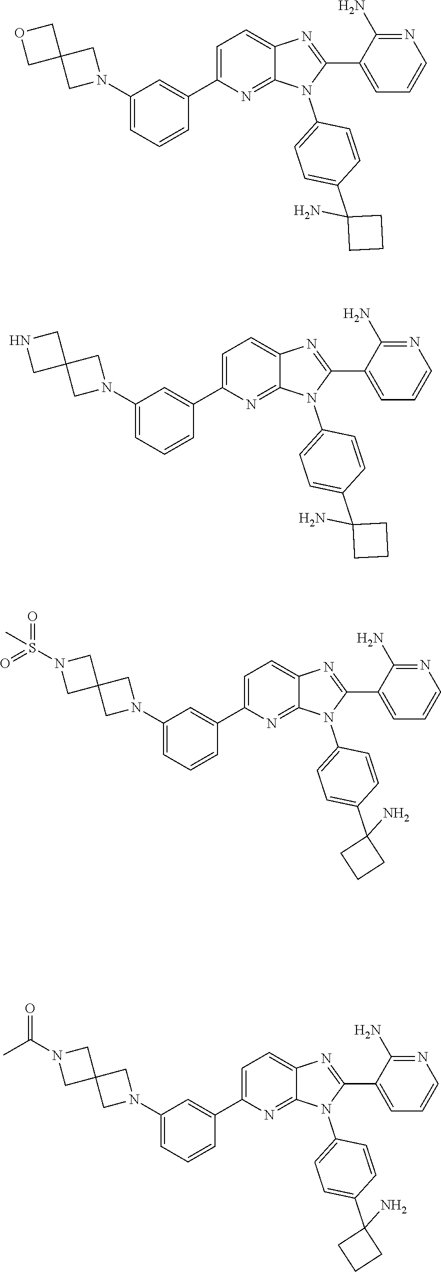 [SMILES: Nc1ncccc1-c1nc2ccc(-c3cccc(N4CC5(COC5)C4)c3)nc2n1-c1ccc(C2(N)CCC2)cc1 ; Nc1ncccc1-c1nc2ccc(-c3cccc(N4CC5(CNC5)C4)c3)nc2n1-c1ccc(C2(N)CCC2)cc1 ; CS(=O)(=O)N1CC2(CN(c3cccc(-c4ccc5nc(-c6cccnc6N)n(-c6ccc(C7(N)CCC7)cc6)c5n4)c3)C2)C1 ; CC(=O)N1CC2(C1)CN(c1cccc(-c3ccc4nc(-c5cccnc5N)n(-c5ccc(C6(N)CCC6)cc5)c4n3)c1)C2]

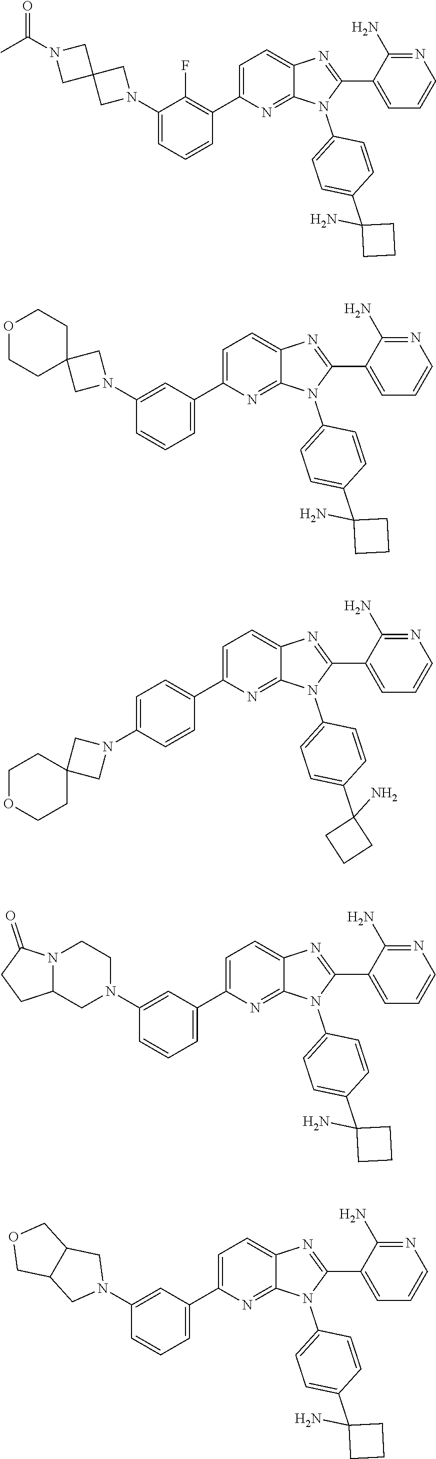 [SMILES: CC(=O)N1CC2(C1)CN(c1cccc(-c3ccc4nc(-c5cccnc5N)n(-c5ccc(C6(N)CCC6)cc5)c4n3)c1F)C2 ; Nc1ncccc1-c1nc2ccc(-c3cccc(N4CC5(CCOCC5)C4)c3)nc2n1-c1ccc(C2(N)CCC2)cc1 ; Nc1ncccc1-c1nc2ccc(-c3ccc(N4CC5(CCOCC5)C4)cc3)nc2n1-c1ccc(C2(N)CCC2)cc1 ; Nc1ncccc1-c1nc2ccc(-c3cccc(N4CCN5C(=O)CCC5C4)c3)nc2n1-c1ccc(C2(N)CCC2)cc1 ; Nc1ncccc1-c1nc2ccc(-c3cccc(N4CC5COCC5C4)c3)nc2n1-c1ccc(C2(N)CCC2)cc1]

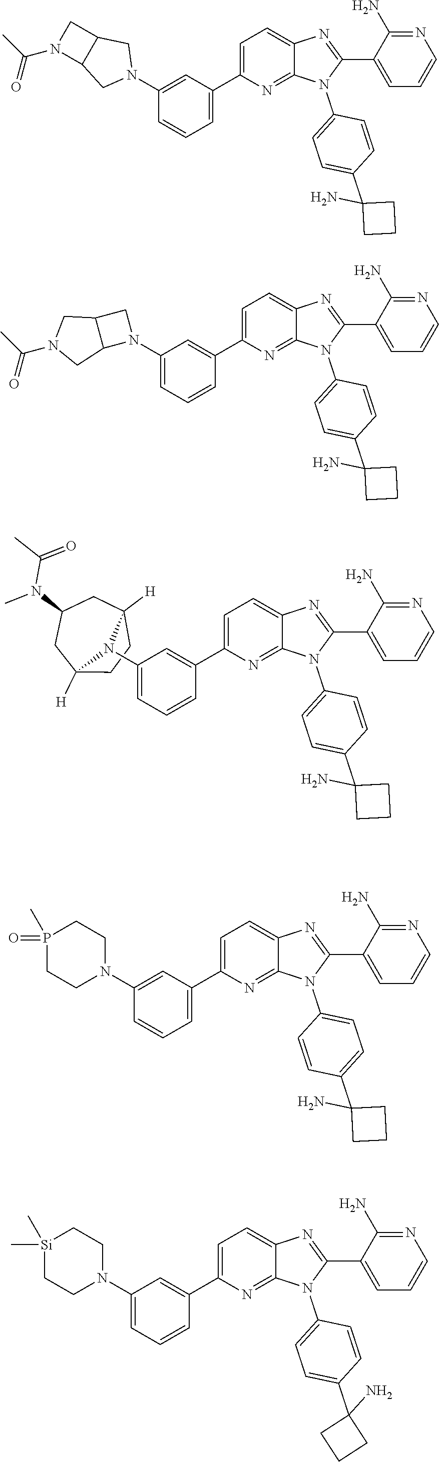

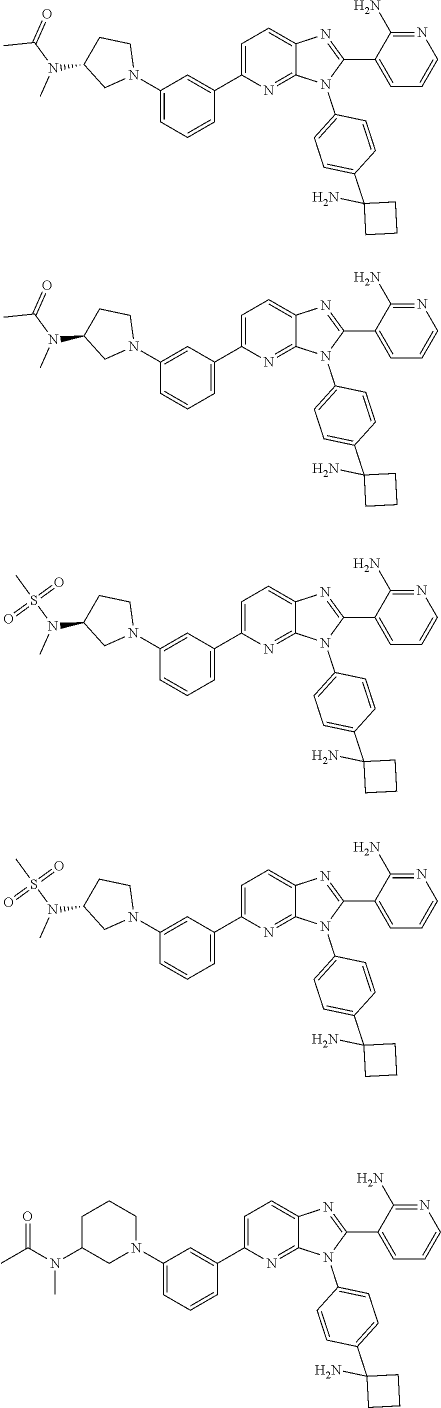

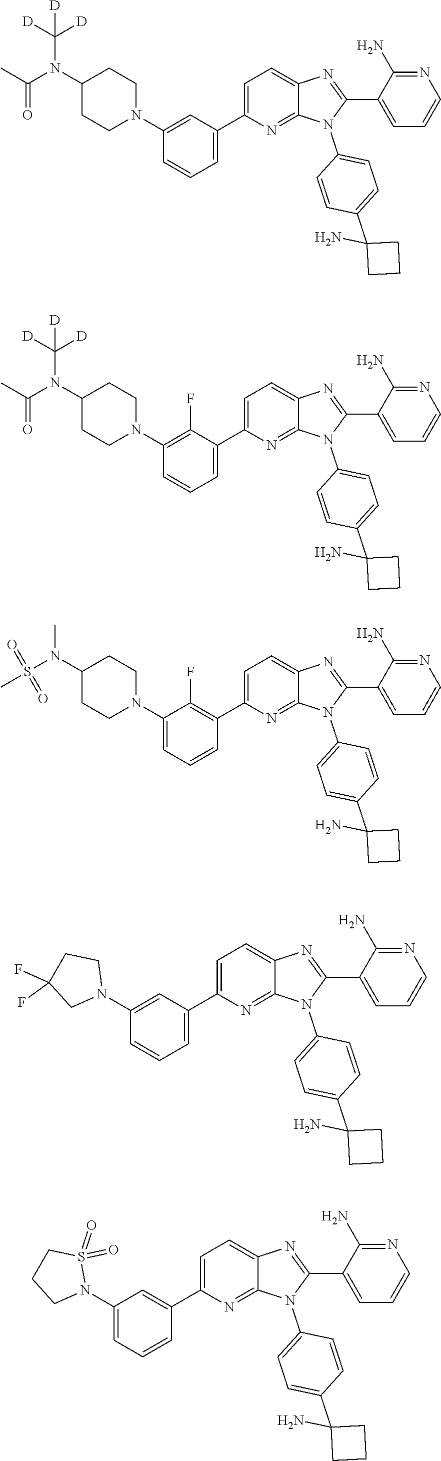 [SMILES: [2H]C([2H])([2H])N(C(C)=O)C1CCN(c2cccc(-c3ccc4nc(-c5cccnc5N)n(-c5ccc(C6(N)CCC6)cc5)c4n3)c2)CC1 ; [2H]C([2H])([2H])N(C(C)=O)C1CCN(c2cccc(-c3ccc4nc(-c5cccnc5N)n(-c5ccc(C6(N)CCC6)cc5)c4n3)c2F)CC1 ; CN(C1CCN(c2cccc(-c3ccc4nc(-c5cccnc5N)n(-c5ccc(C6(N)CCC6)cc5)c4n3)c2F)CC1)S(C)(=O)=O ; Nc1ncccc1-c1nc2ccc(-c3cccc(N4CCC(F)(F)C4)c3)nc2n1-c1ccc(C2(N)CCC2)cc1 ; Nc1ncccc1-c1nc2ccc(-c3cccc(N4CCCS4(=O)=O)c3)nc2n1-c1ccc(C2(N)CCC2)cc1]

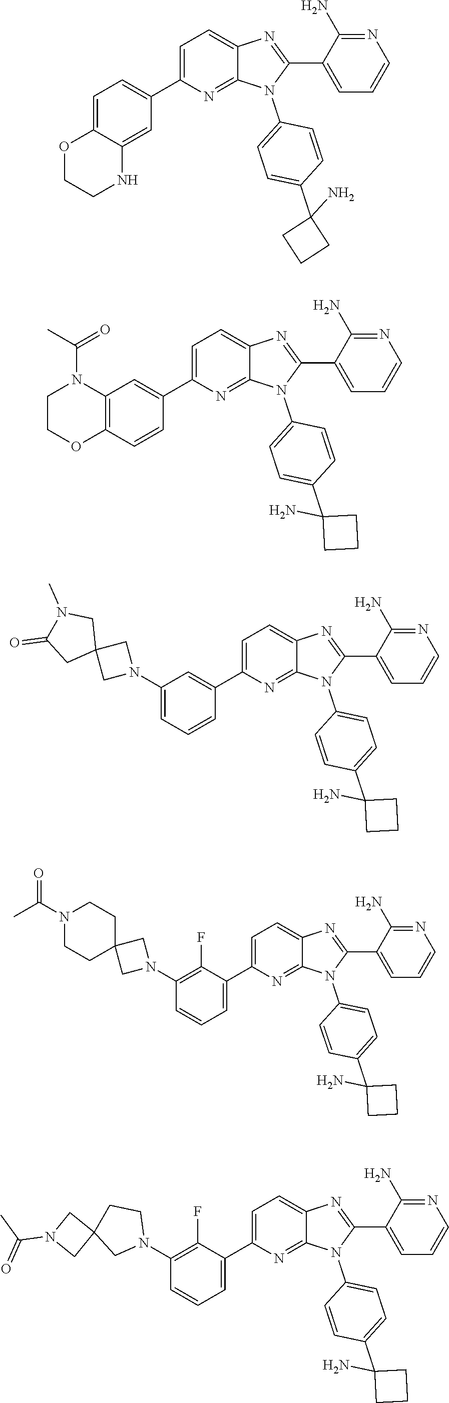 [SMILES: Nc1ncccc1-c1nc2ccc(-c3ccc4c(c3)NCCO4)nc2n1-c1ccc(C2(N)CCC2)cc1 ; CC(=O)N1CCOc2ccc(-c3ccc4nc(-c5cccnc5N)n(-c5ccc(C6(N)CCC6)cc5)c4n3)cc21 ; CN1CC2(CC1=O)CN(c1cccc(-c3ccc4nc(-c5cccnc5N)n(-c5ccc(C6(N)CCC6)cc5)c4n3)c1)C2 ; CC(=O)N1CCC2(CC1)CN(c1cccc(-c3ccc4nc(-c5cccnc5N)n(-c5ccc(C6(N)CCC6)cc5)c4n3)c1F)C2 ; CC(=O)N1CC2(CCN(c3cccc(-c4ccc5nc(-c6cccnc6N)n(-c6ccc(C7(N)CCC7)cc6)c5n4)c3F)C2)C1]

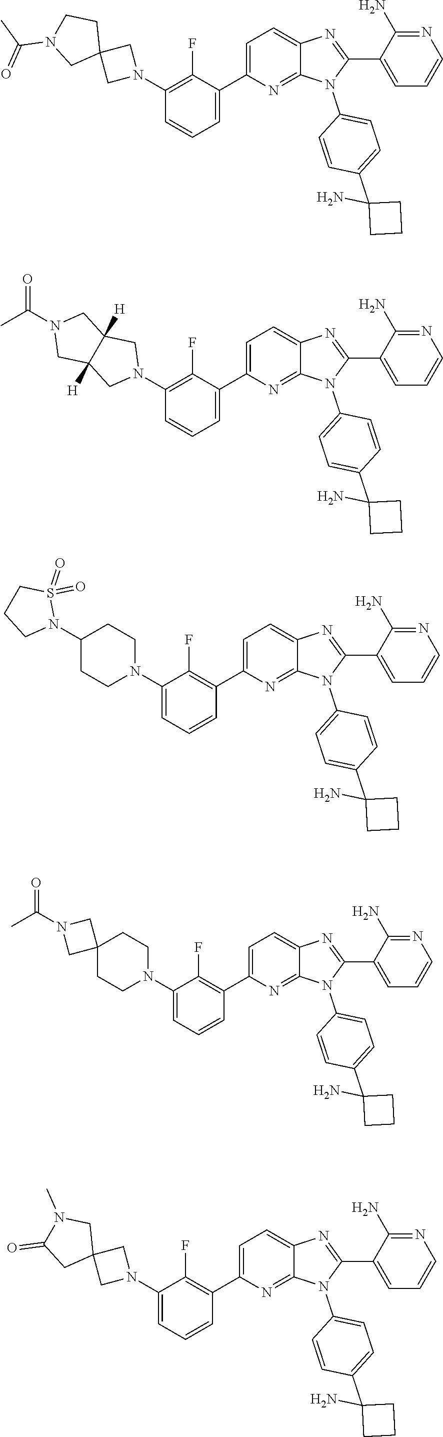 [SMILES: CC(=O)N1CCC2(C1)CN(c1cccc(-c3ccc4nc(-c5cccnc5N)n(-c5ccc(C6(N)CCC6)cc5)c4n3)c1F)C2 ; CC(=O)N1C[C@@H]2CN(c3cccc(-c4ccc5nc(-c6cccnc6N)n(-c6ccc(C7(N)CCC7)cc6)c5n4)c3F)C[C@@H]2C1 ; Nc1ncccc1-c1nc2ccc(-c3cccc(N4CCC(N5CCCS5(=O)=O)CC4)c3F)nc2n1-c1ccc(C2(N)CCC2)cc1 ; CC(=O)N1CC2(CCN(c3cccc(-c4ccc5nc(-c6cccnc6N)n(-c6ccc(C7(N)CCC7)cc6)c5n4)c3F)CC2)C1 ; CN1CC2(CC1=O)CN(c1cccc(-c3ccc4nc(-c5cccnc5N)n(-c5ccc(C6(N)CCC6)cc5)c4n3)c1F)C2]

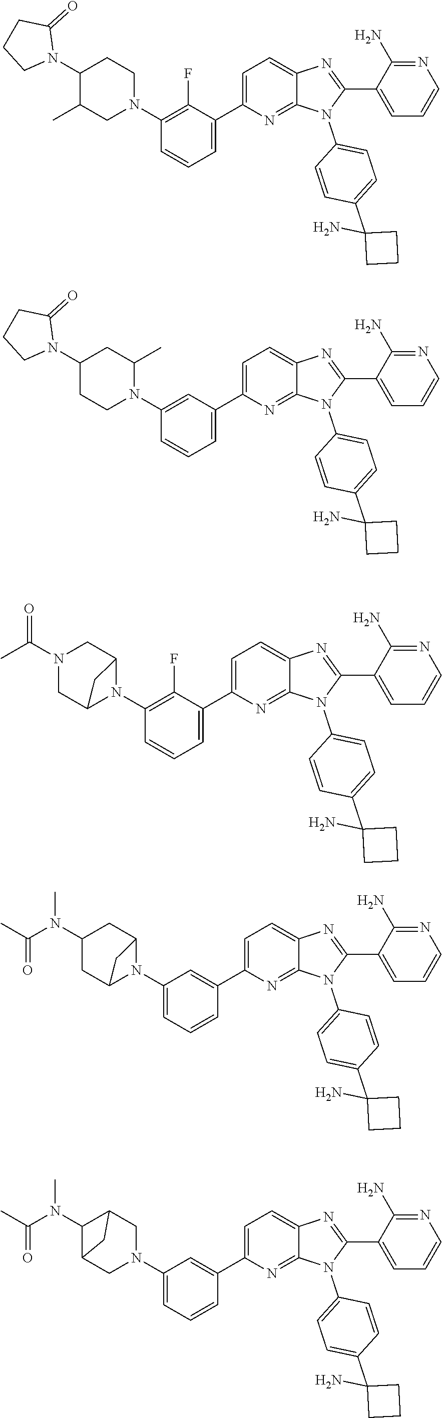

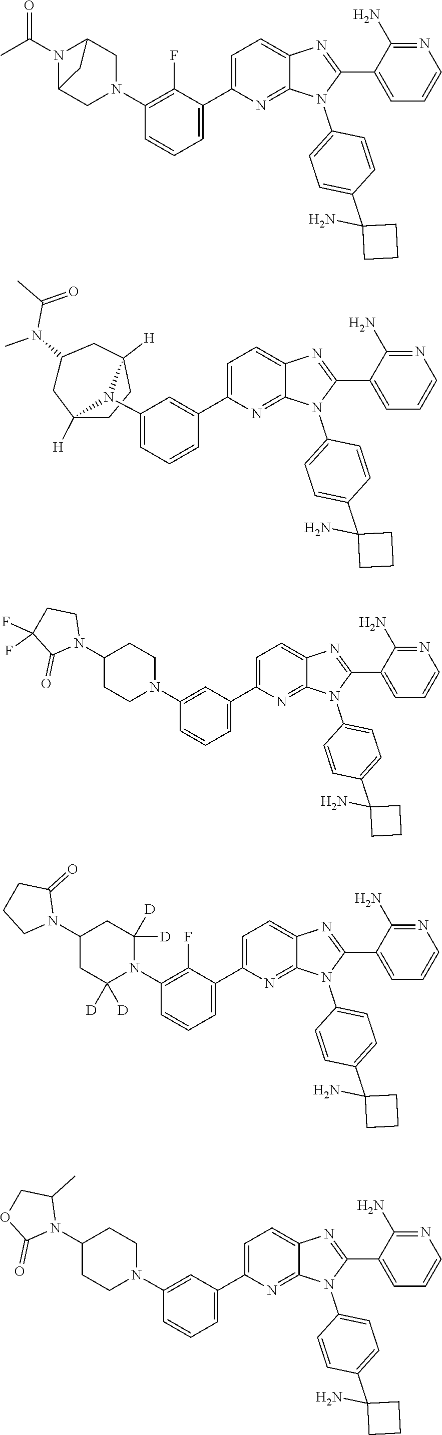

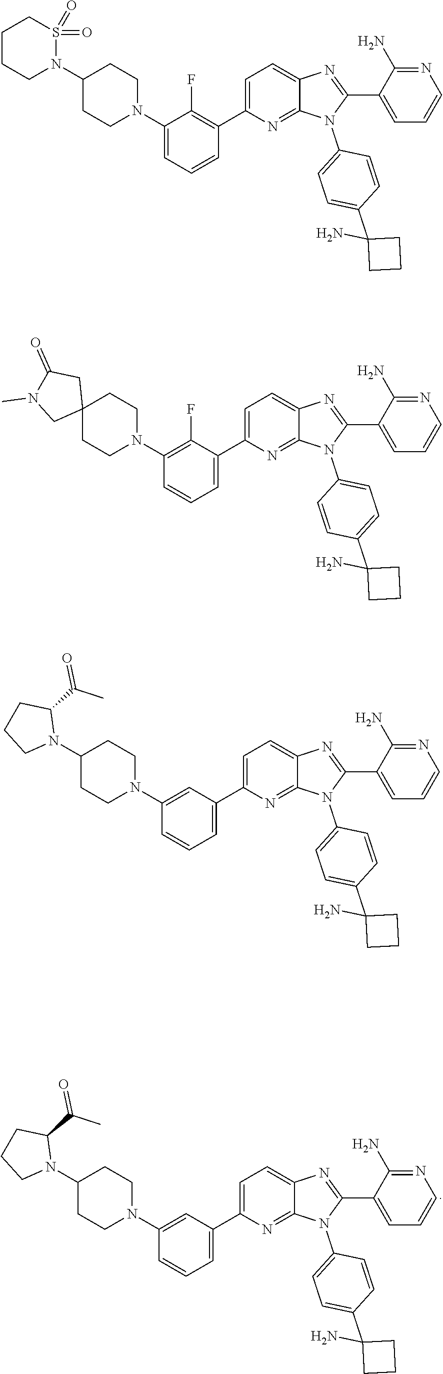 [SMILES: Nc1ncccc1-c1nc2ccc(-c3cccc(N4CCC(N5CCCCS5(=O)=O)CC4)c3F)nc2n1-c1ccc(C2(N)CCC2)cc1 ; CN1CC2(CCN(c3cccc(-c4ccc5nc(-c6cccnc6N)n(-c6ccc(C7(N)CCC7)cc6)c5n4)c3F)CC2)CC1=O ; CC(=O)[C@H]1CCCN1C1CCN(c2cccc(-c3ccc4nc(-c5cccnc5N)n(-c5ccc(C6(N)CCC6)cc5)c4n3)c2)CC1 ; CC(=O)[C@@H]1CCCN1C1CCN(c2cccc(-c3ccc4nc(-c5cccnc5N)n(-c5ccc(C6(N)CCC6)cc5)c4n3)c2)CC1]

In another aspect, the present application further provides a pharmaceutical composition comprising the compound or the pharmaceutically acceptable salt thereof of the present application described above. In some embodiments, the pharmaceutical composition of the present application further comprises a pharmaceutically acceptable excipient.

In another aspect, the present application further provides a method for treating a disease mediated by Akt kinase in a mammal, comprising administering to a mammal, preferably a human, in need of such treatment a therapeutically effective amount of the compound, the pharmaceutically acceptable salt thereof or the pharmaceutical composition thereof described above.

In another aspect, the present application further provides use of the compound, the pharmaceutically acceptable salt thereof or the pharmaceutical composition thereof described above in preparing a medicament for treating a disease mediated by Akt kinase.

In another aspect, the present application further provides use of the compound, the pharmaceutically acceptable salt thereof or the pharmaceutical composition thereof described above in treating a disease mediated by Akt kinase.

In another aspect, the present application further provides the compound, the pharmaceutically acceptable salt thereof or the pharmaceutical composition thereof of the present application described above for use in treating a disease mediated by Akt kinase.

In some embodiments, the disease mediated by Akt kinase is selected from a cancer, e.g., prostate cancer and endometrial cancer.

The compound of the present application has good inhibition effect on phosphorylation of LNcap cells and LNcap cells AKT1(S473), and has good cell activity, low toxicity, good pharmacokinetic property, strong in vivo tumor inhibition effect and the like.

### Definitions

Unless otherwise stated, the following terms used herein shall have the following meanings. A certain term, unless otherwise specifically defined, should not be considered uncertain or unclear, but construed according to its common meaning in the field. When referring to a trade name herein, it is intended to refer to its corresponding commercial product or its active ingredient.

The term “substituted” means that any one or more hydrogen atoms on a specific atom are substituted by substituents, as long as the valence of the specific atom is normal and the resulting compound is stable. When the substituent is oxo (namely ═O), it means that two hydrogen atoms are substituted, and oxo is not available on an aromatic group. There is no keto substitution on the aromatic moiety.

The term “optional” or “optionally” means that the subsequently described event or circumstance may, but not necessarily, occur. The description includes instances where the event or circumstance occurs and instances where the event or circumstance does not occur. For example, an ethyl “optionally” substituted by halogen, means that the ethyl may be unsubstituted (CH₂CH₃), monosubstituted (for example, CH₂CH₂F), polysubstituted (for example, CHFCH₂F, CH₂CHF₂ and the like) or fully substituted (CF₂CF₃). It will be understood by those skilled in the art that for any groups comprising one or more substituents, any substitutions or substituting patterns which may not exist or cannot be synthesized spatially are not introduced.

Cₘ₋ₙ used herein means that the portion has an integer number of carbon atoms in the given range. For example, “C₁₋₆” means that the group may have 1 carbon atom, 2 carbon atoms, 3 carbon atoms, 4 carbon atoms, 5 carbon atoms, or 6 carbon atoms.

When any variable (e.g., R) occurs more than once in the constitution or structure of a compound, the variable is independently defined in each case. Therefore, for example, if a group is substituted with 2 R, the definition of each R is independent.

When a bond of a substituent is cross-linked to two atoms on a ring, the substituent can be bonded to any atom on the ring. For example, structural unit

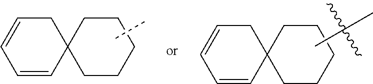

represents that substitution may occur in any one position of cyclohexyl or cyclohexadienyl.

The term “halo-” or “halogen” refers to fluorine, chlorine, bromine and iodine.

The term “hydroxy” refers to —OH group.

The term “amino” refers to —NH₂ group.

The term “alkyl” refers to hydrocarbyl with a general formula of CₙH₂ₙ₊₁, n being an integer from 1 to 20. The alkyl can be linear or branched. The alkyl herein may contain 1 to 10 carbon atoms (C₁-C₁₀ alkyl), 1 to 6 carbon atoms (C₁-C₆ alkyl), 1 to 4 carbon atoms (C₁-C₄ alkyl), or 1 to 3 carbon atoms (C₁-C₃ alkyl). For example, the term “C₁-6 alkyl” refers to alkyl containing 1 to 6 carbon atoms (for example, methyl, ethyl, n-propyl, isopropyl, n-butyl, isobutyl, sec-butyl, tert-butyl, n-pentyl, 1-methylbutyl, 2-methylbutyl, 3-methylbutyl, neopentyl, hexyl and 2-methylpentyl). The alkyl moieties (namely alkyl) of alkoxy, alkylamino, dialkylamino, alkylsulfonyl and alkylthio are similarly defined as above.

The term “alkoxyl” refers to —O-alkyl.

The term “aminoalkyl” refers to -alkyl-NH₂.

The term “alkylacyl” refers to —C(O)-alkyl.

The term “alkylsulfonyl” refers to —SO₂-alkyl.

The term “alkenyl” refers to linear or branched unsaturated aliphatic hydrocarbyl consisting of carbon atoms and hydrogen atoms with at least one double bond. The alkenyl herein may contain 2-10 carbon atoms (C₂-C₁₀ alkenyl), 2-6 carbon atoms (C₂-C₆ alkenyl), or 2-4 carbon atoms (C₂-C₄ alkenyl). Non-limiting examples of alkenyl include, but are not limited to, ethenyl, 1-propenyl, 2-propenyl, 1-butenyl, isobutenyl, 1,3-butadienyl, and the like.

The term “alkynyl” refers to linear or branched unsaturated aliphatic hydrocarbyl consisting of carbon atoms and hydrogen atoms with at least one triple bond. The alkynyl herein may contain 2-10 carbon atoms (C₂-C₁₀ alkynyl), 2-6 carbon atoms (C₂-C₆ alkynyl), or 2-4 carbon atoms (C₂-C₄ alkynyl). Non-limiting examples of alkynyl include, but are not limited to, ethynyl, 1-propinyl, 2-propinyl, 1,3-butadiynyl (—C≡C—C≡CH), and the like.

The term “cycloalkyl” refers to a carbon ring that is fully saturated and may exist as a monocyclic, bridged cyclic or spiro cyclic structure. The cycloalkyl herein may be 3-12 membered cycloalkyl, 3-10 membered cycloalkyl, 5-10 membered cycloalkyl, 3-8 membered cycloalkyl or 3-6 membered cycloalkyl. Non-limiting examples of cycloalkyl include, but are not limited to, cyclopropyl, cyclobutyl, cyclopentyl, cyclohexyl, norbornyl (bicyclo[2.2.1]heptyl), bicyclo[2.2.2]octyl, adamantyl, and the like.

The term “heterocyclyl” refers to a fully saturated or partially unsaturated (but not fully unsaturated heteroaromatic group) nonaromatic ring which may exist in the form of a monocyclic, bridged cyclic, or spiro cyclic structure. The heterocyclyl herein may be a 3-7 membered ring containing 1 to 3 heteroatoms (preferably 1 or 2 heteroatoms) independently selected from the group consisting of sulfur, silicon, phosphorus, oxygen and/or nitrogen. The heterocyclyl herein may be 4-12 membered heterocyclyl, 4-8 membered heterocyclyl, 5-6 membered heterocyclyl, 4-5 membered heterocyclyl or 6 membered heterocyclyl containing 1 to 3 heteroatoms (preferably 1 or 2 heteroatoms) independently selected from the group consisting of sulfur, oxygen and/or nitrogen. The “5-6 membered monoheterocyclyl whose ring atoms consist of a nitrogen atom and a carbon atom” herein may contain 1 or 2 nitrogen atoms. The “5-6 membered monoheterocyclyl whose ring atoms contain a silicon atom or a phosphorus atom” herein may contain 1 silicon atom or 1 phosphorus atom and 1 or 2 heteroatoms selected from the group consisting of N, O and S. Non-limiting examples of heterocyclyl include, but are not limited to, oxiranyl, tetrahydrofuranyl, dihydrofuranyl, pyrrolidinyl, N-methylpyrrolidinyl, dihydropyrrolyl, piperidinyl, piperazinyl, pyrazolidinyl, 4H-pyranyl, morpholinyl, sulfomorpholinyl, tetrahydrothienyl,

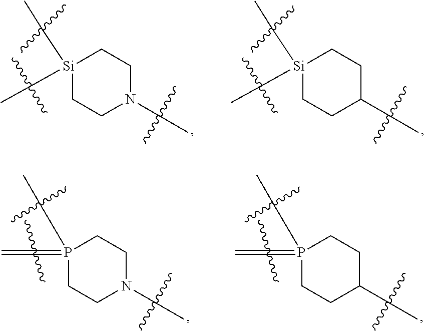 [SMILES: *N1CC[Si](*)(*)CC1 ; *C1CC[Si](*)(*)CC1 ; *N1CCP(*)(=*)CC1 ; *C1CCP(*)(=*)CC1]

and the like.

The term “heterocycloalkyl” refers to a fully saturated cyclic group that may exist in the form of a monocyclic, bridged cyclic, or spiro cyclic structure. The heterocycloalkyl herein may be a 3-7 membered ring containing 1 to 3 heteroatoms (preferably 1 or 2 heteroatoms) independently selected from the group consisting of sulfur, silicon, phosphorus, oxygen and/or nitrogen. The heterocycloalkyl herein may be 4-12 membered heterocycloalkyl, 4-8 membered heterocycloalkyl, 5-6 membered heterocycloalkyl, 4-5 membered heterocycloalkyl or 6 membered heterocycloalkyl containing 1 to 3 heteroatoms (preferably 1 or 2 heteroatoms) independently selected from the group consisting of sulfur, oxygen and/or nitrogen. Examples of 3 membered heterocycloalkyl include, but are not limited to, oxiranyl, thiiranyl, and aziranyl; non-limiting examples of 4 membered heterocycloalkyl include, but are not limited to, azetidinyl, oxetanyl, and thietanyl; examples of 5 membered heterocycloalkyl include, but are not limited to, tetrahydrofuranyl, tetrahydrothienyl, pyrrolidinyl, isoxazolidinyl, oxazolidinyl, isothiazolidinyl, thiazolidinyl, imidazolidinyl, and tetrahydropyrazolyl; examples of 6 membered heterocycloalkyl include, but are not limited to, piperidinyl, tetrahydropyranyl, tetrahydrothiopyranyl, morpholinyl, piperazinyl, 1,4-oxathianyl, 1,4-dioxanyl, thiomorpholinyl, 1,3-dithianyl, 1,4-dithianyl,

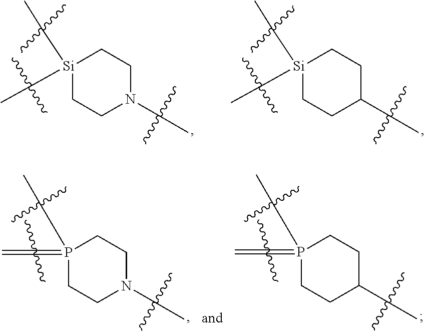 [SMILES: *N1CC[Si](*)(*)CC1 ; *C1CC[Si](*)(*)CC1 ; *N1CCP(*)(=*)CC1 ; *C1CCP(*)(=*)CC1]

examples of 7 membered heterocycloalkyl include, but are not limited to, azacycloheptanyl, oxacycloheptanyl and thiocycloheptanyl. Preferably, the heterocycloalkyl is a monocyclic heterocycloalkyl having 5 or 6 ring atoms.

The term “monoheterocyclyl” refers to a monocyclic ring that is fully saturated or partially unsaturated (but not fully saturated aromatic) and has 3 to 10 ring atoms, or 4 to 6 ring atoms. The monoheterocyclyl herein may be a 3-10 membered ring containing 1 to 3 heteroatoms (preferably 1 or 2 heteroatoms) independently selected from the group consisting of sulfur, silicon, phosphorus, oxygen and/or nitrogen. The monoheterocyclyl herein may be a 3-10 membered ring, a 3-8 membered ring, a 3-7 membered ring or a 4-6 membered ring containing 1 to 3 heteroatoms (preferably 1 or 2 heteroatoms) independently selected from the group consisting of sulfur, oxygen and/or nitrogen.

Non-limiting examples of the mono-heterocyclic ring include an oxirane ring, a tetrahydrofuran ring, a dihydrofuran ring, a 3,4-dihydrofuran ring, a 3,6-dihydrofuran ring, a tetrahydropyrrole ring, a dihydropyrrole ring, a piperidine ring, a piperazine ring, a morpholine ring, a tetrahydropyrazole ring, a tetrahydrothiophene ring, and the like.

The term “monoheterocycloalkyl” refers to a fully saturated monocyclic ring having 3 to 10 ring atoms, or 4 to 6 ring atoms. The monoheterocycloalkyl herein may be a 3-10 membered ring containing 1 to 3 heteroatoms (preferably 1 or 2 heteroatoms) independently selected from the group consisting of sulfur, silicon, phosphorus, oxygen and/or nitrogen. The monoheterocycloalkyl herein may be a 3-10 membered, 3-8 membered, 3-7 membered or 4-6 membered ring containing 1 to 3 heteroatoms (preferably 1 or 2 heteroatoms) independently selected from the group consisting of sulfur, oxygen and/or nitrogen. Non-limiting examples of monoheterocycloalkyl include an oxirane ring, a tetrahydrofuran ring, a tetrahydropyrrole ring, a piperidine ring, a piperazine ring, a morpholine ring, a tetrahydropyrazole ring, a tetrahydrothiophene ring, and the like.

The term “spirocyclyl” refers to a polycyclic ring that is fully saturated or partially unsaturated (but not fully saturated aromatic) and shares a common carbon atom (referred to as a spiro atom) between 5-20 membered monocyclic rings. The spirocyclyl is preferably 6-14 membered, more preferably 6-10 membered, 7-9 membered or 10 membered. According to the number of spiro atoms shared among the rings, the spiro cyclic ring may be a monospiro cyclic ring, a bispiro cyclic ring or a polyspiro cyclic ring, preferably a monospiro cyclic ring or a bispiro cyclic ring, and more preferably a 4 membered/4 membered, 4 membered/5 membered, 4 membered/6 membered, 5 membered/5 membered or 5 membered/6 membered monospiro cyclic ring. Non-limiting examples of spiro cyclic ring include

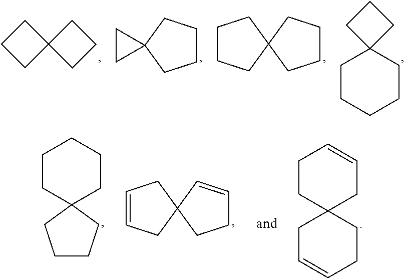

The term “spiro-cycloalkyl” refers to fully saturated spiro-cyclyl.

The term “spiro-heterocyclyl” refers to a spiro ring whose one or more ring atoms are heteroatoms selected from the group consisting of sulfur, silicon, phosphorus, oxygen and/or nitrogen (preferably 1 or 2 heteroatoms, preferably selected from the group consisting of N, O and/or S), the remaining ring atoms being carbon. The spiro-heterocyclyl is preferably 6-14 membered, more preferably 6-10 membered, or 7-9 membered or 10 membered. According to the number of spiro atoms shared among the rings, the spiro heterocyclic ring may be a monospiro heterocyclic ring, a bispiro heterocyclic ring or a polyspiro heterocyclic ring, preferably a monospiro heterocyclic ring or a bispiro heterocyclic ring, and more preferably a 4 membered/4 membered, 4 membered/5 membered, 4 membered/6 membered, 5 membered/5 membered or 5 membered/6 membered monospiro heterocyclic ring, preferably each ring contains 1 heteroatom selected from the group consisting of N, O and/or S. Non-limiting examples of spiro heterocyclic ring include

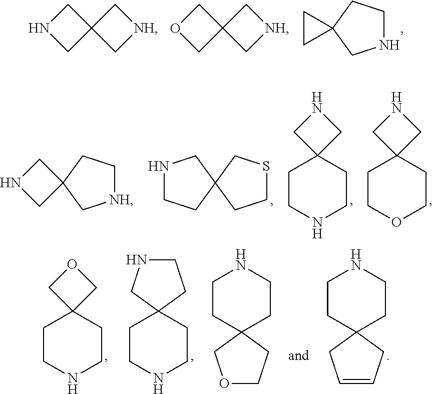

The term “spiro-heterocycloalkyl” refers to fully saturated spiro-heterocyclyl.

The term “bridged cyclyl” refers to an all-carbon polycyclic ring that is fully saturated or partially unsaturated (but not fully saturated aromatic) and has 5 to 20 ring atoms with the two rings sharing 3 or more ring atoms. The bridged cyclyl is preferably a 6-14 membered polycyclic ring, and more preferably a 6-10 membered polycyclic ring. According to the number of the formed rings, the bridged ring may be bicyclic, tricyclic, tetracyclic or polycyclic bridged ring, preferably bicyclic or tricyclic bridged ring, and more preferably bicyclic bridged ring. Non-limiting examples of the bridged ring include

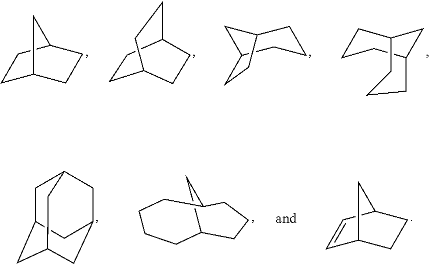

The term “bridged cycloalkyl” refers to a fully saturated bridged cyclyl.

The term “bridged heterocyclyl” refers to a bridged ring in which one or more ring atoms are heteroatoms selected from the group consisting of sulfur, silicon, phosphorus, oxygen and/or nitrogen (preferably 1 or 2 heteroatoms, preferably selected from the group consisting of N, O and/or S), the remaining ring atoms being carbon. The bridged heterocyclyl is preferably 6-14 membered, and more preferably 6-10 membered, 7-9 membered, 7 membered or 8 membered. According to the number of the formed rings, the bridged heterocyclic ring may be bicyclic, tricyclic, tetracyclic or polycyclic bridged heterocyclic ring, preferably bicyclic or tricyclic bridged heterocyclic ring, and more preferably bicyclic bridged heterocyclic ring. Non-limiting examples of bridged heterocyclic ring include

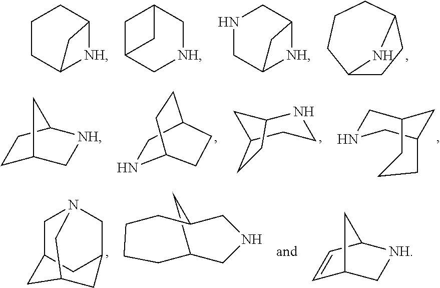

The term “bridged heterocycloalkyl” refers to a fully saturated bridged heterocyclyl.

The term “fused cyclyl” refers to an all-carbon polycyclic ring that is fully saturated or partially unsaturated (but not fully saturated aromatic) and has 5 to 20 ring atoms with the two rings sharing 2 ring atoms. The fused cyclyl is preferably a 6-14 membered polycyclic ring, and more preferably a 6-10 membered polycyclic ring. According to the number of the formed rings, the fused ring may be bicyclic, tricyclic, tetracyclic or polycyclic fused ring, preferably bicyclic or tricyclic fused ring, and more preferably bicyclic fused ring. Non-limiting examples of fused ring include

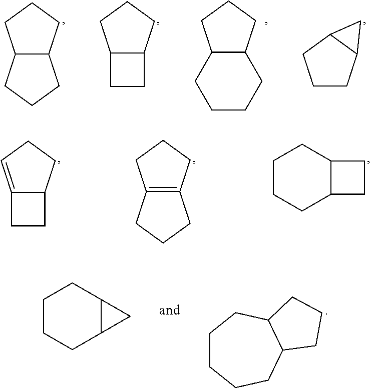

The term “fused cycloalkyl” refers to a fully saturated fused cyclyl.

The term “fused-heterocyclyl” refers to a fused ring in which one or more ring atoms are heteroatoms selected from the group consisting of sulfur, silicon, phosphorus, oxygen and/or nitrogen (preferably 1 or 2 heteroatoms, preferably selected from the group consisting of N, O and/or S), the remaining ring atoms being carbon. The fused-heterocyclyl is preferably 6-14 membered, and more preferably 6-10 membered, 7-10 membered, 7 membered, 8 membered or 9 membered. According to the number of the formed rings, the bridged heterocyclic ring may be bicyclic, tricyclic or polycyclic bridged heterocyclic ring, preferably bicyclic bridged heterocyclic ring. Non-limiting examples of fused ring include

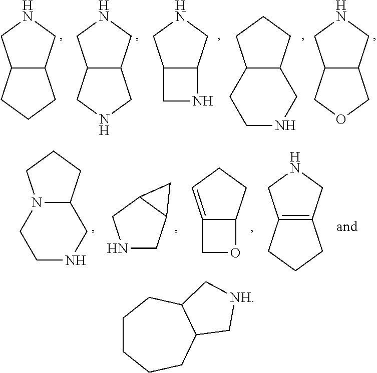

The term “fused heterocycloalkyl” refers to a fully saturated fused-heterocyclyl.

The groups described above each may be optionally substituted with one or more (e.g., 1, 2, or 3) substituents including, but not limited to, deuterium, hydroxy, halogen (F, Cl, Br, or I), [symbol] , C₁-C₆ alkyl, C₁-C₆ alkylsulfonyl, C₁-C₆ alkylacyl, and C₁-C₆ alkylacyl-N(C₁-C₆ alkyl)-.

The term “treating” or “treatment” means administering the compound or formulation described herein to ameliorate or eliminate a disease or one or more symptoms associated with the disease, and includes:
(i) inhibiting a disease or disease state, i.e., arresting its progression; and
(ii) alleviating a disease or disease state, i.e., causing its regression.

The term “therapeutically effective amount” refers to an amount of the compound disclosed herein for (i) treating a specific disease, condition, or disorder, or (ii) alleviating, ameliorating, or eliminating one or more symptoms of a specific disease, condition, or disorder. The amount of the compound of the present application composing the “therapeutically effective amount” varies dependently on the compound, the disease state and its severity, the route of administration, and the age of the mammal to be treated, but can be determined routinely by those skilled in the art in accordance with their knowledge and the present disclosure.

Therapeutic dosages of the compounds disclosed herein may be determined by, for example, the specific use of a treatment, the route of administration of the compound, the health and condition of a patient, and the judgment of a prescribing physician. The proportion or concentration of the compound disclosed herein in a pharmaceutical composition may not be constant and depends on a variety of factors including dosages, chemical properties (e.g., hydrophobicity), and routes of administration. For example, the compound of the present application may be provided for parenteral administration by a physiological buffered aqueous solution containing about 0.1-10% w/v of the compound. Certain typical dosage ranges from about 1 μg/kg body weight/day to about 1 g/body weight/day. In certain embodiments, the dosage ranges from about 0.01 mg/kg body weight/day to about 100 mg/kg body weight/day. The dosage is likely to depend on such variables as the type and degree of progression of the disease or disorder, the general health condition of the particular patient, the relative biological potency of the compound selected, the excipient formulation and its route of administration. Effective doses can be extrapolated from dose-response curves derived from in vitro or animal model test systems.

The term “pharmaceutically acceptable” is used herein for those compounds, materials, compositions, and/or dosage forms which are, within the scope of sound medical judgment, suitable for use in contact with the tissues of human beings and animals without excessive toxicity, irritation, allergic response, or other problems or complications, and commensurate with a reasonable benefit/risk ratio. A pharmaceutically acceptable salt, for example, may be a metal salt, an ammonium salt, a salt formed with an organic base, a salt formed with an inorganic acid, a salt formed with an organic acid, a salt formed with a basic or acidic amino acid, and the like.

The term “pharmaceutical composition” refers to a mixture consisting of one or more of the compounds or the salts thereof disclosed herein and a pharmaceutically acceptable excipient. The pharmaceutical composition is intended to facilitate the administration of the compound to an organic entity. The pharmaceutical compositions of the present invention may be prepared by conventional methods in the art, for example, by mixing the compound of the present application or a salt thereof with one or more pharmaceutically acceptable excipients.

The term “pharmaceutically acceptable excipients” refers to those which do not have a significant irritating effect on an organic entity and do not impair the biological activity and properties of the active compound. Suitable excipients are well known to those skilled in the art, such as carbohydrate, wax, water-soluble and/or water-swellable polymers, hydrophilic or hydrophobic material, gelatin, oil, solvent and water.

The word “comprise” and variations thereof such as “comprises” or “comprising” will be understood in an open, non-exclusive sense, i.e., “including but not limited to”.

The compounds and intermediates disclosed herein may also exist in different tautomeric forms, and all such forms are included within the scope of the present application. The term “tautomer” or “tautomeric form” refers to structural isomers of different energies that can interconvert via a low energy barrier. For example, a proton tautomer (also referred to as prototropic tautomer) includes interconversion via proton transfer, such as keto-enol isomerism and imine-enamine isomerism. A specific example of a proton tautomer is an imidazole moiety where a proton can transfer between two ring nitrogens. A valence tautomer includes the interconversion via recombination of some bonding electrons.

The present application also comprises isotopically labeled compounds which are identical to those recited herein but have one or more atoms replaced by an atom having an atomic mass or mass number different from the atomic mass or mass number usually found in nature. Examples of isotopes that can be incorporated into the compounds disclosed herein include isotopes of hydrogen, carbon, nitrogen, oxygen, phosphorus, sulfur, fluorine, iodine and chlorine, such as ²H, ³H, ¹¹C, ¹³C, ¹⁴C, ¹³N, ¹⁵N, ¹⁵O, ¹⁷O, ¹⁸O, ³⁵S, ³¹P, ³²P, ³⁵S, ¹⁸F, ¹²³I, ¹²⁵I and ³⁶Cl.

Certain isotopically labeled compounds disclosed herein (e.g., those labeled with ³H and ¹⁴C) can be used to analyze compounds and/or substrate tissue distribution. Tritiated (i.e., ³H) and carbon-14 (i.e., ¹⁴C) isotopes are particularly preferred for their ease of preparation and detectability. Positron emitting isotopes, such as ¹⁵O, ¹³N, ¹¹C and ¹⁸F can be used in positron emission tomography (PET) studies to determine substrate occupancy. Isotopically labeled compounds disclosed herein can generally be prepared by following procedures analogous to those disclosed in the schemes and/or examples below while substituting a non-isotopically labeled reagent with an isotopically-labeled reagent.

Furthermore, substitution with heavier isotopes such as deuterium (i.e., ²H) may provide certain therapeutic advantages (e.g., increased in vivo half-life or reduced dosage) resulting from greater metabolic stability and hence may be preferred in some circumstances in which deuterium substitution may be partial or complete, wherein partial deuterium substitution refers to substitution of at least one hydrogen with at least one deuterium. The hydrogens in the exemplary methyl are all deuterated to form methyl-d₃, but not limited thereto.

The compound disclosed herein can be asymmetrical, for example, has one or more stereoisomers.

Unless otherwise stated, all stereoisomers are included, for example, enantiomers and diastereoisomers. The compound with asymmetric carbon atoms disclosed herein can be separated in an optically pure form or in a racemic form. The optically pure form can be separated from a racemic mixture or can be synthesized using a chiral raw material or a chiral reagent. For example, the

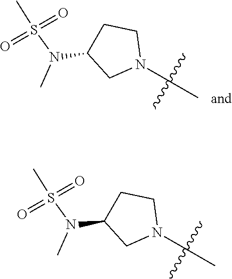 [SMILES: *N1CC[C@@H](N(C)S(C)(=O)=O)C1 ; *N1CC[C@H](N(C)S(C)(=O)=O)C1]

contained in the structure of the compound is the (R)-and (S)-enantiomers.

Unless otherwise stated, the absolute configuration of a stereogenic center is represented by a wedged solid bond ( [symbol] ) and a wedged dashed bond ( [symbol] ), and the relative configuration of a stereogenic center is represented by a straight solid bond ( [symbol] ) and a straight dashed bond ( [symbol] ). A wavy line ( [symbol] ) represents a wedged solid bond ( [symbol] ) or a wedged dashed bond ( [symbol] ), or a wavy line ( [symbol] ) represents a straight solid bond ( [symbol] ) and a straight dashed bond ( [symbol] ).

Unless otherwise stated, when a double bond structure such as a carbon-carbon double bond, a carbon-nitrogen double bond, and a nitrogen-nitrogen double bond is present in the compound, and each atom on the double bond is linked to two different substituents (in the double bond including a nitrogen atom, a lone pair of electrons on the nitrogen atom is regarded as a substituent to which the nitrogen atom is linked), if the atom on the double bond of the compound and its substituents are linked using a wavy line ( [symbol] ), means that the compound exists in the form of a (Z)-type isomer, an (E)-type isomer, or a mixture of the two isomers.

The pharmaceutical composition disclosed herein can be prepared by combining the compound disclosed herein with a suitable pharmaceutically acceptable excipient, and can be formulated, for example, into a solid, semisolid, liquid, or gaseous formulation such as tablet, pill, capsule, powder, granule, ointment, emulsion, suspension, suppository, injection, inhalant, gel, microsphere and aerosol.

Typical routes of administration of the compound or the pharmaceutically acceptable salt thereof or the pharmaceutical composition thereof of the present application include, but are not limited to, oral, rectal, local, inhalation, parenteral, sublingual, intravaginal, intranasal, intraocular, intraperitoneal, intramuscular, subcutaneous and intravenous administration.

The pharmaceutical composition disclosed herein can be manufactured using methods well known in the art, such as conventional mixing, dissolving, granulating, dragee-making, levigating, emulsifying, and lyophilizing.

In some embodiments, the pharmaceutical composition is in an oral form. For oral administration, the pharmaceutical composition can be formulated by mixing the active compounds with pharmaceutically acceptable excipients well known in the art. These excipients enable the compound disclosed herein to be formulated into tablets, pills, pastilles, dragees, capsules, liquids, gels, slurries, suspensions, etc. for oral administration to a patient.

A solid oral composition can be prepared by conventional mixing, filling or tableting. For example, it can be obtained by the following method: mixing the active compounds with solid excipients, optionally grinding the resulting mixture, adding additional suitable excipients if desired, and processing the mixture into granules to get the core parts of tablets or dragees. Suitable excipients include, but are not limited to: binders, diluents, disintegrants, lubricants, glidants, sweeteners or flavoring agents and the like.

The pharmaceutical compositions may also be suitable for parenteral administration, such as sterile solutions, suspensions or lyophilized products in suitable unit dosage forms.

The compounds of the present application can be prepared using a variety of synthetic methods well known to those skilled in the art, including the specific embodiments listed below, embodiments formed by combinations thereof with other chemical synthetic methods, and equivalents thereof known to those skilled in the art. The preferred embodiments include, but are not limited to, the examples of the present application.

The chemical reactions of the embodiments disclosed herein are carried out in a proper solvent that must be suitable for the chemical changes in the present application and the reagents and materials required. In order to acquire the compounds disclosed herein, it is sometimes necessary for those skilled in the art to modify or select a synthesis procedure or a reaction scheme based on the existing embodiments.

An important consideration in synthesis route planning in the art is the selection of suitable protecting groups for reactive functional groups (e.g., amino in the present application). For example, reference may be made to Greene's Protective Groups in Organic Synthesis (4th Ed.) Hoboken, New Jersey: John Wiley & Sons, Inc. All references cited herein are incorporated by reference in their entirety. In some embodiments, the compound of formula (I) disclosed herein may be prepared by one skilled in the art of organic synthesis using standard methods in the art by the following routes:

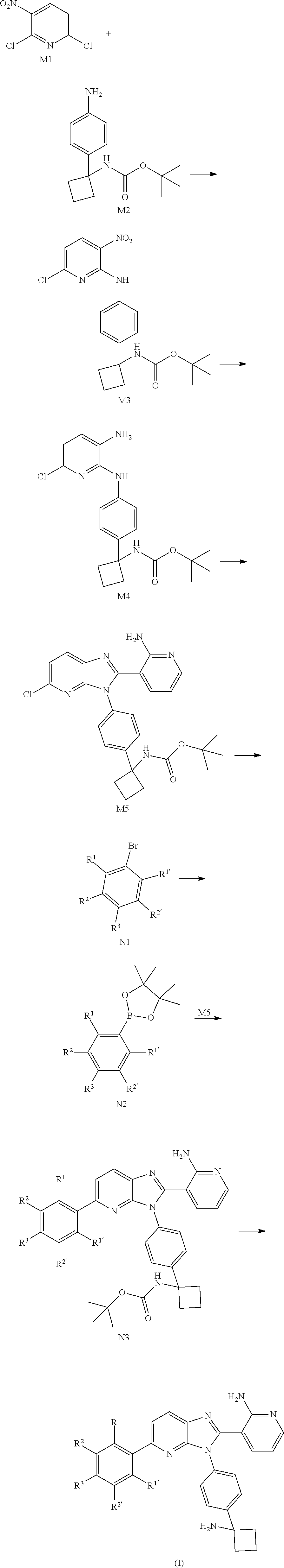

wherein,

R¹, R^{1′}, R², R^{2′} and R³ are defined as above.

### DETAILED DESCRIPTION

For clarity, the present application is further described with the following examples, which are, however, not intended to limit the scope of the invention. It will be apparent to those skilled in the art that various changes and modifications can be made to the specific examples of the present invention without departing from the spirit and scope of the present invention. All reagents are commercially available and can be used without further purification.

### Example 1: Preparation of Compound 1

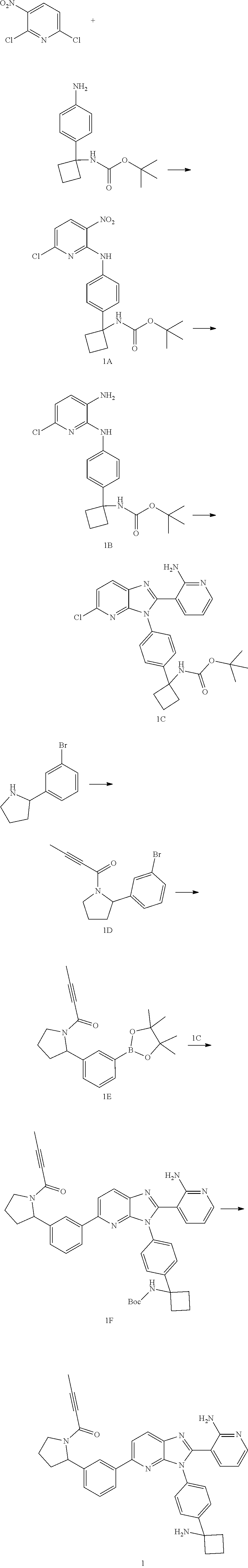

### Step A: Preparation of Compound 1A

N,N-diisopropylethylamine (13.4 g) was added slowly dropwise to a solution of 2,6-dichloro-3-nitropyridine (20.0 g) and tert-butyl (1-(4-aminophenyl)cyclobutyl)carbamate (25.5 g) in tetrahydrofuran at −30° C. under nitrogen atmosphere. After the addition was completed, the reaction solution was stirred at room temperature. After the reaction was completed, the reaction solution was concentrated to give compound 1A (43.4 g).

MS (ESI, [M+Na]⁺) m/z: 441.5.

### Step B: Preparation of Compound 1B

To a reaction flask were added compound 1A (40.0 g), zinc powder (31.2 g), ammonium chloride (5.1 g), ethanol (400 mL) and water (40 mL) successively at room temperature. After the addition was completed, the reaction solution was reacted at 90° C. After the reaction was completed, the reaction solution was filtered, the filtrate was concentrated, and the residue was dissolved in dichloromethane, washed with water, dried over anhydrous sodium sulfate, filtered, and concentrated to give compound 1B (41.0 g).

MS (ESI, [M+H]⁺) m/z: 389.3.

### Step C: Preparation of Compound 1C

To a reaction flask were added compound 1B (34.0 g), 2-aminonicotinaldehyde (11.2 g), sodium perborate (14.3 g), acetic acid (250 mL) and methanol (30 mL) successively at room temperature. After the addition was completed, the reaction solution was stirred at 55° C. After the reaction was completed, the reaction solution was concentrated, and added with ethyl acetate and water; then the pH of the reaction solution was adjusted to 11-12 with an aqueous sodium hydroxide solution. The reaction solution was separated, and the organic phase was dried over anhydrous sodium sulfate, filtered, concentrated, and subjected to column chromatography (dichloromethane/methanol=50/1) to give compound 1C (16.0 g).

MS (ESI, [M+H]⁺) m/z: 491.3.

### Step D: Preparation of Compound 1D

2-butynoic acid (1.00 g), 2-(7-azabenzotriazole)-0′-tetramethyluronium hexafluorophosphate (4.97 g) and dichloromethane (50 mL) were added to a reaction flask successively, and a solution of N,N-diisopropylethylamine (3.07 g) and 2-(3-bromophenyl)-pyrrolidine (2.96 g) in dichloromethane (30 mL) was slowly added dropwise to the reaction flask at 0° C. After the addition was completed, the reaction solution was stirred for reaction at room temperature. After the reaction was completed, a saturated aqueous ammonium chloride solution was added to the reaction solution, the phases were separated, the aqueous phase was extracted with dichloromethane, the organic phases were combined, washed with saturated brine, and dried over anhydrous sodium sulfate. The organic phase was filtered, concentrated, and subjected to column chromatography (petroleum ether/ethyl acetate=1/1) to give compound 1D (3.92 g).

MS (ESI, [M+H]⁺) m/z: 292.0.

### Step E: Preparation of Compound 1E

To a reaction flask were added compound 1D (0.92 g), bis(pinacolato)diboron (1.20 g), potassium acetate (0.93 g), [1,1′-bis(diphenylphosphino)ferrocene]palladium dichloride dichloromethane complex (0.13 g) and 1,4-dioxane (15 mL) successively. The reaction solution was stirred for reaction at 100° C. under nitrogen atmosphere. After the reaction was completed, the reaction solution was filtered and concentrated. The reaction solution was subjected to column chromatography (petroleum ether/ethyl acetate=2/1) to give compound 1E (0.38 g).

MS (ESI, [M+H]⁺) m/z: 340.2.

### Step F: Preparation of Compound 1F

To a microwave tube were added compound 1E (0.17 g), compound 1C (0.12 g), potassium carbonate (0.11 g), tetrakis(triphenylphosphine)palladium (0.03 g), 1,4-dioxane (3 mL) and water (0.5 mL) successively. After the addition was completed, the reaction solution was purged with nitrogen gas, and reacted at 100° C. in microwave reactor for 2 h. After the reaction was completed, the reaction solution was filtered and concentrated. The reaction solution was subjected to column chromatography (dichloromethane/methanol=20/1) to give compound 1F (68 mg).

MS (ESI, [M+Na]⁺) m/z: 690.6.

### Step G: Preparation of Compound 1

Compound 1F (68 mg), methanesulfonic acid (0.1 mL) and dichloromethane (4 mL) were added successively to a reaction flask, and the mixture was stirred at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 1 (18 mg).

HRMS (ESI, [M+H]⁺) m/z: 568.2823.

¹H NMR (500 MHz, DMSO-d₆) δ 8.28 (d, J=8.3 Hz, 1H), 8.05-7.95 (m, 2H), 7.94-7.79 (m, 2H), 7.71-7.63 (m, 2H), 7.58-7.50 (m, 2H), 7.46-7.38 (m, 1H), 7.31-7.16 (m, 2H), 6.93 (d, J=8.5 Hz, 2H), 6.46-6.42 (m, 1H), 5.24-5.02 (m, 1H), 3.83 (t, J=6.9 Hz, 1H), 3.65-3.54 (m, 1H), 2.62-2.52 (m, 2H), 2.45-2.30 (m, 3H), 2.18-2.09 (m, 1H), 1.94-1.73 (m, 4H), 1.87 (s, 3H).

### Example 2: Preparation of Compound 2

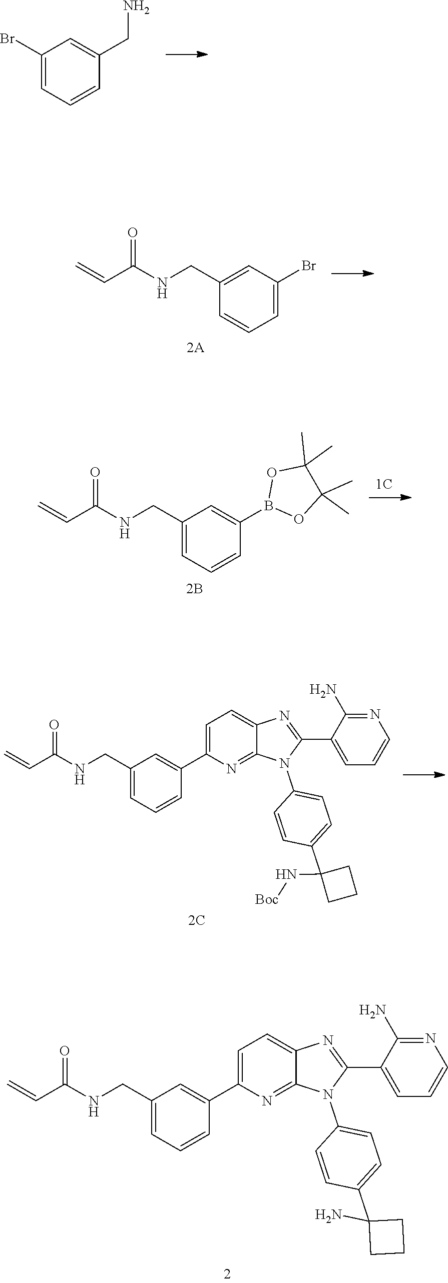

### Step A: Preparation of Compound 2A

A solution of acryloyl chloride (1.08 g) in tetrahydrofuran (10 mL) was slowly added to a reaction solution of 3-bromobenzylamine (2.00 g) and N,N-diisopropylethylamine (1.63 g) in tetrahydrofuran (90 mL) in a reaction flask at −10° C. under nitrogen atmosphere. After the addition was completed, the reaction solution was stirred at room temperature until the reaction was completed. The reaction solution was added with a saturated aqueous ammonium chloride solution and extracted with ethyl acetate; the organic phases were combined, washed with saturated brine, and dried over anhydrous sodium sulfate. The reaction solution was filtered and concentrated to give compound 2A (2.58 g).

MS (ESI, [M+H]⁺) m/z: 240.1.

### Step B: Preparation of Compound 2B

To a reaction flask were added compound 2A (2.00 g), bis(pinacolato)diboron (3.17 g), potassium acetate (2.45 g), [1,1′-bis(diphenylphosphino)ferrocene]palladium dichloride dichloromethane complex (0.68 g) and 1,4-dioxane (30 mL) successively. The reaction solution was stirred at 100° C. under nitrogen atmosphere. After the reaction was completed, the reaction solution was filtered and concentrated. The reaction solution was subjected to column chromatography (petroleum ether/ethyl acetate=1/1) to give compound 2B (1.89 g).

MS (ESI, [M+H]⁺) m/z: 288.1.

### Step C: Preparation of Compound 2C

To a microwave tube were added compound 2B (0.14 g), compound 1C (0.12 g), potassium carbonate (0.11 g), tetrakis(triphenylphosphine)palladium (0.028 g), 1,4-dioxane (3 mL) and water (0.5 mL) successively. After the addition was completed, the reaction solution was purged with nitrogen gas, and reacted at 100° C. in microwave reactor. After the reaction was completed, the reaction solution was filtered and concentrated. The reaction solution was subjected to column chromatography (dichloromethane/methanol=20/1) to give compound 2C (85 mg).

MS (ESI, [M+H]⁺) m/z: 616.5.

¹H NMR (500 MHz, DMSO-d₆) δ 8.28 (d, J=8.3 Hz, 1H), 8.05-7.95 (m, 2H), 7.94-7.79 (m, 2H), 7.71-7.63 (m, 2H), 7.58-7.50 (m, 2H), 7.42 (dt, J=26.0, 7.7 Hz, 1H), 7.31-7.16 (m, 2H), 6.93 (d, J=8.5 Hz, 2H), 6.46-6.42 (m, 1H), 5.24-5.02 (m, 1H), 3.83 (t, J=6.9 Hz, 1H), 3.65-3.54 (m, 1H), 2.62-2.52 (m, 2H), 2.45-2.30 (m, 3H), 2.18-2.09 (m, 1H), 1.94-1.73 (m, 4H), 1.87 (s, 3H).

### Step D: Preparation of Compound 2

Compound 2C (80 mg), methanesulfonic acid (0.1 mL) and dichloromethane (4 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 2 (45 mg).

HRMS (ESI, [M+H]⁺) m/z: 516.2516.

¹H NMR (500 MHz, DMSO-d₆) δ 8.67 (t, J=6.0 Hz, 1H), 8.27 (d, J=8.4 Hz, 1H), 8.01 (dd, J=4.8, 1.8 Hz, 1H), 7.98-7.88 (m, 3H), 7.67-7.59 (m, 2H), 7.50-7.40 (m, 3H), 7.30 (dt, J=7.6, 1.3 Hz, 1H), 7.21 (dd, J=7.6, 1.9 Hz, 1H), 6.94 (s, 2H), 6.42 (dd, J=7.7, 4.9 Hz, 1H), 6.31 (dd, J=17.1, 10.2 Hz, 1H), 6.15 (dd, J=17.1, 2.2 Hz, 1H), 5.65 (dd, J=10.2, 2.2 Hz, 1H), 4.43 (d, J=5.9 Hz, 2H), 2.49-2.43 (m, 2H), 2.23-2.13 (m, 2H), 2.11-2.00 (m, 1H), 1.77-1.69 (m, 1H).

### Example 3: Preparation of Compound 3

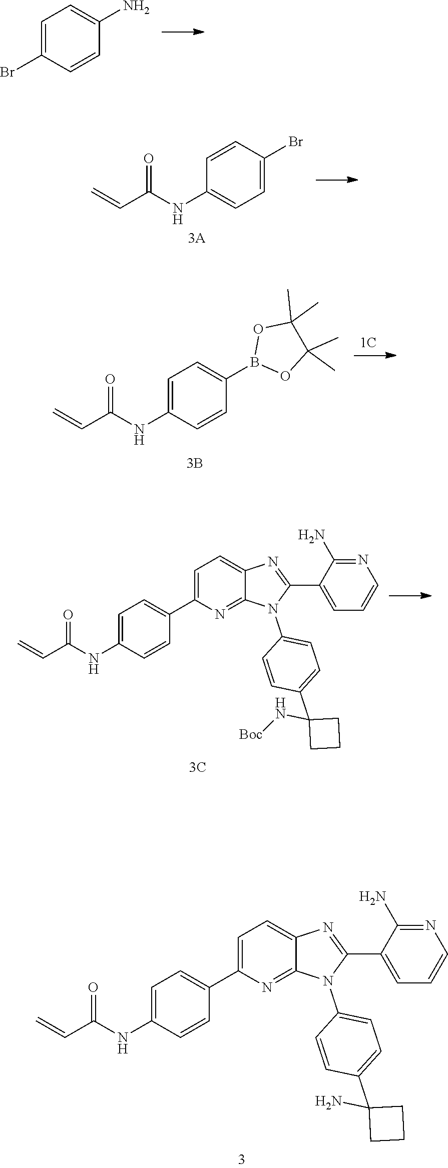

### Step A: Preparation of Compound 3A

A solution of acryloyl chloride (2.98 g) in tetrahydrofuran (10 mL) was slowly added to a stirred solution of p-bromophenylamine (5.00 g) and N,N-diisopropylethylamine (4.51 g) in tetrahydrofuran (90 mL) in a reaction flask at −10° C. under nitrogen atmosphere. After the addition was completed, the reaction solution was stirred at room temperature until the reaction was completed. The reaction solution was added with a saturated aqueous ammonium chloride solution and extracted with ethyl acetate; the organic phases were combined, washed with saturated brine, and dried over anhydrous sodium sulfate. The reaction solution was filtered and concentrated to give compound 3A (5.90 g).

MS (ESI, [M+H]⁺) m/z: 226.1.

### Step B: Preparation of Compound 3B

To a reaction flask were added compound 3A (0.5 g), bis(pinacolato)diboron (0.84 g), potassium acetate (0.65 g), [1,1′-bis(diphenylphosphino)ferrocene]palladium dichloride dichloromethane complex (0.18 g) and 1,4-dioxane (20 mL) successively. The reaction solution was stirred at 100° C. under nitrogen atmosphere. After the reaction was completed, the reaction solution was filtered and concentrated. The reaction solution was subjected to column chromatography (petroleum ether/ethyl acetate=2/1) to give compound 3B (0.26 g).

MS (ESI, [M+H]⁺) m/z: 274.5.

### Step C: Preparation of Compound 3C

To a microwave tube were added compound 3B (0.13 g), compound 1C (0.12 g), potassium carbonate (0.11 g), tetrakis(triphenylphosphine)palladium (0.028 g), 1,4-dioxane (3 mL) and water (0.5 mL) successively. After the addition was completed, the reaction solution was purged with nitrogen gas, and reacted at 100° C. in microwave reactor for 2 h. After the reaction was completed, the reaction solution was filtered and concentrated. The reaction solution was subjected to column chromatography (dichloromethane/methanol=20/1) to give compound 3C (78 mg).

MS (ESI, [M+H]⁺) m/z: 602.4.

### Step D: Preparation of Compound 3

Compound 3C (70 mg), methanesulfonic acid (0.1 mL) and dichloromethane (4 mL) were added successively to a reaction flask, and the mixture was stirred at room temperature. After the reaction was completed, 10% sodium hydroxide solution was added to the reaction solution to adjust the pH to 13, dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 3 (34 mg).

HRMS (ESI, [M+H]⁺) m/z: 502.2343.

¹H NMR (500 MHz, DMSO-d₆) δ 10.26 (s, 1H), 8.17 (d, J=8.3 Hz, 1H), 7.99-7.91 (m, 3H), 7.88 (d, J=8.4 Hz, 1H), 7.70 (d, J=8.5 Hz, 2H), 7.57 (d, J=8.4 Hz, 2H), 7.39 (d, J=8.4 Hz, 2H), 7.12 (dd, J=7.7, 1.8 Hz, 1H), 6.90 (s, 2H), 6.45-6.30 (m, 2H), 6.21 (dd, J=16.9, 2.0 Hz, 1H), 5.70 (dd, J=10.0, 2.0 Hz, 1H), 2.42-2.36 (m, 2H), 2.15-2.07 (m, 2H), 2.05-1.95 (m, 1H), 1.71-1.62 (m, 1H).

### Example 4: Preparation of Compound 4

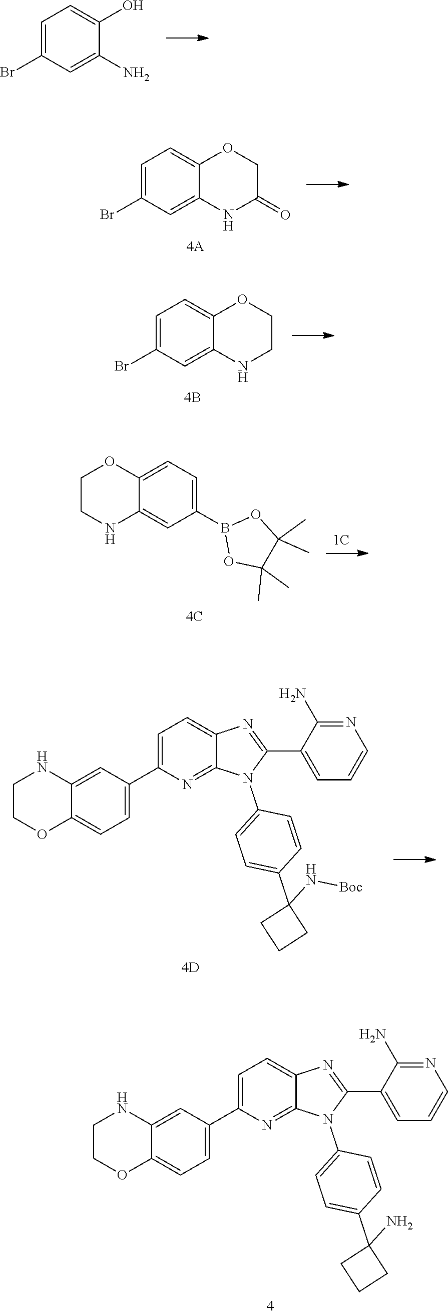 [SMILES: Nc1cc(Br)ccc1O ; O=C1COc2ccc(Br)cc2N1 ; Brc1ccc2c(c1)NCCO2 ; CC1(C)OB(c2ccc3c(c2)NCCO3)OC1(C)C ; CC(C)(C)OC(=O)NC1(c2ccc(-n3c(-c4cccnc4N)nc4ccc(-c5ccc6c(c5)NCCO6)nc43)cc2)CCC1 ; Nc1ncccc1-c1nc2ccc(-c3ccc4c(c3)NCCO4)nc2n1-c1ccc(C2(N)CCC2)cc1]

### Step A: Preparation of Compound 4A

2-Chloroacetyl chloride (3.24 g) was added dropwise to a reaction solution of 2-amino-4-bromophenol (3.60 g) and sodium carbonate (3.22 g) in tetrahydrofuran (60 mL) in an ice-water bath under nitrogen atmosphere. After the addition was completed, the reaction solution was stirred at room temperature. After the reaction was completed, the reaction solution was concentrated and subjected to column chromatography (petroleum ether/ethyl acetate=3/1) to give compound 4A (3.60 g).

MS (ESI, [M−H]⁻) m/z: 226.1; MS (ESI, [M+H]⁺) m/z: 228.0.

### Step B: Preparation of Compound 4B

A solution of 1 M borane in tetrahydrofuran (516 mg) was added slowly to a solution of compound 4A (452 mg) in tetrahydrofuran (10 mL) in an ice-water bath under nitrogen atmosphere. After the addition was completed, the reaction solution was stirred for reaction at 70° C. After the reaction was completed, the reaction was quenched with saturated sodium bicarbonate, and the reaction solution was extracted with ethyl acetate, and the organic phases were combined, washed with saturated brine, dried over anhydrous sodium sulfate, filtered, concentrated, and subjected to column chromatography (petroleum ether/ethyl acetate=4/1) to give compound 4B (380 mg).

MS (ESI, [M+H]⁺) m/z: 214.3.

### Step C: Preparation of Compound 4C

To a reaction flask were added [1,1′-bis(diphenylphosphino)ferrocene]palladium dichloride (123 mg), compound 4B (320 mg), bis(pinacolato)diboron (508 mg), potassium carbonate (294 mg) and dioxane (5 mL) successively. The reaction solution was reacted at 100° C. After the reaction was completed, the reaction solution was concentrated and subjected to column chromatography (petroleum ether/ethyl acetate=3/1) to give compound 4C (242 mg).

MS (ESI, [M+H]⁺) m/z: 262.4.

### Step D: Preparation of Compound 4D

To a reaction flask were added compound 4C (200 mg), potassium carbonate (270 mg), tetrakis(triphenylphosphine)palladium (47 mg), compound 1C (320 mg), water (0.5 mL), and dioxane (5 mL) successively. After the addition was completed, the reaction solution was reacted at 110° C. After the reaction was completed, the reaction solution was concentrated and subjected to column chromatography (dichloromethane/methanol=98/2) to give compound 4D (260 mg).

### Step E: Preparation of Compound 4

To the reaction flask were added compound 4D (260 mg), methanesulfonic acid (0.5 mL), and dichloromethane (5 mL) successively. After the addition was completed, the reaction solution was stirred at room temperature. After the reaction was completed, the pH of the reaction solution was adjusted to 7 to 9 with saturated aqueous sodium bicarbonate, the reaction solution was extracted with dichloromethane, the organic phases were combined, washed with saturated brine, dried over anhydrous sodium sulfate, filtered, concentrated, and subjected to column chromatography (dichloromethane/methanol=95/5) to give compound 4 (135 mg).

HRMS (ESI, [M+H]⁺) m/z: 490.2360.

¹H NMR (500 MHz, DMSO-d₆): δ 8.16 (d, J=5.0 Hz, 1H), 7.99 (dd, J=5.0 Hz, 10.0 Hz, 1H), 7.75 (d, J=10.0 Hz, 1H), 7.61 (d, J=10.0 Hz, 1H), 7.41 (d, J=10.0 Hz, 1H), 7.23 (d, J=5.0 Hz, 1H), 7.16-7.14 (m, 2H), 6.94 (s, 2H), 6.71 (d, J=5.0 Hz, 1H), 6.39 (q, J=5.0 Hz, 1H), 5.88 (s, 1H), 4.14 (t, J=5.0 Hz, 2H), 3.30 (d, J=10.0 Hz, 2H), 2.46-2.14 (m, 2H), 2.13 (br, 2H), 2.12-2.03 (m, 3H), 1.78-1.72 (m, 1H).

### Example 5: Preparation of Compound 5

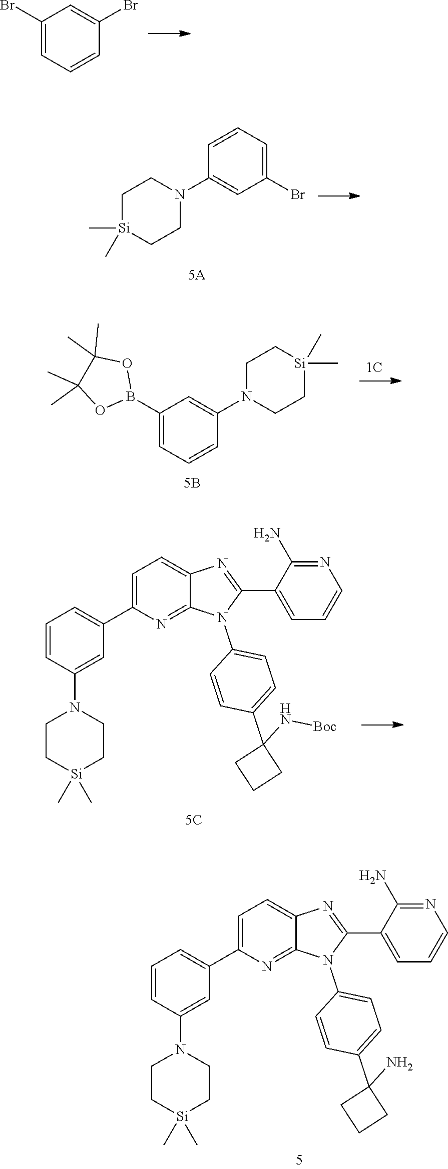

### Step A: Preparation of Compound 5A

To a reaction flask were added 1,1′-binaphthyl-2,2′-diphenyl phosphine (279 mg), tris(dibenzylideneacetone)dipalladium (274 mg), 1,3-dibromobenzene (705 mg), 4,4-dimethyl-1,4-silapiperidine (386 mg), sodium tert-butoxide (431 mg) and toluene (10 mL) successively under nitrogen atmosphere. After the addition was completed, the reaction solution was reacted at 115° C. After the reaction was completed, the reaction solution was concentrated and subjected to column chromatography (petroleum ether/ethyl acetate=95/5) to give compound 5A (475 mg). The compound was directly used in the next step without purification.

### Step B: Preparation of Compound 5B

To a reaction flask were added [1,1′-bis(diphenylphosphino)ferrocene]palladium dichloride (63.9 mg), compound 5A (475 mg), bis(pinacolato)diboron (795 mg), potassium acetate (461 mg) and dioxane (8 mL) successively under nitrogen atmosphere. After the addition was completed, the reaction solution was reacted at 110° C. After the reaction was completed, the reaction solution was concentrated and subjected to column chromatography (petroleum ether/ethyl acetate=9/1) to give compound 5B (450 mg).

MS (ESI, [M+H]⁺) m/z: 332.5.

### Step C: Preparation of Compound 5C

To a reaction flask were added compound 5B (311 mg), potassium carbonate (287 mg), compound 1C (340 mg), water (1 mL), and dioxane (10 mL) successively under nitrogen atmosphere. After the addition was completed, the reaction solution was reacted at 110° C. After the reaction was completed, the reaction solution was concentrated and subjected to column chromatography (dichloromethane/methanol=96/4) to give compound 5C (280 mg).

MS (ESI, [M+H]⁺) m/z: 660.7.

### Step D: Preparation of Compound 5

In a reaction flask, methanesulfonic acid (200 mg) was slowly added to a solution of compound 5C (280 mg) in dichloromethane (5 mL). After the addition was completed, the reaction solution was reacted at room temperature. After the reaction was completed, the pH of the reaction solution was adjusted to 7 to 9 with saturated aqueous sodium bicarbonate, the reaction solution was extracted with dichloromethane, the organic phases were combined, washed with saturated brine, dried over anhydrous sodium sulfate, filtered, concentrated, and subjected to column chromatography (dichloromethane/methanol=95/5) to give compound 5 (120 mg).

MS (ESI, [M+H]⁺) m/z: 560.6.

¹H NMR (500 MHz, DMSO-d₆): δ 8.16 (d, J=10.0 Hz, 1H), 7.96-7.95 (m, 1H), 7.85 (d, J=5.0 Hz, 1H), 7.54 (d, J=10.0 Hz, 1H), 7.47 (s, 1H), 7.38 (d, J=5.0 Hz, 1H), 7.21-7.17 (m, 3H), 6.97 (br, 2H), 6.82 (q, J=5.0 Hz, 1H), 6.37 (q, J=5.0 Hz, 1H), 3.63-3.61 (m, 4H), 2.38-2.32 (m, 2H), 2.17 (br, 2H), 2.09-2.03 (m, 2H), 2.00-1.93 (m, 1H), 1.64-1.61 (m, 1H), 0.67-0.65 (m, 4H), 0.001 (s, 6H).

### Example 6: Preparation of Compound 6

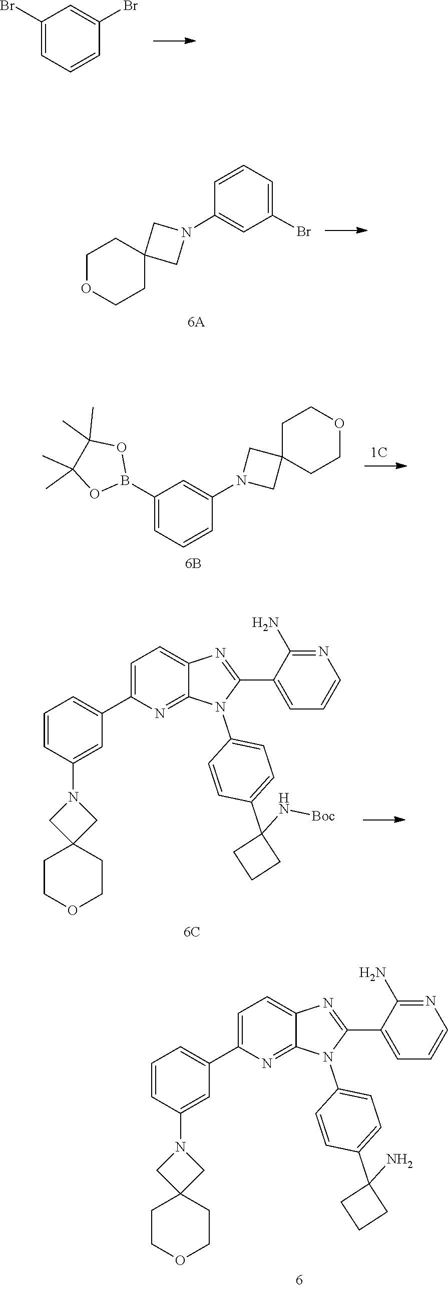 [SMILES: Brc1cccc(Br)c1 ; Brc1cccc(N2CC3(CCOCC3)C2)c1 ; CC1(C)OB(c2cccc(N3CC4(CCOCC4)C3)c2)OC1(C)C ; CC(C)(C)OC(=O)NC1(c2ccc(-n3c(-c4cccnc4N)nc4ccc(-c5cccc(N6CC7(CCOCC7)C6)c5)nc43)cc2)CCC1 ; Nc1ncccc1-c1nc2ccc(-c3cccc(N4CC5(CCOCC5)C4)c3)nc2n1-c1ccc(C2(N)CCC2)cc1]

### Step A: Preparation of Compound 6A

To a reaction flask were added 1,1′-binaphthyl-2,2′-diphenyl phosphine (0.475 g), tris(dibenzylideneacetone)dipalladium (0.466 g), 1,3-dibromobenzene (1.200 g), 7-oxa-2-azaspiro[3.5]nonane (0.647 g), sodium tert-butoxide (0.733 g) and toluene (10 mL) successively under nitrogen atmosphere. After the addition was completed, the reaction solution was reacted at 115° C. After the reaction was completed, the reaction solution was concentrated and subjected to column chromatography (petroleum ether/ethyl acetate=85/15) to give compound 6A (780 mg).

### Step B: Preparation of Compound 6B

To a reaction flask were added [1,1′-bis(diphenylphosphino)ferrocene]palladium dichloride (339 mg), compound 6A (780 mg), bis(pinacolato)diboron (1404 mg), potassium acetate (814 mg) and dioxane (8 mL) successively under nitrogen atmosphere. After the addition was completed, the reaction solution was reacted at 110° C. After the reaction was completed, the reaction solution was concentrated and subjected to column chromatography (petroleum ether/ethyl acetate=9/1) to give compound 6B (520 mg).

MS (ESI, [M+H]⁺) m/z: 330.5.

### Step C: Preparation of Compound 6C

To a reaction flask were added compound 6B (520 mg), potassium carbonate (220 mg), tetratriphenylphosphine (36 mg), compound 1C (260 mg), water (0.50 mL), and dioxane (10 mL) successively under nitrogen atmosphere. After the addition was completed, the reaction solution was reacted at 110° C. After the reaction was completed, the reaction solution was concentrated and subjected to column chromatography (dichloromethane/methanol=98/2) to give compound 6C (240 mg).

MS (ESI, [M+H]⁺) m/z: 658.7.

### Step D: Preparation of Compound 6

To a reaction flask were added methanesulfonic acid (331 mg), compound 6C (240 mg), and dichloromethane (5 mL) successively, and after the addition was completed, the mixture was stirred at room temperature. After the reaction was completed, the pH of the reaction solution was adjusted to 7 to 9 with saturated aqueous sodium bicarbonate, the reaction solution was extracted with dichloromethane, the organic phases were combined, washed with saturated brine, dried over anhydrous sodium sulfate, filtered, concentrated, and subjected to column chromatography (dichloromethane/methanol=95/5) to give compound 6 (140 mg).

MS (ESI, [M+H]⁺) m/z: 558.6.

¹H NMR (500 MHz, DMSO-d₆): δ 8.45 (d, J=5.0 Hz, 1H), 8.22 (t, J=5.0 Hz, 2H), 8.11-8.10 (m, 1H), 8.08 (d, J=10.0 Hz, 1H), 8.01 (dd, J=5.0 Hz, 10.0 Hz, 1H), 7.82-7.78 (m, 2H), 7.72-7.70 (m, 2H), 7.65 (t, J=10.0 Hz, 1H), 7.55 (dd, J=5.0 Hz, 10.0 Hz, 1H), 6.98 (t, J=10.0 Hz, 1H), 4.34 (s, 4H), 3.59-3.57 (m, 4H), 2.66 (t, J=5.0 Hz, 4H), 2.55 (t, J=5.0 Hz, 4H), 1.97 (t, J=5.0 Hz, 4H).

### Example 7: Preparation of Compound 7

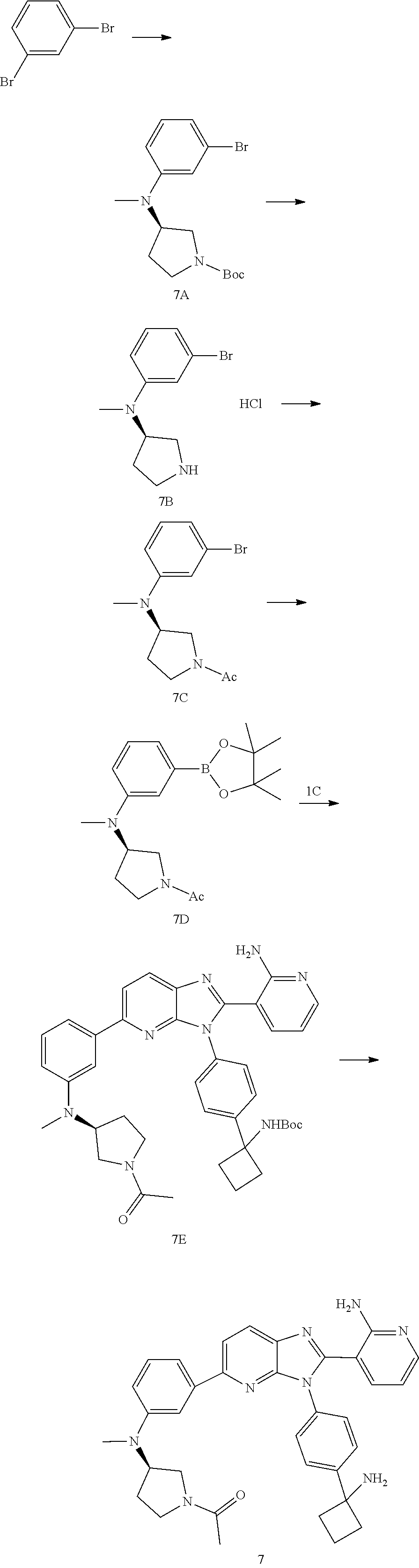

### Step A: Preparation of Compound 7A

1,1′-binaphthyl-2,2′-diphenyl phosphine (367 mg), bis(dibenzylideneacetone)palladium (270 mg), 1,3-dibromobenzene (695 mg), tert-butyl (R)-3-(methylamine)pyrrole-1-carboxylate (590 mg), sodium tert-butoxide (566 mg) and dioxane (15 mL) were successively added to a reaction flask under nitrogen atmosphere. After the addition was completed, the reaction solution was reacted at 120° C. After the reaction was completed, the reaction solution was concentrated and subjected to column chromatography (petroleum ether/ethyl acetate=95/5) to give compound 7A (642 mg).

MS (ESI, [M-55+H]⁺) m/z: 299.1.

### Step B: Preparation of Compound 7B

To a reaction flask were added compound 7A (640 mg), dichloromethane (5 mL) and a solution of 4 M hydrogen chloride in dioxane (3 mL) successively under nitrogen atmosphere. After the addition was completed, the reaction solution was stirred at room temperature until the reaction was completed, and the solvent was removed by distillation under the reduced pressure to give compound 7B (680 mg).

### Step C: Preparation of Compound 7C

To a reaction flask were added compound 7B (680 mg), tetrahydrofuran (20 mL), N-diisopropylethylamine (581 mg), and acetyl chloride (300 mg) separately. After the addition was completed, the reaction solution was reacted at room temperature. After the reaction was completed, the reaction solution was concentrated and subjected to column chromatography (dichloromethane/methanol=20/1) to give compound 7C (430 mg).

MS (ESI, [M+H]⁺) m/z: 297.4.

### Step D: Preparation of Compound 7D

To a reaction flask were added [1,1′-bis(diphenylphosphino)ferrocene]palladium dichloride (154 mg), compound 7C (360 mg), bis(pinacolato)diboron (478 mg), potassium acetate (370 mg) and dioxane (8 mL) successively under nitrogen atmosphere. After the addition was completed, the reaction solution was reacted at 110° C. The reaction was completed, the reaction was quenched by saturated brine, the reaction solution was extracted by ethyl acetate, organic phases were combined, the reaction solution was washed with saturated brine and dried over anhydrous sodium sulfate. The organic phase was filtered, concentrated, and subjected to column chromatography (petroleum ether/ethyl acetate=90/10) to give compound 7D (440 mg).

MS (ESI, [M+H]⁺) m/z: 345.6.

### Step E: Preparation of Compound 7E

To a reaction flask were added tetrakis(triphenylphosphine)palladium (99 mg), compound 1C (420 mg), compound 7D (294 mg), potassium carbonate (236 mg), dioxane (5 mL), and water (0.5 mL) successively. After the addition was completed, the reaction solution was heated and reacted at 120° C. After the starting materials were reacted completely, the reaction solution was concentrated and subjected to column chromatography (dichloromethane/methanol=98/2) to give compound 7E (540 mg).

MS (ESI, [M+H]⁺) m/z: 673.7.

### Step F: Preparation of Compound 7

To the reaction flask were added compound 7E (490 mg), dichloromethane (6 mL) and methanesulfonic acid (420 mg) successively. After the addition was completed, the reaction solution was reacted at room temperature. After the reaction was completed, the pH of the reaction solution was adjusted to be neutral by using sodium bicarbonate, the reaction solution was extracted with dichloromethane, and the organic phase were combined, washed with saturated brine and dried over anhydrous sodium sulfate. The reaction solution was filtered, concentrated, and subjected to column chromatography (dichloromethane/methanol=96/4) to give compound 7 (30 mg).

MS (ESI, [M+H]⁺) m/z: 573.4.

¹H NMR (500 MHz, DMSO-d₆): δ 8.23 (d, J=3.5 Hz, 1H), 8.01-7.96 (m, 2H), 7.62-7.60 (m, 2H), 7.55 (d, J=9.0 Hz, 1H), 7.45 (d, J=6.5 Hz, 1H), 7.29-7.22 (m, 1H), 7.01 (d, J=7.5 Hz, 1H), 6.92 (d, J=7.5 Hz, 1H), 6.42 (t, J=6.5 Hz, 1H), 4.58-4.47 (m, 1H), 3.69-3.40 (m, 3H), 3.28-3.25 (m, 2H), 2.83-2.80 (m, 3H), 2.50 (s, 3H), 2.15-2.08 (m, 3H), 2.06-2.00 (m, 2H), 1.96-1.85 (m, 3H).

### Example 8: Preparation of Compound 8

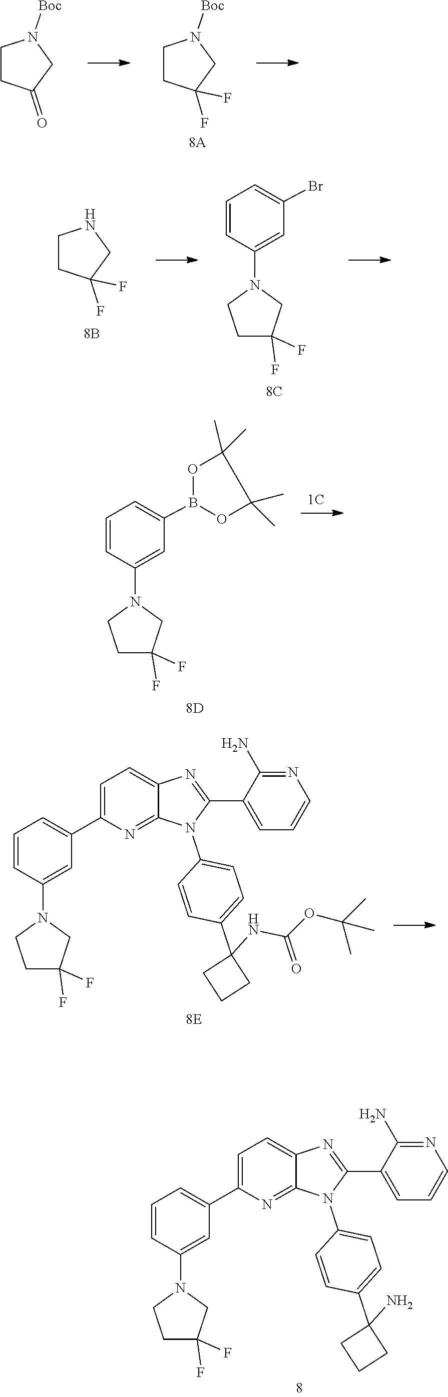

### Step A: Preparation of Compound 8A

A solution of N,N-diethyl-1,1,1-trifluoro-14-thiamine (20.890 g) in 1,2-dichloroethane (25 mL) was slowly added dropwise to a reaction flask containing tert-butyl 3-oxopyrrolidine-1-carboxylate (4.000 g) under nitrogen atmosphere. After the addition was completed, the reaction solution was stirred at room temperature. After the reaction was completed, the reaction solution was concentrated and subjected to column chromatography (petroleum ether/ethyl acetate=4/1) to give compound 8A (2.510 g).

MS (ESI, [M+H]⁺) m/z: 208.0.

### Step B: Preparation of Compound 8B

To a reaction flask were added compound 8A (1.000 g), a solution of 1,4-dioxane (2 mL), and a solution of 4 M hydrochloric acid in 1,4-dioxane (9.50 mL) successively. After the addition was completed, the reaction solution was stirred for reaction at room temperature. After the reaction was completed, the reaction solution was concentrated to give compound 8B (0.512 g).

### Step C: Preparation of Compound 8C

To a reaction flask were added sodium tert-butoxide (0.928 g), tris(dibenzylideneacetone)dipalladium (0.442 g), 3,3-difluoropyrrolidine (0.517 g), 1,3-dibromobenzene (2.279 g), 1,1′-binaphthyl-2,2′-diphenyl phosphine (0.451 g) and toluene (30 mL) successively under nitrogen atmosphere. After the addition was completed, the reaction solution was stirred at 110° C. After the reaction was completed, the reaction solution was concentrated and subjected to column chromatography (petroleum ether/ethyl acetate=95/5) to give compound 8C (1.199 g).

### Step D: Preparation of Compound 8D

To a reaction flask were added compound 8C (1.150 g), potassium carbonate (1.227 g), bis(pinacolato)diboron (1.587 g), [1,1′-bis(diphenylphosphino)ferrocene]palladium dichloride (0.170 g), 1,1′-binaphthyl-2,2′-diphenyl phosphine (0.451 g), and N,N-dimethylformamide (30 mL) successively under nitrogen atmosphere. After the addition was completed, the reaction solution was stirred at 110° C. After the reaction was completed, the reaction solution was concentrated and subjected to column chromatography (petroleum ether/ethyl acetate=90/10) to give compound 8D (0.779 g).

MS (ESI, [M+H]⁺) m/z: 310.2.

### Step E: Preparation of Compound 8E

To a reaction flask were added compound 8D (0.307 g), potassium carbonate (0.115 g), compound 1C (0.210 g), tetrakis(triphenylphosphine)palladium (0.048 g), 1,4-dioxane (16.00 mL), and water (4.00 mL) successively under nitrogen atmosphere. After the addition was completed, the reaction solution was stirred for reaction at 140° C. After the reaction was completed, the reaction solution was concentrated and subjected to column chromatography (dichloromethane/methanol=98/3) to give compound 8E (0.140 g).

MS (ESI, [M+H]⁺) m/z: 638.4.

### Step F: Preparation of Compound 8

To a reaction flask were added compound 8E (0.140 g), dichloromethane (10 mL) and methanesulfonic acid (0.160 g) successively. After the addition was completed, the reaction solution was stirred for reaction at room temperature. After the reaction was completed, the reaction solution was concentrated and subjected to column chromatography (dichloromethane/methanol=93/7) to give compound 8 (98 mg).

MS (ESI, [M+H]⁺) m/z: 538.6.

¹H NMR (500 MHz, DMSO-d₆) δ 8.24 (d, J=8.4 Hz, 1H), 8.00 (t, J=7.6 Hz, 2H), 7.64 (d, J=8.0 Hz, 2H), 7.46 (d, J=8.0 Hz, 2H), 7.40 (d, J=7.7 Hz, 1H), 7.27 (dq, J=13.3, 7.8 Hz, 3H), 7.02 (s, 2H), 6.66 (d, J=8.1 Hz, 1H), 6.43 (dd, J=7.7, 4.7 Hz, 1H), 3.74 (t, J=13.3 Hz, 2H), 3.53 (t, J=7.2 Hz, 2H), 2.55 (dd, J=1.6, 10.3 Hz, 2H), 2.44 (t, J=8.6 Hz, 2H), 2.24-2.10 (m, 2H), 2.11-2.00 (m, 1H), 1.72 (dd, J=11.4, 6.5 Hz, 1H).

### Example 9: Preparation of Compound 9

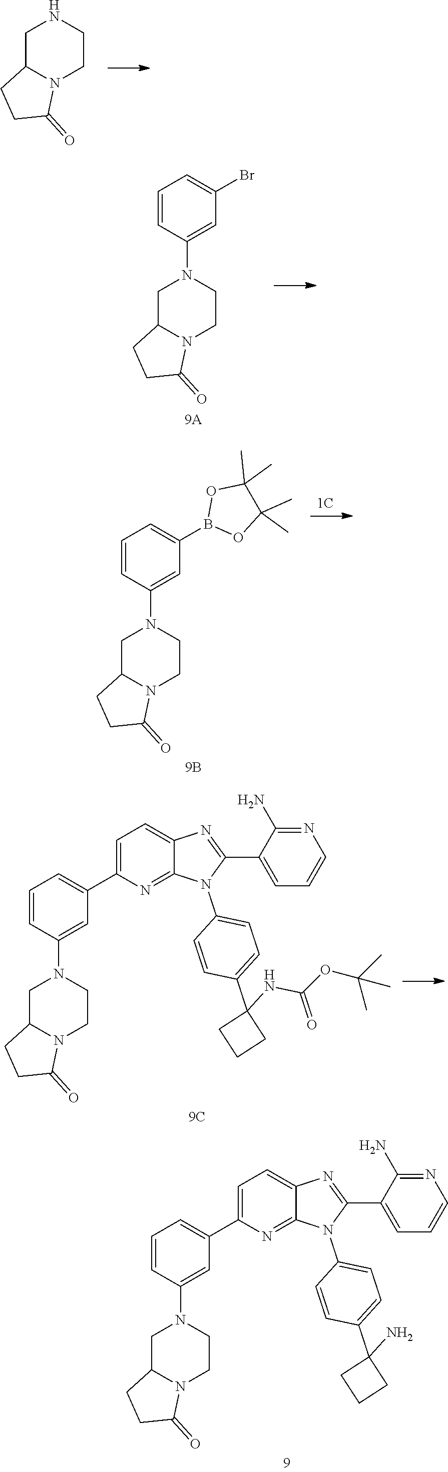 [SMILES: O=C1CCC2CNCCN12 ; O=C1CCC2CN(c3cccc(Br)c3)CCN12 ; CC1(C)OB(c2cccc(N3CCN4C(=O)CCC4C3)c2)OC1(C)C ; CC(C)(C)OC(=O)NC1(c2ccc(-n3c(-c4cccnc4N)nc4ccc(-c5cccc(N6CCN7C(=O)CCC7C6)c5)nc43)cc2)CCC1 ; Nc1ncccc1-c1nc2ccc(-c3cccc(N4CCN5C(=O)CCC5C4)c3)nc2n1-c1ccc(C2(N)CCC2)cc1]

### Step A: Preparation of Compound 9A

Hexahydropyrrolo[1,2-A]pyrazin-6-one hydrochloride (0.230 g), 1,3-dibromobenzene (0.774 g), tris(dibenzylideneacetone)dipalladium (0.150 g), 1,1′-binaphthyl-2,2′-diphenyl phosphine (0.153 g), sodium tert-butoxide (0.552 g) and toluene (7.5 mL) were added successively to a reaction flask under nitrogen atmosphere. After the addition was completed, the reaction solution was stirred at 100° C. After the reaction was completed, the reaction solution was concentrated and subjected to column chromatography (petroleum ether/ethyl acetate=4/6) to give compound 9A (0.100 g).

MS (ESI, [M+H]⁺) m/z: 295.4296.3.

### Step B: Preparation of Compound 9B

To a reaction flask were added potassium carbonate (0.249 g), bis(pinacolato)diboron (0.332 g), [1,1′-bis(diphenylphosphino)ferrocene]palladium dichloride (0.027 g), compound 9A (0.260 g), and N,N-dimethylformamide (20.00 mL) successively under nitrogen atmosphere. After the addition was completed, the reaction solution was stirred at 70° C. After the reaction was completed, the reaction solution was concentrated and subjected to column chromatography (petroleum ether/ethyl acetate=1/1) to give compound 9B (0.112 g).

MS (ESI, [M+H]⁺) m/z: 343.5.

### Step C: Preparation of Compound 9C

To a reaction flask were added compound 9B (0.268 g), compound 1C (0.145 g), potassium carbonate (0.079 g), tetrakis(triphenylphosphine)palladium (0.033 g), 1,4-dioxane (4 mL), and water (7.5 mL) successively under nitrogen atmosphere. After the addition was completed, the reaction solution was stirred at 100° C. After the reaction was completed, the reaction solution was concentrated and subjected to column chromatography (dichloromethane/methanol=93/7) to give compound 9C (0.130 g).

MS (ESI, [M+H]⁺) m/z: 671.7.

### Step D: Preparation of Compound 9

To the reaction flask were added compound 9C (0.130 g), dichloromethane (10 mL) and methanesulfonic acid (0.149 g) successively. After the addition was completed, the reaction solution was stirred at room temperature. After the reaction was completed, the reaction solution was concentrated and subjected to column chromatography (dichloromethane/methanol=93/7) to give compound 9 (0.047 g).

MS (ESI, [M+H]⁺) m/z: 571.5.

¹H NMR (500 MHz, DMSO-d₆) δ 8.25 (d, J=8.4 Hz, 1H), 8.09-7.95 (m, 2H), 7.74-7.60 (m, 3H), 7.53-7.41 (m, 3H), 7.31 (t, J=7.9 Hz, 1H), 7.24 (dd, J=7.6, 1.9 Hz, 1H), 7.14-7.02 (m, 1H), 6.98 (s, 2H), 6.43 (dd, J=7.7, 4.8 Hz, 1H), 4.55 (s, 2H), 3.95-3.85 (m, 2H), 3.81-3.73 (m, 1H), 3.69 (dtd, J=10.8, 7.1, 3.7 Hz, 1H), 2.93 (td, J=12.6, 3.8 Hz, 1H), 2.61 (td, J=12.2, 3.6 Hz, 1H), 2.49-2.42 (m, 2H), 2.35-2.21 (m, 4H), 2.16 (tdd, J=13.9, 7.7, 3.8 Hz, 1H), 2.09 (ddd, J=11.1, 5.2, 2.4 Hz, 1H), 1.75 (dtt, J=11.0, 9.0, 6.2 Hz, 1H), 1.69-1.59 (m, 1H), 0.90-0.80 (m, 1H).

### Example 10: Preparation of Compound 10

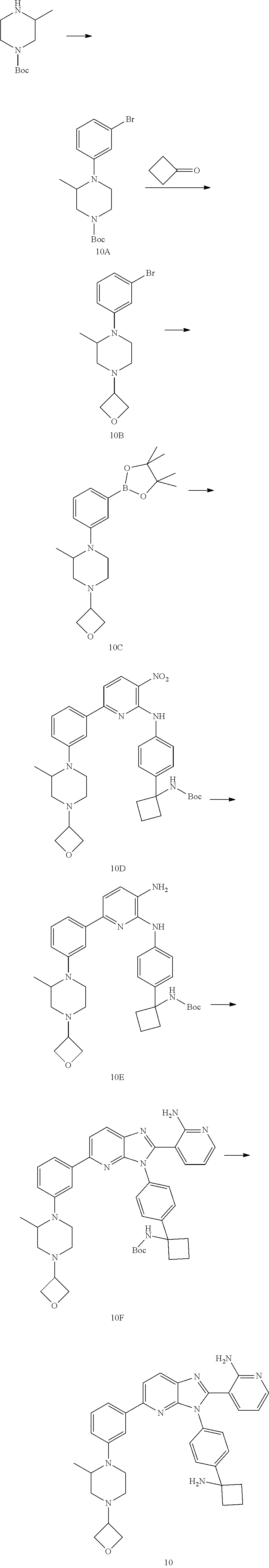

### Step A: Preparation of Compound 10A

To a reaction flask were added 1,3-dibromobenzene (2.356 g), tert-butyl 3-methylpiperazine-1-carboxylate (1.000 g), 2,2′-bis(diphenylphosphino)-1,1′-binaphthyl (0.280 g), tris(dibenzylideneacetone)dipalladium (0.320 g), sodium tert-butoxide (1.440 g) and toluene (30 mL) successively under nitrogen atmosphere. After the addition was completed, the reaction solution was stirred at 70° C. After the reaction was completed, the reaction solution was concentrated and subjected to column chromatography (petroleum ether/ethyl acetate=10/1) to give compound 10A (0.927 g).

MS (ESI, [M+H]⁺) m/z: 355.3.

### Step B: Preparation of Compound 10B

To a reaction flask were added compound 10A (0.494 g), trifluoroacetic acid (6.440 g), 3-oxetanone (0.150 g), sodium cyanoborohydride (0.175 g), dichloromethane (2 mL) and methanol (2 mL) successively at room temperature. After the addition was completed, the reaction solution was stirred for reaction. After the reaction was completed, the reaction solution was concentrated and subjected to column chromatography (dichloromethane/methanol=9/1) to give compound 10B (0.300 g).

### Step C: Preparation of Compound 10C

To a reaction flask were added compound 10B (0.300 g), potassium carbonate (0.275 g), bis(pinacolato)diboron (0.356 g), [1,1′-bis(diphenylphosphino)ferrocene]palladium dichloride (0.035 g) and N,N-dimethylformamide (34.00 mL) successively under nitrogen atmosphere. After the addition was completed, the reaction solution was stirred at 70° C. After the reaction was completed, the reaction solution was concentrated and subjected to column chromatography (petroleum ether/ethyl acetate=1/1) to give compound 10C (0.403 g).

MS (ESI, [M+H]⁺) m/z: 359.5.

### Step D: Preparation of Compound 10D

To a reaction flask were added compound 10C (0.394 g), tert-butyl (1-(4-(((6-chloro-3-nitropyridin-2-yl)amino)phenyl)cyclobutyl)carbamate (0.522 g), tetrakis(triphenylphosphine)palladium (0.059 g), saturated sodium bicarbonate solution (2.50 mL), ethanol (20 mL) and toluene (20 mL) successively under nitrogen atmosphere. After the addition was completed, the reaction solution was stirred at 100° C. After the reaction was completed, the reaction solution was concentrated and subjected to column chromatography (petroleum ether/ethyl acetate=1/1) to give compound 10D (0.579 g).

MS (ESI, [M+H]⁺) m/z: 615.6.

### Step E: Preparation of Compound 10E

To a reaction flask were added compound 10D (0.579 g), palladium/carbon (0.022 g), and tetrahydrofuran (15 mL) successively under hydrogen protection. After the addition was completed, the reaction solution was stirred for reaction at 30° C. After the reaction was completed, the reaction solution was concentrated and subjected to column chromatography (petroleum ether/ethyl acetate=1/2) to give compound 10E (0.401 g).

MS (ESI, [M+H]⁺) m/z: 585.6.

### Step F: Preparation of Compound 10F

To a reaction flask were added compound 10E (0.100 g), 2-aminonicotine (0.021 g), sodium perborate tetrahydrate (0.014 g), methanol (5 mL) and acetic acid (2 mL) successively under nitrogen atmosphere. After the addition was completed, the reaction solution was stirred at 50° C. After the reaction was completed, the reaction solution was concentrated and subjected to column chromatography (dichloromethane/methanol=96/4) to give compound 10F (0.078 g).

MS (ESI, [M+H]⁺) m/z: 687.5.

### Step G: Preparation of Compound 10

To a reaction flask were added compound 10F (0.050 g), methanesulfonic acid (0.035 g), and dichloromethane (5 mL) successively at room temperature. After the addition was completed, the reaction solution was stirred for reaction at room temperature. After the reaction was completed, the reaction solution was concentrated and subjected to column chromatography (dichloromethane/methanol=99/1) to give compound 10 (33 mg).

MS (ESI, [M+H]⁺) m/z: 587.5.

¹H NMR (500 MHz, CDCl₃) δ 8.07 (dd, J=32.5, 6.3 Hz, 2H), 7.76 (d, J=8.5 Hz, 1H), 7.64-7.47 (m, 4H), 7.38-7.28 (m, 2H), 7.20 (d, J=7.8 Hz, 1H), 6.96 (d, J=8.3 Hz, 2H), 6.63 (s, 2H), 6.41 (t, J=6.5 Hz, 1H), 4.70 (dd, J=15.0, 7.8 Hz, 2H), 4.62 (d, J=6.7 Hz, 1H), 3.86 (s, 2H), 3.60-3.41 (m, 2H), 3.28 (d, J=11.6 Hz, 1H), 3.18 (t, J=10.8 Hz, 1H), 2.63 (dt, J=18.5, 8.5 Hz, 3H), 2.43 (s, 2H), 2.30 (d, J=10.9 Hz, 3H), 2.15 (p, J=9.1, 8.7 Hz, 1H), 1.88-1.78 (m, 1H), 1.11 (d, J=6.4 Hz, 4H).

### Example 11: Preparation of Compound 11

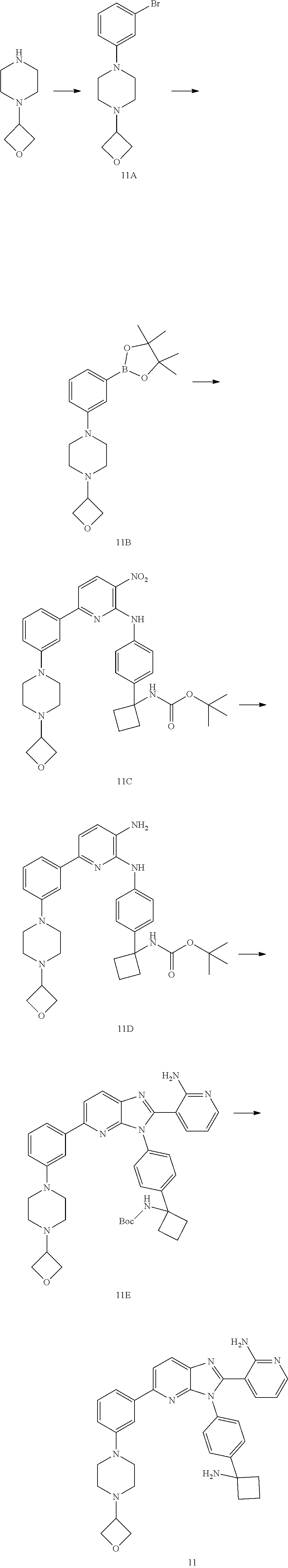

### Step A: Preparation of Compound 11A

To a reaction flask were added 1,3-dibromobenzene (4.240 g), 1-(oxetan-3-yl)piperazine (1.278 g), 1,1′-binaphthyl-2,2′-bis-diphenylphosphine (0.420 g), sodium tert-butoxide (2.590 g), tris(dibenzylideneacetone)dipalladium (0.411 g) and toluene (22 mL) successively under nitrogen atmosphere. After the addition was completed, the reaction solution was stirred for reaction at 70° C. After the reaction was completed, the reaction solution was concentrated and subjected to column chromatography (petroleum ether/ethyl acetate=1/1) to give compound 11A (2.032 g).

MS (ESI, [M+K]⁺) m/z: 335.4.

### Step B: Preparation of Compound 11B

To a reaction flask were added compound 11A (2.032 g), potassium tert-butoxide (2.010 g), bis(pinacolato)diboron (2.600 g), [1,1′-bis(diphenylphosphino)ferrocene]palladium dichloride (0.223 g) and N,N-dimethylformamide (34 mL) successively under nitrogen atmosphere. After the addition was completed, the reaction solution was stirred for reaction at 70° C. After the reaction was completed, the reaction solution was concentrated and subjected to column chromatography (petroleum ether/ethyl acetate=1/1) to give compound 11B (2.016 g).

MS (ESI, [M+H]⁺) m/z: 345.4.

### Step C: Preparation of Compound 11C

To a reaction flask were added tert-butyl (1-(4-(((6-chloro-3-nitropyridin-2-yl)amino)phenyl)cyclobutyl)carbamate (0.568 g), compound 11B (0.576 g), tetrakis(triphenylphosphine)palladium (0.078 g), ethanol (15 mL), saturated sodium bicarbonate (3 mL) and toluene (15 mL) successively under nitrogen atmosphere. After the addition was completed, the reaction solution was stirred at 100° C. After the reaction was completed, the reaction solution was concentrated and subjected to column chromatography (petroleum ether/ethyl acetate=1/1) to give compound 11C (0.732 g).

MS (ESI, [M+H]⁺) m/z: 601.3.

### Step D: Preparation of Compound 11D

Compound 11C (0.037 g) and palladium/carbon (0.001 g) were added to the reaction flask successively under hydrogen protection. After the addition was completed, the reaction solution was stirred for reaction at 30° C. After the reaction was completed, the reaction solution was concentrated and subjected to column chromatography (petroleum ether/ethyl acetate=2/1) to give compound 11D (0.013 g).

MS (ESI, [M+H]⁺) m/z: 571.5.

### Step E: Preparation of Compound 11E

To a reaction flask were added compound 11D (0.100 g), 2-aminonicotine (0.022 g), sodium perborate tetrahydrate (0.014 g), methanol (5 mL) and acetic acid (2 mL) successively under nitrogen atmosphere. After the addition was completed, the reaction solution was stirred for reaction at 50° C. After the reaction was completed, the reaction solution was concentrated and subjected to column chromatography (dichloromethane/methanol=99/1) to give compound 11E (50 mg).

MS (ESI, [M+H]⁺) m/z: 673.6.

### Step F: Preparation of Compound 11

To a reaction flask were added compound 11E (50 mg), methanesulfonic acid (35 mg), and dichloromethane (3 mL) successively at room temperature, and the mixture was stirred for reaction. After the reaction was completed, the reaction solution was concentrated and subjected to column chromatography (dichloromethane/methanol=99/1) to give compound 11 (28 mg).

MS (ESI, [M+H]⁺) m/z: 573.5.

¹H NMR (500 MHz, CDCl₃) δ 8.11 (d, J=8.3 Hz, 1H), 8.07 (tt, J=5.9, 3.2 Hz, 1H), 7.77 (d, J=8.3 Hz, 1H), 7.61 (t, J=2.0 Hz, 1H), 7.59-7.54 (m, 2H), 7.54-7.46 (m, 2H), 7.45-7.41 (m, 2H), 7.34 (t, J=7.8 Hz, 2H), 7.20-7.13 (m, 1H), 6.96 (dd, J=8.3, 2.5 Hz, 1H), 6.63 (s, 2H), 6.45-6.36 (m, 1H), 4.69 (dt, J=19.8, 6.4 Hz, 4H), 3.57 (p, J=6.5 Hz, 1H), 3.29 (t, J=5.0 Hz, 4H), 2.62 (ddd, J=11.9, 8.9, 6.2 Hz, 2H), 2.52 (t, J=4.9 Hz, 4H), 2.25 (q, J=10.5, 9.1 Hz, 2H), 2.18-2.00 (m, 2H).

### Example 12: Preparation of Compound 12

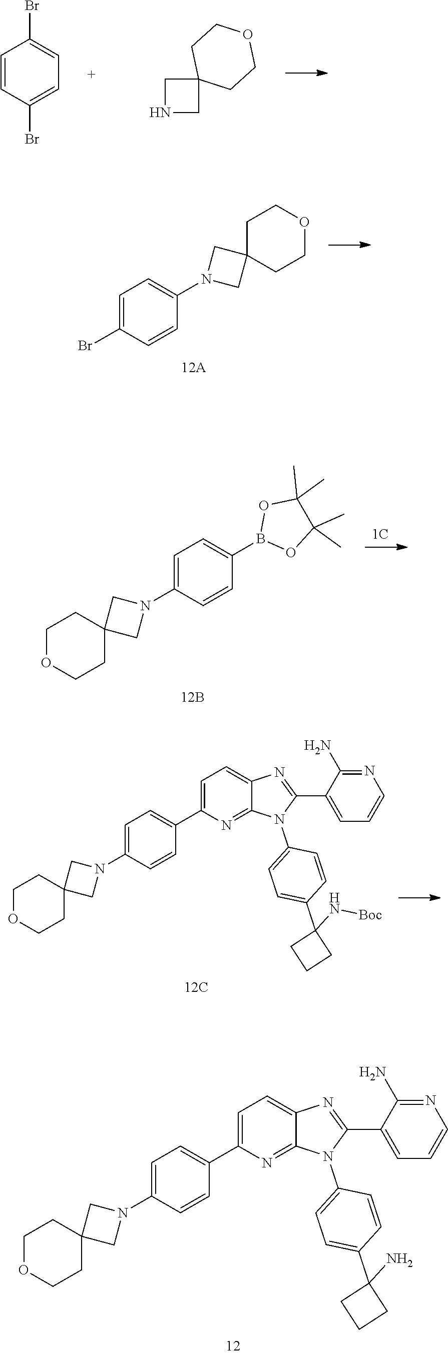 [SMILES: Brc1ccc(Br)cc1 ; C1CC2(CCO1)CNC2 ; Brc1ccc(N2CC3(CCOCC3)C2)cc1 ; CC1(C)OB(c2ccc(N3CC4(CCOCC4)C3)cc2)OC1(C)C ; CC(C)(C)OC(=O)NC1(c2ccc(-n3c(-c4cccnc4N)nc4ccc(-c5ccc(N6CC7(CCOCC7)C6)cc5)nc43)cc2)CCC1 ; Nc1ncccc1-c1nc2ccc(-c3ccc(N4CC5(CCOCC5)C4)cc3)nc2n1-c1ccc(C2(N)CCC2)cc1]

### Step A: Preparation of Compound 12A

7-Oxa-2-azaspiro[3.5]nonane (0.809 g), 1,4-dibromobenzene (1.500 g), tris(dibenzylideneacetone)dipalladium (0.582 g), 1,1′-binaphthyl-2,2′-diphenyl phosphine (0.594 g), sodium tert-butoxide (0.917 g) and toluene (20 mL) were added to a reaction flask successively under nitrogen atmosphere. After the addition was completed, the reaction solution was stirred at 120° C. After the reaction was completed, the reaction solution was concentrated and subjected to column chromatography (petroleum ether/ethyl acetate=17/3) to give compound 12A (1.257 g).

¹H NMR (500 MHz, DMSO-d₆) δ 7.27-7.29 (m, 2H), 6.35-6.37 (m, 2H), 3.56 (s, 4H), 3.53 (t, J=5.2 Hz, 4H), 1.71 (t, J=5.2 Hz, 4H).

### Step B: Preparation of Compound 12B

To a reaction flask were added potassium acetate (1.263 g), bis(pinacolato)diboron (2.178 g), [1,1′-bis (diphenylphosphino)ferrocene]palladium dichloride (0.525 g), compound 12A (0.452 g) and dioxane (15 mL) successively under nitrogen atmosphere. After the addition was completed, the reaction solution was stirred at 110° C. After the reaction was completed, the reaction solution was concentrated and subjected to column chromatography (petroleum ether/ethyl acetate=9/1) to give compound 12B (1.500 g).

MS (ESI, [M+H]⁺) m/z: 330.5.

### Step C: Preparation of Compound 12C

To a reaction flask were added compound 12B (0.419 g), compound 1C (0.500 g), potassium carbonate (0.422 g), tetrakis(triphenylphosphine)palladium (0.058 g), 1,4-dioxane (10 mL), and water (5 mL) successively under nitrogen atmosphere. After the addition was completed, the reaction solution was stirred at 110° C. After the reaction was completed, the reaction solution was concentrated and subjected to column chromatography (dichloromethane/methanol=98:2) to give compound 12C (0.114 g).

MS (ESI, [M+H]⁺) m/z: 658.7.

### Step D: Preparation of Compound 12

To a reaction flask were added compound 12C (0.114 g), dichloromethane (10 mL) and methanesulfonic acid (0.081 g) successively. After the addition was completed, the reaction solution was stirred for reaction at room temperature. After the reaction was completed, 20% aqueous sodium hydroxide solution was added, and the reaction solution was stirred under an ice bath. The pH of the reaction solution was adjusted to 12, the phases were separated, the aqueous phase was extracted by dichloromethane, and the organic phases were combined, washed with saturated brine and dried over anhydrous sodium sulfate. The reaction solution was filtered, concentrated, and subjected to column chromatography (dichloromethane/methanol=90/10) to give compound 12 (28 mg).

MS (ESI, [M+H]⁺) m/z: 558.6.

¹H NMR (500 MHz, DMSO-d₆) δ7.96-7.98 (m, 2H), 7.83-7.85 (m, 2H), 7.61-7.62 (m, 1H), 7.47-7.48 (m, 2H), 7.33-7.35 (m, 2H), 7.02-7.04 (m, 1H), 6.49 (s, 2H), 6.40-6.42 (m, 2H), 6.30-6.32 (m, 1H), 3.61 (s, 4H), 3.59 (t, J=5.2 Hz, 4H), 3.59 (q, J=9.25 Hz, 2H), 2.18 (q, J=10.5 Hz, 2H), 2.03-2.11 (m, 2H), 1.76 (t, J=5.2 Hz, 4H).

### Example 13: Preparation of Compound 13

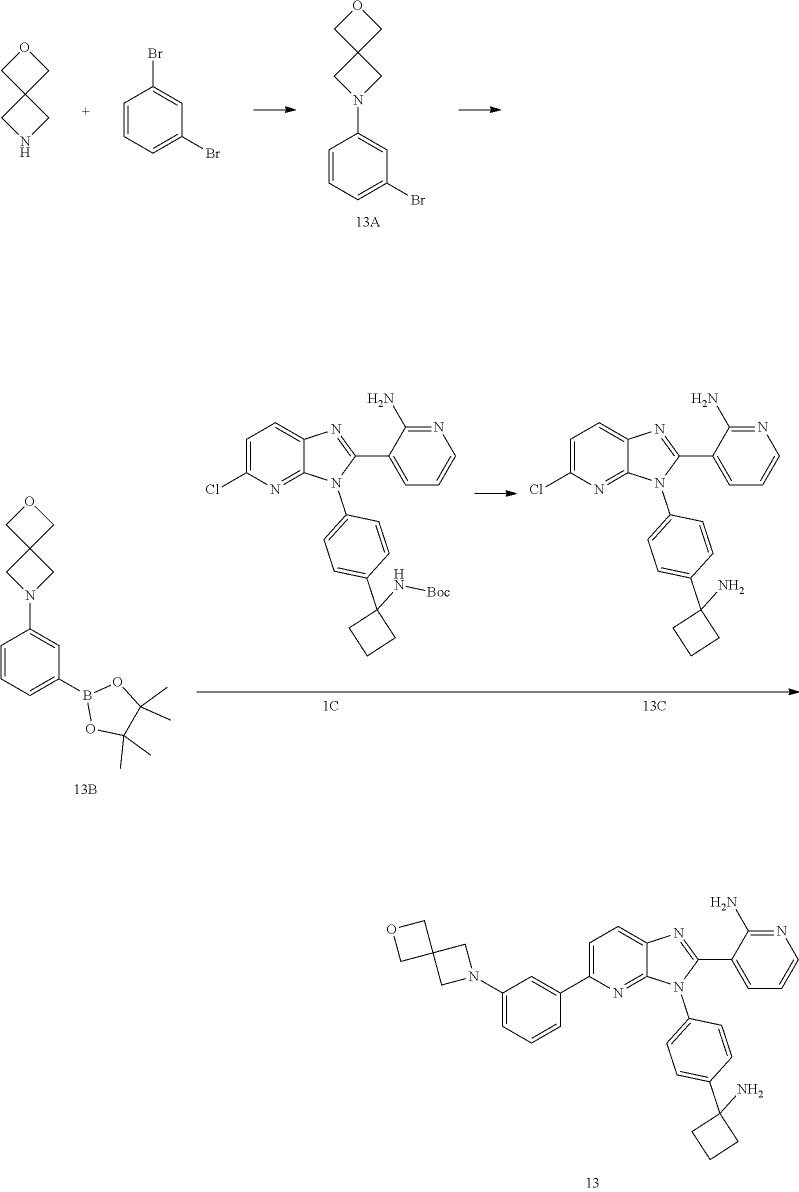

### Step A: Preparation of Compound 13A

To a reaction flask were added 2-oxa-6-azaspiro[3,3]heptane (1.00 g), m-dibromobenzene (2.49 g), (tris(dibenzylideneacetone)dipalladium (0.24 g), 1,1′-binaphthyl-2,2′-diphenyl phosphine (0.16 g), tetrahydrofuran (50 mL) and sodium tert-butoxide (2.03 g) successively, and the reaction solution was stirred at 80° C. under nitrogen atmosphere. After the reaction was completed, the reaction solution was filtered, concentrated, and subjected to column chromatography (petroleum ether/ethyl acetate=10/1) to give compound 13A (1.24 g).

MS (ESI, [M+H]⁺) m/z: 254.3.

¹H NMR (500 MHz, DMSO-d₆): δ 7.09 (t, J=8.0 Hz, 1H), 6.81 (dd, J=1.0, 8.0 Hz, 1H), 6.56 (t, J=2.0 Hz, 1H), 6.40 (dd, J=2.0, 8.0 Hz, 1H), 4.70 (s, 4H), 3.97 (m, 4H).

### Step B: Preparation of Compound 13B

To a reaction flask containing compound 13A (1.13 g) were added bis(pinacolato)diboron (1.69 g), [1,1′-bis(diphenylphosphino)ferrocene]palladium dichloride dichloromethane complex (0.18 g), 1,4-dioxane (50 mL) and potassium acetate (1.30 g) successively, and after the addition was completed, the reaction solution was stirred for reaction at 120° C. under nitrogen atmosphere. After the reaction was completed, the reaction solution was filtered, concentrated and subjected to column chromatography (petroleum ether/ethyl acetate=9/1) to give compound 13B (1.22 g).

MS (ESI, [M+H]⁺) m/z: 302.2.

¹H NMR (500 MHz, DMSO-d₆): δ 7.18 (t, J=7.5 Hz, 1H), 7.01 (d, J=7.0 Hz, 1H), 6.69 (d, J=2.5 Hz, 1H), 6.55 (d, J=2.0, 8.0 Hz, 1H), 4.70 (s, 4H), 3.95 (s, 4H), 1.28 (s, 12H).

¹³C NMR (125 MHz, DMSO-d₆): δ 151.34, 128.76, 123.98, 117.41, 115.17, 83.97, 80.38, 61.48, 38.96, 25.12.

### Step C: Preparation of Compound 13C

To a reaction flask containing compound 1C (0.495 g) were added methylene chloride (20 mL) and methanesulfonic acid (1.480 g) successively, and after the addition was completed, the reaction solution was stirred for reaction at room temperature. After the reaction was completed, the reaction solution was poured into water, the pH of the reaction solution was adjusted to 9 with an aqueous sodium hydroxide solution (20% w/w), and the reaction solution was extracted with dichloromethane, dried over anhydrous sodium sulfate, filtered and concentrated to give compound 13C (0.393 g).

MS (ESI, [M+H]⁺) m/z: 391.2.

### Step D: Preparation of Compound 13

To a microwave tube were added compound 13B (0.20 g), compound 13C (0.30 g), potassium carbonate (0.28 g), tetrakis(triphenylphosphine)palladium (0.16 g), 1,4-dioxane (10 mL), and water (1 mL) successively. After the addition was completed, the reaction solution was purged with nitrogen gas, and reacted at 120° C. in microwave reactor for 2 h. After the reaction was completed, the reaction solution was filtered and concentrated. The reaction solution was subjected to column chromatography (dichloromethane/methanol=20/1) to give compound 13 (135 mg).

MS (ESI, [M+H]⁺) m/z: 530.7.

¹H NMR (500 MHz, DMSO-d₆): δ 8.09 (d, J=8.0 Hz, 1H), 7.96 (d, J=4.5 Hz, 1H), 7.73 (d, J=8.0 Hz, 1H), 7.55 (d, J=8.0 Hz, 2H), 7.42 (d, J=8.0 Hz, 2H), 7.36 (d, J=7.5 Hz, 1H), 7.34 (t, J=8.0 Hz, 1H), 7.12 (d, J=7.5 Hz, 1H), 7.01 (s, 1H), 6.46-6.44 (m, 1H), 6.39-6.36 (m, 1H), 4.81 (s, 4H), 4.02 (s, 4H), 3.43-3.33 (m, 4H), 2.66-2.60 (m, 2H), 2.41-2.36 (m, 2H), 2.22-2.13 (m, 1H), 1.88-1.80 (m, 1H).

### Example 14: Preparation of Compound 14

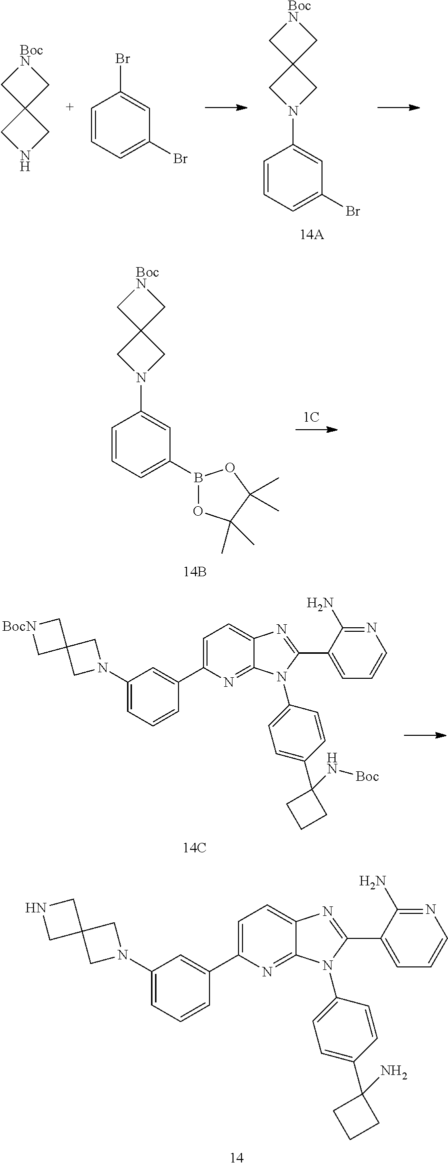

### Step A: Preparation of Compound 14A

To a reaction flask were added tert-butyl 2,6-diazaspiro[3.3]heptane-2-carboxylate (0.10 g), m-dibromobenzene (0.45 g), tris(dibenzylideneacetone)dipalladium (32 mg), 1,1′-binaphthyl-2,2′-diphenyl phosphine (21 mg), tetrahydrofuran (20 mL) and sodium tert-butoxide (0.20 g) successively, and the reaction solution was stirred for reaction at 80° C. under nitrogen atmosphere. After the reaction was completed, the reaction solution was filtered, concentrated, and subjected to column chromatography (petroleum ether/ethyl acetate=10/1) to give compound 14A (108 mg).

¹H NMR (500 MHz, DMSO-d₆): δ 7.09 (t, J=8.0 Hz, 1H), 6.81 (dd, J=1.0, 8.0 Hz, 1H), 6.55 (t, J=2.0 Hz, 1H), 6.39 (dd, J=2.0, 8.0 Hz, 1H), 4.01 (s, 4H), 3.93 (s, 4H), 1.38 (s, 9H).

### Step B: Preparation of Compound 14B

To a reaction flask containing compound 14A (152 mg) were added bis(pinacolato)diboron (164 mg), [1,1′-bis(diphenylphosphino)ferrocene]palladium dichloride dichloromethane complex (50 mg), 1,4-dioxane (5 mL) and potassium acetate (127 mg) successively, and after the addition was completed, the reaction solution was stirred for reaction at 120° C. under nitrogen atmosphere. After the reaction was completed, the reaction solution was filtered, concentrated and subjected to column chromatography (petroleum ether/ethyl acetate=9/1) to give compound 14B (53 mg).

MS (ESI, [M+H]⁺) m/z: 401.5.

### Step C: Preparation of Compound 14C

To a microwave tube were added compound 14B (50 mg), compound 1C (67 mg), potassium carbonate (70 mg), tetrakis(triphenylphosphine)palladium (40 mg), 1,4-dioxane (4 mL), and water (0.5 mL) successively. After the addition was completed, the reaction solution was purged with nitrogen gas, and reacted at 120° C. in microwave reactor for 2 h. After the reaction was completed, the reaction solution was filtered and concentrated. The reaction solution was subjected to column chromatography (dichloromethane/methanol=10/1) to give compound 14C (80 mg).

MS (ESI, [M+H]⁺) m/z: 729.7.

¹H NMR (500 MHz, DMSO-d₆): δ 8.23 (d, J=8.5 Hz, 1H), 8.00 (dd, J=1.5, 5.0 Hz, 1H), 7.92 (d, J=8.5 Hz, 1H), 7.69-7.54 (m, 3H), 7.46-7.34 (m, 3H), 7.24-7.17 (m, 2H), 7.10-7.07 (m, 3H), 6.46 (dd, J=1.5, 7.5 Hz, 1H), 6.33 (dd, J=5.0, 7.5 Hz, 1H), 4.03 (s, 4H), 3.95 (s, 4H), 2.46-2.41 (m, 4H), 2.02 (brs, 1H), 1.83 (brs, 1H), 1.39 (s, 9H), 1.36 (s, 9H).

### Step D: Preparation of Compound 14

To a reaction flask were added compound 14C (69 mg), methanesulfonic acid (0.1 mL) and dichloromethane (10 mL) successively, and the reaction solution was stirred for reaction at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 14 (25 mg).

HRMS (ESI, [M+H]⁺) m/z: 529.2814.

¹H NMR (500 MHz, DMSO-d₆): δ 8.23 (d, J=8.0 Hz, 1H), 8.01 (dd, J=2.0, 5.0 Hz, 1H), 7.91 (d, J=8.5 Hz, 1H), 7.64 (d, J=8.5 Hz, 2H), 7.43 (d, J=8.5 Hz, 2H), 7.34 (d, J=8.0 Hz, 1H), 7.25-7.21 (m, 2H), 7.06-7.02 (m, 3H), 6.46-6.44 (m, 1H), 6.43-6.41 (m, 1H), 3.90 (s, 4H), 3.64 (s, 4H), 3.43-3.33 (m, 3H), 2.46-2.41 (m, 2H), 2.16-2.11 (m, 2H), 2.07-2.01 (m, 1H), 1.76-1.67 (m, 1H).

### Example 15: Preparation of Compound 15

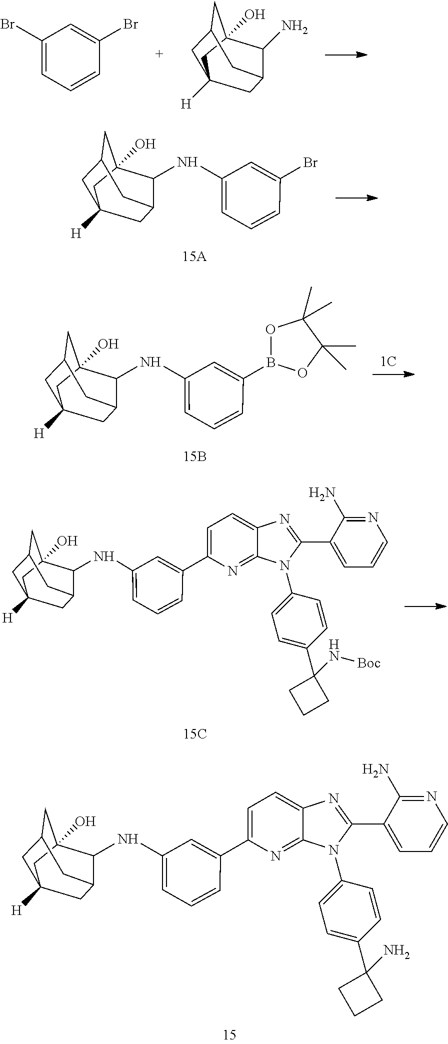 [SMILES: Brc1cccc(Br)c1 ; NC1C2CC3C[C@@H](C2)C[C@@]1(O)C3 ; O[C@@]12CC3CC(C[C@H](C3)C1)C2Nc1cccc(Br)c1 ; CC1(C)OB(c2cccc(NC3C4CC5C[C@@H](C4)C[C@@]3(O)C5)c2)OC1(C)C ; CC(C)(C)OC(=O)NC1(c2ccc(-n3c(-c4cccnc4N)nc4ccc(-c5cccc(NC6C7CC8C[C@@H](C7)C[C@@]6(O)C8)c5)nc43)cc2)CCC1 ; Nc1ncccc1-c1nc2ccc(-c3cccc(NC4C5CC6C[C@@H](C5)C[C@@]4(O)C6)c3)nc2n1-c1ccc(C2(N)CCC2)cc1]

### Step A: Preparation of Compound 15A

To a reaction flask were added (5S,7S)-2-aminoadamantan-1-ol (1.17 g), 1,3-dibromobenzene (1.65 g), tris(dibenzylideneacetone)dipalladium (0.64 g), 1,1′-binaphthyl-2,2′-diphenyl phosphine (0.65 g), sodium tert-butoxide (1.00 g) and toluene (10 mL) successively under nitrogen atmosphere. After the addition was completed, the reaction solution was stirred for reaction at 120° C. After the reaction was completed, the reaction solution was concentrated and subjected to column chromatography (petroleum ether/ethyl acetate=4/1) to give compound 15A (1.15 g).

MS (ESI, [M+H]⁺) m/z: 322.2.

### Step B: Preparation of Compound 15B

To a reaction flask were added potassium acetate (0.795 g), bis(pinacolato)diboron (1.372 g), [1,1′-bis (diphenylphosphino)ferrocene]palladium dichloride (0.331 g), compound 15A (1.154 g) and dioxane (30 mL) successively under nitrogen atmosphere. After the addition was completed, the reaction solution was stirred at 110° C. After the reaction was completed, the reaction solution was concentrated and subjected to column chromatography (petroleum ether/ethyl acetate=4/1) to give compound 15B (0.750 g).

MS (ESI, [M+H]⁺) m/z: 370.6.

### Step C: Preparation of Compound 15C

To a reaction flask were added compound 15B (0.750 g), compound 1C (0.612 g), potassium carbonate (0.495 g), tetrakis(triphenylphosphine)palladium (0.069 g), 1,4-dioxane (20 mL), and water (10 mL) successively under nitrogen atmosphere. After the addition was completed, the reaction solution was stirred for reaction at 110° C. After the reaction was completed, the reaction solution was concentrated and subjected to column chromatography (dichloromethane/methanol=3/2) to give compound 15C (1.215 g).

MS (ESI, [M+H]⁺) m/z: 698.8.

### Step D: Preparation of Compound 15

To a reaction flask were added compound 15C (1.215 g), dichloromethane (20 mL) and methanesulfonic acid (0.694 g) successively. After the addition was completed, the reaction solution was stirred for reaction at room temperature. After the reaction was completed, 20% aqueous sodium hydroxide solution was added, and the reaction solution was stirred under an ice bath. The pH of the reaction solution was adjusted to 12, the phases were separated, the aqueous phase was extracted by dichloromethane, and the organic phases were combined, washed with saturated brine and dried over anhydrous sodium sulfate. The reaction solution was filtered, concentrated, and subjected to column chromatography (dichloromethane/methanol=4/1) to give compound 15 (0.388 g).

MS (ESI, [M+H]⁺) m/z: 598.7.

¹H NMR (500 MHz, DMSO-d₆) δ 8.21 (d, 1H), 7.99-8.00 (m, 1H), 7.83 (d, 1H), 7.62 (d, 2H), 7.43 (d, 2H), 7.28 (s, 1H), 7.19-7.20 (m, 1H), 7.12-7.13 (m, 2H), 6.96 (s, 2H), 6.69 (d, 1H), 6.39-6.42 (s, 1H), 5.61 (d, 1H), 2.41-2.46 (m, 3H), 1.93-2.16 (m, 9H), 1.63-1.77 (m, 7H), 1.30 (d, 2H).

### Example 16: Preparation of Compound 16

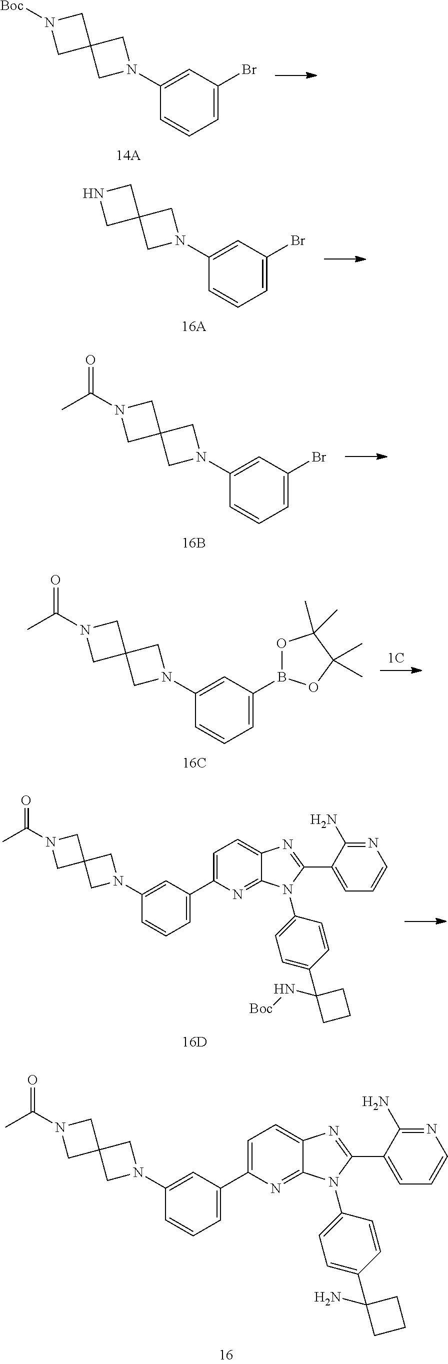

### Step A: Preparation of Compound 16A

Compound 14A (1.00 g) was weighed out and added to a reaction flask, methylene chloride (100 mL) was added, the mixture was stirred and dissolved, followed by addition of methanesulfonic acid (0.82 g), and after the addition was completed, the reaction solution was stirred for reaction at room temperature. After the reaction was completed, triethylamine (2.5 mL) was added to terminate the reaction. Dichloromethane was added to adjust the volume of the reaction solution to 120 mL to obtain a solution of compound 16A. The reaction solution was not concentrated and was directly fed for the next step.

### Step B: Preparation of Compound 16B

A solution of compound 16A (35 mL) was added to the acetic anhydride (169 mg), and after the addition was completed, the reaction solution was stirred for reaction at room temperature. After the reaction was completed, the reaction solution was concentrated and subjected to column chromatography (dichloromethane/methanol=96/4) to give compound 16B (219 mg).

MS (ESI, [M+H]⁺) m/z: 295.1.

¹H NMR (500 MHz, CDCl₃): δ 7.06 (t, J=8.0 Hz, 1H), 6.90-6.88 (m, 1H), 6.57 (d, J=4.0 Hz, 1H), 6.37-6.34 (m, 1H), 4.29 (s, 2H), 4.16 (s, 2H), 3.99 (s, 4H), 1.88 (s, 3H).

### Step C: Preparation of Compound 16C

To a reaction flask containing compound 16B (210 mg) were added bis(pinacolato)diboron (271 mg), [1,1′-bis(diphenylphosphino)ferrocene]palladium dichloride dichloromethane complex (29 mg), 1,4-dioxane (20 mL) and potassium acetate (209 mg) successively, and after the addition was completed, the reaction solution was stirred for reaction at 120° C. under nitrogen atmosphere. After the reaction was completed, the reaction solution was filtered, concentrated and subjected to column chromatography (dichloromethane/methanol=98/2) to give compound 16C (185 mg).

MS (ESI, [M+H]⁺) m/z: 343.3.

### Step D: Preparation of Compound 16D

To a microwave tube were added compound 16C (140 mg), compound 1C (276 mg), potassium carbonate (239 mg), tetrakis(triphenylphosphine)palladium (75 mg), 1,4-dioxane (10 mL), and water (1 mL) successively. After the addition was completed, the reaction solution was purged with nitrogen gas, and reacted at 120° C. in microwave reactor for 2 h. After the reaction was completed, the reaction solution was filtered and concentrated. The reaction solution was subjected to column chromatography (dichloromethane/methanol=96/4) to give compound 16D (280 mg).

MS (ESI, [M+H]⁺) m/z: 671.7.

### Step E: Preparation of Compound 16

To a reaction flask were added compound 16D (260 mg), methanesulfonic acid (0.25 mL) and dichloromethane (20 mL) successively, and the reaction solution was stirred for reaction at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 16 (125 mg).

MS (ESI, [M+H]⁺) m/z: 571.6.

¹H NMR (500 MHz, CDCl₃): 8.11-8.04 (d, J=8.5 Hz, 1H), 8.06-7.96 (dd, J=1.0, 4.5 Hz, 1H), 7.76-7.67 (d, J=8.5 Hz, 1H), 7.65-7.48 (d, J=8.5 Hz, 2H), 7.43-7.35 (m, 3H), 7.26-7.25 (m, 1H), 7.15-6.98 (m, 2H), 6.61-6.57 (m, 2H), 6.44-6.32 (m, 1H), 6.39-6.25 (m, 1H), 4.29-4.26 (m, 2H), 4.20-4.16 (m, 2H), 4.05-3.92 (m, 4H), 2.69-2.64 (m, 2H), 2.58-2.52 (m, 2H), 2.46-2.26 (m, 3H), 1.90-1.86 (m, 4H).

### Example 17: Preparation of Compound 17

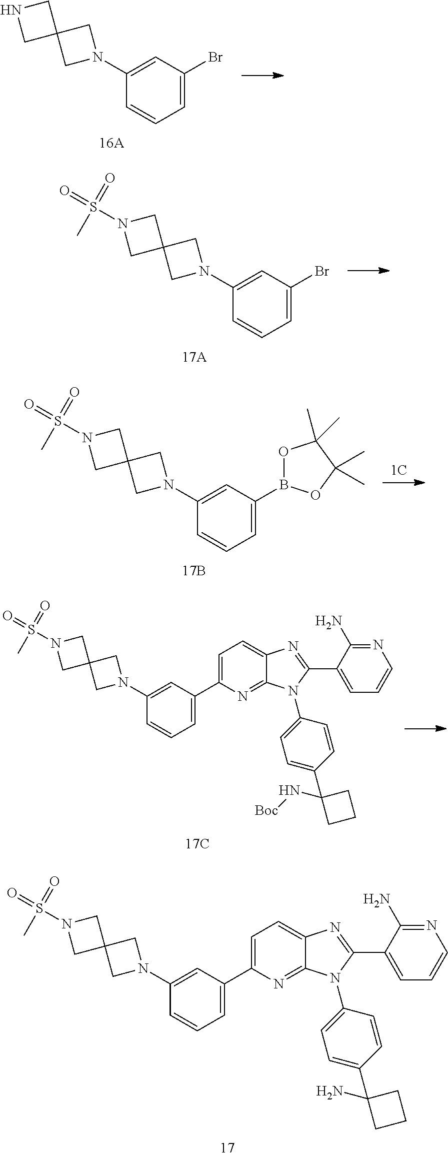 [SMILES: Brc1cccc(N2CC3(CNC3)C2)c1 ; CS(=O)(=O)N1CC2(CN(c3cccc(Br)c3)C2)C1 ; CC1(C)OB(c2cccc(N3CC4(C3)CN(S(C)(=O)=O)C4)c2)OC1(C)C ; CC(C)(C)OC(=O)NC1(c2ccc(-n3c(-c4cccnc4N)nc4ccc(-c5cccc(N6CC7(C6)CN(S(C)(=O)=O)C7)c5)nc43)cc2)CCC1 ; CS(=O)(=O)N1CC2(CN(c3cccc(-c4ccc5nc(-c6cccnc6N)n(-c6ccc(C7(N)CCC7)cc6)c5n4)c3)C2)C1]

### Step A: Preparation of Compound 17A

A solution of compound 16A (35 mL) was added to the methanesulfonyl chloride (189 mg), after the addition was completed, the reaction solution was stirred for reaction at room temperature. After the reaction was completed, the reaction solution was concentrated and subjected to column chromatography (petroleum ether/ethyl acetate=3/1) to give compound 17A (227 mg).

MS (ESI, [M+H]⁺) m/z: 331.1.

¹H NMR (500 MHz, CDCl₃): δ 7.06 (t, J=3.0 Hz, 1H), 6.90-6.88 (m, 1H), 6.57 (t, J=2.0 Hz, 1H), 6.36-6.34 (m, 1H), 4.10 (s, 4H), 3.98 (s, 4H), 2.88 (s, 3H).

### Step B: Preparation of Compound 17B

To a reaction flask containing compound 17A (220 mg) were added bis(pinacolato)diboron (253 mg), [1,1′-bis(diphenylphosphino)ferrocene]palladium dichloride dichloromethane complex (27 mg), 1,4-dioxane (20 mL) and potassium acetate (196 mg) successively, and after the addition was completed, the reaction solution was stirred for reaction at 120° C. under nitrogen atmosphere. After the reaction was completed, the reaction solution was filtered, concentrated and subjected to column chromatography (petroleum ether/ethyl acetate=1/1) to give compound 17B (157 mg).

MS (ESI, [M+H]⁺) m/z: 379.3.

### Step C: Preparation of Compound 17C

To a microwave tube were added compound 17B (132 mg), compound 1C (223 mg), potassium carbonate (193 mg), tetrakis(triphenylphosphine)palladium (81 mg), 1,4-dioxane (10 mL), and water (1 mL) successively. After the addition was completed, the reaction solution was purged with nitrogen gas, and reacted at 120° C. in microwave reactor for 2 h. After the reaction was completed, the reaction solution was filtered, concentrated and subjected to column chromatography (dichloromethane/methanol=98/2) to give compound 17C (146 mg).

MS (ESI, [M+H]⁺) m/z: 707.7.

### Step D: Preparation of Compound 17

To a reaction flask were added compound 17C (150 mg), methanesulfonic acid (0.15 mL) and dichloromethane (15 mL) successively, and the reaction solution was stirred for reaction at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=95/5) to give compound 17 (47 mg).

HRMS (ESI, [M+H]⁺) m/z: 607.2606.

¹H NMR (500 MHz, CDCl₃): 8.05-7.91 (m, 2H), 7.69-7.60 (m, 1H), 7.52-7.41 (m, 2H), 7.37-7.26 (m, 3H), 7.23-7.20 (m, 1H), 7.09-6.93 (m, 2H), 6.53, 6.44 (s, 2H), 6.42-6.18 (m, 2H), 4.04, 4.02 (s, 4H), 3.96, 3.85 (s, 4H), 2.81, 2.80 (s, 3H), 2.58-2.36 (m, 3H), 2.22-2.02 (m, 3H).

### Example 18: Preparation of Compound 18

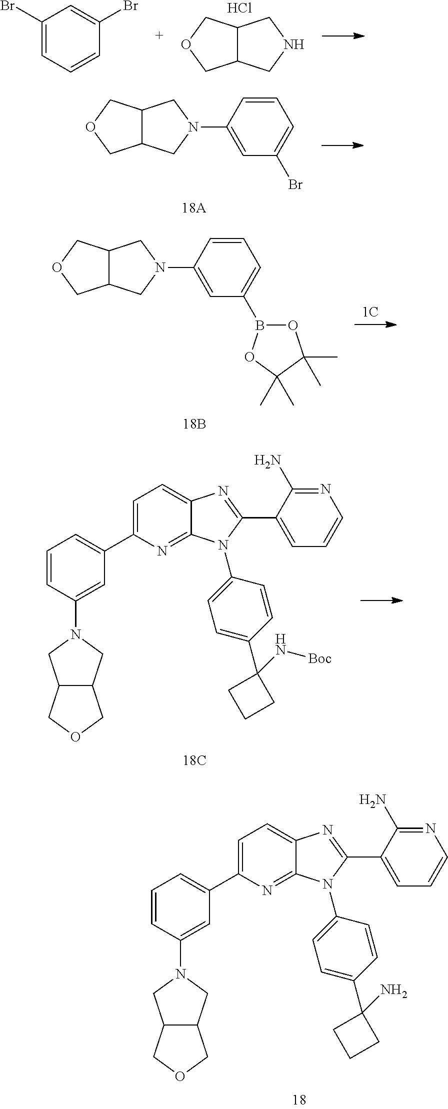

### Step A: Preparation of Compound 18A

To a reaction flask were added hexahydro-1H-furo[3,4-c]pyrrole hydrochloride (0.824 g), 1,3-dibromobenzene (1.300 g), tris(dibenzylideneacetone)dipalladium (0.505 g), 1,1′-binaphthyl-2,2′-diphenyl phosphine (0.515 g), sodium tert-butoxide (0.794 g) and toluene (10 mL) successively under nitrogen atmosphere. After the addition was completed, the reaction solution was stirred for reaction at 120° C. After the reaction was completed, the reaction solution was concentrated and subjected to column chromatography (petroleum ether/ethyl acetate=19/1) to give compound 18A (0.452 g).

MS (ESI, [M+H]⁺) m/z: 268.4.

### Step B: Preparation of Compound 18B

To a reaction flask were added potassium acetate (0.496 g), bis(pinacolato)diboron (0.809 g), [1,1′-bis (diphenylphosphino)ferrocene]palladium dichloride (0.206 g), compound 18A (0.452 g) and dioxane (20 mL) successively under nitrogen atmosphere. After the addition was completed, the reaction solution was stirred for reaction at 110° C. After the reaction was completed, the reaction solution was concentrated and subjected to column chromatography (petroleum ether/ethyl acetate=19/1) to give compound 18B (0.490 g).

MS (ESI, [M+H]⁺) m/z: 316.5.

### Step C: Preparation of Compound 18C

To a reaction flask were added compound 18B (0.210 g), compound 1C (0.327 g), potassium carbonate (0.276 g), tetrakis(triphenylphosphine)palladium (0.038 g), 1,4-dioxane (15 mL), and water (7.5 mL) successively under nitrogen atmosphere. After the addition was completed, the reaction solution was stirred for reaction at 110° C. After the reaction was completed, the reaction solution was concentrated and subjected to column chromatography (dichloromethane/methanol=1/1) to give compound 18C (0.400 g).

MS (ESI, [M+H]⁺) m/z: 644.8.

### Step D: Preparation of Compound 18

To the reaction flask were added compound 18C (0.400 g), dichloromethane (10 mL) and methanesulfonic acid (0.149 g) successively. After the addition was completed, the reaction solution was stirred for reaction at room temperature. After the reaction was completed, 20% aqueous sodium hydroxide solution was added, and the reaction solution was stirred under an ice bath. The pH of the reaction solution was adjusted to 12, the phases were separated, the aqueous phase was extracted by dichloromethane, and the organic phases were combined, washed with saturated brine and dried over anhydrous sodium sulfate. The reaction solution was filtered, concentrated, and subjected to column chromatography (dichloromethane/methanol=7/3) to give compound 18 (0.285 g).

MS (ESI, [M+H]⁺) m/z: 544.7.

¹H NMR (500 MHz, DMSO-d₆) δ 8.43 (d, 1H), 8.11-8.21 (m, 3H), 8.00 (d, 1H), 7.92 (d, 1H), 7.80 (d, 2H), 7.71 (d, 2H), 7.48-7.58 (m, 2H), 6.98 (t, 1H), 3.70-3.88 (m, 6H), 3.21 (s, 2H), 2.65-2.68 (m, 4H), 2.21-2.28 (m, 1H), 1.82-1.90 (m, 1H), 1.17-1.29 (m, 2H).

### Example 19: Preparation of Compound 19

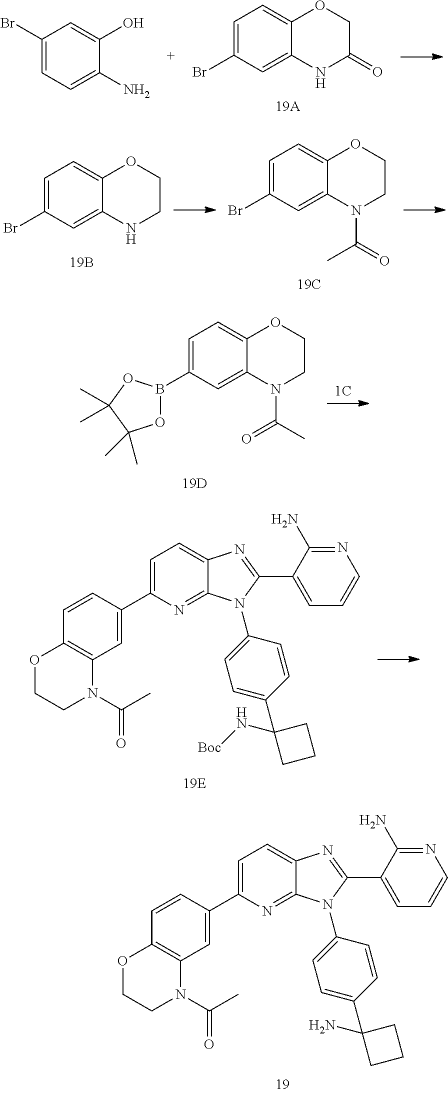

### Step A: Preparation of Compound 19A

In a reaction flask, a solution of 2-chloroacetyl chloride (3.24 g) in tetrahydrofuran (10 mL) was slowly added dropwise to a reaction solution of 2-amino-4-bromophenol (3.60 g) and sodium carbonate (3.22 g) in tetrahydrofuran (60 mL) under nitrogen atmosphere at 0° C. After the addition was completed, the reaction solution was stirred at room temperature. Potassium carbonate (5.29 g) was added to the reaction solution, and the reaction solution was stirred for reaction at 66° C., filtered, concentrated, and subjected to column chromatography (petroleum ether/ethyl acetate=3/1) to give compound 19A (3.4 g).

MS (ESI, [M−H]⁻) m/z: 226.4.

### Step B: Preparation of Compound 19B

In a reaction flask, a solution (15 mL) of 2 M borane in toluene-dimethylsulfide was added slowly under nitrogen atmosphere at 0° C. to a solution of compound 19A (2.28 g) in tetrahydrofuran (10 mL). After the addition was completed, the mixture was stirred at room temperature and reacted at 66° C. After the reaction was completed, the reaction was quenched with a saturated aqueous sodium hydroxide solution, the reaction solution was extracted with ethyl acetate, and the organic phases were combined, washed with saturated brine, dried over anhydrous sodium sulfate, filtered, concentrated, and subjected to column chromatography (petroleum ether/ethyl acetate=4/1) to give compound 19B (2.10 g). The reaction product from this step was used directly in the next step.

### Step C: Preparation of Compound 19C

In a reaction flask, acetyl chloride (0.733 g) was slowly added to a solution of compound 19B (2.000 g), N,N-diisopropylethylamine (1.208 g) in tetrahydrofuran (12 mL) under nitrogen atmosphere a 0° C. After the addition was completed, the reaction solution was stirred at room temperature. After the reaction was completed, the reaction was quenched with saturated sodium carbonate solution, the reaction solution was extracted with ethyl acetate, and the organic phases were combined, washed with saturated brine, dried over anhydrous sodium sulfate, filtered, concentrated, and subjected to column chromatography (petroleum ether/ethyl acetate=95/5) to give compound 19C (2.200 g). The reaction product from this step was used directly in the next step.

### Step D: Preparation of Compound 19D

To a reaction flask were added [1,1′-bis(diphenylphosphino)ferrocene]palladium dichloride (0.702 g), compound 19C (2.200 g), bis(pinacolato)diboron (4.360 g), potassium acetate (1.686 g) and dioxane (8 mL) successively under nitrogen atmosphere; after the addition was completed, the reaction solution was reacted at 110° C. After the reaction was completed, the reaction solution was extracted with ethyl acetate, and organic phases were combined, washed with saturated brine, dried over anhydrous sodium sulfate, filtered, concentrated, and subjected to column chromatography (petroleum ether/ethyl acetate=90/10) to give compound 19D (2.410 g).

MS (ESI, [M+H]⁺) m/z: 304.5.

### Step E: Preparation of Compound 19E

To a reaction flask were added compound 19D (324 mg), compound 1C (350 mg), potassium carbonate (197 mg), tetrakis(triphenylphosphine)palladium (82 mg), and dioxane (10 mL) successively under nitrogen atmosphere, and after the addition was completed, the reaction solution was reacted at 110° C. After the reaction was completed, the reaction solution was concentrated and subjected to column chromatography (petroleum ether/ethyl acetate=95/5) to give compound 19E (320 mg).

MS (ESI, [M+H]⁺) m/z: 632.7.

### Step F: Preparation of Compound 19

To the reaction flask were added compound 19E (320 mg), dichloromethane (10 mL) and methanesulfonic acid (48.7 mg) successively, and the reaction solution was stirred for reaction at room temperature. The raw materials were reacted completely. The reaction was quenched with saturated aqueous sodium carbonate (30 mL), the reaction solution was extracted with dichloromethane, and the organic phases were combined, washed with saturated brine, dried over anhydrous sodium sulfate, filtered, concentrated, and subjected to column chromatography (dichloromethane/methanol=94/6) to give compound 19 (120 mg).

MS (ESI, [M+H]⁺) m/z: 532.7.

¹H NMR (500 MHz, DMSO-d₆): δ 8.22 (d, J=8.0 Hz, 1H), 8.00 (s, 1H), 7.87 (br, 1H), 7.63-7.62 (m, 2H), 7.45-7.44 (m, 2H), 7.21 (d, J=7.5 Hz, 1H), 7.00-7.69 (m, 3H), 6.41 (t, J=5.0 Hz, 1H), 4.29 (s, 2H), 3.88 (s, 2H), 2.50-2.43 (m, 2H), 2.24 (s, 3H), 2.19-2.15 (m, 2H), 2.06-2.04 (m, 1H), 1.75-1.72 (m, 1H).

### Example 20: Preparation of Compound 20

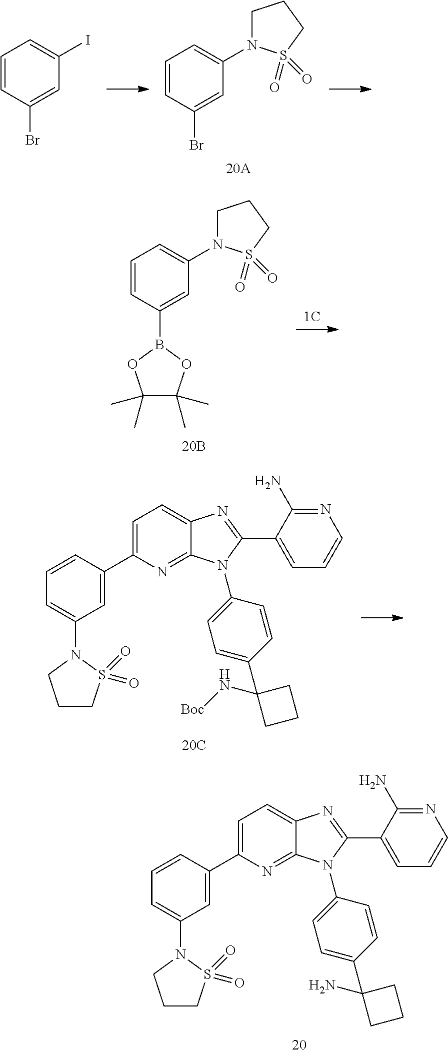 [SMILES: Brc1cccc(I)c1 ; O=S1(=O)CCCN1c1cccc(Br)c1 ; CC1(C)OB(c2cccc(N3CCCS3(=O)=O)c2)OC1(C)C ; CC(C)(C)OC(=O)NC1(c2ccc(-n3c(-c4cccnc4N)nc4ccc(-c5cccc(N6CCCS6(=O)=O)c5)nc43)cc2)CCC1 ; Nc1ncccc1-c1nc2ccc(-c3cccc(N4CCCS4(=O)=O)c3)nc2n1-c1ccc(C2(N)CCC2)cc1]

### Step A: Preparation of Compound 20A

To a reaction flask were added 1-bromo-3-iodobenzene (1.180 g), isothiazole-1,1-dioxide (0.720 g), cuprous iodide (0.159 g), potassium carbonate (1.153 g), (1S,2S)-N1,N2-dimethylcyclohexyl-1,2-diamine (0.119 g), and dimethyl sulfoxide (10 mL) successively under nitrogen atmosphere, and the reaction solution was reacted at 120° C. After the reaction was completed, the reaction solution was concentrated and subjected to column chromatography (petroleum ether/ethyl acetate=90/10) to give compound 20A (640 mg).

MS(ESI, [M+H]⁺) m/z: 276.2.

### Step B: Preparation of Compound 20B

To a reaction flask were added [1,1′-bis(diphenylphosphino)ferrocene]palladium dichloride (151 mg), compound 20A (640 mg), bis(pinacolato)diboron (1177 mg), potassium acetate (455 mg) and dioxane (8 mL) successively under nitrogen atmosphere; after the addition was completed, the reaction solution was reacted at 110° C. After the reaction was completed, the reaction solution was concentrated and subjected to column chromatography (petroleum ether/ethyl acetate=80/20) to give compound 20B (620 mg).

MS(ESI, [M+H]⁺) m/z: 324.5.

### Step C: Preparation of Compound 20C

To a reaction flask were added tetrakis(triphenylphosphine)palladium (99 mg), compound 1C (420 mg), compound 20B (415 mg), potassium carbonate (236 mg), dioxane (5 mL) and water (0.5 mL) successively, and the reaction solution was heated at 120° C. After the reaction was completed, the reaction solution was concentrated and subjected to column chromatography (petroleum ether/ethyl acetate=60/40) to give compound 20C (380 mg).

MS(ESI, [M+H]⁺) m/z: 652.5.

### Step D: Preparation of Compound 20

To the reaction flask were added compound 20C (150 mg), dichloromethane (6 mL) and methanesulfonic acid (133 mg) successively. After the addition was completed, the reaction solution was reacted at room temperature. After the reaction was completed, the pH of the reaction solution was adjusted to be neutral with saturated aqueous sodium bicarbonate, the reaction solution was extracted with dichloromethane, the organic phases were combined, washed with saturated brine, dried over anhydrous sodium sulfate, filtered, concentrated, and subjected to column chromatography (dichloromethane/methanol=97/3) to give compound 20 (27 mg).

MS(ESI, [M+H]⁺) m/z: 552.5.

¹H NMR (500 MHz, DMSO-d₆): δ 8.29 (d, J=8.0 Hz, 1H), 8.01 (dd, J=3.0 Hz, 6.0 Hz, 1H), 7.97 (d, J=8.5 Hz, 1H), 7.88 (s, 1H), 7.77 (d, J=7.5 Hz, 1H), 7.64 (d, J=9.0 Hz, 1H), 7.50-7.45 (m, 3H), 7.28-7.23 (m, 2H), 6.96 (br, 2H), 6.44 (q, J=7.5 Hz, 1H), 3.81 (t, J=7.5 Hz, 2H), 3.54 (t, J=7.5 Hz, 2H), 2.43 (t, J=7.5 Hz, 1H), 2.34 (s, 2H), 2.26-2.21 (m, 2H), 2.09 (m, 1H), 1.77-1.75 (m, 1H).

### Example 21: Preparation of Compound 21

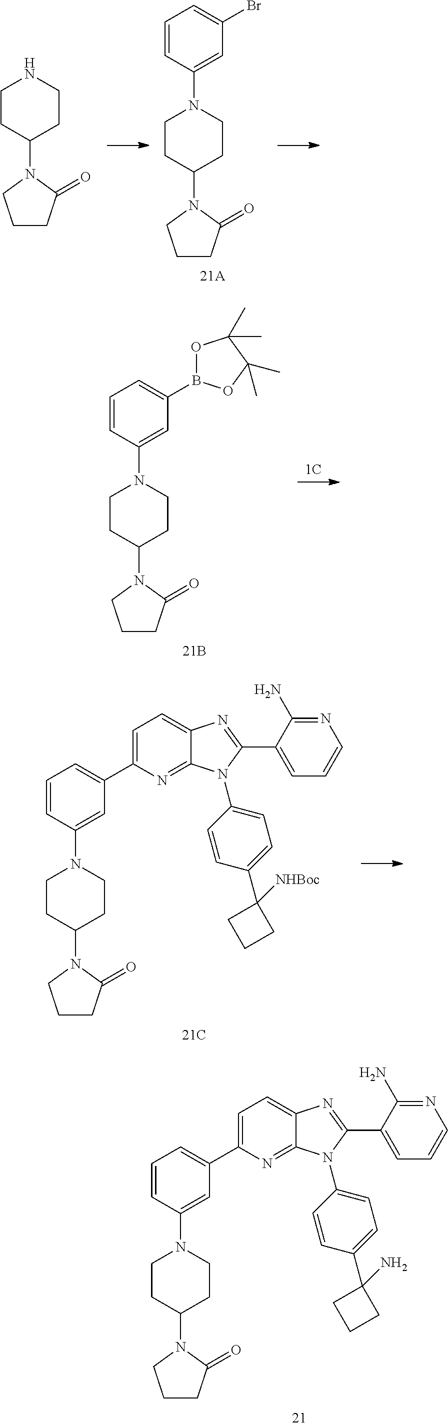 [SMILES: O=C1CCCN1C1CCNCC1 ; O=C1CCCN1C1CCN(c2cccc(Br)c2)CC1 ; CC1(C)OB(c2cccc(N3CCC(N4CCCC4=O)CC3)c2)OC1(C)C ; CC(C)(C)OC(=O)NC1(c2ccc(-n3c(-c4cccnc4N)nc4ccc(-c5cccc(N6CCC(N7CCCC7=O)CC6)c5)nc43)cc2)CCC1 ; Nc1ncccc1-c1nc2ccc(-c3cccc(N4CCC(N5CCCC5=O)CC4)c3)nc2n1-c1ccc(C2(N)CCC2)cc1]

### Step A: Preparation of Compound 21A

To a reaction flask were added sodium tert-butoxide (0.179 g), 1,1′-binaphthyl-2,2′-diphenyl phosphine (0.040 g), tris(dibenzylideneacetone)dipalladium (0.039 g), 1,3-dibromobenzene (0.200 g), 1-(piperidin-4-yl)pyrrolidin-2-one hydrochloride (0.174 g) and toluene (10 mL) successively, and the reaction solution was stirred for reaction at 80° C. under nitrogen atmosphere. After the reaction was completed, the reaction solution was concentrated, added with 5 mL of saturated brine and extracted with ethyl acetate (10 mL×3), and the organic phases were combined and dried over anhydrous sodium sulfate. The reaction solution was filtered and concentrated. The reaction solution was subjected to column chromatography (dichloromethane/methanol=60/1) to give compound 21A (0.270 g).

MS (ESI, [M+H]⁺) m/z: 323.4.

### Step B: Preparation of Compound 21B

To a reaction flask were added compound 21A (0.270 g), potassium acetate (0.270 g), [1,1′-bis(diphenylphosphino)ferrocene]palladium dichloride dichloromethane complex (0.037 g), bis(pinacolato)diboron (0.280 g), 1,4-dioxane (25 mL) and water (5 mL) successively, and the reaction solution was stirred for reaction at 100° C. under nitrogen atmosphere. After the reaction was completed, the reaction solution was concentrated, added with saturated brine and extracted with ethyl acetate, and the organic phases were combined and dried over anhydrous sodium sulfate. The reaction solution was filtered, concentrated, and subjected to column chromatography (dichloromethane/methanol=60/1) to give compound 21B (0.300 g).

MS (ESI, [M+H]⁺) m/z: 371.6.

### Step C: Preparation of Compound 21C

To a reaction flask were added compound 1C (2.40 g), compound 21B (0.30 g), potassium carbonate (0.17 g), tetrakis(triphenylphosphine)palladium (0.057 g), 1,4-dioxane (25 mL) and water (5 mL) successively, and the reaction solution was stirred for reaction at 100° C. under nitrogen atmosphere. After the reaction was completed, the reaction solution was concentrated, added with saturated brine and extracted with ethyl acetate, and the organic phases were combined and dried over anhydrous sodium sulfate. The reaction solution was filtered, concentrated, and subjected to column chromatography (dichloromethane/methanol=65/1) to give compound 21C (0.25 g).

MS (ESI, [M+H]⁺) m/z: 699.8.

### Step D: Preparation of Compound 21

To a reaction flask were added compound 21C (0.25 g), dichloromethane (25 mL) and methanesulfonic acid (0.30 g) successively. The reaction solution was stirred at 30° C. for reaction; after the reaction was completed, the pH of the reaction solution was adjusted to be alkaline, the reaction solution was added with saturated brine and extracted with dichloromethane, and the organic phases were combined, and dried over anhydrous sodium sulfate. The reaction solution was filtered, concentrated, and subjected to column chromatography (dichloromethane/methanol=10/1) to give compound 21 (0.19 g).

HRMS (ESI, [M+H]⁺) m/z: 599.3237.

¹H NMR (500 MHz, CDCl₃) δ 7.99 (dd, J=11.1, 8.5 Hz, 1H), 7.90 (t, J=4.6 Hz, 1H), 7.64 (t, J=8.2 Hz, 1H), 7.57 (d, J=8.2 Hz, 2H), 7.48 (s, 1H), 7.47-7.22 (m, 4H), 7.01 (t, J=7.6 Hz, 1H), 6.80 (dd, J=28.6, 9.7 Hz, 1H), 6.55 (d, J=15.2 Hz, 2H), 6.27 (ddd, J=58.5, 7.7, 5.0 Hz, 1H), 4.05 (ddd, J=16.5, 10.7, 3.8 Hz, 1H), 3.65 (t, J=16.0 Hz, 2H), 3.28 (dt, J=14.1, 6.9 Hz, 2H), 2.76 (dt, J=24.5, 12.3 Hz, 2H), 2.62-2.46 (m, 4H), 2.44-2.22 (m, 4H), 1.99-1.86 (m, 3H), 1.83-1.65 (m, 5H).

### Example 22: Preparation of Compound 22

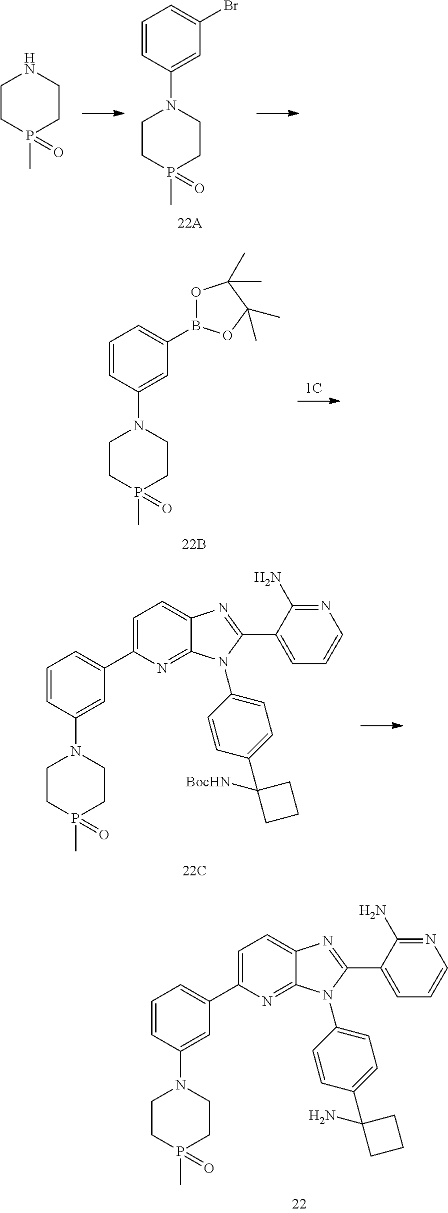

### Step A: Preparation of Compound 22A

To a reaction flask were added sodium tert-butoxide (0.180 g), 1,1′-binaphthyl-2,2′-diphenyl phosphine (0.040 g), tris(dibenzylideneacetone)dipalladium (0.039 g), 1,3-dibromobenzene (0.200 g), 4-methyl-4-oxo-1,4-azaphosphine (0.11 g) and toluene (10 mL) successively. The reaction solution was stirred for reaction at 80° C. under nitrogen atmosphere. After the reaction was completed, the reaction solution was concentrated, added with saturated brine and extracted with ethyl acetate, and the organic phases were combined and dried over anhydrous sodium sulfate. The reaction solution was filtered, concentrated, and subjected to column chromatography (dichloromethane/methanol=60/1) to give compound 22A (0.230 g).

MS (ESI, [M+H]⁺) m/z: 288.4.

### Step B: Preparation of Compound 22B

To a reaction flask were added compound 22A (0.23 g), potassium acetate (0.28 g), [1,1′-bis(diphenylphosphino)ferrocene]palladium dichloride dichloromethane complex (39 mg), bis(pinacolato)diboron (0.29 g), 1,4-dioxane (25 mL) and water (5 mL) successively. The reaction solution was stirred for reaction at 100° C. under nitrogen atmosphere. After the reaction was completed, the reaction solution was concentrated, condition with saturated brine and extracted with ethyl acetate, and the organic phases were combined and dried over anhydrous sodium sulfate. The reaction solution was filtered, concentrated, and subjected to column chromatography (dichloromethane/methanol=60/1) to give compound 22B (0.28 g).

MS (ESI, [M+H]⁺) m/z: 336.6.

### Step C: Preparation of Compound 22C

To a reaction flask were added compound 1C (2.30 g), compound 22B (0.28 g), potassium carbonate (0.16 g), tetrakis(triphenylphosphine)palladium (55 mg), 1,4-dioxane (25 mL) and water (5 mL) successively, and the reaction solution was stirred for reaction at 100° C. under nitrogen atmosphere. After the reaction was completed, the reaction solution was concentrated, added with saturated brine and extracted with ethyl acetate, and the organic phases were combined and dried over anhydrous sodium sulfate. The reaction solution was filtered, concentrated, and subjected to column chromatography (dichloromethane/methanol=65/1) to give compound 22C (0.24 g).

MS (ESI, [M+H]⁺) m/z: 664.7.

### Step D: Preparation of Compound 22

To a reaction flask were added compound 22C (0.24 g), dichloromethane (25 mL) and methanesulfonic acid (0.30 g) successively. The reaction solution was stirred at 30° C. for reaction; after the reaction was completed, the pH of the reaction solution was adjusted to be alkaline, the reaction solution was added with saturated brine and extracted with dichloromethane, and the organic phases were combined, and dried over anhydrous sodium sulfate. The reaction solution was filtered, concentrated, and subjected to column chromatography (dichloromethane/methanol=10/1) to give compound 22 (0.16 g).

HRMS (ESI, [M+H]⁺) m/z: 564.2634.

¹H NMR (500 MHz, DMSO-d₆) δ 8.24 (d, J=8.3 Hz, 1H), 8.02 (dd, J=4.7, 1.6 Hz, 1H), 7.97 (d, J=8.4 Hz, 1H), 7.64 (d, J=8.4 Hz, 2H), 7.61 (s, 1H), 7.49 (d, J=8.5 Hz, 2H), 7.38 (d, J=7.6 Hz, 1H), 7.30 (t, J=7.9 Hz, 1H), 7.26 (dd, J=7.6, 1.6 Hz, 1H), 7.06-6.96 (m, 3H), 6.43 (dd, J=7.6, 4.8 Hz, 1H), 4.48 (s, 2H), 4.03-3.85 (m, 2H), 3.57-3.46 (m, 2H), 2.49-2.43 (m, 2H), 2.28-2.20 (m, 2H), 2.13-2.03 (m, 1H), 1.88 (t, J=13.6 Hz, 2H), 1.75 (ddt, J=20.2, 14.2, 6.4 Hz, 3H), 1.53 (d, J=13.0 Hz, 3H).

### Example 23: Preparation of Compound 23

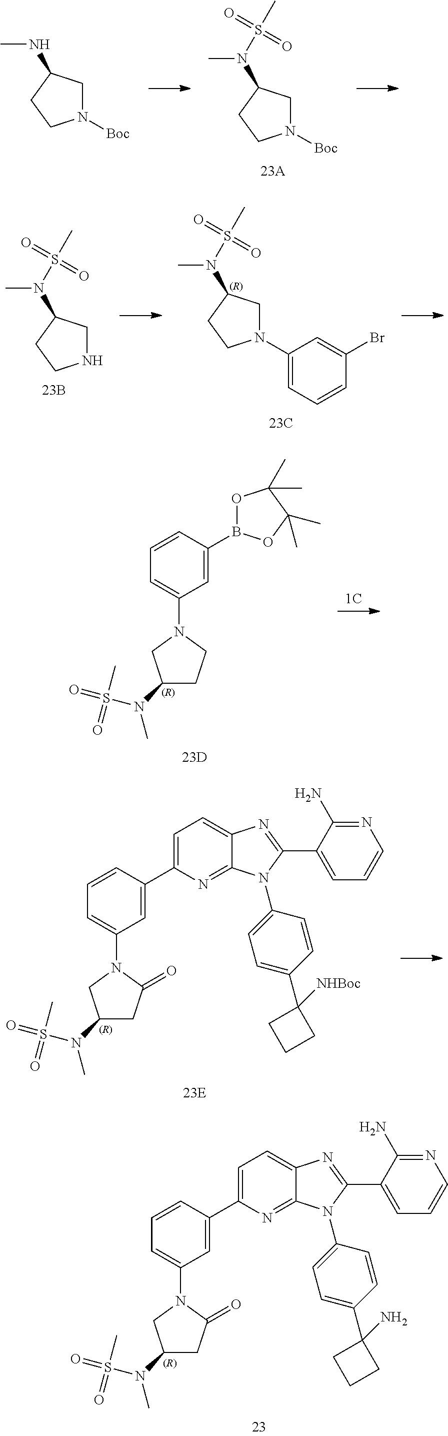

### Step A: Preparation of Compound 23A

In a reaction flask, methanesulfonyl chloride (0.572 g) was added at 0° C. to a solution of tert-butyl (R)-3-(methylamine)pyrrole-1-carboxylate (1.000 g) and N,N-diisopropylethylamine (0.645 g) in dichloromethane (15 mL) under nitrogen atmosphere, and the reaction solution was stirred at room temperature overnight. After the reaction was completed, the reaction was quenched by saturated aqueous sodium carbonate solution, the reaction solution was extracted with dichloromethane, and the organic phases were combined, washed with saturated brine, dried over anhydrous sodium sulfate, filtered, concentrated, and subjected to column chromatography (petroleum ether/ethyl acetate=1/1) to give compound 23A (1.160 g).

### Step B: Preparation of Compound 23B

In a reaction flask, a solution of 4 M hydrogen chloride in dioxane (4 mL) was added slowly to a solution of compound 23A (590 mg) in dichloromethane (10 mL) under an ice-water bath. After the addition was completed, the reaction solution was stirred for reaction at room temperature. After the reaction was completed, the reaction solution was directly distilled under reduced pressure to remove the solvent to obtain compound 23B, which was used directly in the next step.

### Step C: Preparation of Compound 23C

1,1′-binaphthyl-2,2′-diphenyl phosphine (1320 mg), sodium tert-butoxide (204 mg), 1,3-dibromobenzene (500 mg) and dioxane (10 mL) were added successively to a reaction flask containing compound 23B. After the addition was completed, the reaction solution was stirred for reaction at 120° C. After the reaction was completed, the reaction solution was concentrated and subjected to column chromatography (petroleum ether/ethyl acetate=80/20) to give compound 23C (380 mg).

MS (ESI, [M+H]⁺) m/z: 333.3.

### Step D: Preparation of Compound 23D

To a reaction flask were added [1,1′-bis(diphenylphosphino)ferrocene]palladium dichloride (93 mg), compound 23C (380 mg), bis(pinacolato)diboron (434 mg), potassium acetate (336 mg) and dioxane (8 mL) successively under nitrogen atmosphere. After the addition was completed, the reaction solution was reacted at 110° C. After the reaction was completed, the reaction solution was concentrated and subjected to column chromatography (petroleum ether/ethyl acetate=80/20) to give compound 23D (280 mg).

MS (ESI, [M+H]⁺) m/z: 381.3.

### Step E: Preparation of Compound 23E

To a reaction flask were added tetrakis(triphenylphosphine)palladium (99 mg), compound 1C (420 mg), compound 23D (325 mg), potassium acetate (236 mg), dioxane (5 mL), and water (0.5 mL) successively. After the addition was completed, the reaction solution was stirred for reaction at 120° C. After the reaction was completed, the reaction solution was concentrated and subjected to column chromatography (petroleum ether/ethyl acetate=60/40) to give compound 23E (154 mg).

MS (ESI, [M+H]⁺) m/z: 709.8.

### Step F: Preparation of Compound 23

To the reaction flask were added compound 23E (100 mg), dichloromethane (6 mL) and methanesulfonic acid (600 mg) successively. After the addition was completed, the reaction solution was stirred for reaction at room temperature. After the reaction was completed, the pH of the reaction solution was adjusted to be neutral by sodium bicarbonate, the reaction solution was extracted with dichloromethane, and the organic phase were combined, washed with saturated brine and dried over anhydrous sodium sulfate. The reaction solution was filtered, concentrated, and subjected to column chromatography (dichloromethane/methanol=97/3) to give compound 23 (60 mg).

MS (ESI, [M+H]⁺) m/z: 609.6.

¹H NMR (500 MHz, DMSO-d₆): δ 8.23 (d, J=3.5 Hz, 1H), 8.01-7.96 (m, 2H), 7.62 (d, J=7.5 Hz, 1H), 7.44 (d, J=7.5 Hz, 2H), 7.32 (d, J=7.0 Hz, 2H), 7.27-7.22 (m, 3H), 7.01 (s, 1H), 6.43 (d, J=7.0 Hz, 2H), 6.42-6.41 (m, 1H), 4.54 (t, J=7.5 Hz, 1H), 3.47 (q, J=7.5 Hz, 1H), 3.24 (t, J=7.5 Hz, 1H), 2.99 (s, 3H), 2.78 (s, 3H), 2.44-2.42 (m, 2H), 2.22-2.20 (s, 6H).

### Example 24: Preparation of Compound 24

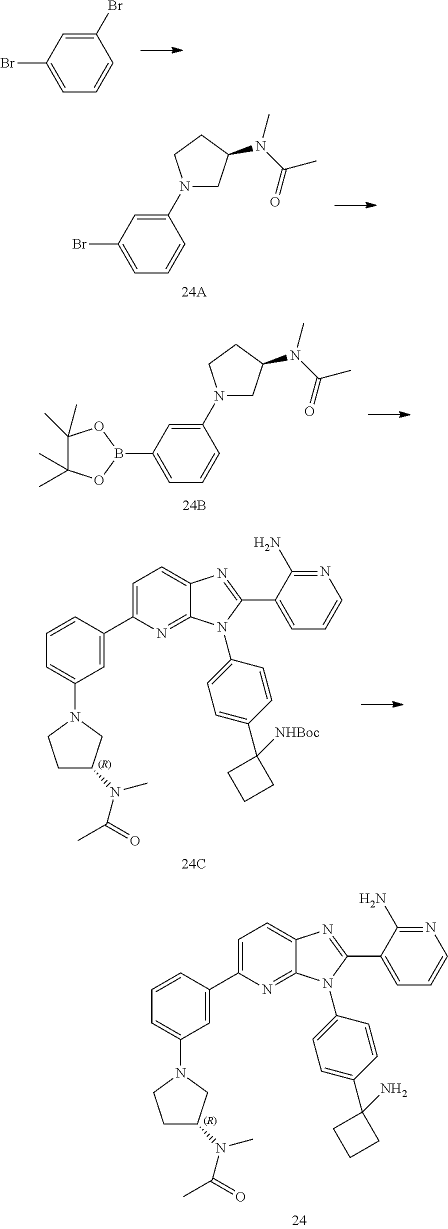

### Step A: Preparation of Compound 24A

To a reaction flask were added 1,1′-binaphthyl-2,2′-diphenyl phosphine (349 mg), bis(dibenzylideneacetone)palladium (256 mg), (R)-N-methyl-N-(pyrrol-3-yl)acetamide hydrochloride (500 mg), 1,3-dibromobenzene (660 mg), sodium tert-butoxide (807 mg) and dioxane (15 mL) successively under nitrogen atmosphere. After the addition was completed, the reaction solution was heated and reacted at 120° C. After the reaction was completed, the reaction solution was concentrated and subjected to column chromatography (petroleum ether/ethyl acetate=60/40) to give compound 24A (740 mg).

MS (ESI, [M+H]⁺) m/z: 297.4.

### Step B: Preparation of Compound 24B

To a reaction flask [1,1′-bis(diphenylphosphino)ferrocene]palladium dichloride (179 mg), compound 24A (650 mg), bis(pinacolato)diboron (1111 mg), potassium acetate (644 mg) and dioxane (15 mL) successively under nitrogen atmosphere; after the addition was completed, the reaction solution was reacted at 110° C. After the reaction was completed, the reaction solution was concentrated and subjected to column chromatography (petroleum ether/ethyl acetate=80/20) to give compound 24B (740 mg).

MS (ESI, [M+H]⁺) m/z: 345.6.

### Step C: Preparation of Compound 24C

To a reaction flask were added tetrakis(triphenylphosphine)palladium (208 mg), compound 1C, compound 24B (620 mg), potassium carbonate (498 mg), dioxane (15 mL), and water (0.5 mL) successively. After the addition was completed, the reaction solution was heated and reacted at 120° C. After the reaction was completed, the reaction solution was concentrated and subjected to column chromatography (dichloromethane/methanol=98/2) to give compound 24C (470 mg).

MS (ESI, [M+H]⁺) m/z: 673.6.

### Step D: Preparation of Compound 24

To the reaction flask were added compound 24C (420 mg), dichloromethane (6 mL) and methanesulfonic acid (360 mg) successively. After the addition was completed, the reaction solution was stirred for reaction at room temperature. After the reaction was completed, the pH of the reaction solution was adjusted to be neutral with saturated aqueous sodium bicarbonate, the reaction solution was extracted with dichloromethane, the organic phases were combined, washed with saturated brine, dried over anhydrous sodium sulfate, filtered, concentrated, and subjected to column chromatography (dichloromethane/methanol=96/4) to give compound 24 (140 mg).

MS (ESI, [M+H]⁺) m/z: 573.7.

¹H NMR (500 MHz, DMSO-d₆): δ 8.22 (d, J=8.0 Hz, 1H), 8.00 (dd, J=3.5 Hz, 7.0 Hz, 1H), 7.62 (d, J=7.5 Hz, 1H), 7.44 (d, J=7.5 Hz, 1H), 7.30-7.14 (m, 4H), 7.02 (s, 2H), 6.61 (br, 1H), 6.43-6.41 (m, 1H), 5.20 (t, J=7.5 Hz, 1H), 4.68 (t, J=7.5 Hz, 1H), 3.49-3.48 (m, 1H), 3.37-3.23 (m, 6H), 2.89 (s, 2H), 2.43-2.42 (m, 2H), 2.15-2.12 (m, 4H), 2.03 (s, 2H).

### Example 25: Preparation of Compound 25

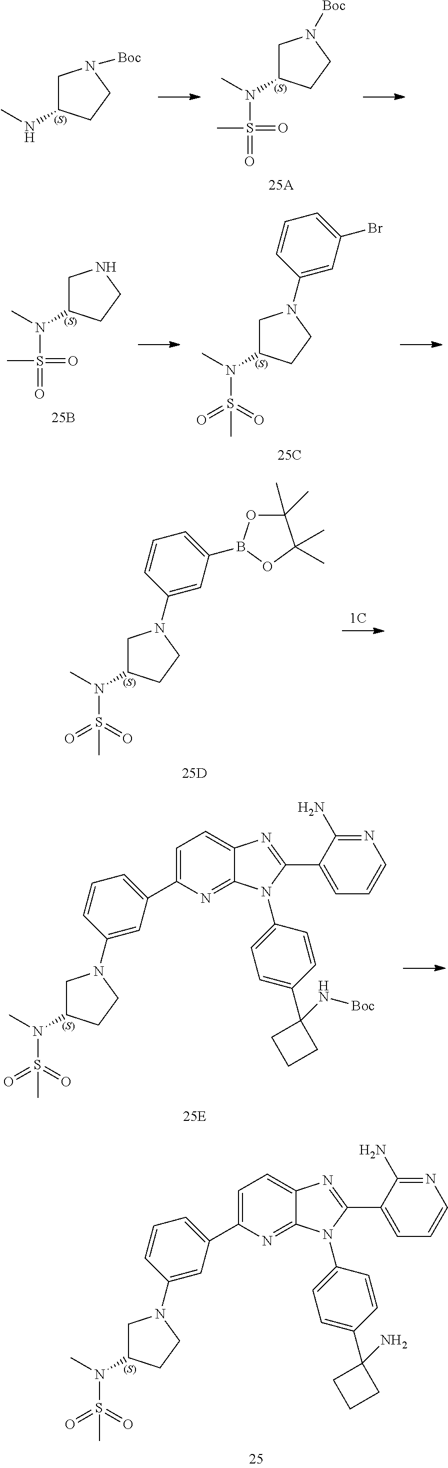

### Step A: Preparation of Compound 25A

To a reaction flask were added tert-butyl (S′)-3-(methylamino)pyrrolidine-1-carboxylate (1.60 g), N-diisopropylethylamine (1.24 g), and methanesulfonyl chloride (0.91 g) successively. After the addition was completed, the reaction solution was stirred for reaction at room temperature. After the reaction was completed, the reaction solution was concentrated to give compound 25A (2.53 g).

### Step B: Preparation of Compound 25B

To a reaction flask were added a solution (6 mL) of hydrochloric acid in dioxane, compound 25A (2.53 g), and dichloromethane (20 mL) successively. The reaction solution was reacted at room temperature; after the reaction was completed, the reaction solution was concentrated to give compound 25B (1.58 g).

### Step C: Preparation of Compound 25C

To a reaction flask were added compound 25B (0.700 g), 1,3-dibromobenzene (0.949 g), tris(dibenzylideneacetone)dipalladium (0.368 g), 1,1′-binaphthyl-2,2′-diphenyl phosphine (0.500 g), sodium tert-butoxide (1.500 g), and toluene (20 mL) successively under nitrogen atmosphere. After the addition was completed, the reaction solution was stirred for reaction at 120′° C. After the reaction was completed, the reaction solution was concentrated and subjected to column chromatography (petroleum ether/ethyl acetate=4/1) to give compound 25C (0.456 g).

MS (ESI, [M+H]⁺) m/z: 333.3.

### Step D: Preparation of Compound 25D

To a reaction flask were added potassium acetate (0.385 g), bis(pinacolato)diboron (0.627 g), [1,1′-bis (diphenylphosphino)ferrocene]palladium dichloride (0.160 g), compound 25C (0.430 g) and dioxane (20 mL) successively under nitrogen atmosphere. After the addition was completed, the reaction solution was stirred for reaction at 110° C. After the starting materials were reacted completely, the reaction solution was concentrated and subjected to column chromatography (petroleum ether/ethyl acetate=7/3) to give compound 25D (0.166 g).

MS (ESI, [M+H]⁺) m/z: 381.6.

### Step E: Preparation of Compound 25E

To a reaction flask were added compound 25D (0.165 g), compound 1C (0.213 g), potassium carbonate (0.180 g), tetrakis(triphenylphosphine)palladium (0.025 g), 1,4-dioxane (10 mL), and water (5 mL) successively under nitrogen atmosphere. After the addition was completed, the reaction solution was stirred for reaction at 110° C. After the reaction was completed, the reaction solution was concentrated and subjected to column chromatography (dichloromethane/methanol=17/3) to give compound 25E (0.075 g).

MS (ESI, [M+H]⁺) m/z: 709.7.

### Step F: Preparation of Compound 25

To a reaction flask were added compound 25E (0.075 g), dichloromethane (10 mL) and methanesulfonic acid (0.050 g) successively. After the addition was completed, the reaction solution was stirred for reaction at room temperature. After the reaction was completed, 20% aqueous sodium hydroxide solution was added, and the reaction solution was stirred under an ice bath. The pH of the reaction solution was adjusted to 12, the phases were separated, the aqueous phase was extracted by dichloromethane, and the organic phases were combined, washed with saturated brine and dried over anhydrous sodium sulfate. The reaction solution was filtered, concentrated, and subjected to column chromatography (dichloromethane/methanol=3/7) to give compound 25 (0.040 g).

MS (ESI, [M+H]⁺) m/z: 609.7.

¹H NMR (500 MHz, DMSO-d₆) δ8.41 (d, 1H), 8.20 (d, 1H), 7.96 (d, 1H), 7.84 (d, 1H), 7.75 (d, 2H), 7.67 (d, 2H), 7.41-7.45 (m, 2H), 7.29 (m, 1H), 6.96 (m, 1H), 4.26 (s, 4H), 4.00 (m, 1H), 3.53 (d, 2H), 3.08 (t, 2H), 2.63 (s, 4H), 2.25 (t, 3H), 1.71-2.01 (m, 7H).

### Example 26: Preparation of Compound 26

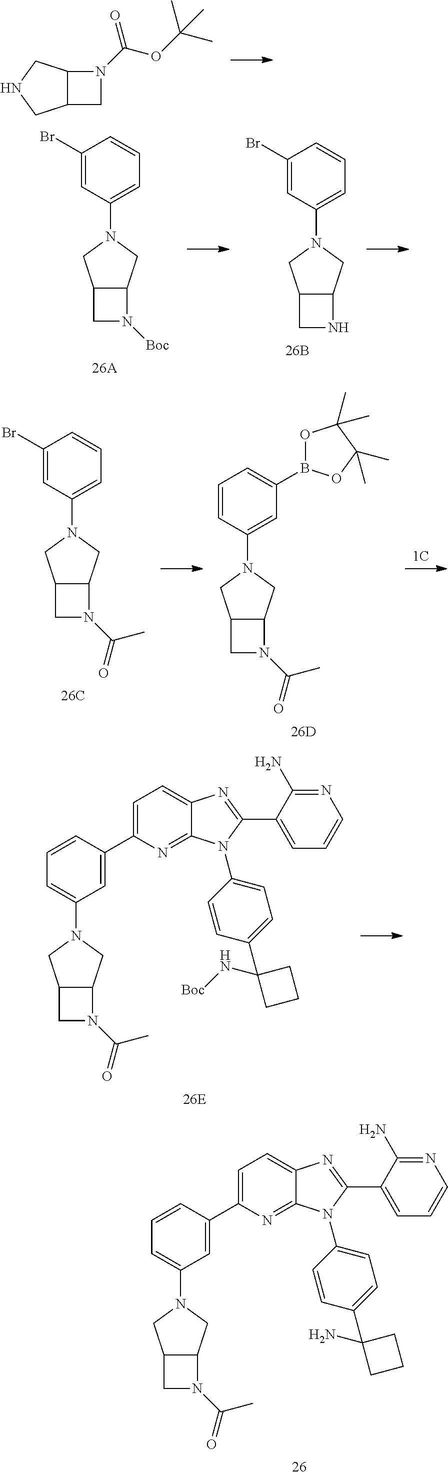

### Step A: Preparation of Compound 26A

To a reaction flask were added 1,3-dibromobenzene (0.54 g), tert-butyl 3,6-diazabicyclo[3.2.0]heptane-6-carboxylate (0.30 g), sodium tert-butoxide (0.29 g), tris(dibenzylideneacetone)dipalladium (0.14 g), 1,1′-binaphthyl-2,2′-diphenyl phosphine (0.09 g) and 1,4-dioxane (12 mL) successively. The reaction solution was stirred for reaction at 100° C. under nitrogen atmosphere. After the reaction was completed, the reaction solution was filtered, concentrated, and subjected to column chromatography (petroleum ether/ethyl acetate=5/1) to give compound 26A (0.51 g).

MS (ESI, [M-Boc+H]⁺) m/z: 253.4.

### Step B: Preparation of Compound 26B

To a reaction flask were added compound 26A (0.46 g) and a solution of 4 M hydrogen chloride in dioxane (8 mL) successively, and the reaction solution was stirred for reaction at room temperature. After the reaction was completed, the reaction solution was concentrated to give compound 26B (0.43 g).

MS (ESI, [M+H]⁺) m/z: 253.4.

### Step C: Preparation of Compound 26C

Acetyl chloride (2.98 g) was slowly added to a solution of compound 26B (0.43 g) and triethylamine (0.69 g) in dichloromethane (20 mL) in a reaction flask at 0° C. under nitrogen atmosphere. After the addition was completed, the reaction solution was stirred for reaction at room temperature. After the reaction was completed, the reaction solution was added with a saturated aqueous sodium bicarbonate solution (75 mL) and extracted with dichloromethane (50 mL×3), and the organic phases were combined, washed with saturated brine (100 mL), and dried over anhydrous sodium sulfate. The organic phase was filtered, concentrated, and subjected to column chromatography (petroleum ether/ethyl acetate=1/4) to give compound 26C (0.25 g).

MS (ESI, [M+H]⁺) m/z: 295.4.

### Step D: Preparation of Compound 26D

To a 100 mL reaction flask were added compound 26C (0.25 g), bis(pinacolato)diboron (0.32 g), potassium acetate (0.25 g), [1,1′-bis(diphenylphosphino)ferrocene]palladium dichloride dichloromethane complex (0.069 g) and 1,4-dioxane (15 mL) successively. The reaction solution was stirred for reaction at 100° C. under nitrogen atmosphere. After the reaction was completed, the reaction solution was filtered, concentrated, and subjected to column chromatography (petroleum ether/ethyl acetate=1/4) to give compound 26D (0.22 g).

MS (ESI, [M+H]⁺) m/z: 343.6.

### Step E: Preparation of Compound 26E

To a 25 mL microwave tube were added compound 26D (0.21 g), compound 1C (0.20 g), potassium carbonate (0.17 g), tetrakis(triphenylphosphine)palladium (0.047 g), 1,4-dioxane (10 mL) and water (1.5 mL) successively. After the addition was completed, the reaction solution was purged with nitrogen gas, and reacted at 140° C. in microwave reactor for 2 h. After the reaction was completed, the reaction solution was filtered, concentrated, and subjected to column chromatography (dichloromethane/methanol=20/1) to give compound 26E (0.22 g).

MS (ESI, [M+H]⁺) m/z: 671.7.

### Step F: Preparation of Compound 26

To a 50 mL reaction flask were added compound 26E (183 mg), methanesulfonic acid (0.2 mL), and dichloromethane (10 mL) successively. The reaction solution was stirred for reaction at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 26 (106 mg).

HRMS (ESI, [M+H]⁺) m/z: 571.2924.

¹H NMR (500 MHz, CDCl₃) δ 8.11 (dd, J=8.3, 5.2 Hz, 1H), 8.07 (dt, J=4.9, 1.7 Hz, 1H), 7.79 (dd, J=8.4, 3.6 Hz, 1H), 7.60-7.54 (m, 2H), 7.53-7.41 (m, 4H), 7.36-7.30 (m, 1H), 7.16 (ddd, J=14.3, 7.8, 1.8 Hz, 1H), 6.82-6.76 (m, 1H), 6.62 (s, 2H), 6.40 (dt, J=7.8, 5.3 Hz, 1H), 4.94 (dd, J=6.9, 4.5 Hz, 1H), 4.30-4.10 (m, 2H), 3.98-3.83 (m, 1H), 3.76 (dd, J=22.2, 10.2 Hz, 1H), 3.29-3.18 (m, 1H), 3.08-2.88 (m, 2H), 2.68-2.56 (m, 2H), 2.32-2.05 (m, 4H), 1.84 (s, 3H).

### Example 27: Preparation of Compound 27

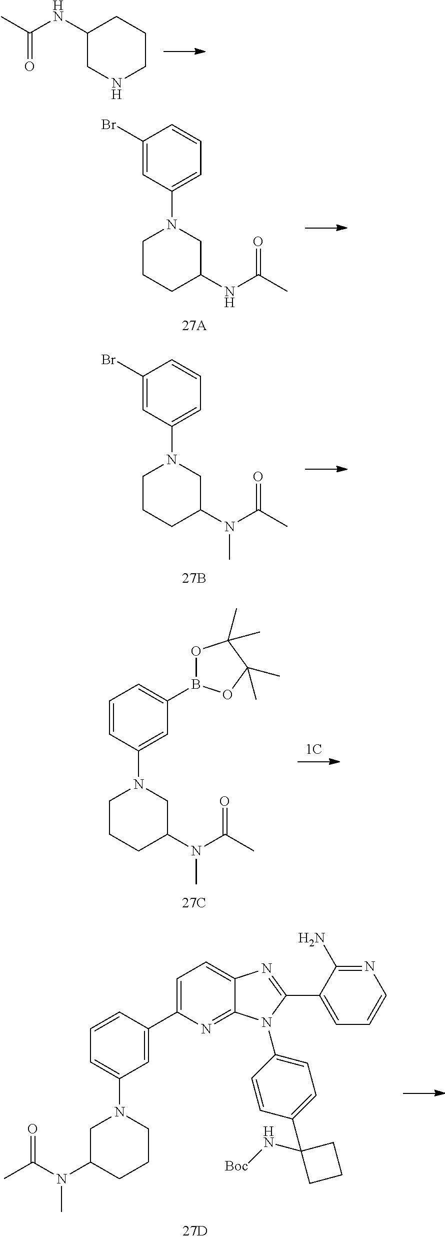

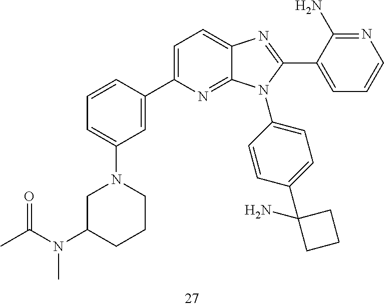 [SMILES: CC(=O)N(C)C1CCCN(c2cccc(-c3ccc4nc(-c5cccnc5N)n(-c5ccc(C6(N)CCC6)cc5)c4n3)c2)C1]

### Step A: Preparation of Compound 27A

To a reaction flask were added 1,3-dibromobenzene (4.98 g), tert-butyl 3,6-diazabicyclo[3.2.0]heptane-6-carboxylate (2.00 g), sodium tert-butoxide (2.70 g), tris(dibenzylideneacetone)dipalladium (0.64 g), 1,1′-binaphthyl-2,2′-diphenyl phosphine (0.66 g) and 1,4-dioxane (100 mL) successively. The reaction solution was stirred for reaction at 100° C. under nitrogen atmosphere; after the reaction was completed, the reaction solution was filtered, concentrated, and subjected to column chromatography (petroleum ether/ethyl acetate=1/1) to give compound 27A (2.78 g).

MS (ESI, [M+H]⁺) m/z: 297.4.

### Step B: Preparation of Compound 27B

Sodium hydride (1.87 g) was slowly added to a solution of compound 27A (2.78 g) in tetrahydrofuran (60 mL) at 0° C. under nitrogen atmosphere. After the addition was completed, the mixture was stirred at room temperature for 0.5 h, and methyl iodide (6.64 g) was added thereto for reaction. After the reaction was completed, a saturated aqueous ammonium chloride solution was added to the reaction solution in an ice bath, followed by extraction with ethyl acetate, and the organic phases were combined, washed with saturated brine, and dried over anhydrous sodium sulfate. The organic phase was filtered, concentrated, and subjected to column chromatography (petroleum ether/ethyl acetate=1/2) to give compound 27B (1.91 g).

MS (ESI, [M+H]⁺) m/z: 311.4.

### Step C: Preparation of Compound 27C

To a reaction flask were added compound 27B (1.91 g), bis(pinacolato)diboron (2.34 g), potassium acetate (1.81 g), [1,1′-bis(diphenylphosphino)ferrocene]palladium dichloride dichloromethane complex (0.50 g) and 1,4-dioxane (120 mL) successively. The reaction solution was stirred for reaction at 100° C. under nitrogen atmosphere. After the reaction was completed, the reaction solution was filtered, concentrated, and subjected to column chromatography (petroleum ether/ethyl acetate=1/1) to give compound 27C (2.11 g).

MS (ESI, [M+H]⁺) m/z: 359.6.

### Step D: Preparation of Compound 27D

To a microwave tube were added compound 27C (0.33 g), compound 1C (0.30 g), potassium carbonate (0.25 g), tetrakis(triphenylphosphine)palladium (71 mg), 1,4-dioxane (14 mL), and water (2 mL) successively. After the addition was completed, the reaction solution was purged with nitrogen gas, and reacted at 140° C. in microwave reactor. After the reaction was completed, the reaction solution was filtered, concentrated, and subjected to column chromatography (dichloromethane/methanol=20/1) to give compound 27D (0.38 g).

MS (ESI, [M+H]⁺) m/z: 687.8.

### Step E: Preparation of Compound 27

Compound 27D (370 mg), methanesulfonic acid (0.3 mL) and dichloromethane (16 mL) were added successively to a reaction flask. The reaction solution was stirred for reaction at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 27 (157 mg).

HRMS (ESI, [M+H]⁺) m/z: 587.3278.

¹H NMR (500 MHz, CDCl₃) δ 8.10 (dd, J=9.5, 8.3 Hz, 1H), 8.06 (dd, J=4.8, 1.8 Hz, 1H), 7.79 (dd, J=8.4, 1.3 Hz, 1H), 7.67-7.57 (m, 3H), 7.48 (dt, J=7.8, 1.0 Hz, 1H), 7.46-7.41 (m, 2H), 7.31 (dt, J=18.8, 7.9 Hz, 1H), 7.12 (ddd, J=29.3, 7.8, 1.8 Hz, 1H), 6.93 (td, J=7.6, 2.4 Hz, 1H), 6.60 (s, 2H), 6.39 (ddd, J=7.8, 5.9, 4.8 Hz, 1H), 4.77-4.68 (m, 1H), 3.86-3.72 (m, 1H), 3.72-3.56 (m, 1H), 2.91 (d, J=18.3 Hz, 3H), 2.75-2.56 (m, 4H), 2.47-2.35 (m, 2H), 2.26-2.18 (m, 1H), 2.15 (s, 3H), 1.92-1.70 (m, 5H).

### Example 28: Preparation of Compound 28

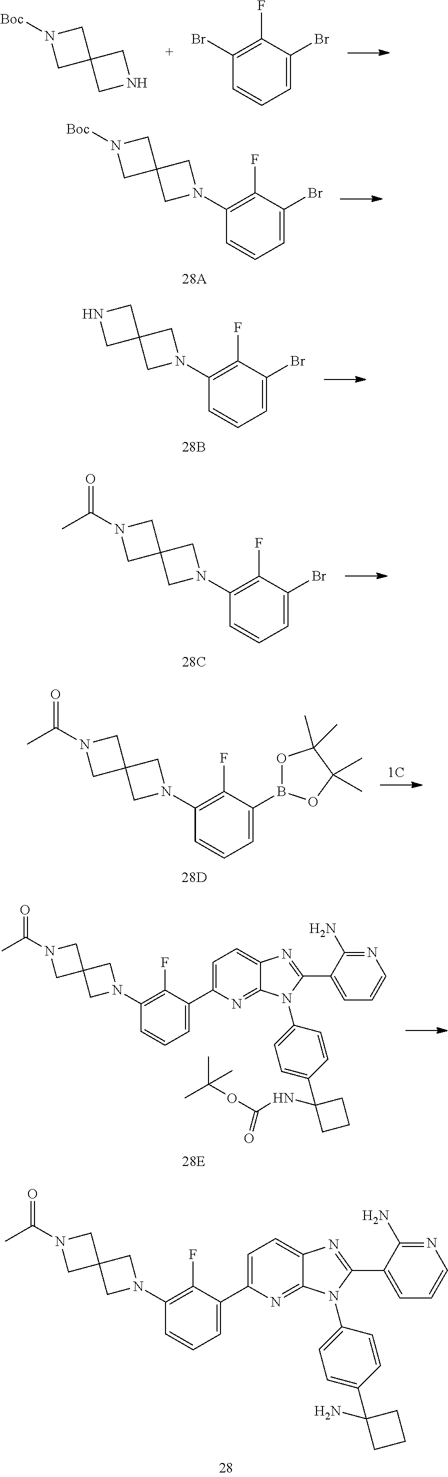

### Step A: Preparation of Compound 28A

To a reaction flask were added tert-butyl 2,6-diazaspiro[3.3]heptane-2-carboxylate (0.50 g), 1,3-dibromofluorobenzene (0.66 g), tris(dibenzylideneacetone)dipalladium (80 mg), 1,1′-binaphthyl-2,2′-diphenyl phosphine (60 mg), tetrahydrofuran (50 mL) and sodium tert-butoxide (1.00 g) successively. After the addition was completed, the reaction solution was stirred for reaction at 80° C. under nitrogen atmosphere. After the reaction was completed, the reaction solution was filtered, concentrated, and subjected to column chromatography (petroleum ether/ethyl acetate=10/1) to give compound 28A (0.51 g).

### Step B: Preparation of Compound 28B

Compound 28A (0.50 g) was weighed out and added to a reaction flask, methylene chloride (10 mL) was added, the mixture was stirred and dissolved, followed by addition of methanesulfonic acid (0.39 g), and after the addition was completed, the reaction solution was stirred for reaction at room temperature. After the reaction was completed, triethylamine (2 mL) was added to terminate the reaction to give a solution of compound 28B. The reaction solution was not concentrated and was directly fed for the next step.

### Step C: Preparation of Compound 28C

Acetic anhydride (206 mg) was added to the above solution of compound 28B, and the mixture was stirred at room temperature for reaction. After the reaction was completed, the reaction solution was concentrated and subjected to column chromatography (dichloromethane/methanol=96:4) to give compound 28C (313 mg).

MS (ESI, [M+H]⁺) m/z: 313.79.

### Step D: Preparation of Compound 28D

To a reaction flask containing compound 28C (300 mg) were added bis(pinacolato)diboronate (365 mg), [1,1′-bis(diphenylphosphino)ferrocene]palladium dichloride dichloromethane complex (39 mg), 1,4-dioxane (30 ml) and potassium acetate (282 mg) successively. After the addition was completed, the reaction solution was stirred for reaction at 120° C. under nitrogen atmosphere. After the reaction was completed, the reaction solution was filtered, concentrated and subjected to column chromatography (dichloromethane/methanol=98/2) to give compound 28D (278 mg).

MS (ESI, [M+H]⁺) m/z: 361.04.

### Step E: Preparation of Compound 28E

To a microwave tube were added compound 28D (300 mg), compound 1C (409 mg), potassium carbonate (460 mg), tetrakis(triphenylphosphine)palladium (48 mg), 1,4-dioxane (10 mL), and water (1 mL) successively. After the addition was completed, the reaction solution was purged with nitrogen gas, and reacted at 120° C. in microwave reactor. After the reaction was completed, the reaction solution was filtered, concentrated and subjected to column chromatography (dichloromethane/methanol=96/4) to give compound 28E (252 mg).

MS (ESI, [M+H]⁺) m/z: 689.5.

### Step F: Preparation of Compound 28

Compound 28E (250 mg), methanesulfonic acid (349 mg) and dichloromethane (30 mL) were added successively to a reaction flask. The reaction solution was stirred for reaction at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 28 (70 mg).

MS (ESI, [M+H]⁺) m/z: 589.5.

¹H NMR (500 MHz, CDCl₃): 8.25 (d, J=8.5 Hz, 1H), 8.00 (dd, J=2.0, 5.0 Hz, 1H), 7.68 (dd, J=2.0, 8.0 Hz, 1H), 7.60 (d, J=8.5 Hz, 2H), 7.42 (d, J=8.5 Hz, 2H), 7.20 (dd, J=2.0, 8.0 Hz, 1H), 7.10-7.44 (m, 2H), 6.94 (m, 2H), 6.61-6.57 (m, 1H), 6.42-6.40 (m, 1H), 4.29 (s, 2H), 4.08 (s, 4H), 4.02 (s, 2H), 2.44-2.37 (m, 2H), 2.13-2.06 (m, 2H), 2.06-1.98 (m, 1H), 1.75 (s, 3H), 1.73-1.65 (m, 1H).

### Example 29: Preparation of Compound 29

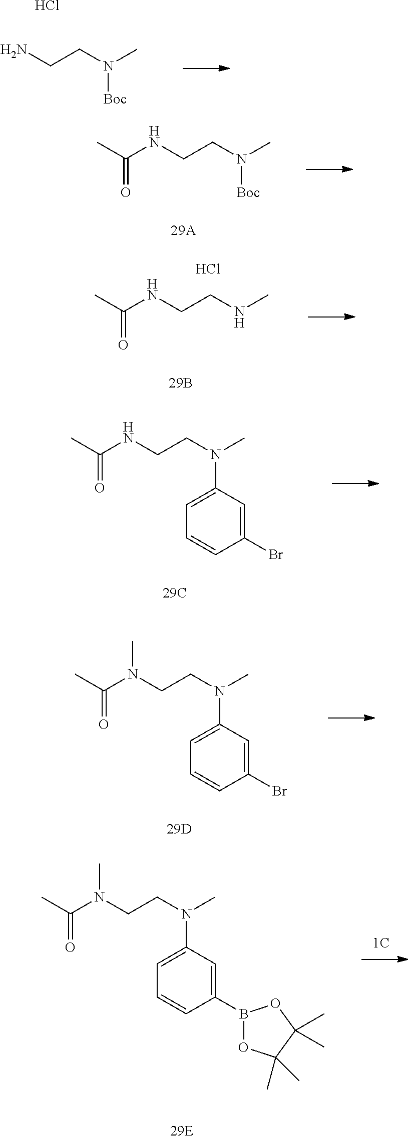

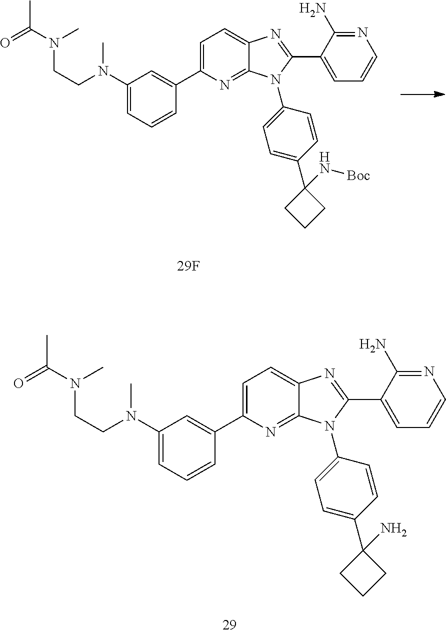 [SMILES: CC(=O)N(C)CCN(C)c1cccc(-c2ccc3nc(-c4cccnc4N)n(-c4ccc(C5(NC(=O)OC(C)(C)C)CCC5)cc4)c3n2)c1 ; CC(=O)N(C)CCN(C)c1cccc(-c2ccc3nc(-c4cccnc4N)n(-c4ccc(C5(N)CCC5)cc4)c3n2)c1]

### Step A: Preparation of Compound 29A

N-tert-butoxycarbonyl-N-methylethylenediamine (1.00 g), methylene chloride (30 mL), anhydrous potassium carbonate (1.59 g) and acetic anhydride (0.64 g) were added to the reaction flask successively. After the addition was completed, the reaction solution was stirred for reaction at room temperature. After the reaction was completed, The reaction solution was washed with water and saturated brine successively, and dried over anhydrous sodium sulfate. The reaction solution was filtered and concentrated to give compound 29A (1.13 g).

### Step B: Preparation of Compound 29B

To a reaction flask containing compound 29A (1.13 g) was added a solution of hydrogen chloride (4 M) in 1,4-dioxane (20 mL), and after the addition was completed, the reaction solution was stirred for reaction at room temperature. After the reaction was completed, the reaction solution was concentrated to give compound 29B (0.87 g).

### Step C: Preparation of Compound 29C

To a reaction flask were added compound 29B (0.87 g), 1,3-dibromofluorobenzene (5.42 g), tris(dibenzylideneacetone)dipalladium (263 mg), 1,1′-binaphthyl-2,2′-diphenyl phosphine (179 mg), tetrahydrofuran (50 mL) and sodium tert-butoxide (3.31 g) successively. After the addition was completed, the reaction solution was stirred for reaction at 80° C. under nitrogen atmosphere. After the reaction was completed, the reaction solution was filtered, concentrated, and subjected to column chromatography (ethyl acetate) to give compound 29C (0.48 g).

MS (ESI, [M+H]⁺) m/z: 271.1.

### Step D: Preparation of Compound 29D

Tetrahydrofuran (10 mL) was added to a reaction flask containing compound 29C (0.48 g), and after the mixture was dissolved with stirring, sodium hydride (0.28 g) was added after the reaction solution was cooled with ice/ethanol. After the addition was completed, the reaction solution was stirred for 10 min, methyl iodide (0.76 g) was added, and after the addition was completed, the reaction solution was stirred for reaction at room temperature. The reaction was completed. The reaction solution was poured into aqueous saturated ammonium chloride solution, stirred, and extracted with dichloromethane, and the organic phases were combined. The reaction solution was dried over anhydrous sodium sulfate. The reaction solution was filtered and concentrated to give compound 29D (0.64 g).

MS (ESI, [M+H]⁺) m/z: 285.1.

### Step E: Preparation of Compound 29E

To a reaction flask containing compound 29D (509 mg) were added bis(pinacolato)diboron (680 mg), [1,1′-bis(diphenylphosphino)ferrocene]palladium dichloride dichloromethane complex (73 mg), 1,4-dioxane (20 mL) and potassium acetate (525 mg) successively. After the addition was completed, the reaction solution was stirred for reaction at 120° C. under nitrogen atmosphere. After the reaction was completed, the reaction solution was filtered, concentrated and subjected to column chromatography (petroleum ether/ethyl acetate=1/2) to give compound 29E (441 mg).

MS (ESI, [M+H]⁺) m/z: 333.3.

### Step E: Preparation of Compound 29F

To a microwave tube were added compound 29E (440 mg), compound 1C (716 mg), potassium carbonate (550 mg), tetrakis(triphenylphosphine)palladium (77 mg), 1,4-dioxane (10 mL), and water (1 mL) successively. After the addition was completed, the reaction solution was purged with nitrogen gas, and reacted at 120° C. in microwave reactor for 2 h. After the reaction was completed, the reaction solution was filtered, concentrated and subjected to column chromatography (dichloromethane/methanol=96/4) to give compound 29F (400 mg).

MS (ESI, [M+H]⁺) m/z: 661.39.

### Step G: Preparation of Compound 29

To a reaction flask were added compound 29F (400 mg), methanesulfonic acid (582 mg) and dichloromethane (30 mL) successively, and the reaction solution was stirred for reaction at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 29 (387 mg).

MS (ESI, [M+H]⁺) m/z: 561.34.

¹H NMR (500 MHz, CDCl₃): 8.29 (d, J=8.5 Hz, 1H), 8.01 (dd, J=1.5, 5.0 Hz, 1H), 7.96 (t, J=8.5 Hz, 1H), 7.62 (d, J=8.0 Hz, 2H), 7.47-7.40 (m, 3H), 7.31-7.22 (m, 2H), 7.01 (s, 2H), 6.79-6.74 (m, 1H), 6.43-6.41 (m, 1H), 3.56-3.54 (m, 1H), 3.49-3.46 (m, 1H), 3.43-3.33 (m, 6H), 2.94 (s, 2H), 2.90 (s, 1H), 2.78 (s, 1H), 2.46-2.41 (m, 2H), 2.18-2.13 (m, 2H), 2.08-2.02 (m, 1H), 1.88 (s, 2H), 1.79 (s, 2H), 1.75-1.66 (m, 1H).

### Example 30: Preparation of Compound 30

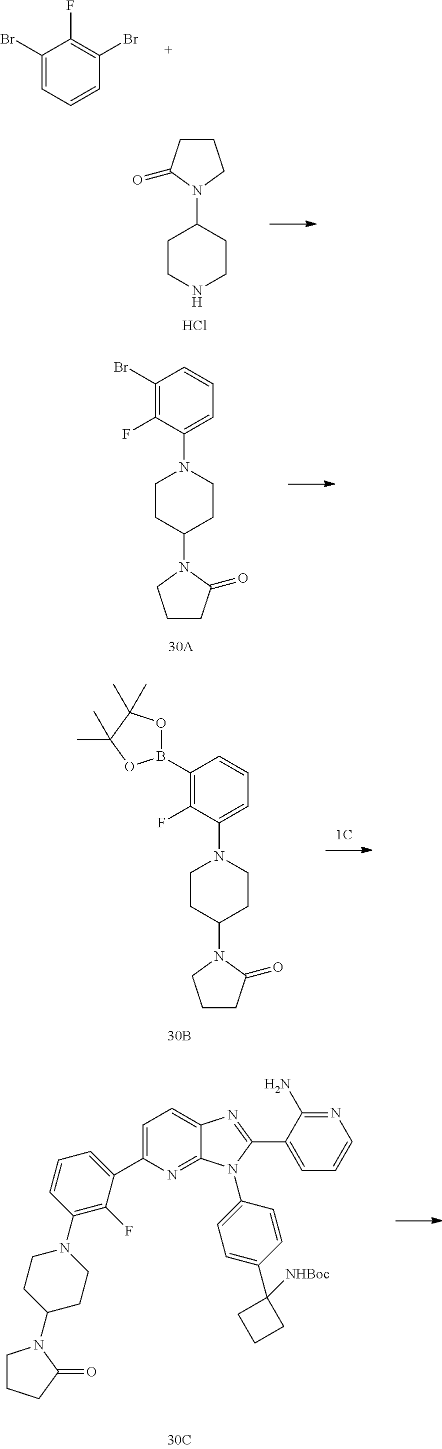 [SMILES: Fc1c(Br)cccc1Br ; O=C1CCCN1C1CCNCC1 ; Cl ; O=C1CCCN1C1CCN(c2cccc(Br)c2F)CC1 ; CC1(C)OB(c2cccc(N3CCC(N4CCCC4=O)CC3)c2F)OC1(C)C ; CC(C)(C)OC(=O)NC1(c2ccc(-n3c(-c4cccnc4N)nc4ccc(-c5cccc(N6CCC(N7CCCC7=O)CC6)c5F)nc43)cc2)CCC1]

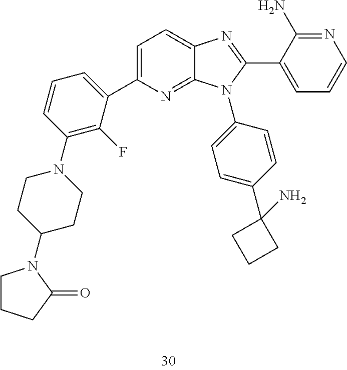 [SMILES: Nc1ncccc1-c1nc2ccc(-c3cccc(N4CCC(N5CCCC5=O)CC4)c3F)nc2n1-c1ccc(C2(N)CCC2)cc1]

### Step A: Preparation of Compound 30A

1-(Piperidin-4-yl)pyrrolidin-2-one hydrochloride (0.32 g), 1,3-dibromofluorobenzene (0.40 g), tris(dibenzylideneacetone)dipalladium (0.07 g), 1,1′-binaphthyl-2,2′-diphenyl phosphine (0.10 g), sodium tert-butoxide (0.33 g) and toluene (20 mL) were added to a reaction flask successively under nitrogen atmosphere. After the addition was completed, the reaction solution was stirred for reaction at 120° C. After the starting materials were reacted completely, the reaction solution was concentrated and subjected to column chromatography (dichloromethane/methanol=65/1) to give compound 30A (0.20 g).

### Step B: Preparation of Compound 30B

To a reaction flask were added potassium acetate (0.06 g), bis(pinacolato)diboron (0.10 g), [1,1′-bis (diphenylphosphino)ferrocene]palladium dichloride (25 mg), compound 30A (0.20 g) and dioxane (20 mL) successively under nitrogen atmosphere. After the addition was completed, the reaction solution was stirred for reaction at 110° C. After the starting materials were reacted completely, the reaction solution was concentrated and subjected to column chromatography (dichloromethane/methanol=65/1) to give compound 30B (0.21 g).

### Step C: Preparation of Compound 30C

To a reaction flask were added compound 30B (0.21 g), compound 1C (0.11 g), potassium carbonate (0.06 g), tetrakis(triphenylphosphine)palladium (13 mg), 1,4-dioxane (15 mL), and water (7.5 mL) successively under nitrogen atmosphere. After the addition was completed, the reaction solution was stirred for reaction at 110° C. After the reaction was completed, the reaction solution was concentrated and subjected to column chromatography (dichloromethane/methanol=3/7) to give compound 30C (0.16 g).

MS (ESI, [M+H]⁺) m/z: 717.8.

### Step D: Preparation of Compound 30

To a reaction flask were added compound 30C (0.16 g), dichloromethane (10 mL) and methanesulfonic acid (0.21 g) successively. After the addition was completed, the reaction solution was stirred for reaction at room temperature. After the reaction was completed, 20% aqueous sodium hydroxide solution was added, and the reaction solution was stirred under an ice bath. The pH of the reaction solution was adjusted to 12, the phases were separated, the aqueous phase was extracted by dichloromethane, and the organic phases were combined, washed with saturated brine and dried over anhydrous sodium sulfate. The reaction solution was filtered, concentrated, and subjected to column chromatography (dichloromethane/methanol=2/3) to give compound 30 (0.08 g).

MS (ESI, [M+H]⁺) m/z: 617.7.

¹H NMR (500 MHz, DMSO-d₆) δ 8.23 (d, 1H), 7.96-8.02 (m, 2H), 7.63 (d, 2H), 7.47 (d, 2H), 7.22-7.33 (m, 3H), 6.98 (s, 2H), 6.62-6.64 (m, 1H), 6.41-6.43 (m, 1H), 4.51-4.57 (m, 1H), 3.47 (q, 2H), 3.24 (q, 2H), 2.99 (s, 3H), 2.78 (s, 3H), 2.44-2.48 (m, 2H), 2.02-2.25 (m, 5H), 1.69-1.77 (m, 1H), 1.23 (s, 2H).

### Example 31: Preparation of Compound 31

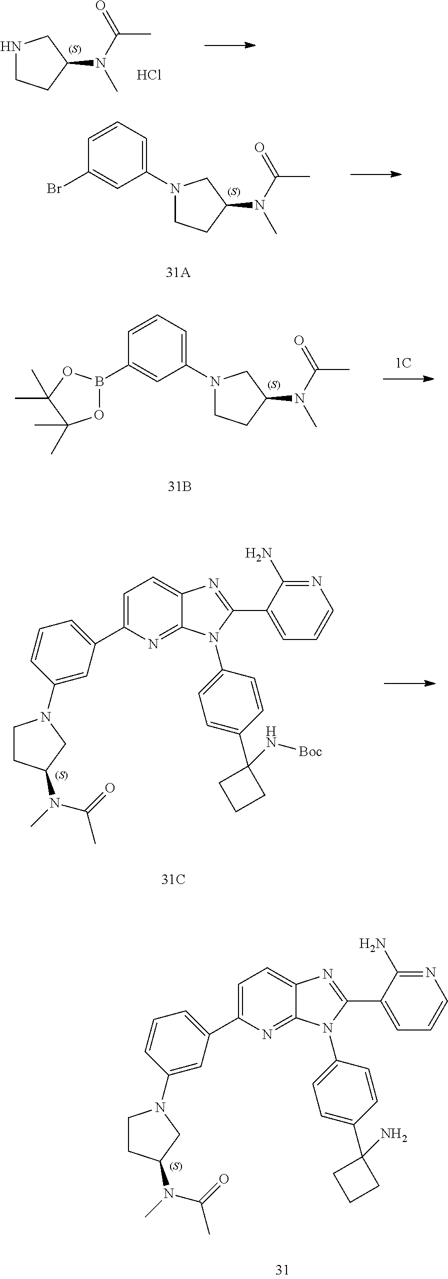

### Step A: Preparation of Compound 31A

1-(Piperidin-4-yl)pyrrolidin-2-one hydrochloride (0.61 g), 1,3-dibromobenzene (0.80 g), tris(dibenzylideneacetone)dipalladium (0.31 g), 1,1′-binaphthyl-2,2′-diphenyl phosphine (0.32 g), sodium tert-butoxide (0.49 g) and toluene (10 mL) were added to a reaction flask successively under nitrogen atmosphere. After the addition was completed, the reaction solution was stirred for reaction at 120° C. After the starting materials were reacted completely, the reaction solution was concentrated and subjected to column chromatography (petroleum ether/ethyl acetate=2/3) to give compound 31A (0.50 g). MS (ESI, [M+H]⁺) m/z: 297.1.

### Step B: Preparation of Compound 31B

To a reaction flask were added potassium acetate (0.489 g), bis(pinacolato)diboron (0.811 g), [1,1′-bis (diphenylphosphino)ferrocene]palladium dichloride (0.207 g), compound 31A (0.502 g) and dioxane (20 mL) successively under nitrogen atmosphere. After the addition was completed, the reaction solution was stirred for reaction at 110° C. After the starting materials were reacted completely, the reaction solution was concentrated and subjected to column chromatography (petroleum ether/ethyl acetate=7/3) to give compound 31B (0.293 g).

MS (ESI, [M+H]⁺) m/z: 345.6.

### Step C: Preparation of Compound 31C

To a reaction flask were added compound 31B (0.293 g), compound 1C (0.320 g), potassium carbonate (0.270 g), tetrakis(triphenylphosphine)palladium (0.040 g), 1,4-dioxane (15 mL), and water (7.5 mL) successively under nitrogen atmosphere. After the addition was completed, the reaction solution was stirred for reaction at 110° C. After the reaction was completed, the reaction solution was concentrated and subjected to column chromatography (dichloromethane/methanol=7/3) to give compound 31C (0.294 g).

MS (ESI, [M+H]⁺) m/z: 673.4.

### Step D: Preparation of Compound 31

To a reaction flask were added compound 31C (0.294 g), dichloromethane (10 mL) and methanesulfonic acid (0.210 g) successively. After the addition was completed, the reaction solution was stirred for reaction at room temperature. After the reaction was completed, 20% aqueous sodium hydroxide solution was added, and the reaction solution was stirred under an ice bath. The pH of the reaction solution was adjusted to 12, the phases were separated, the aqueous phase was extracted by dichloromethane, and the organic phases were combined, washed with saturated brine and dried over anhydrous sodium sulfate. The reaction solution was filtered, concentrated, and subjected to column chromatography (dichloromethane/methanol=2/3) to give compound 31 (0.183 g).

MS (ESI, [M+H]⁺) m/z: 573.7.

¹H NMR (500 MHz, DMSO-d₆) δ 8.21 (d, 1H), 7.95-8.01 (m, 2H), 7.62 (d, 2H), 7.44 (d, 2H), 7.23-7.32 (m, 4H), 7.02 (s, 2H), 6.60 (d, 1H), 6.41-6.43 (m, 1H), 3.36-3.49 (m, 1H), 3.21-3.35 (m, 4H), 2.89 (s, 3H), 2.42-2.43 (m, 2H), 2.03-2.20 (m, 8H), 1.7 (m, 1H).

### Example 32: Preparation of Compound 32

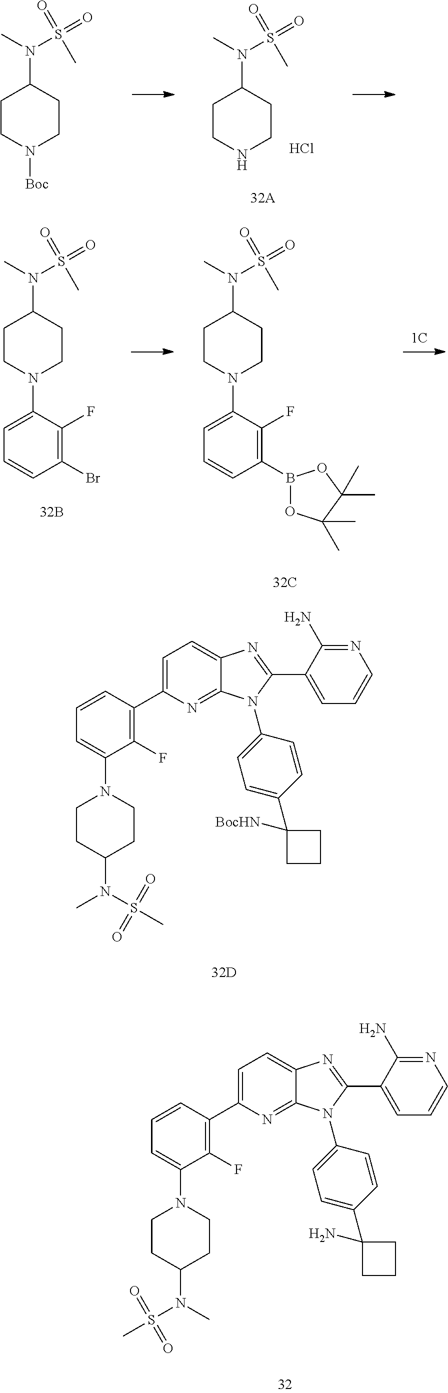

### Step A: Preparation of Compound 32A

To a 100 mL single-neck flask were added tert-butyl 4-(N-methylmethanesulfonamide)piperidine-1-carbamate (1.8 g), methylene chloride (5 mL), and a solution of 4 M hydrogen chloride (2 mL) in dioxane successively, and after the addition was completed, the reaction solution was stirred for reaction at room temperature. After the reaction was completed, the reaction solution was distilled under reduced pressure to give compound 32A (1.26 g), which was used directly in the next step.

### Step B: Preparation of Compound 32B

To a 100 mL single-neck flask were added 2,2′-(3,3′-dichloro-1,1′-biphenyl-4,4′-bisazo)bis(N-phenyl-3-oxo-butyramide) (0.491 g), bis(dibenzylideneacetone)palladium (0.361 g), 1,3-dibromo-2-fluorobenzene (1 g), compound 32A (1.2 g), sodium tert-butoxide (0.757 g), and dioxane (15 mL) successively under nitrogen atmosphere, and the mixture was heated to 120° C. for reaction. After the reaction was completed, the reaction solution was distilled under reduced pressure to remove the solvent, concentrated, and then subjected to column chromatography (petroleum ether:ethyl acetate=85:15) to give compound 32B (680 mg).

MS (ESI, [M+H]⁺) m/z: 365.1.

### Step C: Preparation of Compound 32C

To a 100 mL one-neck flask were added [1,1′-bis(diphenylphosphino)ferrocene]palladium dichloride dichloromethane complex (139 mg), compound 32B (620 mg), bis(pinacolato)diboron (862 mg), potassium acetate (333 mg), and dioxane (15 mL) successively, and the mixture was heated to 120° C. under nitrogen atmosphere for reaction. After the reaction was completed, the reaction solution was concentrated and subjected to column chromatography (petroleum ether:ethyl acetate=75:25) to give compound 32C (420 mg).

MS (ESI, [M+H]⁺) m/z: 413.3.

### Step D: Preparation of Compound 32D

To a 100 mL single-neck flask were added tetrakis(triphenylphosphine)palladium (51.8 mg), compound 32C (220 mg), compound 1C (220 mg), potassium carbonate (124 mg), dioxane (5 mL) and water (0.5 mL) successively, and the mixture was transferred to a 100° C. oil bath and heated for reaction. After the reaction was completed, the reaction solution was concentrated and subjected to column chromatography (dichloromethane:methanol=94:6) to give 32D (180 mg).

MS (ESI, [M+H]⁺) m/z: 741.4.

### Step E: Preparation of Compound 32

To a 50 mL single-necked flask were added compound 32D (180 mg), dichloromethane (10 mL) and methanesulfonic acid (46.7 mg) successively, and after the addition was completed, the reaction solution was stirred for reaction at room temperature. After the reaction was completed, the pH of the reaction solution was adjusted to be neutral with a saturated aqueous sodium carbonate solution, extracted with dichloromethane, washed with saturated brine, dried over anhydrous sodium sulfate, filtered, concentrated, and subjected to column chromatography (dichloromethane:methanol=96:4) to give compound 32 (120 mg).

MS (ESI, [M+H]⁺) m/z: 641.7.

¹H NMR (500 MHz, CDCl₃): δ 8.12 (d, J=7.5 Hz, 1H), 8.07 (dd, J=5.0, 10.0 Hz, 1H), 7.80 (dd, J=5.0, 10.0 Hz, 1H), 7.56-7.51 (m, 3H), 7.40 (d, J=10.0 Hz, 1H), 7.12 (q, J=10.0 Hz, 1H), 6.99 (t, J=7.5 Hz, 1H), 6.59 (br, 2H), 6.39 (q, J=8.0 Hz, 1H), 3.93-3.91 (m, 1H), 3.51 (d, J=10.0 Hz, 2H), 2.88-2.87 (m, 6H), 2.81 (t, J=10.0 Hz, 2H), 2.63-2.58 (m, 2H), 2.24-2.19 (m, 1H), 2.12-2.06 (m, 2H), 1.83 (br, 6H).

### Example 33: Preparation of Compound 33

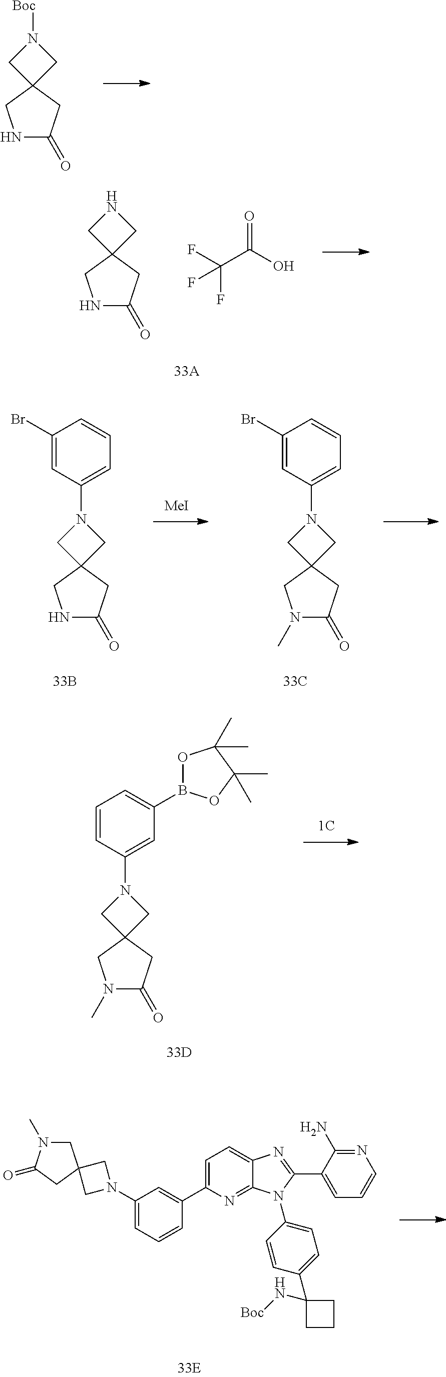

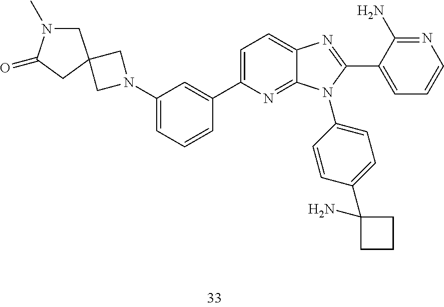 [SMILES: CN1CC2(CC1=O)CN(c1cccc(-c3ccc4nc(-c5cccnc5N)n(-c5ccc(C6(N)CCC6)cc5)c4n3)c1)C2]

### Step A: Preparation of Compound 33A

Trifluoroacetic acid (2 mL) was slowly added dropwise to a reaction solution of tert-butyl 7-oxo-2,6-diazaspiro[3,4]octane-2-carboxylate (0.5 g) in dichloromethane (10 mL) at 0° C. After the addition was completed, the reaction solution was stirred at room temperature. After the reaction was completed, the reaction solution was concentrated to give compound 33A (0.78 g).

MS (ESI, [M+H]⁺) m/z: 127.4.

### Step B: Preparation of Compound 33B

To a reaction flask were added compound 33A (0.5 g), 1,3-dibromobenzene (0.74 g), (tris(dibenzylideneacetone)dipalladium (0.19 g), 1,1′-binaphthyl-2,2′-diphenyl phosphine (0.13 g), sodium tert-butoxide (1 g) and 1,4-dioxane (30 mL) successively. After the addition was completed, the reaction solution was stirred for reaction at 100° C. under nitrogen atmosphere. After the reaction was completed, the reaction solution was filtered, concentrated, and subjected to column chromatography (dichloromethane/methanol=50/1) to give compound 33B (0.54 g).

MS (ESI, [M+H]⁺) m/z: 281.4.

### Step C: Preparation of Compound 33C

Sodium hydride (0.37 g) was slowly added to a solution of compound 33B (0.52 g) in tetrahydrofuran (30 mL) at 0° C. under nitrogen atmosphere, and after the addition was completed, the reaction solution was transferred to the room temperature condition and stirred for 0.5 h, and further added with methyl iodide (1.32 g) for reaction. After the reaction was completed, a saturated aqueous ammonium chloride solution (75 mL) was added to the reaction solution in an ice bath, and the mixture was extracted with ethyl acetate (50 mL×3), and the organic phases were combined, washed with a saturated aqueous sodium chloride solution (100 mL), dried, filtered, and concentrated to give compound 33C (0.50 g).

MS (ESI, [M+H]⁺) m/z: 295.4.

### Step D: Preparation of Compound 33D

To a reaction flask were added compound 33C (0.50 g), bis(pinacolato)diboron (0.65 g), potassium acetate (0.50 g), [1,1′-bis(diphenylphosphino)ferrocene]palladium dichloride dichloromethane complex (0.14 g) and 1,4-dioxane (30 mL) successively. The reaction solution was stirred for reaction at 100° C. under nitrogen atmosphere. After the reaction was completed, the reaction solution was filtered and concentrated. The reaction solution was subjected to column chromatography (dichloromethane/methanol=20/1) to give compound 33D (0.12 g).

MS (ESI, [M+H]⁺) m/z: 343.5.

### Step E: Preparation of Compound 33E

To a microwave tube were added compound 33D (0.12 g), compound 1C (0.14 g), potassium carbonate (0.12 g), tetrakis(triphenylphosphine)palladium (0.03 g), 1,4-dioxane (4 mL) and water (0.5 mL) successively. After the addition was completed, the reaction solution was purged with nitrogen gas, and reacted at 100° C. in microwave reactor. After the reaction was completed, the reaction solution was filtered and concentrated. The reaction solution was subjected to column chromatography (dichloromethane/methanol=20/1) to give compound 33E (0.18 g).

MS (ESI, [M+H]⁺) m/z: 671.4.

### Step F: Preparation of Compound 33

Compound 33E (176 mg), methanesulfonic acid (0.15 mL) and dichloromethane (10 mL) were added successively to a reaction flask, and the mixture was stirred at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 33 (69 mg).

MS (ESI, [M+H]⁺) m/z: 571.6.

¹H NMR (500 MHz, CDCl₃) δ 8.14 (dd, J=8.4, 2.8 Hz, 1H), 8.02 (dd, J=5.0, 1.9 Hz, 1H), 7.78 (dd, J=8.5, 2.7 Hz, 1H), 7.60-7.55 (m, 2H), 7.48-7.42 (m, 3H), 7.30 (t, J=7.9 Hz, 1H), 7.17 (dt, J=7.8, 1.8 Hz, 1H), 7.09 (d, J=2.2 Hz, 1H), 6.52 (dd, J=7.9, 2.4 Hz, 1H), 6.42 (dd, J=7.7, 4.8 Hz, 1H), 3.91 (s, 4H), 3.69 (d, J=2.6 Hz, 2H), 2.89 (s, 3H), 2.72 (s, 2H), 2.68-2.61 (m, 2H), 2.35-2.28 (m, 2H), 2.20-2.02 (m, 1H), 1.90-1.80 (m, 1H).

### Example 34: Preparation of Compound 34

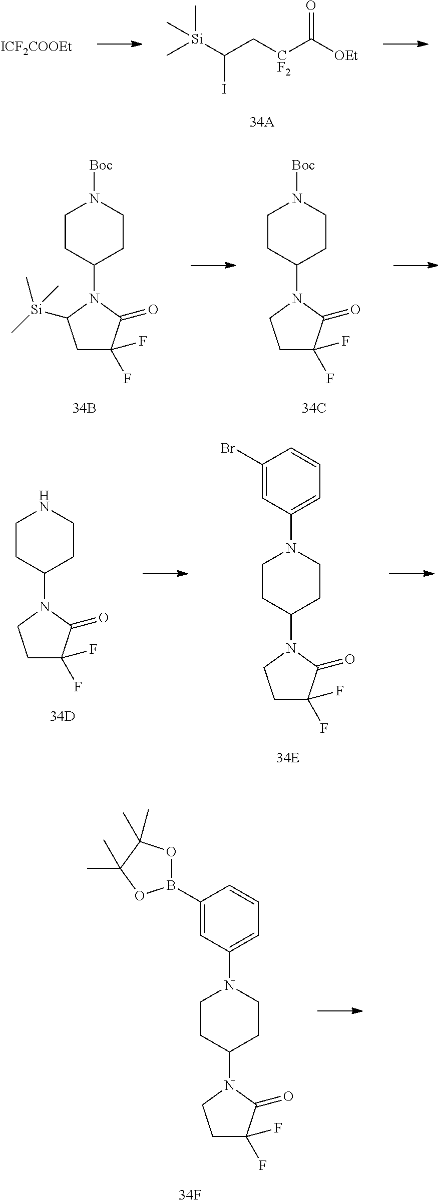

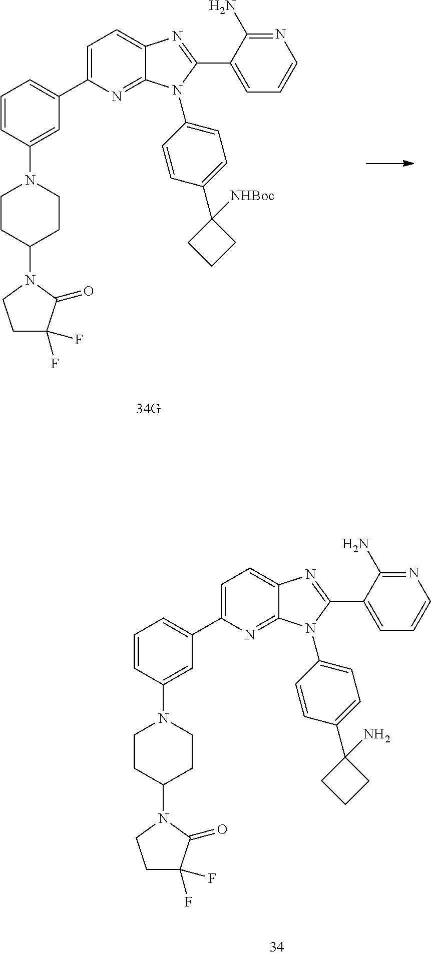 [SMILES: CC(C)(C)OC(=O)NC1(c2ccc(-n3c(-c4cccnc4N)nc4ccc(-c5cccc(N6CCC(N7CCC(F)(F)C7=O)CC6)c5)nc43)cc2)CCC1 ; Nc1ncccc1-c1nc2ccc(-c3cccc(N4CCC(N5CCC(F)(F)C5=O)CC4)c3)nc2n1-c1ccc(C2(N)CCC2)cc1]

### Step A: Preparation of Compound 34A

To the reaction flask were added ethyl 2,2-difluoro-2-iodoacetate (10 g), vinyltrimethylsilane (8.9 g), copper powder (0.13 g) and acetonitrile (50 mL) successively. After the addition was completed, the reaction solution was stirred at 65° C. After the reaction was completed, the reaction solution was distilled under reduced pressure to remove the solvent and concentrated to give compound 34A (8.34 g).

MS (ESI, [M+H]⁺) m/z: 351.0.

### Step B: Preparation of Compound 34B

To a reaction flask were added compound 34A (3.0 g), tert-butyl 4-aminopiperidine-1-carboxylate (1.1 g) and ethanol (20 mL) successively. The mixture was stirred at room temperature. After the reaction was completed, the reaction solution was concentrated and subjected to column chromatography (petroleum ether/ethyl acetate=8/2) to give compound 34B (1.0 g).

MS (ESI, [M+K]⁺) m/z: 415.4.

### Step C: Preparation of Compound 34C

To a reaction flask were added compound 34B (1.0 g), potassium fluoride (1.0 g) and dimethyl sulfoxide (20 mL) successively. The mixture was stirred at room temperature. After the reaction was completed, the reaction solution was concentrated to give compound 34C (0.76 g).

MS (ESI, [M+H]⁺) m/z: 305.4.

### Step D: Preparation of Compound 34D

To the reaction flask were added compound 34C (3.0 g), a solution of 4 M hydrochloric acid in dioxane (6.2 mL), and dichloromethane (20 mL) successively. The mixture was stirred at room temperature. After the reaction was completed, the reaction solution was concentrated to give compound 34D (0.63 g).

MS (ESI, [M+H]⁺) m/z: 205.4.

### Step E: Preparation of Compound 34E

To a reaction flask were added 1,1′-binaphthyl-2,2′-diphenyl phosphine (0.33 g), tris(dibenzylideneacetone)dipalladium (0.37 g), 1,3-dibromo-2-fluorobenzene (4.5 g), compound 34D (0.63 g), sodium tert-butoxide (0.28 g) and dioxane (20 mL) successively under nitrogen atmosphere. After the addition was completed, the reaction solution was stirred at 100° C. After the reaction was completed, the reaction solution was concentrated and subjected to column chromatography (petroleum ether/ethyl acetate=9/1) to give compound 34E (0.23 g).

MS (ESI, [M+H]⁺) m/z: 359.0.

### Step F: Preparation of Compound 34F

To a reaction flask were added compound 34E (0.43 g), bis(pinacolato)diboron (0.25 g), potassium acetate (0.19 g), [1,1′-bis(diphenylphosphino)ferrocene]palladium dichloride dichloromethane complex (0.10 g) and 1,4-dioxane (10 mL) successively. The reaction solution was stirred at 100° C. under nitrogen atmosphere. After the reaction was completed, the reaction solution was filtered and concentrated. The reaction solution was subjected to column chromatography (dichloromethane/methanol=20/1) to give compound 34F (0.17 g).

MS (ESI, [M+H]⁺) m/z: 407.12.

### Step G: Preparation of Compound 34G

To a reaction flask were added compound 34F (0.17 g), compound 1C (0.12 g), potassium carbonate (0.090 g), tetrakis(triphenylphosphine)palladium (0.077 g), 1,4-dioxane (10 mL), and water (10 mL) successively. After the addition was completed, the reaction solution was purged with nitrogen gas, and reacted at 100° C. After the reaction was completed, the reaction solution was filtered and concentrated. The reaction solution was subjected to column chromatography (dichloromethane/methanol=20/1) to give compound 34G (0.20 g).

MS (ESI, [M+H]⁺) m/z: 735.7.

### Step H: Preparation of Compound 34

Compound 34G (0.21 g), methanesulfonic acid (0.13 g) and dichloromethane (15 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 34 (0.041 g).

MS (ESI, [M+H]⁺) m/z: 635.5.

¹H NMR (500 MHz, CDCl₃) δ 8.48 (d, J=8.6 Hz, 1H), 8.31 (d, J=5.1 Hz, 1H), 8.14 (d, J=8.5 Hz, 1H), 7.97 (d, J=8.2 Hz, 2H), 7.92-7.83 (m, 3H), 7.80 (d, J=7.8 Hz, 1H), 7.64 (t, J=8.0 Hz, 1H), 7.53 (d, J=7.7 Hz, 1H), 7.33 (d, J=8.4 Hz, 1H), 6.77 (t, J=6.4 Hz, 1H), 4.46-4.33 (m, 1H), 4.14 (d, J=12.3 Hz, 2H), 3.82 (t, J=6.7 Hz, 2H), 3.21 (t, J=12.2 Hz, 2H), 3.13-3.02 (m, 2H), 2.94 (q, J=10.3, 9.6 Hz, 2H), 2.86 (dd, J=14.7, 7.7 Hz, 2H), 2.32-2.24 (m, 2H), 2.18 (d, J=13.6 Hz, 2H), 1.59 (d, J=17.5 Hz, 2H).

### Example 35: Preparation of Compound 35

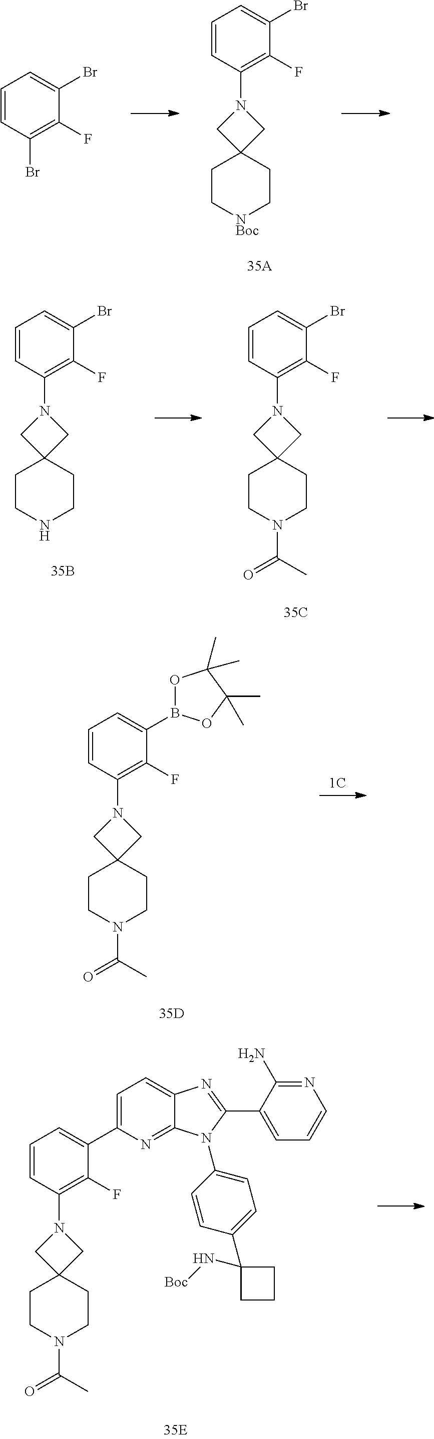 [SMILES: Fc1c(Br)cccc1Br ; CC(C)(C)OC(=O)N1CCC2(CC1)CN(c1cccc(Br)c1F)C2 ; Fc1c(Br)cccc1N1CC2(CCNCC2)C1 ; CC(=O)N1CCC2(CC1)CN(c1cccc(Br)c1F)C2 ; CC(=O)N1CCC2(CC1)CN(c1cccc(B3OC(C)(C)C(C)(C)O3)c1F)C2 ; CC(=O)N1CCC2(CC1)CN(c1cccc(-c3ccc4nc(-c5cccnc5N)n(-c5ccc(C6(NC(=O)OC(C)(C)C)CCC6)cc5)c4n3)c1F)C2]

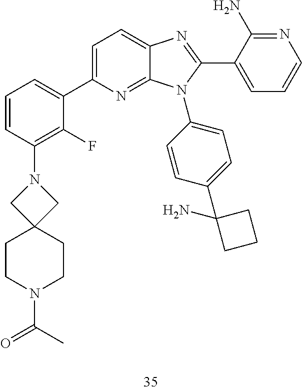 [SMILES: CC(=O)N1CCC2(CC1)CN(c1cccc(-c3ccc4nc(-c5cccnc5N)n(-c5ccc(C6(N)CCC6)cc5)c4n3)c1F)C2]

### Step A: Preparation of Compound 35A

To a reaction flask were added 1,1′-binaphthyl-2,2′-bis-diphenylphosphine (0.21 g), tris(dibenzylideneacetone)dipalladium (0.20 g), 1,3-dibromo-2-fluorobenzene (1.68 g), tert-butyl 2,7-diazaspiro[3.5]nonane-7-carbonate (1.00 g), sodium tert-butoxide (1.70 g), and tetrahydrofuran (25 mL) successively under nitrogen atmosphere. After the addition was completed, the reaction solution was stirred at 85° C. After the reaction was completed, the reaction solution was concentrated and subjected to column chromatography (petroleum ether/ethyl acetate=5/1) to give compound 35A (1.51 g).

MS (ESI, [M-100+H]⁺) m/z: 299.4.

### Step B: Preparation of Compound 35B

Compound 35A (1.51 g), methanesulfonic acid (3.62 g) and dichloromethane (60 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered and concentrated to give compound 35B (1.32 g).

MS (ESI, [M+H]⁺) m/z: 299.4.

### Step C: Preparation of Compound 35C

To a reaction flask were added compound 35B (1.32 g), acetic anhydride (0.67 g), triethylamine (1.32 g) and dichloromethane (50 mL) successively. The mixture was stirred at room temperature. After the reaction was completed, water and dichloromethane were added to extract the reaction solution, and the reaction solution was washed with saturated brine and dried over anhydrous sodium sulfate. The reaction solution was filtered and concentrated to give compound 35C (1.32 g).

MS (ESI, [M+H]⁺) m/z: 341.4.

### Step D: Preparation of Compound 35D

To a reaction flask were added compound 35C (1.32 g), bis(pinacolato)diboron (1.47 g), potassium acetate (1.14 g), [1,1′-bis(diphenylphosphino)ferrocene]palladium dichloride dichloromethane complex (0.32 g) and 1,4-dioxane (50 mL) successively. The reaction solution was stirred at 100° C. under nitrogen atmosphere. After the reaction was completed, the reaction solution was filtered and concentrated. The reaction solution was subjected to column chromatography (dichloromethane/methanol=20/1) to give compound 35D (1.27 g).

MS (ESI, [M+H]⁺) m/z: 389.6.

### Step E: Preparation of Compound 35E

To a reaction flask were added compound 35D (0.59 g), compound 1C (0.5 g), potassium carbonate (0.42 g), tetrakis(triphenylphosphine)palladium (0.12 g), 1,4-dioxane (25 mL), and water (5 mL) successively. After the addition was completed, the reaction solution was purged with nitrogen gas, and reacted at 100° C. After the reaction was completed, the reaction solution was filtered and concentrated. The reaction solution was subjected to column chromatography (dichloromethane/methanol=20/1) to give compound 35E (0.64 g).

MS (ESI, [M+H]⁺) m/z: 717.8.

### Step F: Preparation of Compound 35

Compound 35E (0.63 g), methanesulfonic acid (0.84 g) and dichloromethane (30 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 35 (0.32 g).

MS (ESI, [M+H]⁺) m/z: 617.7.

¹H NMR (500 MHz, CDCl₃) δ 8.09 (d, J=8.3 Hz, 1H), 8.05 (dd, J=4.8, 1.5 Hz, 1H), 7.76 (dd, J=8.3, 2.0 Hz, 1H), 7.53 (d, J=8.4 Hz, 2H), 7.38 (d, J=8.4 Hz, 2H), 7.24 (t, J=6.7 Hz, 1H), 7.09 (dd, J=7.8, 1.6 Hz, 1H), 7.03 (t, J=7.8 Hz, 1H), 6.59 (s, 2H), 6.46 (t, J=8.1 Hz, 1H), 6.36 (dd, J=7.8, 4.9 Hz, 1H), 3.77 (s, 4H), 3.60-3.54 (m, 2H), 3.44-3.38 (m, 2H), 2.59 (ddd, J=11.8, 8.9, 6.6 Hz, 2H), 2.19 (ddd, J=11.5, 9.1, 6.0 Hz, 2H), 2.09 (s, 4H), 2.03 (s, 1H), 1.92 (s, 2H), 1.87-1.83 (m, 2H), 1.81-1.77 (m, 2H).

### Example 36: Preparation of Compound 36

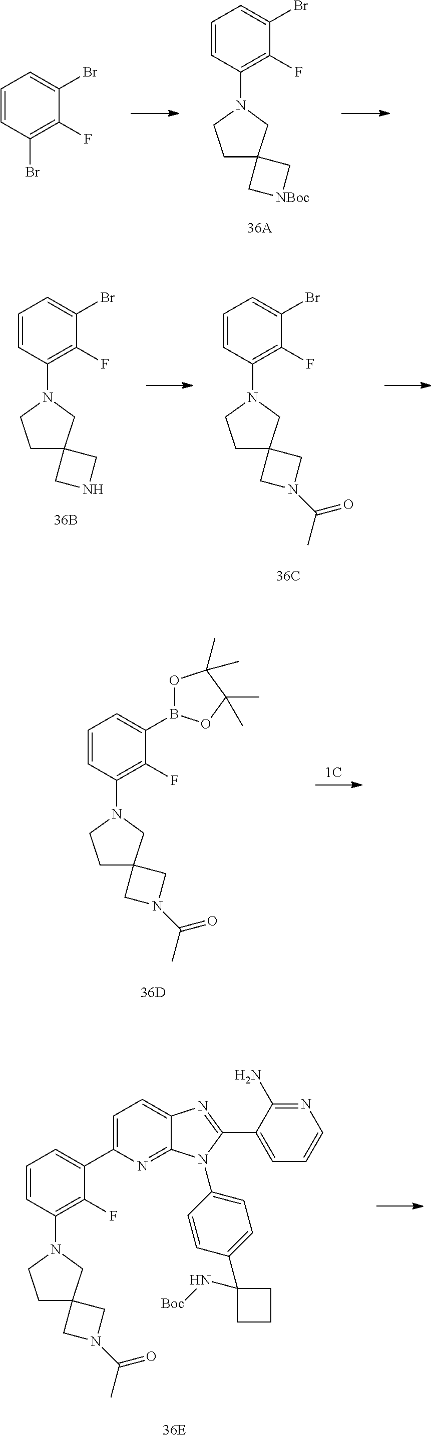 [SMILES: Fc1c(Br)cccc1Br ; CC(C)(C)OC(=O)N1CC2(CCN(c3cccc(Br)c3F)C2)C1 ; Fc1c(Br)cccc1N1CCC2(CNC2)C1 ; CC(=O)N1CC2(CCN(c3cccc(Br)c3F)C2)C1 ; CC(=O)N1CC2(CCN(c3cccc(B4OC(C)(C)C(C)(C)O4)c3F)C2)C1 ; CC(=O)N1CC2(CCN(c3cccc(-c4ccc5nc(-c6cccnc6N)n(-c6ccc(C7(NC(=O)OC(C)(C)C)CCC7)cc6)c5n4)c3F)C2)C1]

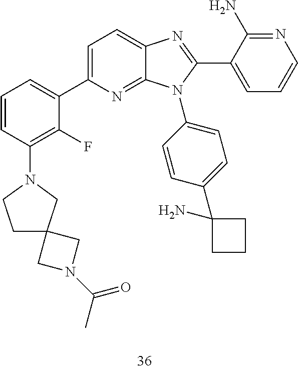 [SMILES: CC(=O)N1CC2(CCN(c3cccc(-c4ccc5nc(-c6cccnc6N)n(-c6ccc(C7(N)CCC7)cc6)c5n4)c3F)C2)C1]

### Step A: Preparation of Compound 36A

To a reaction flask were added 1,1′-binaphthyl-2,2′-bis-diphenylphosphine (0.088 g), tris(dibenzylideneacetone)dipalladium (0.086 g), 1,3-dibromo-2-fluorobenzene (0.72 g), tert-butyl 2,6-diaza-spiro[3.4]octane-2-carbonate (0.40 g), sodium tert-butoxide (0.72 g), and tetrahydrofuran (25 mL) successively under nitrogen atmosphere. After the addition was completed, the reaction solution was stirred at 85° C. After the reaction was completed, the reaction solution was concentrated and subjected to column chromatography (petroleum ether/ethyl acetate=9/1) to give compound 36A (620 mg).

MS (ESI, [M+H]⁺) m/z: 385.4.

### Step B: Preparation of Compound 36B

Compound 36A (600 mg), methanesulfonic acid (1.55 g) and dichloromethane (30 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered and concentrated to give compound 36B (420 mg).

MS (ESI, [M+H]⁺) m/z: 285.3.

### Step C: Preparation of Compound 36C

To a reaction flask were added compound 36B (0.42 g), acetic anhydride (0.23 g), triethylamine (0.45 g) and dichloromethane (20 mL) successively. The mixture was stirred at room temperature. After the reaction was completed, water and dichloromethane were added to extract the reaction solution, and the reaction solution was washed with saturated brine and dried over anhydrous sodium sulfate. The reaction solution was filtered and concentrated to give compound 36C (440 mg).

MS (ESI, [M+H]⁺) m/z: 327.4.

### Step D: Preparation of Compound 36D

To a reaction flask were added compound 36C (0.44 g), bis(pinacolato)diboron (0.51 g), potassium acetate (0.40 g), [1,1′-bis(diphenylphosphino)ferrocene]palladium dichloride dichloromethane complex (0.11 g) and 1,4-dioxane (30 mL) successively. The reaction solution was stirred at 100° C. under nitrogen atmosphere. After the reaction was completed, the reaction solution was filtered and concentrated. The reaction solution was subjected to column chromatography (dichloromethane/methanol=40/1) to give compound 36D (0.63 g).

MS (ESI, [M+H]⁺) m/z: 375.5.

### Step E: Preparation of Compound 36E

To a reaction flask were added compound 36D (0.74 g), compound 1C (0.5 g), potassium carbonate (0.42 g), tetrakis(triphenylphosphine)palladium (0.12 g), 1,4-dioxane (25 mL), and water (5 mL) successively. After the addition was completed, the reaction solution was purged with nitrogen gas, and reacted at 100° C. After the reaction was completed, the reaction solution was filtered and concentrated. The reaction solution was subjected to column chromatography (dichloromethane/methanol=20/1) to give compound 36E (500 mg).

MS (ESI, [M+H]⁺) m/z: 703.4.

### Step F: Preparation of Compound 36

Compound 36E (500 mg), methanesulfonic acid (0.7 g) and dichloromethane (20 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 36 (190 mg).

MS (ESI, [M+H]⁺) m/z: 603.6.

¹H NMR (500 MHz, CDCl₃) δ 8.12 (d, J=8.3 Hz, 1H), 8.06 (dd, J=4.8, 1.7 Hz, 1H), 7.76 (dd, J=8.3, 2.4 Hz, 1H), 7.54 (d, J=8.4 Hz, 2H), 7.40 (d, J=8.4 Hz, 2H), 7.27-7.18 (m, 1H), 7.10 (dd, J=7.8, 1.6 Hz, 1H), 7.06 (t, J=7.9 Hz, 1H), 6.78-6.64 (m, 1H), 6.59 (s, 2H), 6.38 (dd, J=7.8, 4.9 Hz, 1H), 4.11 (d, J=8.4 Hz, 1H), 4.05 (d, J=8.4 Hz, 1H), 4.01 (d, J=9.9 Hz, 1H), 3.96 (d, J=9.9 Hz, 1H), 3.60 (qd, J=9.8, 2.0 Hz, 2H), 3.49 (ddq, J=14.4, 9.5, 7.3 Hz, 2H), 2.66-2.54 (m, 2H), 2.27-2.17 (m, 4H), 2.16-1.94 (m, 4H), 1.89 (s, 3H).

### Example 37: Preparation of Compound 37

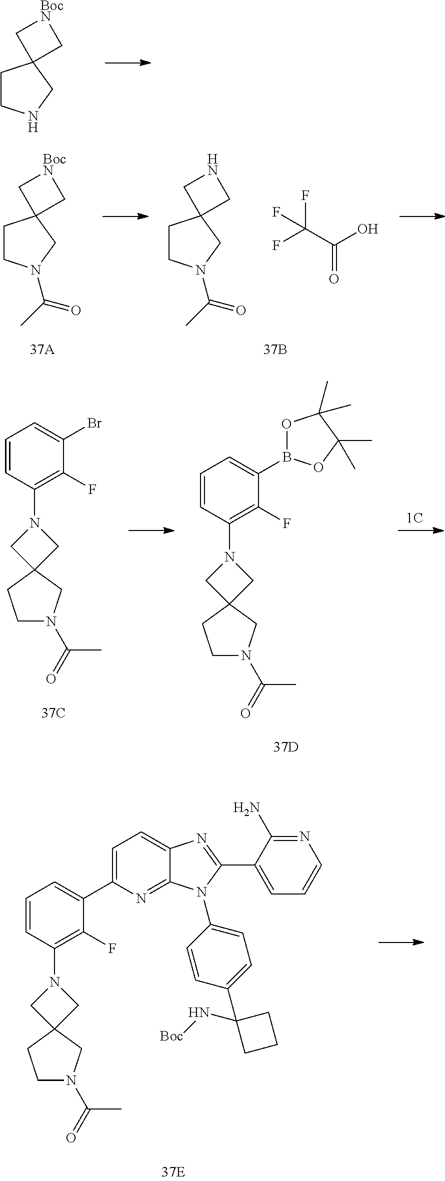

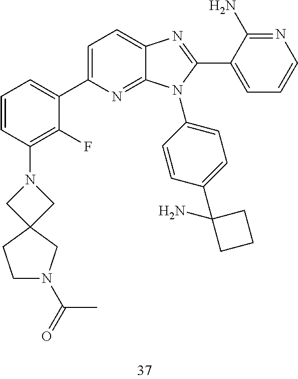 [SMILES: CC(=O)N1CCC2(C1)CN(c1cccc(-c3ccc4nc(-c5cccnc5N)n(-c5ccc(C6(N)CCC6)cc5)c4n3)c1F)C2]

### Step A: Preparation of Compound 37A

To a reaction flask were added tert-butyl 2,6-diaza-spiro[3.4]octane-2-carbonate (0.60 g), acetic anhydride (0.44 g), triethylamine (0.86 g) and methylene chloride (40 mL) successively. The mixture was stirred at room temperature. After the reaction was completed, water and dichloromethane were added to extract the reaction solution, and the reaction solution was washed with saturated brine and dried over anhydrous sodium sulfate. The reaction solution was filtered and concentrated to give compound 37A (0.82 g).

MS (ESI, [M+H]⁺) m/z: 255.4.

### Step B: Preparation of Compound 37B

To a reaction flask were added compound 37A (0.82 g), trifluoroacetic acid (6.14 g), and dichloromethane (50 mL) successively. The mixture was stirred at room temperature. After the reaction was completed, the reaction solution was concentrated to give compound 37B (1.68 g).

MS (ESI, [M+H]⁺) m/z: 155.4.

### Step C: Preparation of Compound 37C

To a reaction flask were added 1,1′-binaphthyl-2,2′-diphenyl phosphine (0.22 g), tris(dibenzylideneacetone)dipalladium (0.22 g), 1,3-dibromo-2-fluorobenzene (1.68 g), compound 37B (1.20 g), sodium tert-butoxide (0.88 g) and tetrahydrofuran (100 mL) successively under nitrogen atmosphere. After the addition was completed, the reaction solution was stirred at 85° C. After the reaction was completed, the reaction solution was concentrated and subjected to column chromatography (petroleum ether/ethyl acetate=9/1) to give compound 37C (960 mg).

MS (ESI, [M+H]⁺) m/z: 327.4.

### Step D: Preparation of Compound 37D

To a reaction flask were added compound 37C (0.78 g), bis(pinacolato)diboron (0.92 g), potassium acetate (0.80 g), [1,1′-bis(diphenylphosphino)ferrocene]palladium dichloride dichloromethane complex (0.19 g) and 1,4-dioxane (60 mL) successively. The reaction solution was stirred at 100° C. under nitrogen atmosphere. After the reaction was completed, the reaction solution was filtered and concentrated. The reaction solution was subjected to column chromatography (dichloromethane/methanol=20/1) to give compound 37D (0.64 g).

MS (ESI, [M+H]⁺) m/z: 375.5.

### Step E: Preparation of Compound 37E

To a reaction flask were added compound 37D (0.53 g), compound 1C (0.32 g), potassium carbonate (0.27 g), tetrakis(triphenylphosphine)palladium (0.075 g), 1,4-dioxane (25 mL), and water (5 mL) successively. After the addition was completed, the reaction solution was purged with nitrogen gas, and reacted at 100° C. After the reaction was completed, the reaction solution was filtered and concentrated. The reaction solution was subjected to column chromatography (dichloromethane/methanol=20/1) to give compound 37E (0.43 g).

MS (ESI, [M+H]⁺) m/z: 703.7.

### Step F: Preparation of Compound 37

Compound 37E (0.43 g), methanesulfonic acid (0.58 g) and dichloromethane (25 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 37 (0.10 g).

MS (ESI, [M+H]⁺) m/z: 603.6.

¹H NMR (500 MHz, CDCl₃) δ 8.26-7.99 (m, 2H), 7.76 (d, J=8.1 Hz, 1H), 7.54 (d, J=6.2 Hz, 2H), 7.39 (d, J=7.5 Hz, 2H), 7.33-7.26 (m, 1H), 7.22-6.93 (m, 2H), 6.59 (s, 2H), 6.48 (dd, J=13.6, 6.9 Hz, 1H), 6.38 (dd, J=7.1, 5.1 Hz, 1H), 3.93 (s, 3H), 3.66 (d, J=10.6 Hz, 2H), 3.54 (dt, J=10.4, 6.9 Hz, 2H), 2.60 (dd, J=17.4, 8.9 Hz, 2H), 2.27 (t, J=6.7 Hz, 1H), 2.21 (dd, J=15.6, 10.0 Hz, 2H), 2.12 (dd, J=14.1, 7.5 Hz, 2H), 2.05 (t, J=6.6 Hz, 3H), 1.98 (s, 3H), 1.87-1.75 (m, 1H).

### Example 38: Preparation of Compound 38

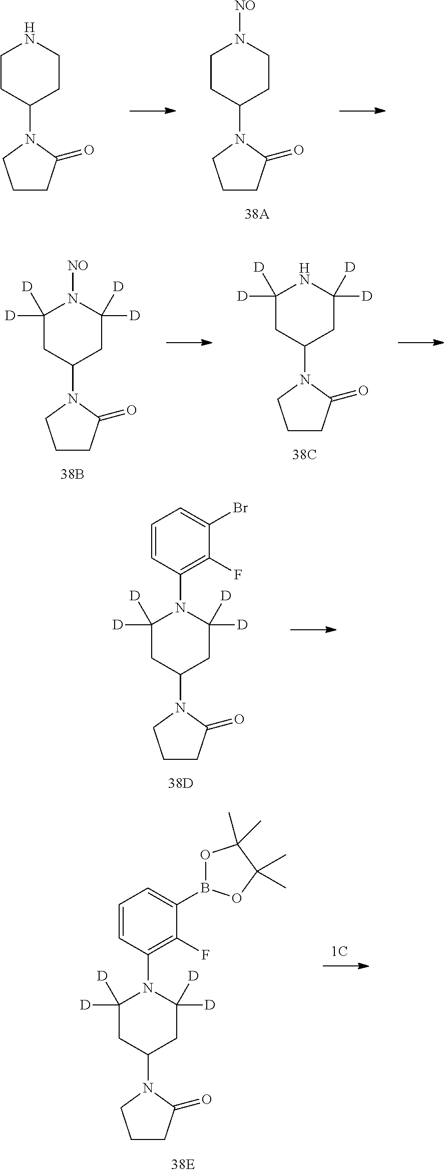

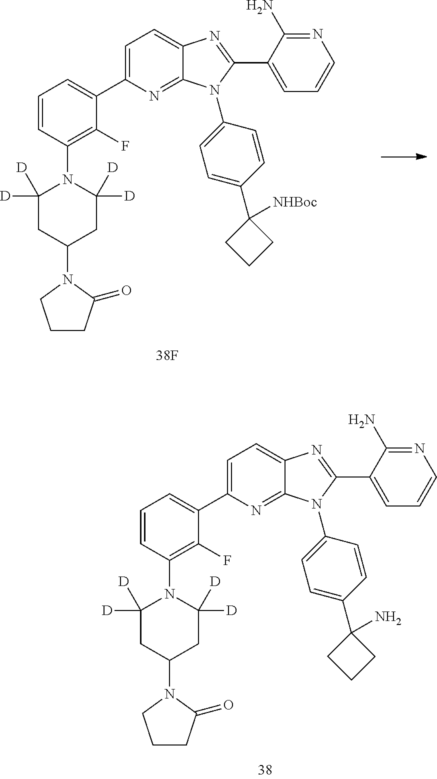 [SMILES: [2H]C1([2H])CC(N2CCCC2=O)CC([2H])([2H])N1c1cccc(-c2ccc3nc(-c4cccnc4N)n(-c4ccc(C5(NC(=O)OC(C)(C)C)CCC5)cc4)c3n2)c1F ; [2H]C1([2H])CC(N2CCCC2=O)CC([2H])([2H])N1c1cccc(-c2ccc3nc(-c4cccnc4N)n(-c4ccc(C5(N)CCC5)cc4)c3n2)c1F]

### Step A: Preparation of Compound 38A

Diluted hydrochloric acid (0.433 g) was added dropwise to 1-(piperidin-4-yl)pyrrolidin-2-one (1 g) and water (10 mL) at 0° C., and after the addition was completed, an aqueous sodium nitrite solution (0.533 g) was slowly added dropwise and stirred at room temperature. After the reaction was completed, the reaction solution was extracted with dichloromethane, dried over anhydrous sodium sulfate, filtered and concentrated to give compound 38A (0.7 g).

MS (ESI, [M+H]⁺) m/z: 198.4.

### Step B: Preparation of Compound 38B

Heavy water (8 mL) was slowly added to compound 38A (0.7 g) and sodium methoxide (0.553 g) under nitrogen atmosphere, and after the addition was completed, the mixture was stirred at 80° C. Deuterated ethanol (5 mL) was added slowly to the reaction flask, and then the reaction solution was stirred at 80° C. After the reaction was completed, the reaction solution was extracted with dichloromethane, dried over anhydrous sodium sulfate, filtered under vacuum and concentrated to give compound 38B (0.45 g).

¹H NMR (500 MHz, CDCl₃) δ 4.40-4.29 (m, 1H), 3.33-3.21 (m, 2H), 2.00 (t, J=7.0 Hz, 2H), 1.97-1.92 (m, 1H), 1.82-1.71 (m, 2H), 1.44 (tt, J=14.8, 7.3 Hz, 1H), 1.23 (s, 1H), 0.92-0.75 (in, 1H).

### Step C: Preparation of Compound 38C

Sodium methoxide (0.362 g) was added to a solution of compound 38B (0.45 g) in deuterated ethanol (2.5 mL) and heavy water (5.00 mL) at room temperature, and after the addition was completed, nickel-aluminum alloy (1.35 g) was added to the reaction flask, and the temperature was controlled at 30-40° C. After the addition was completed, the reaction solution was stirred at 35° C. After the reaction was completed, the reaction solution was filtered and concentrated, and the residue was extracted with methylene chloride, dried over anhydrous sodium sulfate, filtered and concentrated to give compound 38C (0.23 g).

MS (ESI, [M+H]⁺) m/z: 173.5.

### Step D: Preparation of Compound 38D

To a microwave tube were added sodium tert-butoxide (0.352 g), 1,1′-binaphthyl-2,2′-bis-diphenylphosphine (0.152 g), tris(dibenzylideneacetone)dipalladium (0.224 g), 1,3-dibromo-2-fluorobenzene (0.31 g), compound 38C and 1,4-dioxane (10 mL) successively, and the reaction solution was stirred for reaction under nitrogen atmosphere at 100° C. After the reaction solution was completed, the reaction solution was filtered and concentrated. The reaction solution was subjected to column chromatography (dichloromethane/methanol=40/1) to give compound 38D (0.14 g).

MS (ESI, [M+H]⁺) m/z: 345.3.

### Step E: Preparation of Compound 38E

To a reaction flask were added compound 38D (0.14 g), bis(pinacolato)diboron (0.21 g), potassium acetate (0.12 g), [1,1′-bis(diphenylphosphino)ferrocene]palladium dichloride dichloromethane complex (0.066 g) and 1,4-dioxane (15 mL) successively. The reaction solution was stirred for reaction at 100° C. under nitrogen atmosphere. After the reaction was completed, the reaction solution was filtered and concentrated. The reaction solution was subjected to column chromatography (dichloromethane/methanol=40/1) to give compound 38E (0.13 g).

MS (ESI, [M+H]⁺) m/z: 393.4.

### Step F: Preparation of Compound 38F

To a reaction flask were added compound 38E (0.13 g), compound 1C (0.1 g), potassium carbonate (0.056 g), tetrakis(triphenylphosphine)palladium (0.047 g), 1,4-dioxane (10 mL), and water (2 mL) successively. After the addition was completed, the reaction solution was purged with nitrogen gas, and stirred for reaction at 100° C. After the reaction was completed, the reaction solution was filtered and concentrated. The reaction solution was subjected to column chromatography (dichloromethane/methanol=30/1) to give compound 38F (0.03 g).

MS (ESI, [M+H]⁺) m/z: 721.4.

### Step G: Preparation of Compound 38

To a reaction flask were added compound 38F (0.028 g), methanesulfonic acid (0.019 g) and dichloromethane (20 mL) successively, and the reaction solution was stirred for reaction at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 38 (0.013 g).

MS (ESI, [M+H]⁺) m/z: 621.5.

¹H NMR (500 MHz, CDCl₃) δ 8.11 (d, J=8.3 Hz, 1H), 8.06 (d, J=3.6 Hz, 1H), 7.81 (d, J=6.9 Hz, 1H), 7.55 (d, J=8.2 Hz, 2H), 7.51 (t, J=7.1 Hz, 1H), 7.40 (d, J=8.2 Hz, 2H), 7.12 (dd, J=12.9, 7.2 Hz, 2H), 7.00 (t, J=7.6 Hz, 1H), 6.57 (s, 2H), 6.39 (dd, J=7.5, 4.9 Hz, 1H), 4.15 (t, J=12.1 Hz, 1H), 3.48 (s, 1H), 3.41 (t, J=6.9 Hz, 2H), 2.65-2.57 (m, 2H), 2.41 (dd, J=16.7, 8.4 Hz, 1H), 2.28-2.21 (m, 2H), 2.20-2.09 (m, 2H), 2.03 (dd, J=13.9, 7.0 Hz, 2H), 1.93 (dd, J=16.1, 7.5 Hz, 2H), 1.78 (d, J=12.3 Hz, 2H).

### Example 39: Preparation of Compound 39

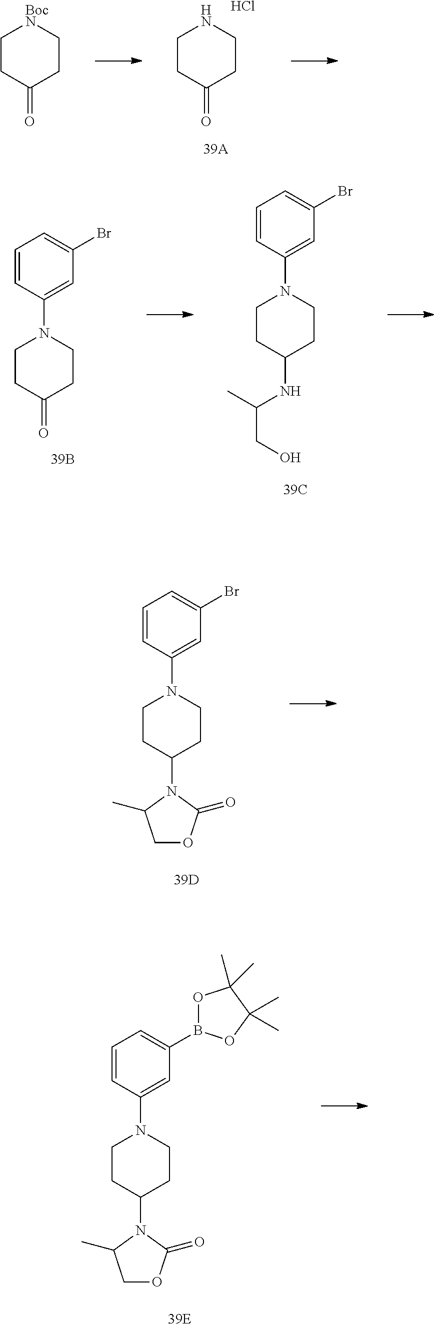

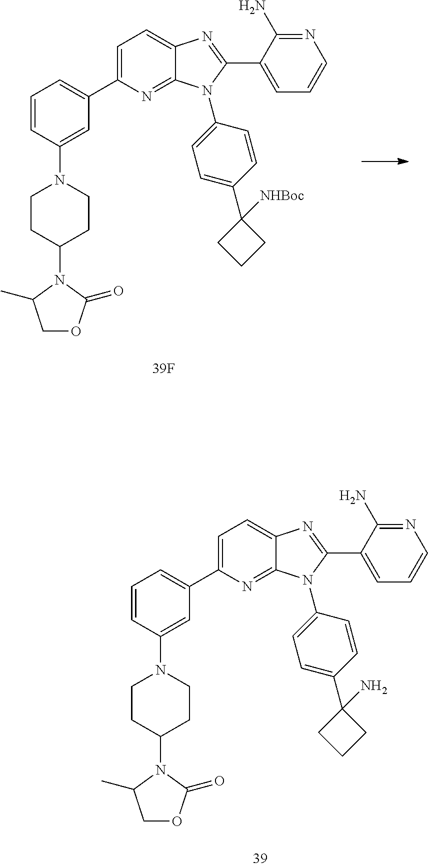 [SMILES: CC1COC(=O)N1C1CCN(c2cccc(-c3ccc4nc(-c5cccnc5N)n(-c5ccc(C6(NC(=O)OC(C)(C)C)CCC6)cc5)c4n3)c2)CC1 ; CC1COC(=O)N1C1CCN(c2cccc(-c3ccc4nc(-c5cccnc5N)n(-c5ccc(C6(N)CCC6)cc5)c4n3)c2)CC1]

### Step A: Preparation of Compound 39A

Diluted hydrochloric acid (0.914 g) was slowly added to a solution of tert-butyl 4-oxopiperidine-1-carboxylate (1 g) in methylene chloride (20 mL) at room temperature, and after the addition was completed, the mixture was stirred at room temperature. After the reaction was completed, the reaction solution was concentrated to give compound 39A (0.7 g).

MS (ESI, [M+H]⁺) m/z: 100.1.

### Step B: Preparation of Compound 39B

To a reaction flask were added compound 39A (0.7 g), 1-bromo-3-iodobenzene (1.461 g), potassium carbonate (2.85 g), cuprous iodide (0.098 g), L-alanine (0.119 g), and dimethyl sulfoxide (10 mL) successively, and the reaction solution was stirred for reaction at 100° C. under nitrogen atmosphere.

After the reaction was completed, the reaction solution was filtered, concentrated, and subjected to column chromatography (petroleum ether/ethyl acetate=5/1) to give compound 39B (0.088 g).

MS (ESI, [M+H]⁺) m/z: 254.1.

### Step C: Preparation of Compound 39C

To a reaction flask were added compound 39B (0.3 g), 2-aminopropan-1-ol (0.177 g), acetic acid (0.142 g) and methanol (10 mL) successively, and the reaction solution was stirred for 1 h at room temperature under nitrogen atmosphere. Sodium cyanoborohydride (0.148 g) was added to the reaction flask, and the reaction solution was stirred at room temperature. After the reaction was completed, the reaction solution was concentrated and subjected to column chromatography (dichloromethane/methanol=25/1) to give compound 39C (0.23 g) MS (ESI, [M+H]⁺) m/z: 313.4.

### Step D: Preparation of Compound 39D

To a reaction flask were added N,N-carbonyldiimidazole (0.179 g), compound 39C (0.23 g), toluene (10 mL) successively, and the reaction solution was stirred at room temperature for 1 h under nitrogen atmosphere and then stirred at 110° C. After the reaction was completed, the reaction solution was concentrated, and subjected to column chromatography (petroleum ether/ethyl acetate=5/1) to give compound 39D (0.16 g).

MS (ESI, [M+H]⁺) m/z: 339.1.

### Step E: Preparation of Compound 39E

To a reaction flask were added compound 39D (0.16 g), bis(pinacolato)diboron (0.24 g), potassium acetate (0.14 g), [1,1′-bis(diphenylphosphino)ferrocene]palladium dichloride dichloromethane complex (0.039 g) and 1,4-dioxane (20 mL) successively. The reaction solution was stirred for reaction at 100° C. under nitrogen atmosphere. After the reaction was completed, the reaction solution was filtered and concentrated. The reaction solution was subjected to column chromatography (dichloromethane/methanol=50/1) to give compound 39E (0.06 g).

MS (ESI, [M+H]⁺) m/z: 387.6.

### Step F: Preparation of Compound 39F

To a reaction flask were added compound 39E (0.06 g), compound 1C (0.08 g), potassium carbonate (0.045 g), tetrakis(triphenylphosphine)palladium (0.038 g), 1,4-dioxane (10 mL), and water (2 mL) successively. After the addition was completed, the reaction solution was purged with nitrogen gas, and stirred for reaction at 100° C. After the reaction was completed, the reaction solution was filtered and concentrated. The reaction solution was subjected to column chromatography (dichloromethane/methanol=30/1) to give compound 39F (65 mg).

MS (ESI, [M+H]⁺) m/z: 715.5.

### Step G: Preparation of Compound 39

To a reaction flask were added compound 39F (0.065 g), methanesulfonic acid (0.051 g) and dichloromethane (20 mL) successively, and the reaction solution was stirred for reaction at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=20/1) to give compound 39 (0.032 g).

MS (ESI, [M+H]⁺) m/z: 615.7.

¹H NMR (500 MHz, CDCl₃) δ 8.05 (d, J=8.3 Hz, 1H), 7.91 (d, J=4.0 Hz, 1H), 7.71 (d, J=8.4 Hz, 1H), 7.66 (d, J=8.2 Hz, 2H), 7.49 (s, 1H), 7.45 (d, J=7.6 Hz, 1H), 7.40 (d, J=8.2 Hz, 2H), 7.24 (d, J=7.9 Hz, 1H), 7.09 (d, J=7.5 Hz, 1H), 6.88 (d, J=7.9 Hz, 3H), 6.41 (dd, J=7.2, 5.3 Hz, 1H), 4.35 (t, J=8.3 Hz, 1H), 4.00-3.90 (m, 1H), 3.82 (dd, J=8.2, 5.6 Hz, 1H), 3.77-3.65 (m, 3H), 2.86-2.72 (m, 4H), 2.68 (s, 4H), 2.37 (d, J=7.5 Hz, 1H), 2.07 (qd, J=12.1, 3.7 Hz, 1H), 1.99-1.92 (m, 2H), 1.88 (d, J=7.1 Hz, 1H), 1.81 (d, J=10.8 Hz, 1H), 1.32 (d, J=6.1 Hz, 3H).

### Example 40: Preparation of Compound 40

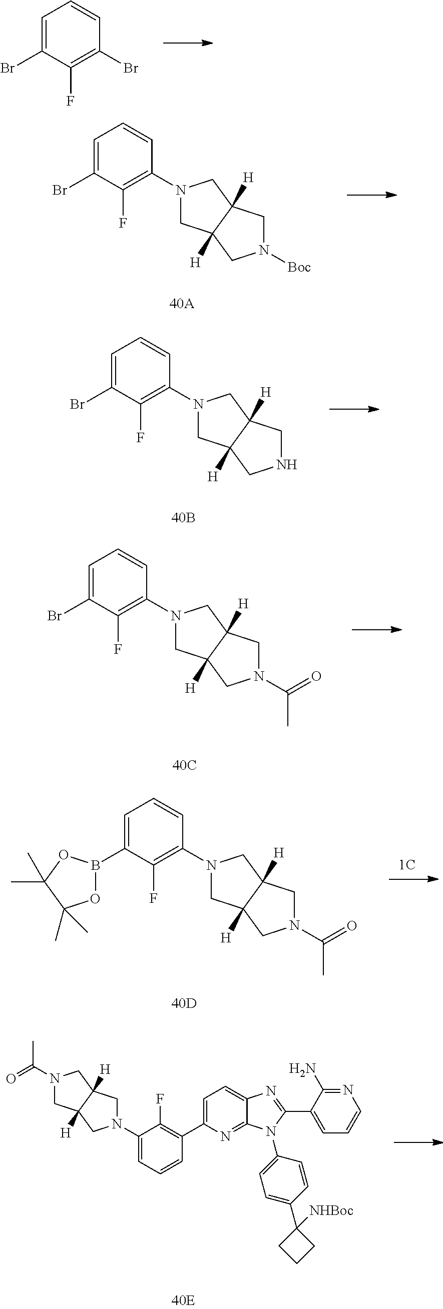

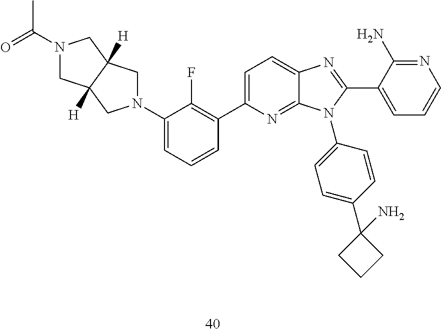 [SMILES: CC(=O)N1C[C@@H]2CN(c3cccc(-c4ccc5nc(-c6cccnc6N)n(-c6ccc(C7(N)CCC7)cc6)c5n4)c3F)C[C@@H]2C1]

### Step A: Preparation of Compound 40A

To a reaction flask were added Cis-2-Boc-hexahydropyrrolo[3,4-c]pyrrole (250 mg), 1,3-dibromo-2-fluorobenzene (448 mg), tris(dibenzylideneacetone)dipalladium (55 mg), 1,1′-binaphthyl-2,2′-diphenyl phosphine (40 mg), tetrahydrofuran (30 mL) and sodium tert-butoxide (509 mg) successively, and the reaction solution was stirred for reaction at 80° C. under nitrogen atmosphere. After the reaction was completed, the reaction solution was concentrated and subjected to column chromatography (petroleum ether/ethyl acetate=5/1) to give compound 40A (164 mg).

MS (ESI, [M+H]⁺) m/z: 385.0.

### Step B: Preparation of Compound 40B

To a reaction flask containing compound 40A (164 mg) were added dichloromethane (15 mL) and methanesulfonic acid (130 mg) successively, and after the addition was completed, the reaction solution was stirred for reaction at room temperature. After the reaction was completed, and the reaction solution was directly used in the next step without treatment.

### Step C: Preparation of Compound 40C

To the reaction solution containing compound 40B were added triethylamine (42.9 mg) and acetic anhydride (43.3 mg), and after the addition was completed, the reaction solution was stirred for reaction at room temperature. After the reaction was completed, the reaction solution was washed with water 2 times and dried over anhydrous sodium sulfate. The reaction solution was filtered, and the filtrate was concentrated to give compound 40C (142 mg).

MS (ESI, [M+H]⁺) m/z: 327.4.

### Step D: Preparation of Compound 40D

To a reaction flask were added compound 40C (142 mg), bis(pinacolato)diboron (164 mg), potassium acetate (127 mg), [1,1′-bis(diphenylphosphino)ferrocene]palladium dichloride dichloromethane complex (50 mg) and 1,4-dioxane (10 mL) successively. The reaction solution was stirred for reaction at 100° C. under nitrogen atmosphere. After the reaction was completed, the reaction solution was filtered and concentrated. The reaction solution was subjected to column chromatography (petroleum ether/ethyl acetate=5/1) to give compound 40D (101 mg).

MS (ESI, [M+H]⁺) m/z: 375.4.

### Step E: Preparation of Compound 40E

To a microwave tube were added compound 40D (100 mg), compound 1C (135 mg), potassium carbonate (150 mg), tetrakis(triphenylphosphine)palladium (20 mg), 1,4-dioxane (3 mL) and water (0.3 mL) successively. After the addition was completed, the reaction solution was purged with nitrogen gas, and reacted at 120° C. in microwave reactor. After the reaction was completed, the reaction solution was filtered and concentrated. The reaction solution was subjected to column chromatography (dichloromethane/methanol=20/1) to give compound 40E (101 mg).

MS (ESI, [M+H]⁺) m/z: 703.5.

### Step F: Preparation of Compound 40

To a reaction flask were added compound 40E (101 mg), methanesulfonic acid (138 mg) and dichloromethane (10 mL) successively, and after the addition was completed, the reaction solution was stirred for reaction at room temperature. After the reaction was completed, a saturated aqueous sodium carbonate solution was added to the reaction solution to adjust the pH to 9, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=20/1) to give compound 40 (42 mg).

MS (ESI, [M+H]⁺) m/z: 603.7.

¹H NMR (500 MHz, CDCl₃) δ 8.12 (d, J=8.5 Hz, 1H), 8.05 (d, J=1.0 Hz, 1H), 7.76 (d, J=7.0 Hz, 1H), 7.54 (d, J=8.0 Hz, 2H), 7.40 (d, J=8.0 Hz, 2H), 7.26-7.24 (m, 1H), 7.10 (d, J=7.5 Hz, 1H), 7.06 (t, J=8.0 Hz, 1H), 6.71 (t, J=8.0 Hz, 1H), 6.58 (s, 2H), 6.39 (dd, J=5.0, 7.5 Hz, 1H), 3.80-3.75 (m, 2H), 3.61-3.56 (m, 2H), 3.54-3.51 (m, 1H), 3.43-3.40 (m, 1H), 3.37-3.32 (m, 2H), 3.10-3.04 (m, 1H), 3.02-2.96 (m, 1H), 2.63-2.58 (m, 2H), 2.27-2.24 (m, 2H), 2.17-2.12 (m, 2H), 2.06 (s, 3H), 2.06-2.04 (m, 1H), 1.86-1.78 (m, 1H).

### Example 41: Preparation of Compound 41

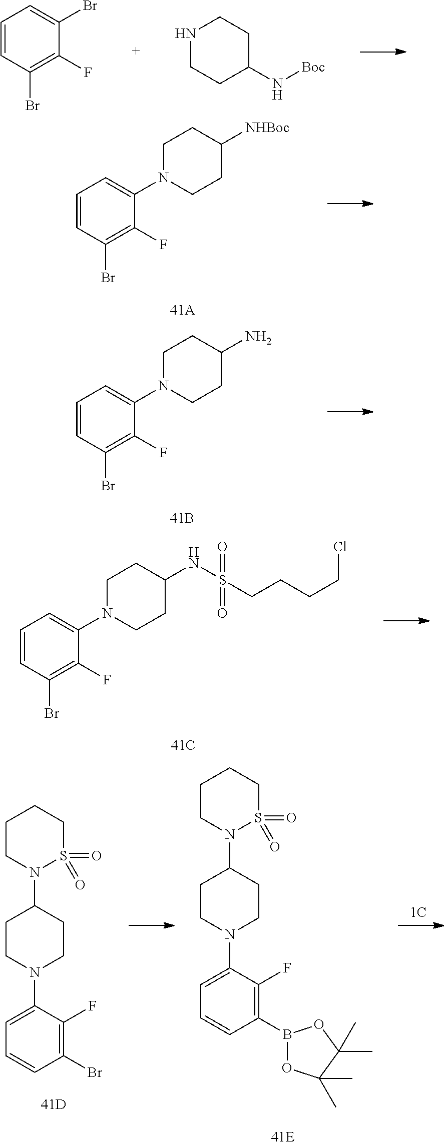

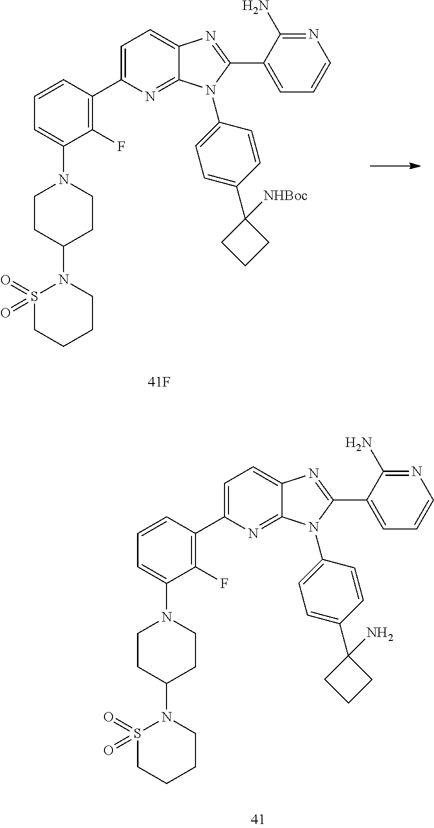 [SMILES: CC(C)(C)OC(=O)NC1(c2ccc(-n3c(-c4cccnc4N)nc4ccc(-c5cccc(N6CCC(N7CCCCS7(=O)=O)CC6)c5F)nc43)cc2)CCC1 ; Nc1ncccc1-c1nc2ccc(-c3cccc(N4CCC(N5CCCCS5(=O)=O)CC4)c3F)nc2n1-c1ccc(C2(N)CCC2)cc1]

### Step A: Preparation of Compound 41A

To a reaction flask were added 2,2′-bis(diphenylphosphino)-1,1′-binaphthyl (2.94 g), bis(benzhydrylacetylene)palladium (2.164 g), 1,3-dibromo-2-fluorobenzene (6 g), tert-butylpiperidin-4-ylcarbamate (4.73 g), sodium tert-butoxide (4.54 g), dioxane (20 mL) successively, and the mixture was stirred overnight at 100° C. under nitrogen atmosphere. After the reaction was completed, the reaction solution was filtered, concentrated, and subjected to column chromatography (dichloromethane/methanol=50/1) to give compound 41A (2.6 g).

MS (ESI, [M+H]⁺) m/z: 373.2.

### Step B: Preparation of Compound 41B

To a reaction flask were added compound 41A (2.6 g), dichloromethane (30 mL) and a solution (7.37 mL) of hydrochloric acid in dioxane successively, and the mixture was stirred at room temperature for 1 h. 10% aqueous sodium hydroxide solution was added to the reaction solution to adjust the pH to 13, water and dichloromethane were added to extract the reaction solution the reaction solution was washed with saturated brine and dried over anhydrous sodium sulfate. The reaction solution was filtered and concentrated to give compound 41B (2 g).

MS (ESI, [M+H]⁺) m/z: 273.3.

### Step C: Preparation of Compound 41C

In an ice-water bath, 4-chlorobutane-1-sulfonyl chloride (0.611 g) was slowly added to a solution of compound 41B (0.873 g) in N,N-dimethylformamide (15 mL), and after the addition was completed, the reaction solution was slowly warmed to room temperature and stirred. After the reaction was completed, the reaction solution was added to 30 mL of ice water, extracted with ethyl acetate, washed with saturated brine, dried over anhydrous sodium sulfate, filtered, concentrated, and subjected to column chromatography (petroleum ether/ethyl acetate=2/1) to give compound 41C (0.826 g).

MS (ESI, [M+H]⁺) m/z: 427.4.

### Step D: Preparation of Compound 41D

After compound 41C (0.826 g) was dissolved in N,N-dimethylformamide (15 mL), sodium hydrogen (153 mg) was added under an ice-bath condition, and after the addition was completed, the reaction solution was refluxed at 80° C. overnight. After the reaction was completed, the reaction solution was poured into 30 mL of ice water, extracted with ethyl acetate, washed with saturated brine, dried over anhydrous sodium sulfate, filtered, concentrated, and subjected to column chromatography (petroleum ether/ethyl acetate=2/3) to give compound 41D (0.3 g).

MS (ESI, [M+H]⁺) m/z: 391.6.

### Step E: Preparation of Compound 41E

To a reaction flask were added compound 41D (0.3 g), bis(pinacolato)diboron (0.389 g), potassium acetate (0.15 g), [1,1′-bis(diphenylphosphino)ferrocene]palladium dichloride dichloromethane complex (0.125 g) and 1,4-dioxane (15 mL) successively. The reaction solution was stirred for reaction at 100° C. under nitrogen atmosphere. After the reaction was completed, the reaction solution was filtered and concentrated. The reaction solution was subjected to column chromatography (petroleum ether/ethyl acetate=7/3) to give compound 41E (0.265 g).

MS (ESI, [M+H]⁺) m/z: 439.5.

### Step F: Preparation of Compound 41F

To a reaction flask were added compound 41E (0.265 g), compound 1C (0.187 g), potassium carbonate (0.105 g), tetrakis(triphenylphosphine)palladium (0.088 g), 1,4-dioxane (10 mL), and water (2 mL) successively. After the addition was completed, the reaction solution was purged with nitrogen gas, and stirred for reaction at 110° C. After the reaction was completed, the reaction solution was filtered and concentrated. The reaction solution was subjected to column chromatography (dichloromethane/10% methanol=4/1) to give compound 41F (184 mg).

MS (ESI, [M+H]⁺) m/z: 767.7.

### Step G: Preparation of Compound 41

Compound 41F (171 mg), methanesulfonic acid (0.142 mL) and dichloromethane (10 mL) were added successively to a reaction flask, and the mixture was stirred at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/10% methanol=1/1) to give compound 41 (80 mg).

MS (ESI, [M+H]⁺) m/z: 667.6.

¹H NMR (500 MHz, DMSO-d₆) δ 8.26 (d, 1H), 8.00 (s, 1H), 7.73 (s, 1H), 7.60 (s, 2H), 7.44 (s, 2H), 7.29 (s, 1H), 7.09-7.19 (m, 3H), 6.94 (s, 2H), 6.41 (s, 1H), 3.86 (s, 1H), 3.56 (s, 2H), 3.42 (s, 2H), 3.06 (s, 2H), 2.77 (s, 2H), 2.43 (s, 2H), 2.17 (s, 2H), 2.04 (s, 3H), 1.89 (d, 2H), 1.72 (s, 2H), 1.60 (s, 2H).

### Example 42: Preparation of Compound 42

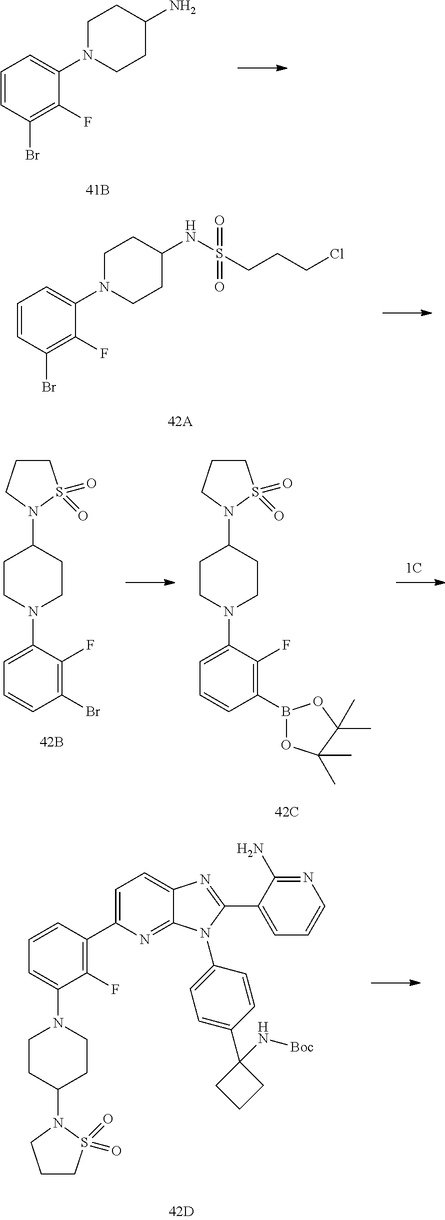 [SMILES: NC1CCN(c2cccc(Br)c2F)CC1 ; O=S(=O)(CCCCl)NC1CCN(c2cccc(Br)c2F)CC1 ; O=S1(=O)CCCN1C1CCN(c2cccc(Br)c2F)CC1 ; CC1(C)OB(c2cccc(N3CCC(N4CCCS4(=O)=O)CC3)c2F)OC1(C)C ; CC(C)(C)OC(=O)NC1(c2ccc(-n3c(-c4cccnc4N)nc4ccc(-c5cccc(N6CCC(N7CCCS7(=O)=O)CC6)c5F)nc43)cc2)CCC1]

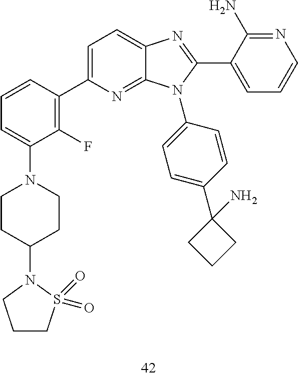 [SMILES: Nc1ncccc1-c1nc2ccc(-c3cccc(N4CCC(N5CCCS5(=O)=O)CC4)c3F)nc2n1-c1ccc(C2(N)CCC2)cc1]

### Step A: Preparation of Compound 42A

In an ice-water bath, 3-chloropropane-1-sulfonyl chloride (0.538 g) was slowly added to a solution of compound 41B (0.83 g) in N,N-dimethylformamide (15 mL), and after the addition was completed, the reaction solution was slowly warmed to room temperature and stirred. After the reaction was completed, the reaction solution was added to 30 mL of ice water, extracted with ethyl acetate, washed with saturated brine, dried over anhydrous sodium sulfate, filtered, concentrated, and subjected to column chromatography (petroleum ether/ethyl acetate=13/7) to give compound 42A (0.212 g).

MS (ESI, [M+H]⁺) m/z: 413.4.

### Step B: Preparation of Compound 42B

After compound 42A (0.212 g) was dissolved in N,N-dimethylformamide (15 mL), sodium hydrogen (146 mg) was added under an ice-bath condition, and after the addition was completed, the reaction solution was refluxed at 80° C. overnight. After the reaction was completed, the reaction solution was poured into 30 mL of ice water, extracted with ethyl acetate, washed with saturated brine, dried over anhydrous sodium sulfate, filtered, concentrated, and subjected to column chromatography (petroleum ether/ethyl acetate=2/3) to give compound 42B (0.249 g).

MS (ESI, [M+H]⁺) m/z: 377.3.

### Step C: Preparation of Compound 42C

To a reaction flask were added compound 42B (0.249 g), bis(pinacolato)diboron (0.335 g), potassium acetate (0.13 g), [1,1′-bis(diphenylphosphino)ferrocene]palladium dichloride dichloromethane complex (0.054 g) and 1,4-dioxane (15 mL) successively. The reaction solution was stirred for reaction at 100° C. under nitrogen atmosphere. After the reaction was completed, the reaction solution was filtered and concentrated. The reaction solution was subjected to column chromatography (petroleum ether/ethyl acetate=1/1) to give compound 42C (0.21 g).

MS (ESI, [M+H]⁺) m/z: 425.3.

### Step D: Preparation of Compound 42D

To a reaction flask were added compound 42C (0.21 g), compound 1C (0.243 g), potassium carbonate (0.137 g), tetrakis(triphenylphosphine)palladium (0.057 g), 1,4-dioxane (10 mL), and water (2 mL) successively. After the addition was completed, the reaction solution was purged with nitrogen gas, and stirred for reaction at 110° C. After the reaction was completed, the reaction solution was filtered and concentrated. The reaction solution was subjected to column chromatography (dichloromethane/10% methanol=3/7) to give compound 42D (185 mg).

MS (ESI) m/z: 753.7 [M+H]⁺.

### Step E: Preparation of Compound 42

Compound 42D (185 mg), methanesulfonic acid (0.16 mL) and dichloromethane (10 mL) were added successively to a reaction flask, and the mixture was stirred at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/10% methanol=2/3) to give compound 42 (40 mg).

MS (ESI, [M+H]⁺) m/z: 653.6.

¹H NMR (500 MHz, DMSO-d₆) δ 8.26 (d, 1H), 8.00 (d, 1H), 7.7 (d, 1H), 7.59 (d, 2H), 7.42 (d, 2H), 7.28 (t, 1H), 7.08-7.20 (m, 3H), 6.90 (s, 2H), 6.41 (t, 1H), 3.4 (d, 2H), 3.28 (t, 3H), 3.19 (t, 2H), 2.8 (m, 2H), 2.42 (q, 2H), 2.2 (m, 2H), 2.02-2.13 (m, 3H), 1.88 (s, 4H), 1.70 (m, 1H).

### Example 43: Preparation of Compound 43

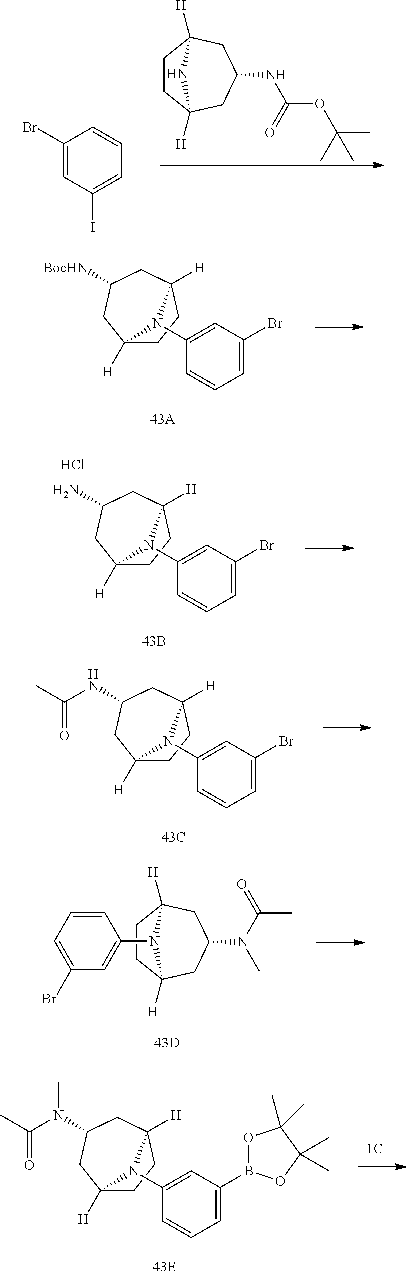

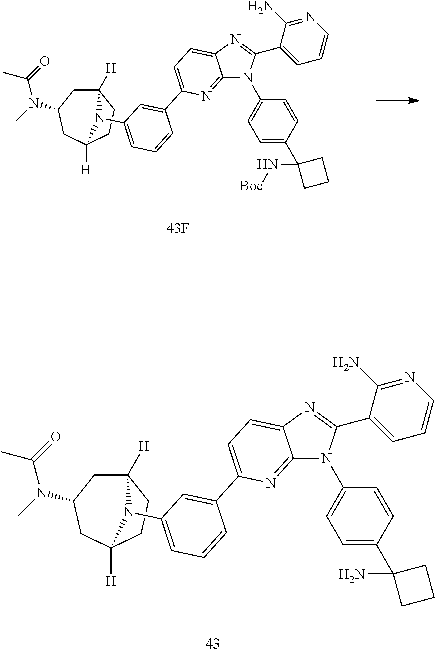 [SMILES: CC(=O)N(C)[C@@H]1C[C@H]2CC[C@@H](C1)N2c1cccc(-c2ccc3nc(-c4cccnc4N)n(-c4ccc(C5(NC(=O)OC(C)(C)C)CCC5)cc4)c3n2)c1 ; CC(=O)N(C)[C@@H]1C[C@H]2CC[C@@H](C1)N2c1cccc(-c2ccc3nc(-c4cccnc4N)n(-c4ccc(C5(N)CCC5)cc4)c3n2)c1]

### Step A: Preparation of Compound 43A

To a reaction flask were added tert-butyl exo-8-azabicyclo[3.2.1]octan-3-carbamate (0.5 g), 1-bromo-3-iodobenzene (0.75 g), potassium carbonate (0.92 g), cuprous iodide (0.05 g), L-proline (0.02 g), and dimethyl sulfoxide (10 mL) successively. After the addition was completed, the reaction solution was stirred for reaction at 100° C. under nitrogen atmosphere. After the reaction was completed, the reaction solution was filtered, concentrated, and subjected to column chromatography (petroleum ether/ethyl acetate=5/1) to give compound 43A (0.14 g).

MS (ESI, [M+H]⁺) m/z: 381.5.

### Step B: Preparation of Compound 43B

To a reaction flask were added compound 43A (0.14 g) and a solution of 4 M dioxane in hydrogen chloride (4 mL) successively, and the mixture was stirred at room temperature. After the reaction was completed, the reaction solution was concentrated to give compound 43B (0.15 g).

MS (ESI, [M+H]⁺) m/z: 281.8.

### Step C: Preparation of Compound 43C

Acetic anhydride (0.07 g) was slowly added to a reaction solution of compound 43B (0.15 g) and triethylamine (0.24 g) in dichloromethane (6 mL) at 0° C., and after the addition was completed, the reaction solution was transferred to the room temperature condition and stirred for reaction. After the reaction was completed, a saturated aqueous sodium bicarbonate solution (50 mL) was added to the reaction solution, and the reaction solution was extracted with methylene chloride (30 mL×3), and the organic phases were combined, washed with a saturated aqueous sodium chloride solution (50 mL) and dried. The reaction solution was filtered and concentrated to give compound 43C (0.11 g).

MS (ESI, [M+H]⁺) m/z: 323.4.

### Step D: Preparation of Compound 43D

Sodium hydride (0.07 g) was slowly added to a solution of compound 43C (0.22 g) in tetrahydrofuran (12 mL) at 0° C. under nitrogen atmosphere, and after the addition was completed, the reaction solution was transferred to the room temperature condition and stirred for 0.5 h, and further added with methyl iodide (0.48 g) for reaction. After the reaction was completed, a saturated aqueous ammonium chloride solution (50 mL) was added to the reaction solution in an ice bath, and the reaction solution was extracted with ethyl acetate (50 mL×3), and the organic phases were combined, washed with a saturated aqueous sodium chloride solution (100 mL), and dried. The reaction solution was filtered and concentrated to give compound 43D (0.22 g).

MS (ESI, [M+H]⁺) m/z: 336.8.

### Step E: Preparation of Compound 43E

To a reaction flask were added compound 43D (0.22 g), bis(pinacolato)diboron (0.25 g), potassium acetate (0.20 g), [1,1′-bis(diphenylphosphino)ferrocene]palladium dichloride dichloromethane complex (0.05 g) and 1,4-dioxane (10 mL) successively. The reaction solution was stirred for reaction at 100° C. under nitrogen atmosphere. After the reaction was completed, the reaction solution was filtered and concentrated. The reaction solution was subjected to column chromatography (dichloromethane/methanol=20/1) to give compound 43E (0.1 g).

MS (ESI, [M+H]⁺) m/z: 385.6.

### Step F: Preparation of Compound 43F

To a microwave tube were added compound 43E (0.10 g), compound 1C (0.11 g), potassium carbonate (0.09 g), tetrakis(triphenylphosphine)palladium (0.03 g), 1,4-dioxane (4 mL) and water (0.5 mL) successively. After the addition was completed, the reaction solution was purged with nitrogen gas, and reacted at 100° C. in microwave reactor. After the reaction was completed, the reaction solution was filtered and concentrated. The reaction solution was subjected to column chromatography (dichloromethane/methanol=20/1) to give compound 43F (0.10 g).

MS (ESI, [M+H]⁺) m/z: 713.7.

### Step F: Preparation of Compound 43

Compound 43F (100 mg), methanesulfonic acid (0.10 mL) and dichloromethane (6 mL) were added successively to a reaction flask, and the mixture was stirred at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 43 (26 mg).

MS (ESI, [M+H]⁺) m/z: 613.7.

¹H NMR (500 MHz, CDCl₃) δ 8.06 (dt, J=36.6, 6.5 Hz, 2H), 7.76 (q, J=6.6, 5.8 Hz, 1H), 7.58 (t, J=7.4 Hz, 2H), 7.53-7.39 (m, 3H), 7.39-7.24 (m, 2H), 7.15 (q, J=6.9 Hz, 1H), 6.79 (t, J=7.5 Hz, 1H), 6.61 (s, 2H), 6.39 (q, J=6.4 Hz, 1H), 5.18 (dq, J=12.7, 6.5 Hz, 1H), 4.35 (d, J=14.0 Hz, 2H), 2.61 (q, J=9.4, 8.6 Hz, 3H), 2.55-2.30 (m, 6H), 2.25-2.05 (m, 4H), 2.05-1.77 (m, 6H), 1.53-1.20 (m, 3H).

### Example 44: Preparation of Compound 44

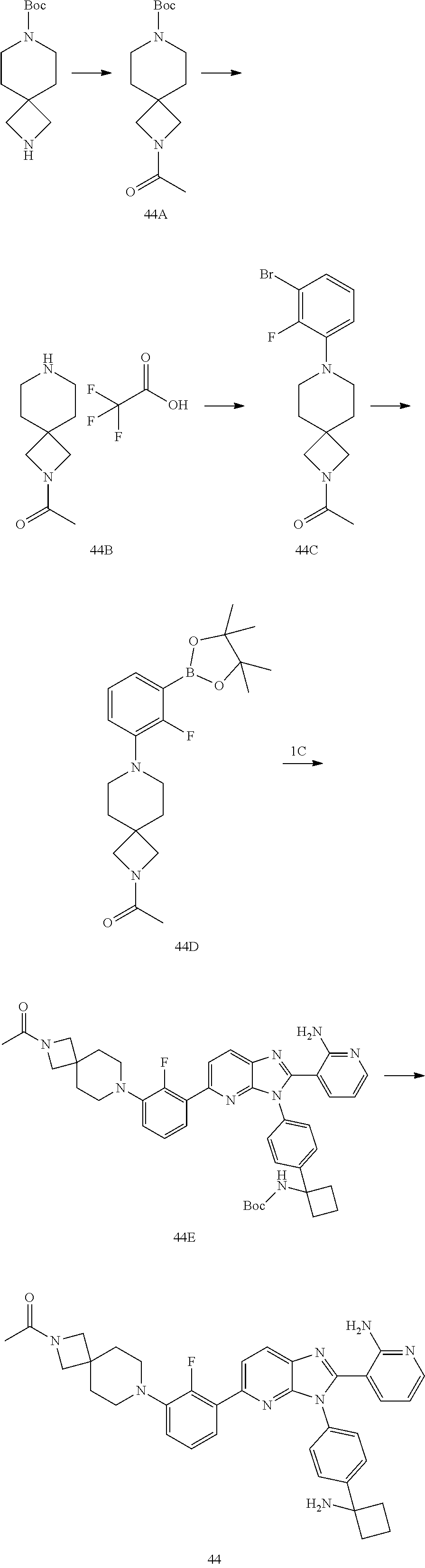

### Step A: Preparation of Compound 44A

Acetic anhydride (0.68 g) was slowly added to a reaction solution of tert-butyl 2,7-diazaspiro[3.5]nonane-7-carboxylate (1 g) and triethylamine (2.24 g) in methylene chloride (20 mL) at 0° C., and after the addition was completed, the reaction solution was transferred to the room temperature condition and stirred for reaction. After the reaction was completed, the reaction solution was extracted with saturated aqueous sodium bicarbonate and dichloromethane, washed with saturated brine, and dried over anhydrous sodium sulfate. The reaction solution was filtered and concentrated to give compound 44A (1.18 g).

MS (ESI, [M+H]⁺) m/z: 269.5.

### Step B: Preparation of Compound 44B

Trifluoroacetic acid (2 mL) was added dropwise slowly to a reaction solution of compound 44A (0.5 g) in dichloromethane (10 mL) at 0° C. After the addition was completed, the reaction solution was stirred at room temperature. After the reaction was completed, the reaction solution was concentrated to give compound 44B (0.85 g).

MS (ESI, [M+H]⁺) m/z: 169.4.

### Step C: Preparation of Compound 44C

To a reaction flask were added compound 44B (0.5 g), 1,3-dibromofluorobenzene (0.68 g), (tris(dibenzylideneacetone)dipalladium (0.16 g), 1,1′-binaphthyl-2,2′-diphenyl phosphine (0.11 g), sodium tert-butoxide (0.85 g) and 1,4-dioxane (25 mL) successively. After the addition was completed, the reaction solution was stirred for reaction at 100° C. under nitrogen atmosphere. After the reaction was completed, the reaction solution was filtered, concentrated, and subjected to column chromatography (dichloromethane/methanol=50/1) to give compound 44C (0.22 g).

MS (ESI, [M+H]⁺) m/z: 341.4.

### Step D: Preparation of Compound 44D

To a reaction flask were added compound 44C (0.22 g), bis(pinacolato)diboron (0.24 g), potassium acetate (0.19 g), [1,1′-bis(diphenylphosphino)ferrocene]palladium dichloride dichloromethane complex (0.05 g) and 1,4-dioxane (20 mL) successively. The reaction solution was stirred for reaction at 100° C. under nitrogen atmosphere. After the reaction was completed, the reaction solution was filtered and concentrated. The reaction solution was subjected to column chromatography (dichloromethane/methanol=20/1) to give compound 44D (98 mg).

MS (ESI, [M+H]⁺) m/z: 389.5.

### Step E: Preparation of Compound 44E

To a microwave tube were added compound 44D (98 mg), compound 1C (103 mg), potassium carbonate (87 mg), tetrakis(triphenylphosphine)palladium (24 mg), 1,4-dioxane (4 mL) and water (0.5 mL) successively. After the addition was completed, the reaction solution was purged with nitrogen gas, and reacted at 100° C. in microwave reactor. After the reaction was completed, the reaction solution was filtered and concentrated. The reaction solution was subjected to column chromatography (dichloromethane/methanol=20/1) to give compound 44E (84 mg).

MS (ESI, [M+H]⁺) m/z: 717.4.

### Step F: Preparation of Compound 44

Compound 44E (84 mg), methanesulfonic acid (0.10 mL) and dichloromethane (6 mL) were added successively to a reaction flask, and the mixture was stirred at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 44 (37 mg).

MS (ESI, [M+H]⁺) m/z: 617.6.

¹H NMR (500 MHz, CDCl₃) δ 8.11 (d, J=8.3 Hz, 1H), 8.05-7.99 (m, 1H), 7.79 (dd, J=8.4, 2.1 Hz, 1H), 7.56 (d, J=8.0 Hz, 2H), 7.49 (t, J=7.3 Hz, 1H), 7.38 (d, J=8.0 Hz, 2H), 7.14-7.06 (m, 2H), 6.95 (t, J=7.9 Hz, 1H), 6.59 (s, 2H), 6.39 (dd, J=7.7, 4.8 Hz, 1H), 3.80 (d, J=49.5 Hz, 4H), 3.06-2.92 (m, 4H), 2.64-2.58 (m, 2H), 2.40-2.32 (m, 2H), 2.24-2.14 (m, 1H), 1.98-1.92 (m, 4H), 1.89 (s, 3H), 1.85-1.80 (m, 1H).

### Example 45: Preparation of Compound 45

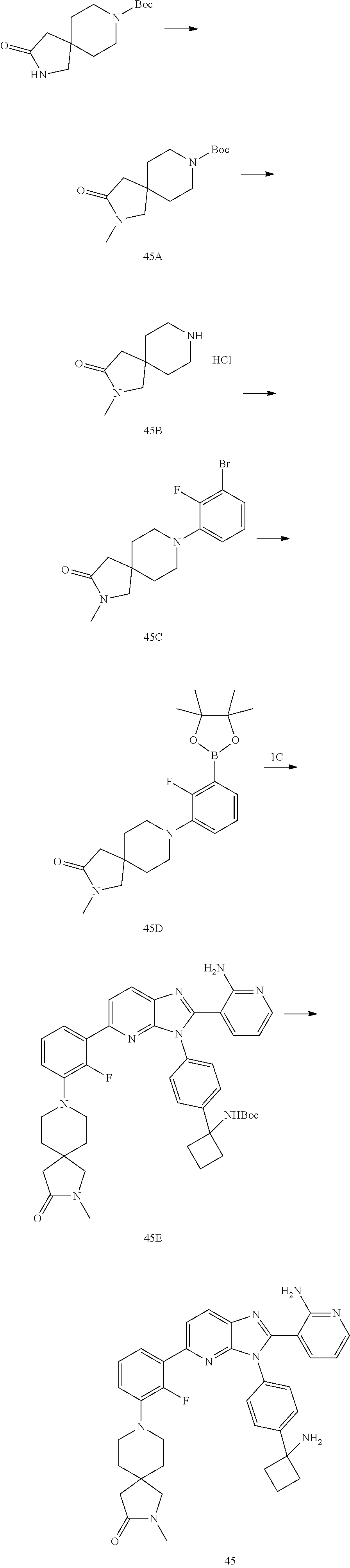

### Step A: Preparation of Compound 45A

Sodium hydride (7.9 g) was slowly added to a solution of tert-butyl 3-oxo-2,8-diazospiro[4.5]decane-8-carboxylate (10 g) in tetrahydrofuran at 0° C. under nitrogen atmosphere, and the mixture was stirred at room temperature for 1 h. Methyl iodide (27.9 g) was added dropwise to the reaction flask at 0° C. and then stirred at room temperature. After the reaction was completed, the reaction was quenched with aqueous ammonium chloride solution, and the reaction solution was extracted with ethyl acetate, dried over anhydrous sodium sulfate, filtered, and concentrated to give compound 45A (13.0 g).

MS (ESI, [M+H]⁺) m/z: 269.5.

### Step B: Preparation of Compound 45B

To a reaction flask were added compound 45A (13.0 g), dichloromethane (260 mL) and diluted hydrochloric acid (12.4 g) successively, and the mixture was stirred at room temperature. After the reaction was completed, the reaction solution was filtered and dried to give compound 45B (8.9 g).

MS (ESI, [M+H]⁺) m/z: 169.4.

### Step C: Preparation of Compound 45C

To a reaction flask were added sodium tert-butoxide (11.3 g), 1,1′-binaphthyl-2,2′-bis-diphenylphosphine (1.8 g), tris(dibenzylideneacetone)dipalladium (2.7 g), 1,3-dibromo-2-fluorobenzene (11.2 g), compound 45B (8.9 g) and 1,4-dioxane (100 mL) successively, and the reaction solution was stirred for reaction under nitrogen atmosphere at 100° C. After the reaction solution was completed, the reaction solution was filtered and concentrated. The reaction solution was subjected to column chromatography (dichloromethane/methanol=100/1) to give compound 45C (4.8 g).

MS (ESI, [M+H]⁺) m/z: 341.4.

### Step D: Preparation of Compound 45D

To a reaction flask were added compound 45C (3.0 g), bis(pinacolato)diboron (3.4 g), potassium acetate (2.6 g), [1,1′-bis(diphenylphosphino)ferrocene]palladium dichloride dichloromethane complex (1.4 g) and 1,4-dioxane (100 mL) successively. The reaction solution was stirred for reaction at 100° C. under nitrogen atmosphere. After the reaction was completed, the reaction solution was filtered and concentrated. The reaction solution was subjected to column chromatography (dichloromethane/methanol=100/1) to give compound 45D (3.0 g).

MS (ESI, [M+H]⁺) m/z: 389.5.

### Step E: Preparation of Compound 45E

To a reaction flask were added compound 45D (2.8 g), compound 1C (2.3 g), potassium carbonate (1.3 g), tetrakis(triphenylphosphine)palladium (1.1 g), 1,4-dioxane (50 mL), and water (10 mL) successively. After the addition was completed, the reaction solution was purged with nitrogen gas, and stirred for reaction at 100° C. After the reaction was completed, the reaction solution was filtered and concentrated. The reaction solution was subjected to column chromatography (dichloromethane/methanol=20/1) to give compound 45E (2.1 g).

MS (ESI, [M+H]⁺) m/z: 717.5.

### Step F: Preparation of Compound 45

To a reaction flask were added compound 45E (2.1 g), methanesulfonic acid (2.3 g) and dichloromethane (80 mL) successively, and the reaction solution was stirred for reaction at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=20/1) to give compound 45 (0.43 g).

MS (ESI, [M+H]⁺) m/z: 617.6.

¹H NMR (500 MHz, CDCl₃) δ 8.19-7.88 (m, 2H), 7.73 (d, J=8.5 Hz, 1H), 7.60-7.26 (m, 5H), 7.05 (s, 2H), 6.94 (d, J=8.8 Hz, 1H), 6.53 (d, J=10.6 Hz, 1H), 6.33 (s, 1H), 3.19 (s, 2H), 3.02 (s, 4H), 2.94 (d, J=9.5 Hz, 2H), 2.79 (s, 3H), 2.60-2.52 (m, 1H), 2.27 (d, J=14.2 Hz, 3H), 2.10 (s, 1H), 1.75 (s, 4H), 1.19 (d, J=14.4 Hz, 1H).

### Example 46: Preparation of Compound 46

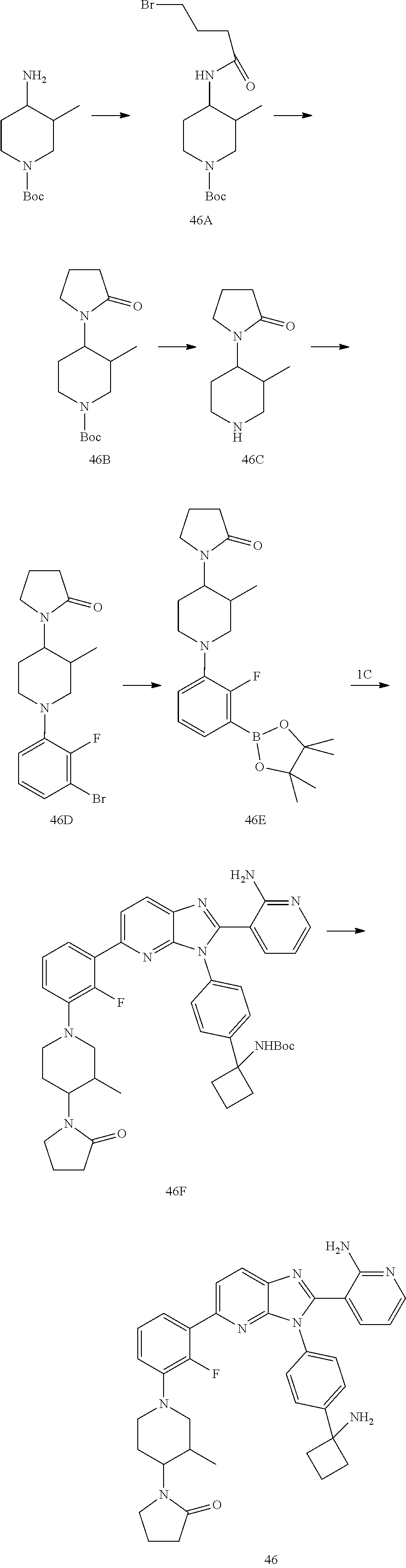

### Step A: Preparation of Compound 46A

To a reaction flask were added tert-butyl 4-amino-3-methylpiperidine-1-carboxylate oxalate (1.4 g), triethylamine (0.93 g), 4-bromobutyryl chloride (0.93 g) and tetrahydrofuran (25 mL) successively. After the addition was completed, the reaction solution was stirred at room temperature. After the reaction was completed, the reaction solution was distilled under reduced pressure to remove the solvent and concentrated to give compound 46A (0.84 g).

MS (ESI, [M+H]⁺) m/z: 363.1.

### Step B: Preparation of Compound 46B

To a reaction flask were added compound 46A (3.0 g), sodium hydride (0.088 g), and tetrahydrofuran (25 mL) successively. The mixture was stirred at room temperature. After the reaction was completed, the reaction solution was concentrated to give compound 46B (0.18 g).

MS (ESI, [M+H]⁺) m/z: 283.5.

### Step C: Preparation of Compound 46C

To a reaction flask were added compound 46B (1.0 g), a solution of 4 M hydrochloric acid in 1,4-dioxane (3.7 mL), and a solution of 1,4-dioxane (20 mL) successively. The mixture was stirred at room temperature. After the reaction was completed, the reaction solution was concentrated to give compound 46C (0.83 g).

MS (ESI, [M+H]⁺) m/z: 183.5.

### Step D: Preparation of Compound 46D

To a reaction flask were added compound 46C (3.0 g), cesium carbonate (2.3 g), tris(dibenzylideneacetone)dipalladium-chloroform adduct (0.21 g), 1-(3-methylpiperidin-4-yl)pyrrolidin-2-one hydrochloride (0.51 g), 1,3-dibromom-fluorobenzene (0.89 g), 1,1′-binaphthyl-2,2′-diphenyl phosphine (0.14 g), and dioxane (10 mL) successively. The reaction solution was stirred at 100° C. After the reaction was completed, the reaction solution was concentrated and subjected to column chromatography (petroleum ether/ethyl acetate=9/1) to give compound 46D (0.38 g).

MS (ESI, [M+H]⁺) m/z: 355.4.

### Step E: Preparation of Compound 46E

To a reaction flask were added 46D (0.38 g), potassium carbonate (0.21 g), bis(pinacolato)diboron (0.41 g), [1,1′-bis(diphenylphosphino)ferrocene]palladium dichloride (0.17 g) and dioxane (20 mL) successively under nitrogen atmosphere. After the addition was completed, the reaction solution was stirred at 100° C. After the reaction was completed, the reaction solution was concentrated and subjected to column chromatography (petroleum ether/ethyl acetate=9/1) to give compound 46E (0.16 g).

MS (ESI, [M+H]⁺) m/z: 403.6.

### Step F: Preparation of Compound 46F

To a reaction flask were added compound 46E (0.16 g), compound 1C (0.18 g), potassium carbonate (0.087 g), tetrakis(triphenylphosphine)palladium (0.075 g), 1,4-dioxane (10 mL), and water (2.0 mL) successively. After the addition was completed, the reaction solution was purged with nitrogen gas, and reacted at 100° C. After the reaction was completed, the reaction solution was filtered and concentrated. The reaction solution was subjected to column chromatography (dichloromethane/methanol=20/1) to give compound 46F (0.20 g).

MS (ESI, [M+H]⁺) m/z: 731.8.

### Step G: Preparation of Compound 46

Compound 46F (0.20 g), methanesulfonic acid (0.16 g) and dichloromethane (15 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 46 (0.078 g).

MS (ESI, [M+H]⁺) m/z: 631.7.

¹H NMR (500 MHz, CDCl₃) δ 8.10-8.04 (m, 1H), 7.94 (dd, J=5.0, 2.3 Hz, 1H), 7.74 (dq, J=8.1, 2.7 Hz, 1H), 7.56-7.47 (m, 2H), 7.43-7.33 (m, 3H), 7.05 (tt, J=11.9, 3.0 Hz, 2H), 6.95 (t, J=8.0 Hz, 1H), 6.34 (ddd, J=7.4, 4.9, 2.1 Hz, 1H), 3.71 (td, J=12.1, 4.2 Hz, 1H), 3.46-3.32 (m, 3H), 3.32-3.26 (m, 1H), 2.76 (td, J=11.9, 2.9 Hz, 1H), 2.59 (dddd, J=11.9, 9.0, 6.3, 2.3 Hz, 2H), 2.47-2.41 (m, 1H), 2.37 (dtt, J=11.7, 5.8, 2.6 Hz, 4H), 2.15 (ddtt, J=15.9, 9.6, 6.7, 3.4 Hz, 1H), 1.98 (dp, J=12.3, 5.2, 4.0 Hz, 3H), 1.81 (ddtq, J=12.4, 9.3, 6.3, 3.7, 2.9 Hz, 1H), 1.72-1.60 (m, 1H), 1.34 (d, J=2.3 Hz, 1H), 1.26-1.14 (m, 3H).

### Example 47: Preparation of Compound 47

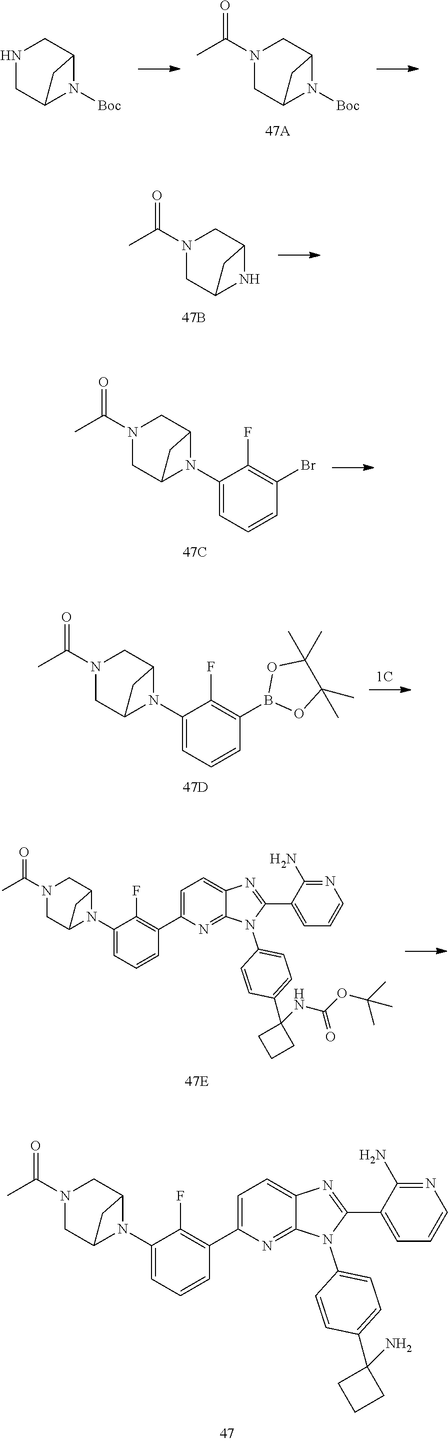

### Step A: Preparation of Compound 47A

To a reaction flask were added tert-butyl 3,6-diazabicyclo[3.1.1]heptane-6-carboxylate (2.5 g), acetic anhydride (1.93 g), triethylamine (6.3 g), and dichloromethane (40 mL) successively. The mixture was stirred at room temperature. After the reaction was completed, water and dichloromethane were added to extract the reaction solution, and the reaction solution was washed with saturated brine and dried over anhydrous sodium sulfate. The reaction solution was filtered and concentrated to give compound 47A (3.0 g).

MS (ESI, [M+Na]⁺) m/z: 241.1.

### Step B: Preparation of Compound 47B

To a reaction flask were added compound 47A (3.01 g), trifluoroacetic acid (2.2 g), and dichloromethane (50 mL) successively. The mixture was stirred at room temperature. After the reaction was completed, the reaction solution was concentrated to give compound 47B (2.3 g).

MS (ESI, [M+H]⁺) m/z: 141.1.

### Step C: Preparation of Compound 47C

To a reaction flask were added 1,1′-binaphthyl-2,2′-diphenyl phosphine (0.73 g), tris(dibenzylideneacetone)dipalladium (0.26 g), 1,3-dibromo-2-fluorobenzene (4.5 g), compound 47B (1.6 g), sodium tert-butoxide (11 g) and dioxane (20 mL) successively under nitrogen atmosphere. After the addition was completed, the reaction solution was stirred at 85° C. After the reaction was completed, the reaction solution was concentrated and subjected to column chromatography (petroleum ether/ethyl acetate=9/1) to give compound 47C (0.43 g).

MS (ESI, [M+H]⁺) m/z: 312.9.

### Step D: Preparation of Compound 47D

To a reaction flask were added compound 47C (0.43 g), bis(pinacolato)diboron (0.52 g), potassium acetate (0.22 g), [1,1′-bis(diphenylphosphino)ferrocene]palladium dichloride dichloromethane complex (0.270 g) and 1,4-dioxane (60 mL) successively. The reaction solution was stirred at 100° C. under nitrogen atmosphere. After the reaction was completed, the reaction solution was filtered concentrated. The reaction solution was subjected to column chromatography (dichloromethane/methanol=20/1) to give compound 47D (0.13 g).

MS (ESI, [M+H]⁺) m/z: 361.6.

### Step E: Preparation of Compound 47E

To a reaction flask were added compound 47D (0.13 g), compound 1C (0.12 g), potassium carbonate (0.068 g), tetrakis(triphenylphosphine)palladium (0.057 g), 1,4-dioxane (25 mL), and water (5 mL) successively. After the addition was completed, the reaction solution was purged with nitrogen gas, and reacted at 100° C. After the reaction was completed, the reaction solution was filtered and concentrated. The reaction solution was subjected to column chromatography (dichloromethane/methanol=20/1) to give compound 47E (0.21 g).

MS (ESI, [M+H]⁺) m/z: 689.44.

### Step F: Preparation of Compound 47

Compound 47E (0.21 g), methanesulfonic acid (0.58 g) and dichloromethane (25 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 47 (0.11 g).

MS (ESI, [M+H]⁺) m/z: 589.6.

¹H NMR (500 MHz, CDCl₃) δ 8.07-7.89 (m, 2H), 7.61 (d, J=8.4 Hz, 1H), 7.46 (dd, J=17.7, 8.1 Hz, 2H), 7.28 (t, J=12.7 Hz, 2H), 7.15 (t, J=7.4 Hz, 1H), 7.05 (d, J=7.8 Hz, 1H), 6.97 (q, J=7.7 Hz, 1H), 6.48 (dt, J=26.1, 8.2 Hz, 2H), 6.33 (dd, J=7.8, 4.8 Hz, 1H), 4.46-4.25 (m, 2H), 3.86 (dd, J=18.6, 12.8 Hz, 2H), 3.53 (d, J=13.7 Hz, 1H), 3.47-3.28 (m, 1H), 2.78 (q, J=6.9 Hz, 1H), 2.48 (dq, J=53.9, 11.6, 10.3 Hz, 4H), 2.28-2.09 (m, 1H), 1.91-1.79 (m, 3H), 1.54 (d, J=8.6 Hz, 1H), 1.35 (s, 1H).

### Example 48: Preparation of Compound 48

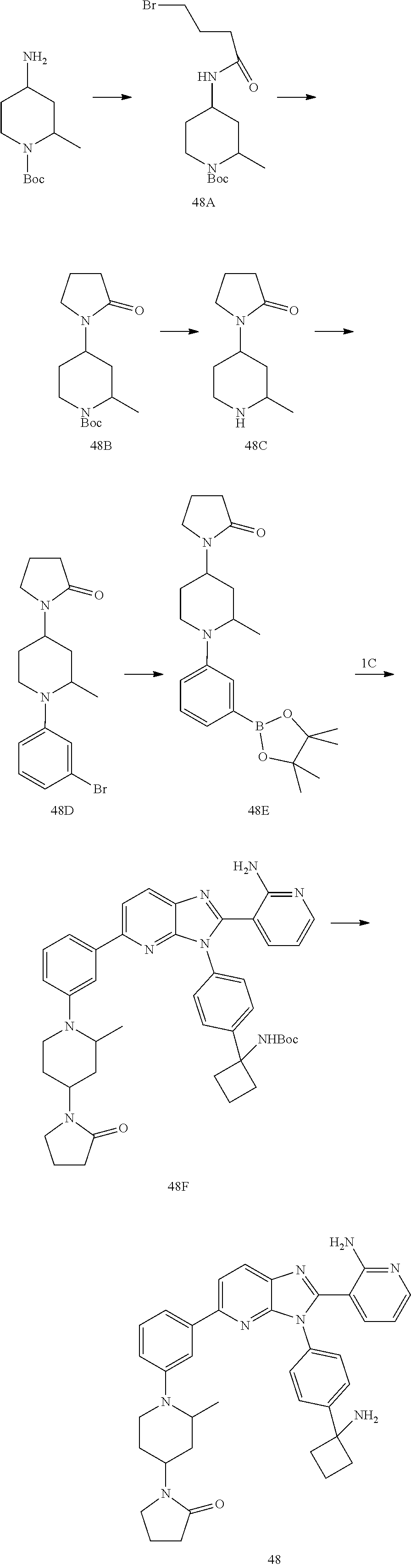

### Step A: Preparation of Compound 48A

To a reaction flask were added tert-butyl 4-amino-3-methylpiperidine-1-carboxylate oxalate (3.5 g), triethylamine (2.9 g), 4-bromobutyryl chloride (3.3 g) and tetrahydrofuran (25 mL) successively. After the addition was completed, the reaction solution was stirred at room temperature. After the reaction was completed, the reaction solution was distilled under reduced pressure to remove the solvent and concentrated to give compound 48A (0.78 g).

MS (ESI, [M+H]⁺) m/z: 363.1.

### Step B: Preparation of Compound 48B

To a reaction flask were added compound 48A (1.4 g), sodium hydride (0.088 g), and tetrahydrofuran (25 mL) successively. The mixture was stirred at room temperature. After the reaction was completed, the reaction solution was concentrated to give compound 48B (0.52 g).

MS (ESI, [M+H]⁺) m/z: 283.5.

### Step C: Preparation of Compound 48C

To a reaction flask were added compound 48B (0.52 g), a solution of 4 M hydrochloric acid in 1,4-dioxane (0.34 mL), and a solution of 1,4-dioxane (20 mL) successively. The mixture was stirred at room temperature. After the reaction was completed, the reaction solution was concentrated to give compound 48C (0.41 g).

MS (ESI, [M+H]⁺) m/z: 183.5.

### Step D: Preparation of Compound 48D

To a reaction flask were added compound 48C (0.41 g), cesium carbonate (2.2 g), tris(dibenzylideneacetone)dipalladium-chloroform adduct (0.21 g), 1-(3-methylpiperidin-4-yl)pyrrolidin-2-one hydrochloride (0.51 g), 1,3-dibromom-fluorobenzene (0.89 g), 1,1′-binaphthyl-2,2′-diphenyl phosphine (0.14 g), and dioxane (10 mL) successively. The reaction solution was stirred at 100° C. After the reaction was completed, the reaction solution was concentrated and subjected to column chromatography (petroleum ether/ethyl acetate=9/1) to give compound 48D (0.28 g).

MS (ESI, [M+H]⁺) m/z: 337.1.

### Step E: Preparation of Compound 48E

To a reaction flask were added 48D (0.28 g), potassium carbonate (0.24 g), bis(pinacolato)diboron (0.32 g), [1,1′-bis(diphenylphosphino)ferrocene]palladium dichloride (0.138 g) and dioxane (20 mL) successively under nitrogen atmosphere. After the addition was completed, the reaction solution was stirred at 100° C. After the reaction was completed, the reaction solution was concentrated and subjected to column chromatography (petroleum ether/ethyl acetate=9/1) to give compound 48E (0.087 g).

MS (ESI, [M+H]⁺) m/z: 385.3.

### Step F: Preparation of Compound 48F

To a reaction flask were added compound 48E (0.087 g), compound 1C (0.075 g), potassium carbonate (0.087 g), tetrakis(triphenylphosphine)palladium (0.075 g), 1,4-dioxane (10 mL), and water (2 mL) successively. After the addition was completed, the reaction solution was purged with nitrogen gas, and reacted at 100° C. After the reaction was completed, the reaction solution was filtered and concentrated. The reaction solution was subjected to column chromatography (dichloromethane/methanol=20/1) to give compound 48F (0.080 g).

MS (ESI, [M+H]⁺) m/z: 713.1.

### Step G: Preparation of Compound 48

Compound 48F (0.080 g), methanesulfonic acid (0.16 g) and dichloromethane (15 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 48 (0.021 g).

MS (ESI, [M+H]⁺) m/z: 613.1.

¹H NMR (500 MHz, CDCl₃) δ 8.09-7.94 (m, 2H), 7.70 (d, J=8.5 Hz, 1H), 7.48 (h, J=9.0, 8.5 Hz, 3H), 7.36 (d, J=7.8 Hz, 2H), 7.23 (t, J=7.9 Hz, 1H), 7.09 (d, J=7.7 Hz, 1H), 6.84 (dd, J=8.2, 2.4 Hz, 1H), 6.59 (d, J=20.1 Hz, 2H), 6.33 (dd, J=7.8, 4.8 Hz, 1H), 4.39 (ddt, J=12.9, 9.3, 4.6 Hz, 1H), 4.26 (dt, J=22.0, 11.0 Hz, 1H), 3.47 (dt, J=12.6, 3.7 Hz, 1H), 3.29 (t, J=7.0 Hz, 2H), 3.16-3.03 (m, 1H), 2.54 (dt, J=12.0, 8.5 Hz, 3H), 2.35 (t, J=8.1 Hz, 3H), 2.16 (tt, J=9.0, 4.5 Hz, 3H), 1.72 (dtd, J=24.1, 12.1, 11.6, 6.7 Hz, 4H), 1.60 (d, J=12.3 Hz, 2H), 1.07 (d, J=6.9 Hz, 3H).

### Example 49: Preparation of Compound 49

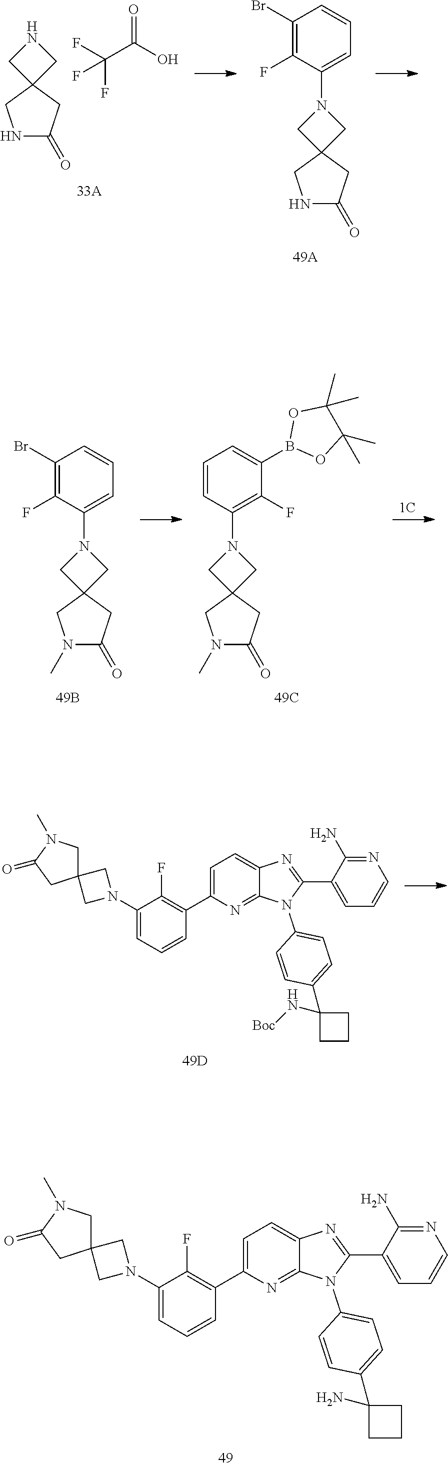

### Step A: Preparation of Compound 49A

To a reaction flask were added 33A (0.52 g), 1,3-dibromofluorobenzene (0.83 g), (tris(dibenzylideneacetone)dipalladium (0.20 g), 1,1′-binaphthyl-2,2′-diphenyl phosphine (0.14 g), sodium tert-butoxide (1.04 g) and 1,4-dioxane (30 mL) successively. After the addition was completed, the reaction solution was stirred for reaction at 100° C. under nitrogen atmosphere. After the reaction was completed, the reaction solution was filtered, concentrated, and subjected to column chromatography (dichloromethane/methanol=50/1) to give compound 49A (0.22 g).

MS (ESI, [M+H]⁺) m/z: 299.3.

### Step B: Preparation of Compound 49B

Sodium hydride (0.11 g) was slowly added to a solution of compound 49A (0.16 g) in tetrahydrofuran (10 mL) at 0° C. under nitrogen atmosphere, and after the addition was completed, the reaction solution was transferred to the room temperature condition and stirred for 0.5 h, and further added with methyl iodide (0.23 g) for reaction. After the reaction was completed, a saturated aqueous ammonium chloride solution was added to the reaction solution in an ice bath, followed by extraction with ethyl acetate, and the reaction solution was washed with saturated brine, and dried over anhydrous sodium sulfate. The reaction solution was filtered and concentrated to give compound 49B (0.18 g).

MS (ESI, [M+H]⁺) m/z: 313.3.

### Step C: Preparation of Compound 49C

To a reaction flask were added compound 49B (0.17 g), bis(pinacolato)diboron (0.20 g), potassium acetate (0.16 g), [1,1′-bis(diphenylphosphino)ferrocene]palladium dichloride dichloromethane complex (0.04 g) and 1,4-dioxane (10 mL) successively. The reaction solution was stirred for reaction at 100° C. under nitrogen atmosphere. After the reaction was completed, the reaction solution was filtered and concentrated. The reaction solution was subjected to column chromatography (dichloromethane/methanol=20/1) to give compound 49C (0.19 g).

MS (ESI, [M+H]⁺) m/z: 361.5.

### Step D: Preparation of Compound 49D

To a microwave tube were added compound 49C (0.14 g), compound 1C (0.16 g), potassium carbonate (0.14 g), tetrakis(triphenylphosphine)palladium (0.04 g), 1,4-dioxane (4 mL) and water (0.5 mL) successively. After the addition was completed, the reaction solution was purged with nitrogen gas, and reacted at 100° C. in microwave reactor. After the reaction was completed, the reaction solution was filtered and concentrated. The reaction solution was subjected to column chromatography (dichloromethane/methanol=20/1) to give compound 49D (0.14 g).

MS (ESI, [M+H]⁺) m/z: 689.7.

### Step E: Preparation of Compound 49

Compound 49D (135 mg), methanesulfonic acid (0.10 mL) and dichloromethane (6 mL) were added successively to a reaction flask, and the mixture was stirred at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 49 (49 mg).

MS (ESI, [M+H]⁺) m/z: 589.4.

¹H NMR (500 MHz, CDCl₃) δ 8.14 (dd, J=8.5, 3.4 Hz, 1H), 8.02 (dt, J=4.9, 2.5 Hz, 1H), 7.83-7.75 (m, 1H), 7.55 (dt, J=8.4, 2.4 Hz, 2H), 7.42 (dt, J=8.3, 2.4 Hz, 2H), 7.32-7.26 (m, 1H), 7.17-7.13 (m, 1H), 7.07 (dd, J=9.5, 6.0 Hz, 1H), 6.57-6.49 (m, 1H), 6.45-6.39 (m, 1H), 4.10-3.96 (m, 4H), 3.71 (d, J=3.3 Hz, 2H), 2.89 (d, J=3.5 Hz, 3H), 2.72 (d, J=3.5 Hz, 2H), 2.70-2.60 (m, 2H), 2.37-2.22 (m, 2H), 2.20-2.08 (m, 1H), 1.90-1.80 (m, 1H).

### Example 50: Preparation of Compound 50

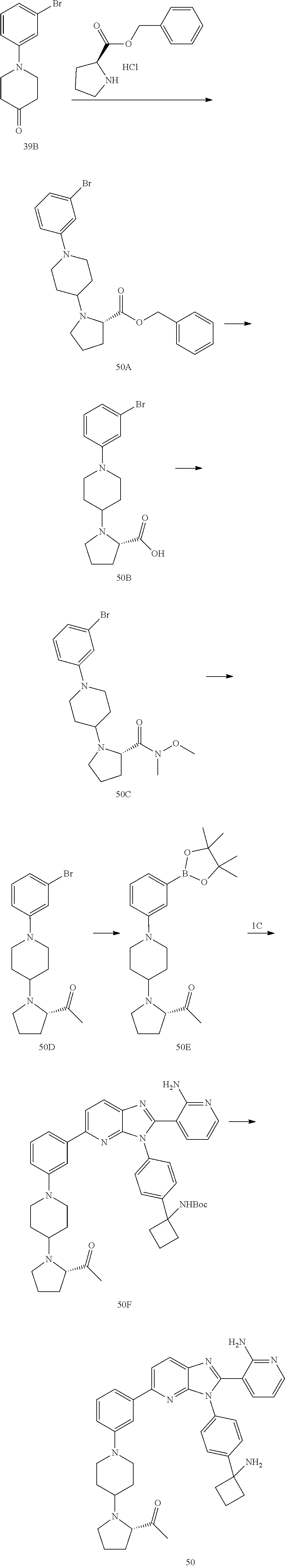

### Step A: Preparation of Compound 50A

To a reaction flask were added compound 39B (1.7 g), L-proline benzyl hydrochloride (2.4 g), acetic acid (0.8 g) and methanol (20 mL) successively, and the mixture was stirred at room temperature under nitrogen atmosphere. Sodium cyanoborohydride (0.84 g) was added thereto, and after the addition was completed, the mixture was stirred at room temperature. After the reaction was completed, a saturated aqueous sodium hydrogencarbonate solution (50 mL) was added to the reaction solution, and the mixture was concentrated; the residue was extracted with dichloromethane, dried over anhydrous sodium sulfate, filtered, and concentrated to give compound 50A (1.3 g).

MS (ESI, [M+H]⁺) m/z: 443.4.

### Step B: Preparation of Compound 50B

To a reaction flask were added compound 50A (1.3 g), sodium hydroxide (0.35 g), tetrahydrofuran (30 mL) and water (10 mL) successively, and the reaction solution was stirred for reaction at 70° C.

After the reaction was completed, the reaction solution was concentrated, the residue was extracted with ethyl acetate, and the pH of the aqueous phase was adjusted to 4-5 with 1 M diluted hydrochloric acid. The aqueous phase was then concentrated, dissolved in methanol (30 mL), filtered through an organic membrane, and further concentrated to give compound 50B (1.0 g).

### Step C: Preparation of Compound 50C

To a reaction flask were added compound 50B (1.0 g), 2-(7-azabenzotriazole)-N,N,N′,N′-tetramethyluronium hexafluorophosphate (2.2 g) and dichloromethane (20 mL) successively, and the mixture was stirred at room temperature under nitrogen atmosphere. N,N-diisopropylethylamine (1.5 g) and N,O-dimethylhydroxylamine hydrochloride (0.4 g) were added to the reaction solution, and after the addition was completed, the reaction solution was stirred at room temperature. After the reaction was completed, the reaction solution was concentrated and subjected to column chromatography (dichloromethane/methanol=100/1) to give compound 50C (1.0 g).

MS (ESI, [M+H]⁺) m/z: 396.2.

### Step D: Preparation of Compound 50D

Methylmagnesium bromide (1.5 g) was added dropwise to a solution of compound 50C (1.0 g) in tetrahydrofuran (20 mL) at −15° C. under nitrogen atmosphere, and after the addition was completed, the mixture was stirred at room temperature. After the reaction was completed, 50 mL of a saturated aqueous ammonium chloride solution was added to the reaction flask, and the reaction solution was extracted with ethyl acetate and concentrated. The reaction solution was subjected to column chromatography (dichloromethane/methanol=40/1) to give compound 50D (0.23 g).

MS (ESI, [M+H]⁺) m/z: 351.1.

### Step E: Preparation of Compound 50E

To a reaction flask were added compound 50D (0.23 g), bis(pinacolato)diboron (0.33 g), potassium acetate (0.2 g), [1,1′-bis(diphenylphosphino)ferrocene]palladium dichloride dichloromethane complex (0.053 g) and 1,4-dioxane (20 mL) successively. The reaction solution was stirred for reaction at 100° C. under nitrogen atmosphere. After the reaction was completed, the reaction solution was filtered, concentrated, and subjected to column chromatography (dichloromethane/methanol=40/1) to give compound 50E (0.054 g).

MS (ESI, [M+H]⁺) m/z: 399.4.

### Step F: Preparation of Compound 50F

To a reaction flask were added compound 50E (0.054 g), compound 1C (0.06 g), potassium carbonate (0.051 g), tetrakis(triphenylphosphine)palladium (0.014 g), 1,4-dioxane (5 mL), and water (1 mL) successively. After the addition was completed, the reaction solution was stirred for reaction at 100° C. under nitrogen atmosphere. After the reaction was completed, the reaction solution was filtered, concentrated, and subjected to column chromatography (dichloromethane/methanol=20/1) to give compound 50F (0.050 g).

MS (ESI, [M+H]⁺) m/z: 727.4.

### Step F: Preparation of Compound 50

To a reaction flask were added compound 50F (0.05 g), methanesulfonic acid (0.066 g) and dichloromethane (6 mL) successively, and the reaction solution was stirred for reaction at room temperature. After the reaction was completed, 10% aqueous sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=20/1) to give compound 50 (0.0050 g).

MS (ESI, [M+H]⁺) m/z: 627.7.

¹H NMR (500 MHz, DMSO) δ 9.36 (d, J=41.5 Hz, 1H), 8.99 (s, 1H), 8.34-8.26 (m, 1H), 8.08 (d, J=6.1 Hz, 1H), 8.03 (d, J=8.4 Hz, 1H), 7.74 (dd, J=33.2, 8.5 Hz, 2H), 7.66-7.57 (m, 3H), 7.48 (d, J=7.1 Hz, 3H), 7.31 (t, J=7.9 Hz, 1H), 7.02 (d, J=6.5 Hz, 1H), 6.61 (s, 1H), 4.88 (d, J=26.5 Hz, 1H), 3.90 (dd, J=20.9, 9.0 Hz, 2H), 3.55 (d, J=34.7 Hz, 2H), 2.72 (s, 3H), 2.63 (t, J=7.8 Hz, 4H), 2.37 (d, J=9.4 Hz, 3H), 2.28-2.09 (m, 3H), 2.07-1.96 (m, 3H), 1.88-1.80 (m, 2H), 1.62 (dd, J=71.1, 14.4 Hz, 3H).

### Example 51: Preparation of Compound 51

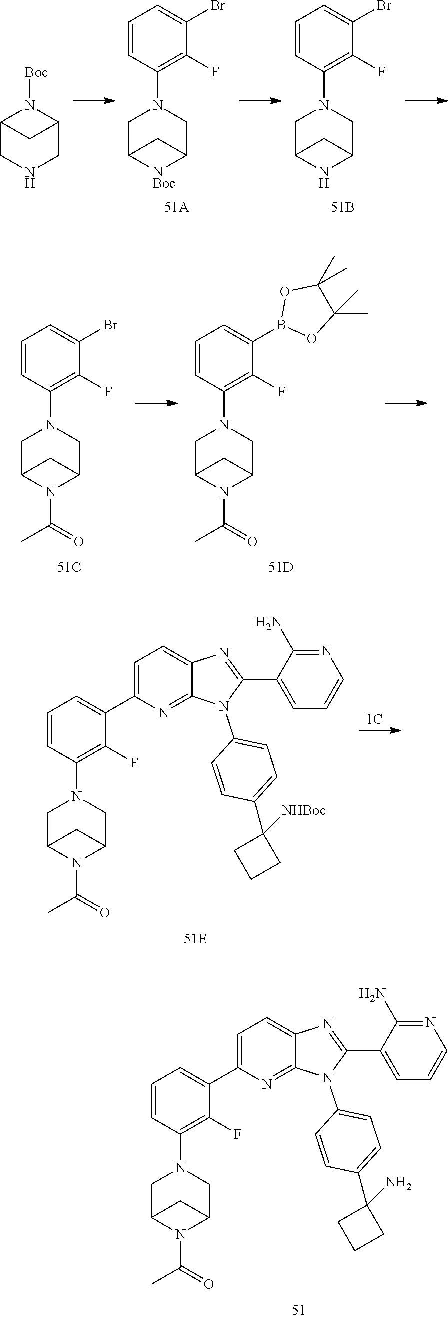

### Step A: Preparation of Compound 51A

To a reaction flask were added 1,1′-binaphthyl-2,2′-bis-diphenylphosphine (0.22 g), tris(dibenzylideneacetone)dipalladium (0.32 g), 1,3-dibromo-2-fluorobenzene (0.89 g), tert-butyl 2,7-diazaspiro[3.5]nonane-7-carbonate (0.70 g), sodium tert-butoxide (1.0 g), and tetrahydrofuran (25 mL) successively under nitrogen atmosphere. After the addition was completed, the reaction solution was stirred at 85° C. After the reaction was completed, the reaction solution was concentrated and subjected to column chromatography (petroleum ether/ethyl acetate=5/1) to give compound 51A (0.37 g).

MS (ESI, [M+H]⁺) m/z: 371.3.

### Step B: Preparation of Compound 51B

Compound 51A (0.37 g), methanesulfonic acid (0.28 g) and dichloromethane (10 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered and concentrated to give compound 51B (0.23 g).

MS (ESI, [M+H]⁺) m/z: 271.3.

### Step C: Preparation of Compound 51C

To a reaction flask were added compound 51B (0.23 g), acetic anhydride (0.13 g), triethylamine (0.87 g) and dichloromethane (50 mL) successively. The mixture was stirred at room temperature. After the reaction was completed, water and dichloromethane were added to extract the reaction solution, and the reaction solution was washed with saturated brine and dried over anhydrous sodium sulfate. The reaction solution was filtered and concentrated to give compound 51C (0.34 g).

MS (ESI, [M+H]⁺) m/z: 313.0.

### Step D: Preparation of Compound 51D

To a reaction flask were added compound 51C (0.34 g), bis(pinacolato)diboron (0.42 g), potassium acetate (0.21 g), [1,1′-bis(diphenylphosphino)ferrocene]palladium dichloride dichloromethane complex (0.18 g) and 1,4-dioxane (50 mL) successively. The reaction solution was stirred at 100° C. under nitrogen atmosphere. After the reaction was completed, the reaction solution was filtered and concentrated. The reaction solution was subjected to column chromatography (dichloromethane/methanol=20/1) to give compound 51D (0.17 g).

MS (ESI, [M+H]⁺) m/z: 361.5.

### Step E: Preparation of Compound 51E

To a reaction flask were added compound 51D (0.17 g), compound 1C (0.16 g), potassium carbonate (0.89 g), tetrakis(triphenylphosphine)palladium (0.74 g), 1,4-dioxane (25 mL), and water (5 mL) successively. After the addition was completed, the reaction solution was purged with nitrogen gas, and reacted at 100° C. After the reaction was completed, the reaction solution was filtered and concentrated. The reaction solution was subjected to column chromatography (dichloromethane/methanol=20/1) to give compound 51E (0.15 g).

MS (ESI, [M+H]⁺) m/z: 689.6.

### Step F: Preparation of Compound 51

Compound 51E (0.15 g), methanesulfonic acid (0.21 g) and dichloromethane (10 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 51 (0.089 g).

HRMS (ESI, [M+H]⁺) m/z: 589.2825.

¹H NMR (500 MHz, CDCl₃) δ 8.09-7.94 (m, 2H), 7.66 (d, J=8.5 Hz, 1H), 7.48 (d, J=7.9 Hz, 2H), 7.32 (d, J=7.8 Hz, 2H), 7.09-7.02 (m, 1H), 6.98 (t, J=7.8 Hz, 1H), 6.59-6.47 (m, 2H), 6.32 (m, 1H), 4.39 (d, J=6.0 Hz, 2H), 3.88 (dd, J=20.6, 12.8 Hz, 2H), 3.54 (d, J=13.7 Hz, 1H), 3.41 (d, J=11.6 Hz, 1H), 2.80 (q, J=7.0 Hz, 1H), 2.54 (q, J=9.1 Hz, 3H), 2.21 (m, 2H), 2.10 (dt, J=19.0, 9.3 Hz, 1H), 1.89 (s, 3H), 1.82-1.70 (m, 1H).

### Example 52: Preparation of Compound 52

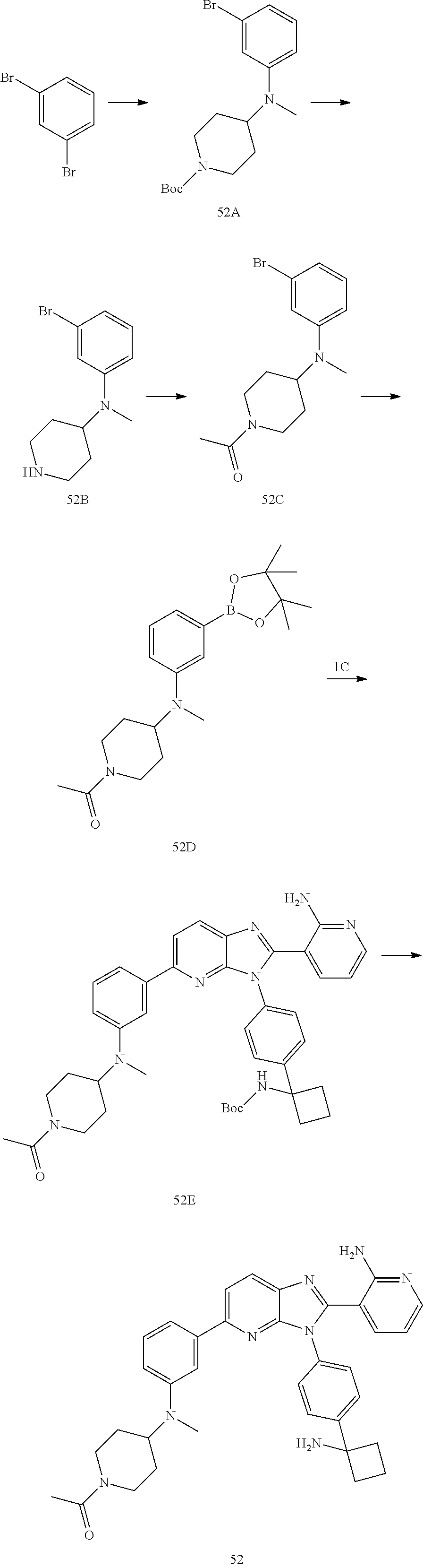

### Step A: Preparation of Compound 52A

To a reaction flask were added 1-tert-butoxycarbonyl-4-methylaminopiperidine (2.00 g), 1,3-dibromobenzene (3.30 g), tris(dibenzylideneacetone)dipalladium (0.43 g), 1,1′-binaphthyl-2,2′-diphenyl phosphine (0.44 g), 1,4-dioxane (100 mL) and sodium tert-butoxide (1.79 g) successively, and after the addition was completed, the reaction solution was stirred for reaction at 100° C. under nitrogen atmosphere. After the reaction was completed, the reaction solution was concentrated and subjected to column chromatography (petroleum ether/ethyl acetate=5/1) to give compound 52A (1.16 g).

MS (ESI, [M-t-Bu+H]⁺) m/z: 313.4.

### Step B: Preparation of Compound 52B

To a reaction flask containing compound 52A (1.15 g) were added methylene chloride (10 mL) and a solution (15 mL) of 4 M hydrogen chloride in dioxane successively, and after the addition was completed, the reaction solution was stirred for reaction at room temperature. After the reaction was completed, the reaction solution was concentrated to give compound 52B (1.17 g).

MS (ESI, [M+H]⁺) m/z: 269.5.

### Step C: Preparation of Compound 52C

To a reaction flask containing compound 52B (1.17 g) were added triethylamine (1.16 g), acetic anhydride (0.78 g) and methylene chloride (40 mL) successively, and after the addition was completed, the reaction solution was stirred for reaction at room temperature. After the reaction was completed, the reaction solution was concentrated and subjected to column chromatography (petroleum ether/ethyl acetate=1/2) to give compound 52C (0.90 g).

MS (ESI, [M+H]⁺) m/z: 311.4.

### Step D: Preparation of Compound 52D

To a reaction flask were added compound 52C (0.88 g), bis(pinacolato)diboron (1.08 g), potassium acetate (0.83 g), [1,1′-bis(diphenylphosphino)ferrocene]palladium dichloride dichloromethane complex (0.23 g) and 1,4-dioxane (50 mL) successively. The reaction solution was stirred for reaction at 100° C. under nitrogen atmosphere. After the reaction was completed, the reaction solution was filtered and concentrated. The reaction solution was subjected to column chromatography (petroleum ether/ethyl acetate=1/2) to give compound 52D (0.90 g).

MS (ESI, [M+H]⁺) m/z: 359.6.

### Step E: Preparation of Compound 52E

To a microwave tube were added compound 52D (200 mg), compound 1C (219 mg), potassium carbonate (169 mg), tetrakis(triphenylphosphine)palladium (47 mg), 1,4-dioxane (10 mL) and water (2 mL) successively. After the addition was completed, the reaction solution was purged with nitrogen gas, and reacted at 140° C. in microwave reactor. After the reaction was completed, the reaction solution was filtered and concentrated. The reaction solution was subjected to column chromatography (dichloromethane/methanol=20/1) to give compound 52E (216 mg).

MS (ESI, [M+H]⁺) m/z: 687.8.

### Step F: Preparation of Compound 52

To a reaction flask were added compound 52E (206 mg), methanesulfonic acid (0.2 mL) and dichloromethane (10 mL) successively, and the reaction solution was stirred for reaction at room temperature. After the reaction was completed, a saturated aqueous sodium carbonate solution was added to the reaction solution to adjust the pH to 9, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 52 (145 mg).

MS (ESI, [M+H]⁺) m/z: 587.7.

¹H NMR (500 MHz, CDCl₃) δ 8.07 (d, J=8.4 Hz, 1H), 7.90 (dd, J=5.2, 1.7 Hz, 1H), 7.72 (d, J=8.4 Hz, 1H), 7.68-7.62 (m, 2H), 7.48-7.43 (m, 2H), 7.39 (s, 1H), 7.29 (d, J=7.7 Hz, 1H), 7.26-7.21 (m, 1H), 7.15 (dd, J=7.8, 1.7 Hz, 1H), 6.82 (dd, J=8.1, 2.5 Hz, 1H), 6.44 (dd, J=7.7, 5.2 Hz, 1H), 4.65-4.57 (m, 1H), 3.89-3.81 (m, 1H), 3.78-3.70 (m, 1H), 3.10-3.02 (m, 1H), 2.81-2.71 (m, 5H), 2.70-2.59 (m, 2H), 2.55-2.47 (m, 1H), 2.38-2.29 (m, 1H), 2.04 (s, 3H), 1.93-1.84 (m, 1H), 1.81-1.71 (m, 2H), 1.66-1.51 (m, 2H).

### Example 53: Preparation of Compound 53

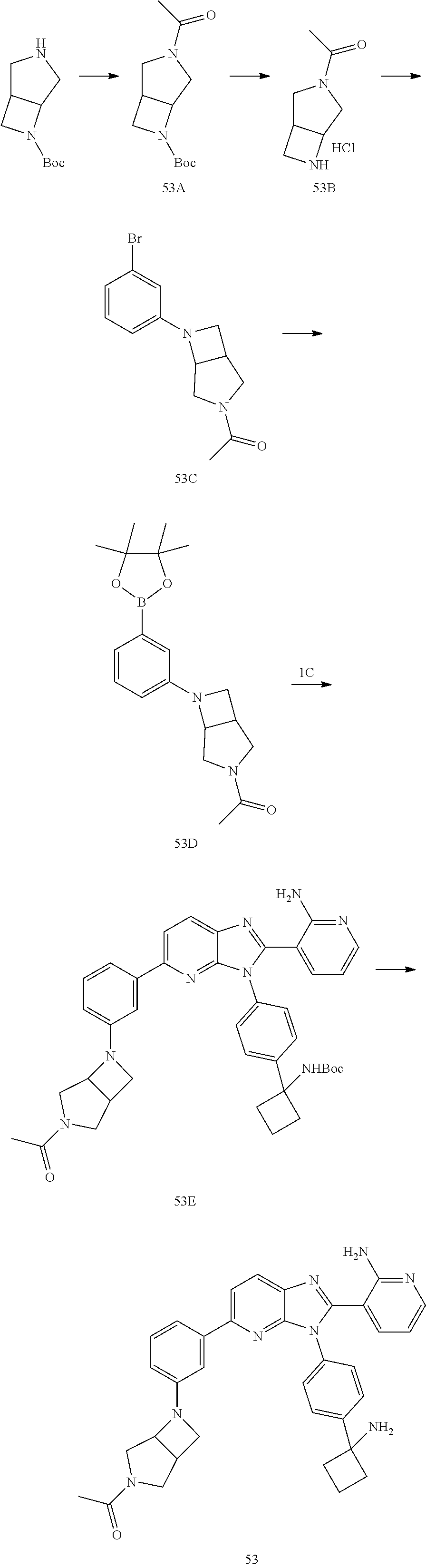

### Step A: Preparation of Compound 53A

To a reaction flask were added tert-butyl 3,6-diazacyclo[3.2.0]heptane-6-carboxylate (0.35 g), acetyl chloride (0.28 g), triethylamine (0.54 g) and dichloromethane (15 mL) successively. The mixture was stirred at room temperature. After the reaction was completed, water and dichloromethane were added to extract the reaction solution, and the reaction solution was washed with saturated brine and dried over anhydrous sodium sulfate. The reaction solution was filtered and concentrated to give compound 53A (0.45 g).

MS (ESI, [M+H]⁺) m/z: 241.1.

### Step B: Preparation of Compound 53B

To a reaction flask were added compound 53A (0.42 g), hydrochloric acid (3.06 g) and dichloromethane (10 mL) successively. The mixture was stirred at room temperature. After the reaction was completed, the reaction solution was concentrated to give compound 53B (0.4 g).

MS (ESI, [M+H]⁺) m/z: 141.1.

### Step C: Preparation of Compound 53C

To a reaction flask were added 1,1′-binaphthyl-2,2′-diphenyl phosphine (0.20 g), tris(dibenzylideneacetone)dipalladium (0.30 g), 1,3-dibromobenzene (0.43 g), compound 53B (0.29 g), sodium tert-butoxide (0.47 g) and dioxane (30 mL) successively under nitrogen atmosphere. After the addition was completed, the reaction solution was stirred at 100° C. After the reaction was completed, the reaction solution was concentrated and subjected to column chromatography (dichloromethane/methanol=50/1) to give compound 53C (0.07 g).

MS (ESI, [M+H]⁺) m/z: 295.4.

### Step D: Preparation of Compound 53D

To a reaction flask were added compound 53C (0.12 g), bis(pinacolato)diboron (0.21 g), potassium acetate (0.12 g), [1,1′-bis(diphenylphosphino)ferrocene]palladium dichloride dichloromethane complex (0.066 g) and 1,4-dioxane (20 mL) successively. The reaction solution was stirred at 100° C. under nitrogen atmosphere. After the reaction was completed, the reaction solution was filtered and concentrated. The reaction solution was subjected to column chromatography (dichloromethane/methanol=40/1) to give compound 53D (0.09 g).

MS (ESI, [M+H]⁺) m/z: 343.6.

### Step E: Preparation of Compound 53E

To a reaction flask were added compound 53D (0.088 g), compound 1C (0.075 g), potassium carbonate (0.042 g), tetrakis(triphenylphosphine)palladium (0.035 g), 1,4-dioxane (10 mL), and water (2 mL) successively. After the addition was completed, the reaction solution was purged with nitrogen gas, and reacted at 100° C. After the reaction was completed, the reaction solution was filtered and concentrated. The reaction solution was subjected to column chromatography (dichloromethane/methanol=40/1) to give compound 53E (0.054 g).

MS (ESI, [M+H]⁺) m/z: 671.8.

### Step F: Preparation of Compound 53

Compound 53E (0.050 g), methanesulfonic acid (0.072 g) and dichloromethane (10 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=40/1) to give compound 53 (0.020 g).

HRMS (ESI, [M+H]⁺) m/z: 571.2924.

¹H NMR (500 MHz, CDCl₃) δ 8.08 (t, J=8.4 Hz, 1H), 8.02 (dd, J=8.7, 4.4 Hz, 1H), 7.73 (d, J=8.3 Hz, 1H), 7.68 (d, J=8.2 Hz, 1H), 7.61 (d, J=8.2 Hz, 1H), 7.41 (t, J=8.5 Hz, 2H), 7.34 (d, J=7.7 Hz, 1H), 7.23 (dd, J=14.2, 7.0 Hz, 2H), 7.13-7.07 (m, 1H), 6.62 (d, J=9.8 Hz, 2H), 6.37 (dd, J=14.0, 8.0 Hz, 2H), 4.67-4.50 (m, 1H), 4.18 (dd, J=19.0, 11.7 Hz, 1H), 3.95-3.76 (m, 3H), 3.67-3.60 (m, 1H), 3.58 (d, J=6.1 Hz, 1H), 3.34 (dd, J=14.7, 6.2 Hz, 1H), 3.31-3.23 (m, 1H), 3.17 (s, 1H), 2.65 (dd, J=39.8, 30.3 Hz, 4H), 2.45 (d, J=39.0 Hz, 2H), 2.12 (s, 1H), 2.00 (s, 1H), 1.85 (s, 1H).

### Example 54: Preparation of Compound 54

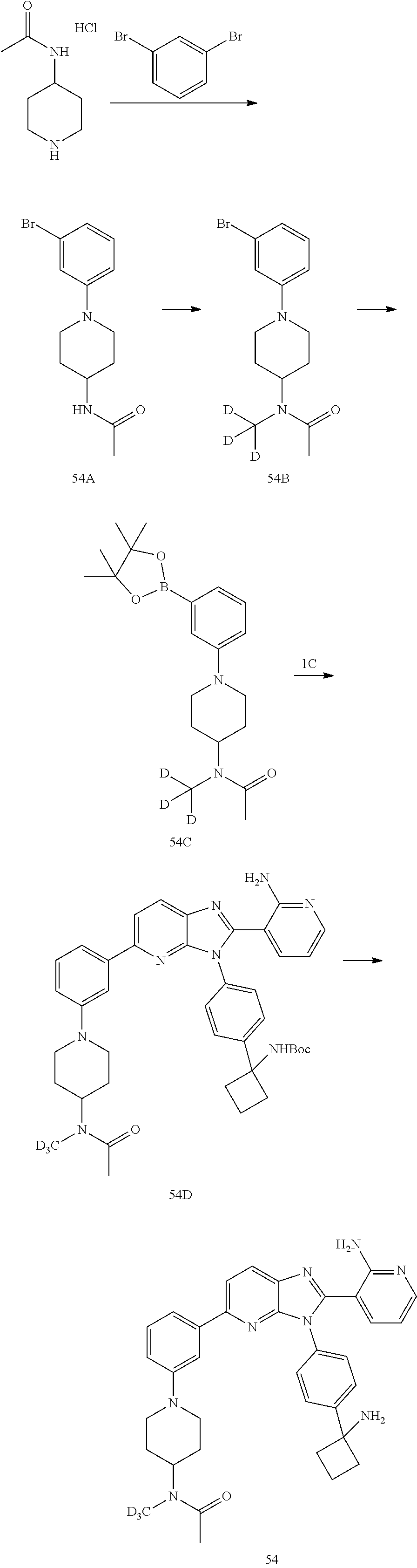

### Step A: Preparation of Compound 54A

To a reaction flask were added 1,1′-binaphthyl-2,2′-diphenyl phosphine (3.3 g), tris(dibenzylideneacetone)dipalladium (4.9 g), 1,3-dibromo-2-fluorobenzene (2.6 g), 4-acetylaminopiperidine hydrochloride (3.0 g), sodium tert-butoxide (3.6 g) and dioxane (30 mL) successively under nitrogen atmosphere. After the addition was completed, the reaction solution was stirred at 100° C. After the reaction was completed, the reaction solution was concentrated and subjected to column chromatography (petroleum ether/ethyl acetate=5/1) to give compound 54A (0.90 g).

MS (ESI, [M+H]⁺) m/z: 297.1.

### Step B: Preparation of Compound 54B

To a reaction flask were added compound 54A (0.90 g), sodium hydride (1.2 g), deuterated iodomethane (3.1 g), and tetrahydrofuran (20 mL) successively. The mixture was stirred at room temperature. After the reaction was completed, a saturated ammonium chloride solution was added to the reaction solution, and water and ethyl acetate were added to extract the reaction solution; the reaction solution was then washed with saturated brine and dried over anhydrous sodium sulfate. The reaction solution was filtered and concentrated to give compound 54B (1.5 g).

MS (ESI, [M+H]⁺) m/z: 314.5.

### Step C: Preparation of Compound 54C

To a reaction flask were added compound 54B (1.5 g), bis(pinacolato)diboron (2.4 g), potassium acetate (1.4 g), [1,1′-bis(diphenylphosphino)ferrocene]palladium dichloride dichloromethane complex (0.57 g) and 1,4-dioxane (50 mL) successively. The reaction solution was stirred at 100° C. under nitrogen atmosphere. After the reaction was completed, the reaction solution was filtered and concentrated. The reaction solution was subjected to column chromatography (dichloromethane/methanol=20/1) to give compound 54C (0.62 g).

MS (ESI, [M+H]⁺) m/z: 362.1.

### Step D: Preparation of Compound 54D

To a reaction flask were added compound 54C (0.33 g), compound 1C (0.30 g), potassium carbonate (0.17 g), tetrakis(triphenylphosphine)palladium (0.21 g), 1,4-dioxane (30 mL), and water (6 mL) successively. After the addition was completed, the reaction solution was purged with nitrogen gas, and reacted at 100° C. After the reaction was completed, the reaction solution was filtered and concentrated. The reaction solution was subjected to column chromatography (dichloromethane/methanol=20/1) to give compound 54D (0.28 g).

MS (ESI, [M+H]⁺) m/z: 690.8.

### Step E: Preparation of Compound 54

Compound 54D (0.28 g), methanesulfonic acid (0.19 g) and dichloromethane (10 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 54 (0.14 g).

HRMS (ESI, [M+H]⁺) m/z: 590.3460.

¹H NMR (500 MHz, CDCl₃) δ 8.08-7.96 (m, 2H), 7.70 (dd, J=8.4, 2.1 Hz, 1H), 7.55 (m, 1H), 7.49 (dd, J=8.2, 5.9 Hz, 2H), 7.43 (dd, J=15.3, 7.7 Hz, 1H), 7.40-7.32 (m, 2H), 7.25 (q, J=8.1 Hz, 1H), 7.08 (dd, J=7.8, 1.8 Hz, 1H), 6.89 (dd, J=8.2, 2.6 Hz, 1H), 6.56 (s, 2H), 6.32 (dd, J=7.8, 4.8 Hz, 1H), 4.59 (tt, J=12.2, 4.1 Hz, 1H), 3.84-3.66 (m, 2H), 2.80 (m, 2H), 2.54 (m, 2H), 2.17 (m, 2H), 2.10 (s, 1H), 2.04 (s, 2H), 1.97 (s, 3H), 1.75 (m, 2H), 1.66-1.59 (m, 1H).

### Example 55: Preparation of Compound 55

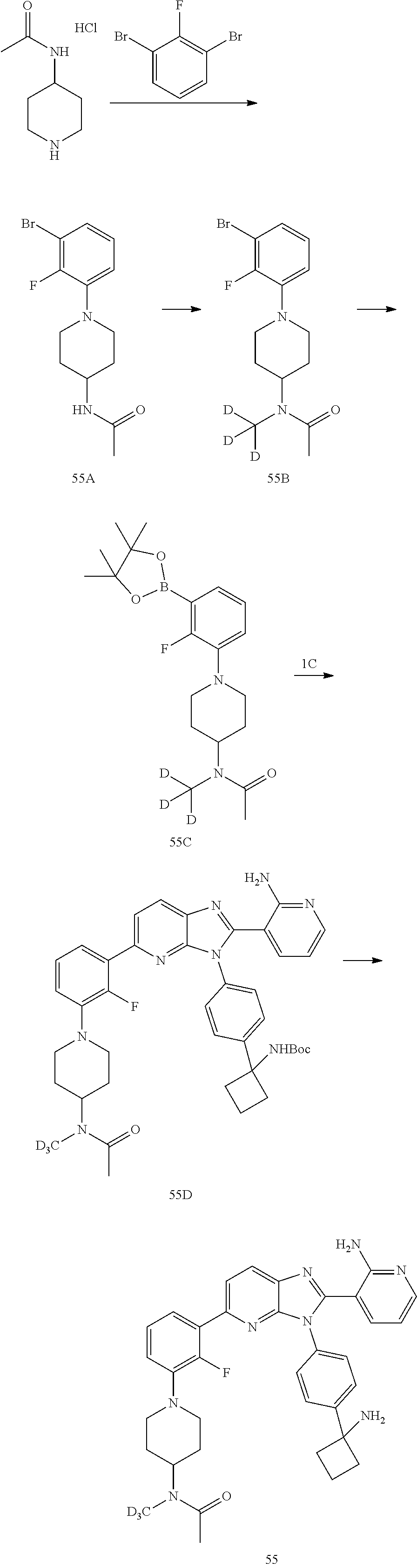

### Step A: Preparation of Compound 55A

To a reaction flask were added 1,1′-binaphthyl-2,2′-diphenyl phosphine (3.1 g), tris(dibenzylideneacetone)dipalladium (2.3 g), 1,3-dibromo-2-fluorobenzene (5.1 g), 4-acetylaminopiperidine hydrochloride (3.0 g), sodium tert-butoxide (3.6 g) and tetrahydrofuran (30 mL) successively under nitrogen atmosphere. After the addition was completed, the reaction solution was stirred at 100° C. After the reaction was completed, the reaction solution was concentrated and subjected to column chromatography (petroleum ether/ethyl acetate=5/1) to give compound 55A (1.0 g).

MS (ESI, [M+H]⁺) m/z: 315.4.

### Step B: Preparation of Compound 55B

To a reaction flask were added compound 55A (1.0 g), sodium hydride (1.1 g), deuterated iodomethane (1.1 g), and tetrahydrofuran (10 mL) successively. The mixture was stirred at room temperature. After the reaction was completed, a saturated ammonium chloride solution was added to the reaction solution, and water and ethyl acetate were added to extract the reaction solution; the reaction solution was then washed with saturated brine and dried over anhydrous sodium sulfate. The reaction solution was filtered and concentrated to give compound 55B (1.4 g).

MS (ESI, [M+H]⁺) m/z: 332.4.

### Step C: Preparation of Compound 55C

To a reaction flask were added compound 55B (1.4 g), bis(pinacolato)diboron (2.0 g), potassium acetate (1.2 g), [1,1′-bis(diphenylphosphino)ferrocene]palladium dichloride dichloromethane complex (0.5 g) and 1,4-dioxane (50 mL) successively. The reaction solution was stirred at 100° C. under nitrogen atmosphere. After the reaction was completed, the reaction solution was filtered and concentrated. The reaction solution was subjected to column chromatography (dichloromethane/methanol=20/1) to give compound 55C (0.75 g).

MS (ESI, [M+H]⁺) m/z: 380.1.

### Step D: Preparation of Compound 55D

To a reaction flask were added compound 55C (0.35 g), compound 1C (0.30 g), potassium carbonate (0.17 g), tetrakis(triphenylphosphine)palladium (0.21 g), 1,4-dioxane (30 mL), and water (6 mL) successively. After the addition was completed, the reaction solution was purged with nitrogen gas, and reacted at 100° C. After the reaction was completed, the reaction solution was filtered and concentrated. The reaction solution was subjected to column chromatography (dichloromethane/methanol=20/1) to give compound 55D (0.28 g).

MS (ESI, [M+H]⁺) m/z: 708.5.

### Step E: Preparation of Compound 55

Compound 55D (0.28 g), methanesulfonic acid (0.19 g) and dichloromethane (10 mL) were added successively to a reaction flask. The mixture was stirred at room temperature. After the reaction was completed, 10% sodium hydroxide solution was added to the reaction solution to adjust the pH to 13, water and dichloromethane were added to extract the reaction solution, and the reaction solution was washed with saturated brine and dried over anhydrous sodium sulfate. The reaction solution was filtered, concentrated, and subjected to column chromatography (dichloromethane/methanol=10/1) to give compound 55 (0.11 g).

HRMS (ESI, [M+H]⁺) m/z: 608.3345.

¹H NMR (500 MHz, CDCl₃) δ 8.05 (dd, J=8.3, 5.6 Hz, 1H), 7.99 (dd, J=4.8, 1.9 Hz, 1H), 7.74 (dd, J=8.4, 2.3 Hz, 1H), 7.48 (dd, J=8.5, 2.5 Hz, 2H), 7.46-7.40 (m, 1H), 7.33 (dd, J=8.4, 2.2 Hz, 2H), 7.09-7.00 (m, 2H), 6.98-6.89 (m, 1H), 6.54 (s, 2H), 6.31 (dd, J=7.8, 4.9 Hz, 1H), 4.57 (tt, J=12.3, 4.2 Hz, 1H), 3.53-3.36 (m, 2H), 2.86-2.66 (m, 2H), 2.53 (dddd, J=12.1, 9.5, 6.2, 2.9 Hz, 2H), 2.20-2.14 (m, 3H), 2.09 (s, 2H), 2.04 (s, 3H), 1.86 (m, 2H), 1.74 (m, 1H), 1.66 (m, 2H).

### Experimental Example 1: Inhibitory Effect of Compound on Proliferation of LNcap Cells

A dish of LNcap cells (human prostate cancer cells with PTEN deficiency) growing at log phase and in good conditions were taken and digested for 3 min by using 1 mL of pancreatin, then the mixture was added with 4 mL of complete culture medium to terminate digestion, and the cells were added to a centrifuge tube. 20 μL of cells were taken and counted, the required number of cells (mL) was pipetted and centrifuged at 1200 rpm for 5 min, the supernatant was discarded, and an appropriate amount of plating medium (RPMI medium+5% FBS+1% sodium pyruvate+1% glutamine) was added to adjust the cell density to 3×10⁴ cells/mL. The cells were seeded on a 96-well plate at 100 μL/well using a multi-channel pipette and cultured in a cell incubator at 37° C., 5% CO₂ and saturated humidity. After incubation overnight, compounds were loaded using a nanoliter pipettor, 2 duplicate wells were set for each concentration, and cells without compound were used as negative controls. After 72 h, CCK-8 was added at 10 μL/well for incubation for 4 h, then absorbance was measured at 450 nm with an Envision plate reader, and inhibition rate was calculated. Inhibition rate (%)=(mean value of negative control group−mean value of experimental group)/(mean value of negative control group−mean value of blank group)×100%. A dose-response curve was fitted by four-parameter analysis, with the logarithm of compound concentration as abscissa and inhibition rate as ordinate, so that IC₅₀ was calculated. The experimental results are shown in Table 1.

TABLE 1

| | LNcap cells |
|---|---|
| Example | IC₅₀ nM |
| 1 | 16 |
| 2 | 34 |
| 3 | 6.5 |
| 6 | 15 |
| 7 | 48 |
| 9 | 24 |
| 10 | 21 |
| 11 | 12 |
| 13 | 20 |
| 16 | 8.7 |
| 17 | 12 |
| 18 | 18 |
| 20 | 15 |
| 21 | 3.0 |
| 23 | 38 |
| 24 | 41 |
| 26 | 14 |
| 27 | 20 |
| 28 | 3.0 |
| 29 | 46 |
| 30 | 4.8 |
| 31 | 28 |
| 32 | 4.3 |
| 33 | 11 |
| 34 | 2.9 |
| 35 | 5.0 |
| 36 | 14 |
| 37 | 7.6 |
| 38 | 11 |
| 39 | 11 |
| 40 | 19 |
| 41 | 10 |
| 42 | 21 |
| 43 | 47 |
| 44 | 1.7 |
| 45 | 1.7 |
| 46 | 7.5 |
| 47 | 81 |
| 48 | 6.9 |
| 49 | 4.0 |
| 54 | 2.2 |
| 55 | 2.1 |

### Experimental Example 2: Inhibitory Effect of Compound on Phosphorylation of LNcap Cells AKT1 (S473)

LNcap cells AKT1 (S473) growing at log phase were taken, digested with 1 mL of pancreatin for 3 min, and added with 4 mL of complete medium to terminate digestion. The cells were added to a centrifuge tube. 20 μL of cells were taken and counted, the required number of cells (mL) was taken and centrifuged at 1200 rpm for 5 min, and a plating medium (2% FBS+phenol-free red 1640 base medium+1% sodium pyruvate+1% glutamine) was added to adjust the cell density to about 1×10E6 cells/mL. The cells were plated (96 wells) at the above cell density (100 μL/well), and cultured in a cell incubator at 37° C., 5% CO₂ overnight; the next day, according to the plate distribution, the corresponding compound was sprayed using a nanoliter pipettor and incubated for 1 h at 37° C. in a cell incubator containing 5% CO₂; the supernatant was discarded, and the plate was added with 40 μL of lysis buffer (1×) containing blocking buffer and incubated by shaking at room temperature for 30 min. After the mixture was mixed, 16 μL of lysate was transferred to another 384-well small-volume white plate. The plate was added with 4 μL of pre-mixed antibody (v/v) in assay buffer, covered, centrifuged to mix well, and incubated overnight at room temperature. The 665 nm/620 nm signal value was detected using a PE Envision multi-functional microplate reader, and IC₅₀ was calculated by four-parameter fitting.

The results of the inhibitory effect of the compound on the phosphorylation of LNcap cells AKT1 (S473) are shown in table 2.

TABLE 2

| | Phosphorylation of AKT1 (S473) |
|---|---|
| Example | IC₅₀ nM |
| 1 | 34 |
| 2 | 17 |
| 3 | 20 |
| 6 | 54 |
| 9 | 16 |
| 10 | 47 |
| 11 | 19 |
| 16 | 19 |
| 17 | 17 |
| 21 | 23 |
| 28 | 28 |
| 30 | 22 |
| 32 | 11 |
| 33 | 17 |
| 34 | 25 |
| 35 | 12 |
| 37 | 11 |
| 38 | 13 |
| 39 | 42 |
| 41 | 41 |
| 44 | 12 |
| 45 | 7.7 |
| 54 | 10 |
| 55 | 7.1. |

### Experimental Example 3: Pharmacokinetic Evaluation of Compound in Mice

ICR mice, weight 18-22 g, after adaption for 3-5 days, were randomly divided into 4 groups of 9 mice, each group was intragastrically (IG) administered with compound 28 or compound 30 at a dose of 10 mg/kg, or intravenously (IV) administered with compound 28 or compound 30 at a dose of 1 mg/kg.

The animals to be tested (ICR mice) were fasted for 12 h before administration and food was provided 4 h after administration, and water was freely drunk before and after the experiment and during the experiment.

After intragastric administration, about 0.1 mL of blood was taken from eye sockets at 15 min, 30 min, 1 h, 2 h, 3 h, 4 h, 6 h, 8 h, 10 h, and 24 h; after intravenous injection, about 0.1 mL of blood was taken from eye sockets at 5 min, 10 min, 30 min, 1 h, 2 h, 4 h, 6 h, 8 h, 10 h, and 24 h; each mouse was collected at 3-4 time points and 3 mice were collected at each time point, whole blood was collected, placed in centrifuge tubes containing EDTA-K₂, and stored at 4° C. within 1 h, and the plasma was separated off by centrifugation at 4° C. and 4000 rpm for 10 min. All the plasma samples were collected and immediately stored at −20° C. for testing. 30 μL of the plasma sample to be tested and a standard curve sample were pipetted and added with 300 μL of acetonitrile solution containing an internal standard (20 ng/mL diazepam), shaken and mixed well for 5 min, and centrifuged at 13,000 rpm for 10 min. 80 μL of supernatant was taken and diluted with 80 μL of ultrapure water. After being mixed well, 2 μL of the resulting sample was taken for liquid chromatography-mass determination, and a chromatogram was recorded.

Oral and intravenous exposure of the compound of the present application was evaluated by in vivo pharmacokinetic experiment in mice. The results are shown in the table 3 below.

TABLE 3
In vivo pharmacokinetic parameters of compound in mice

| Compound | | Compound 28 | | Compound 30 | |
|---|---|---|---|---|---|
| Dosage | | IG 10 mg/kg | IV 1 mg/kg | IG 10 mg/kg | IV 1 mg/kg |
| AUC(0-t) | ng*h/mL | 871 | 246 | 2772 | 476 |
| AUC(0-∞) | ng*h/mL | 896 | 253 | 2884 | 482 |
| MRT(0-t) | h | 5.28 | 1.49 | 5.01 | 1.04 |
| t1/2z | h | 5.31 | 1.74 | 6.58 | 1.48 |
| Tmax | h | 2 | / | 2 | / |
| Cmax | ng/mL | 140 | / | 542 | / |
| Vd | L/kg | / | 9.92 | / | 4.43 |
| Cl | L/kg | / | 3.95 | / | 2.08 |
| Absolute bioavailability F % | | 35.42% | | 58.23% | |
| Compound | | Compound 44 | | Compound 45 | |
| Dosage | | IG 10 mg/kg | IV 1 mg/kg | IG 10 mg/kg | IV 1 mg/kg |
| AUC(0-t) | ng*h/mL | 24990 | 4837 | 14539 | 4243 |
| AUC(0-∞) | ng*h/mL | 25071 | 4845 | 14543 | 4339 |
| MRT(0-t) | h | 3.58 | 0.817 | 3.95 | 1.176 |
| t1/2z | h | 3.61 | 1.34 | 2.06 | 1.159 |
| Tmax | h | 2 | / | 0.25 | / |
| Cmax | ng/mL | 4965 | / | 4016 | / |
| Vd | L/kg | / | 0.398 | / | 5.423 |
| Cl | L/kg | / | 0.206 | / | 0.23 |

| | | |
|---|---|---|
| Absolute bioavailability F % | 51.67% | 34.27%. |

### Experimental Example 4: Pharmacokinetic Evaluation of Compound in Rats

SD rats, weight 180-220 g, after adaption for 7 days, were randomly divided into 4 groups of 3 mice, each group was intragastrically (IG) administered with compound 28 or compound 30 at a dose of 10 mg/kg, or intravenously (IV) administered with compound 28 or compound 30 at a dose of 1 mg/kg. The animals to be tested (SD rats) were fasted for 12 h before administration and food was provided 4 h after administration, and water was freely drunk before and after the experiment and during the experiment.

After intragastric administration, about 0.1 mL of blood was taken from eye sockets at 15 min, 30 min, 1 h, 2 h, 3 h, 4 h, 6 h, 8 h, 10 h, and 24 h; after intravenous injection, about 0.1 mL of blood was taken from eye sockets at 5 min, 10 min, 30 min, 1 h, 2 h, 4 h, 6 h, 8 h, 10 h, and 24 h; 3 rats were collected at each time point, whole blood was collected, placed in centrifuge tubes containing EDTA-K₂, and stored at 4° C. within 1 h, and the plasma was separated off by centrifugation at 4° C. and 4000 rpm for 10 min. All the plasma samples were collected and immediately stored at −20° C. for testing. 30 μL of the plasma sample to be tested and a standard curve sample were pipetted and added with 300 μL of acetonitrile solution containing an internal standard (20 ng/mL diazepam), shaken and mixed well for 5 min, and centrifuged at 13,000 rpm for 10 min. 80 μL of supernatant was taken and diluted with 80 μL of ultrapure water. After being mixed well, 2 μL of the resulting sample was taken for liquid chromatography-mass determination, and a chromatogram was recorded.

Oral and intravenous exposure of the compound of the present application was evaluated by in vivo pharmacokinetic experiment in rats. The results are shown in the table 4 below.

TABLE 4
In vivo pharmacokinetic parameters of compound in rats

| Compound | | Compound 28 | | Compound 30 | |
|---|---|---|---|---|---|
| Theoretical dosage | | IG 10 mg/kg | IV 1 mg/kg | IG 10 mg/kg | IV 1 mg/kg |
| AUC(0-t) | ng*h/mL | 362 ± 195 | 145 ± 11.1 | 358 ± 109 | 127 ± 9.69 |
| AUC(0-∞) | ng*h/mL | 372 ± 197 | 174 ± 4.37 | 383 ± 106 | 139 ± 9.21 |
| MRT(0-t) | h | 7.38 ± 0.43 | 2.12 ± 0.099 | 8.15 ± 0.73 | 5.57 ± 0.053 |
| t1/2z | h | 3.93 ± 1.46 | 5.9 ± 2.11 | 5.43 ± 1.82 | 7.73 ± 0.440 |
| Tmax | h | 4.67 ± 1.15 | / | 4.67 ± 1.15 | / |
| Cmax | ng/mL | 32.6 ± 12.4 | / | 31.2 ± 8.80 | / |
| Vd | L/kg | / | 48.8 ± 16.8 | / | 80.8 ± 8.90 |
| Cl | L/kg | / | 5.75 ± 0.146 | / | 7.23 ± 0.496 |

| | | |
|---|---|---|
| Absolute bioavailability F % | 24.97% | 35.47%. |

### Experimental Example 5: Pharmacodynamics of Compound in Subcutaneous Xenograft Tumor Nude Mouse Model of AN3CA Human Endometrial Cancer Cells

SPF-grade female BALB/C nude mice (from Changzhou Cavens Laboratory Animal Ltd.) were inoculated subcutaneously at the right side armpit with 5×10⁶ AN3CA cells (sourec: Nanjing Cobioer Biosciences Co., Ltd.). When the mean tumor volume reached about 130 mm³, the animals were divided into 3 groups of 6 and dosed as shown in table 5:

TABLE 5
Administration regimen

| | | | | Treat- |
|---|---|---|---|---|
| | Dose | Route of | Frequency of | ment |
| Groups | (mg/kg) | administration | administration | cycle |
| Control group | — | i.g. | qd | 14 d |
| (vehicle control) | | | | |
| Compound 28 group | 20 | i.g. | qd | 14 d |
| Compound 30 group | 20 | i.g. | qd | 14 d |

Note:
i.g. represents intragastric; qd represents once daily.

The day of grouping was determined as do, and the intragastric administration was started on the grouping day, and the administration volume was 10 mL/kg. The solvent was: D5W (5% glucose solution). The tumor volume was measured 2-3 times a week, meanwhile, the mice were weighed and the data were recorded; the general behavior of the mice was observed and recorded every day. After the experiment was completed, the tumors were removed, weighed and photographed.

Calculation formula for tumor volume: Tumor volume (mm³)=½×(a×b²) (where a represents long diameter and b represents short diameter).

Relative tumor proliferation rate (T/C %) refers to the percentage of the relative tumor volume of the treatment and control groups at a certain time point. The calculation formula is as follows: T/C %=TRTV/CRTV×100% (TRTv: mean relative tumor volume (RTV) for treatment group; CRTV: mean RTV for vehicle control group; RTV=TVₜ/TV₀, where TV₀ is the tumor volume at grouping, TVₜ is the tumor volume after treatment).

Tumor growth inhibition rate (TGI %) was calculated according to the following formula: TGI %=(1−tumor weight in treatment group/tumor weight in control group)×100%.

The calculation formula for weight change rate (WCR) (%) is: WCR=(Wtₜ−Wt₀)/Wt₀×100%, where Wt₀ is the body weight of the animal at grouping (i.e., d0), Wtₜ is the body weight of the animal at each measurement.

All experimental results are expressed as mean±SD (mean±standard deviation). The relative tumor volume of the treatment group was compared with that of the control group for any significant difference by T tests, wherein p<0.01 indicates a very significant difference.

The results are shown in table 6, and the in vivo efficacy results show that both compound 28 and compound 30 have strong in vivo tumor inhibition effects.

TABLE 6
Effect of AKT inhibitors on the subcutaneous xenograft
tumor of AN3CA human endometrial cancer cells

| | | On day 14 of experiment | | | | |
|---|---|---|---|---|---|---|
| | | Tumor volume | Relative | | | Weight change |
| | Dosage | (mm³) | tumor volume | | | rate (%) |
| Groups | mg/kg | (mean ± SD) | (mean ± SD) | T/C (%) | TGI (%) | (mean ± SD) |
| Control group | — | 2838 ± 498 | 23.7 ± 8.1 | — | — | 11.0 ± 3.6 |
| Vehicle control | | | | | | |
| Compound 28 group | 20 | 1267 ± 249 | 10.5 ± 3.4 | 44.3% | 53.2%** | 4.9 ± 2.7 |
| Compound 30 group | 20 | 1242 ± 354 | 9.7 ± 2.2 | 40.9% | 58.4%** | 2.3 ± 3.5 |

Note:
comparison with the control group, where ** denotes p < 0.01.

1. A compound of formula (I) or a pharmaceutically acceptable salt thereof, 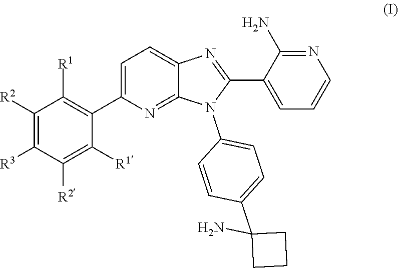
wherein,
R¹ and R^{1′} are each independently selected from the group consisting of hydrogen and halogen;
R², R^{2′} and R³ are each independently selected from the group consisting of hydrogen, amino, amino-C₁-C₆ alkyl-, 7-9 membered spiro-heterocyclyl, 10 membered spiro-heterocyclyl whose ring atoms consist of a nitrogen atom and a carbon atom, 7-10 membered fused-heterocyclyl, 7-10 membered bridged heterocyclyl, 5-6 membered heterocyclyl whose ring atoms consist of a nitrogen atom and a carbon atom, 5-6 membered heterocyclyl whose ring atoms contain a silicon atom or a phosphorus atom, 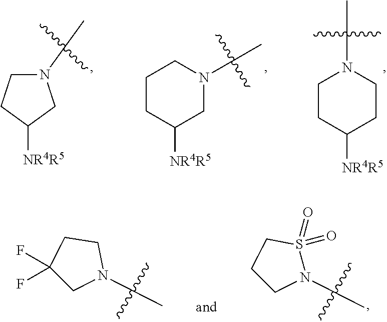
wherein the amino or amino-C₁-C₆ alkyl- is substituted with one or more R²¹, and optionally substituted with one or more R^{21′}, wherein the 5-6 membered heterocyclyl whose ring atoms consist of a nitrogen atom and a carbon atom is substituted with one or more R²², and optionally substituted with one or more R^{22′}, wherein the 7-9 membered spiro-heterocyclyl, 10 membered spiro-heterocyclyl whose ring atoms consist of a nitrogen atom and a carbon atom, 7-10 membered heterocycloalkenyl or 5-6 membered heterocyclyl whose ring atoms contain a silicon atom or a phosphorus atom is optionally substituted with one or more R²³, wherein the 7-10 membered bridged heterocyclyl is substituted with one or more R²⁴,
or, R³ is connected to R² or R^{2′}, and together with the carbon atoms connected thereto, forms morpholinyl optionally substituted with one or more R²⁵,
and R², R^{2′} and R³ are not hydrogen at the same time;
R²¹ is selected from the group consisting of C₂-C₆ alkenyl-C(O)—, C₃-C₁₂ cycloalkyl, 4-12 membered heterocyclyl or C₁-C₆ alkylacylamino-C₁-C₆ alkyl-, wherein the C₃-C₁₂ cycloalkyl, 4-12 membered heterocyclyl or C₁-C₆ alkylacylamino-C₁-C₆ alkyl- is optionally substituted with one or more of the following groups selected from the group consisting of: hydroxy, C₁-C₆ alkyl and C₁-C₆ alkylacyl;
R²² is selected from the group consisting of C₂-C₆ alkynyl-C(O)—, 4-5 membered heterocyclyl or 6 membered heterocyclyl whose ring atoms consist of a nitrogen atom and a sulfur atom, wherein the 4-5 membered heterocyclyl or 6 membered heterocyclyl whose ring atoms consist of a nitrogen atom and a sulfur atom is optionally substituted with one or more of the following groups selected from the group consisting of: [symbol] , halogen, C₁-C₃ alkyl and C₁-C₆ alkylacyl;
R^{21′} and R^{22′} are each independently selected from the group consisting of deuterium and C₁-C₆ alkyl;
R²³ and R²⁴ are each independently selected from the group consisting of [symbol] , C₁-C₆ alkyl, C₁-C₆ alkylsulfonyl, C₁-C₆ alkylacyl and C₁-C₆ alkylacyl-N(C₁-C₆ alkyl)-;
R²⁵ is selected from C₁-C₆ alkylacyl;
R⁴ is selected from the group consisting of C₁-C₆ alkylacyl and C₁-C₆ alkylsulfonyl;
R⁵ is selected from C₁-C₆ alkyl;
R^{5′} is selected from the group consisting of —CD₃ and —CH₃, and when R^{5′} is —CH₃, R¹ or R^{1′} is halogen.

2-19. (canceled)

20. The compound of formula (I) or the pharmaceutically acceptable salt thereof according to claim 1, wherein,
R², R^{2′} and R³ are each independently selected from the group consisting of hydrogen, amino, amino-C₁-C₆ alkyl-, 7-9 membered spiro-heterocyclyl, 7-10 membered fused-heterocyclyl, 7-10 membered bridged heterocyclyl, 5-6 membered heterocyclyl whose ring atoms consist of a nitrogen atom and a carbon atom, 5-6 membered heterocyclyl whose ring atoms contain a silicon atom or a phosphorus atom, 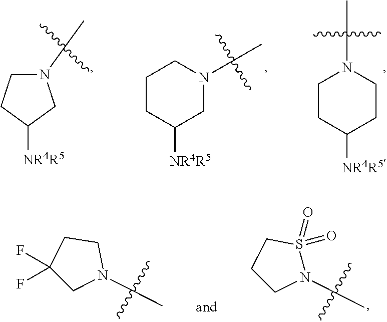
wherein the amino or amino-C₁-C₆ alkyl- is substituted with one or more R²¹, and optionally substituted with one or more R^{21′}, wherein the 5-6 membered heterocyclyl whose ring atoms consist of a nitrogen atom and a carbon atom is substituted with one or more R²², and optionally substituted with one or more R^{22′}, wherein the 7-9 membered spiro-heterocyclyl, 7-10 membered heterocycloalkenyl or 5-6 membered heterocyclyl whose ring atoms contain a silicon atom or a phosphorus atom is optionally substituted with one or more R²³, wherein the 7-10 membered bridged heterocyclyl is substituted with one or more R²⁴,
or, R³, together with R² or R^{2′} and the carbon atoms connected thereto, forms morpholinyl optionally substituted with one or more R²,
and R², R^{2′} and R³ are not hydrogen at the same time;
R²² is selected from the group consisting of C₂-C₆ alkynyl-C(O)— and 4-5 membered heterocyclyl, wherein the 4-5 membered heterocyclyl is optionally substituted with one or more [symbol] ;
R^{21′} and R^{22′} are each independently selected from C₁-C₆ alkyl;
optionally, R², R^{2′} and R³ are each independently selected from the group consisting of hydrogen, amino, amino-C₁-C₄ alkyl-, 7 or 9 membered spiro-heterocycloalkyl, 7, 8 or 9 membered fused-heterocycloalkyl, 8 membered bridged heterocycloalkyl, 5-6 membered monoheterocycloalkyl whose ring atoms consist of a nitrogen atom and a carbon atom, 5-6 membered monoheterocycloalkyl whose ring atoms contain a silicon atom or a phosphorus atom, 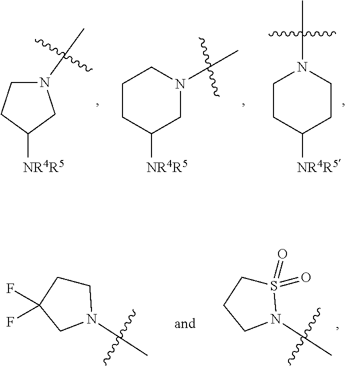
wherein the amino or amino-C₁-C₄ alkyl- is substituted with one or more R²¹, and optionally substituted with one or more R^{21′} wherein the 5-6 membered monoheterocycloalkyl whose ring atoms consist of a nitrogen atom and a carbon atom is substituted with one or more R²², and optionally substituted with one or more R^{22′}, wherein the 7 or 9 membered spiro-heterocycloalkyl, 7, 8 or 9 membered fused-heterocycloalkyl, or 5-6 membered monoheterocycloalkyl whose ring atoms contain a silicon atom or a phosphorus atom is optionally substituted by one or more R²³, wherein the 8 membered bridged heterocycloalkyl is substituted by one or more R²⁴,
or, R³ is connected to R² or R^{2′}, and together with the carbon atoms connected thereto, forms morpholinyl optionally substituted with one or more R²,
and R², R^{2′} and R³ are not hydrogen at the same time;
optionally, R^{21′} and R^{22′} are each independently selected from C₁-C₄ alkyl;
optionally, R^{21′} and R^{22′} are each independently selected from methyl.

21. The compound of formula (I) or the pharmaceutically acceptable salt thereof according to claim 1, wherein R¹ and R^{1′} are each independently selected from the group consisting of hydrogen and fluorine;
or, R¹ and R^{1′} are both selected from hydrogen;
or, R¹ is selected from hydrogen, and R^{1′} is selected from fluorine;
or, R¹ is selected from fluorine, and R^{1′} is selected from hydrogen.

22. The compound of formula (I) or the pharmaceutically acceptable salt thereof according to claim 1, wherein R², R^{2′} and R³ are each independently selected from the group consisting of hydrogen, amino, amino-C₁-C₄ alkyl-, 7, 8 or 9 membered spiro-heterocyclyl, 10 membered spiro-heterocyclyl whose ring atoms consist of a nitrogen atom and a carbon atom, 7, 8 or 9 membered fused-heterocyclyl, 7-8 membered bridged heterocyclyl, 5-6 membered monoheterocyclyl whose ring atoms consist of a nitrogen atom and a carbon atom, 5-6 membered monoheterocyclyl whose ring atoms consist of a silicon atom or a phosphorus atom, 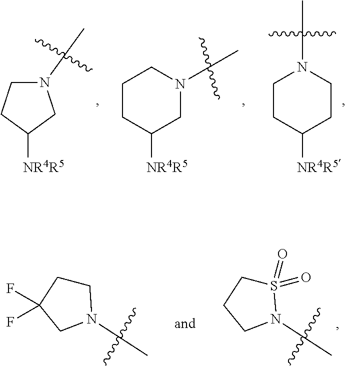
wherein the amino or amino-C₁-C₄ alkyl- is substituted with one or more R²¹, and optionally substituted with one or more R^{21′} wherein the 5-6 membered monoheterocyclyl whose ring atoms consist of a nitrogen atom and a carbon atom is substituted with one or more R²², and optionally substituted with one or more R^{22′}, wherein the 7, 8 or 9 membered spiro-heterocyclyl, 10 membered spiro-heterocyclyl whose ring atoms consist of a nitrogen atom and a carbon atom, 7, 8 or 9 membered fused-heterocyclyl, or 5-6 membered monoheterocyclyl whose ring atoms contain a silicon atom or a phosphorus atom is optionally substituted with one or more R²³, wherein the 7-8 membered bridged heterocyclyl is substituted with one or more R²⁴,
or, R³ is connected to R² or R^{2′}, and together with the carbon atoms connected thereto, forms morpholinyl optionally substituted with one or more R²⁵,
and R², R^{2′} and R³ are not hydrogen at the same time;
or, R², R^{2′} and R³ are each independently selected from the group consisting of hydrogen, amino, amino-C₁-C₄ alkyl-, 7, 8 or 9 membered spiro-heterocycloalkyl, 10 membered spiro-heterocycloalkyl whose ring atoms consist of a nitrogen atom and a carbon atom, 7, 8 or 9 membered fused-heterocycloalkyl, 7-8 membered bridged heterocycloalkyl, 5-6 membered monoheterocycloalkyl whose ring atoms consist of a nitrogen atom and a carbon atom, 5-6 membered monoheterocycloalkyl whose ring atoms contain a silicon atom or a phosphorus atom, 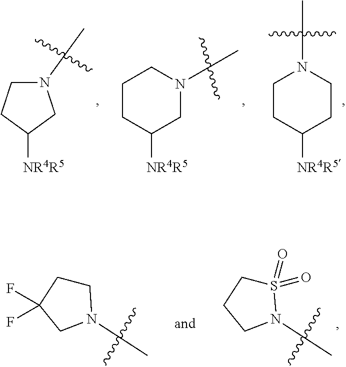
wherein the amino or amino-C₁-C₄ alkyl- is substituted with one or more R²¹, and optionally substituted with one or more R^{21′}, wherein the 5-6 membered monoheterocycloalkyl whose ring atoms consist of a nitrogen atom and a carbon atom is substituted with one or more R²², and optionally substituted with one or more R^{22′}, wherein the 7, 8 or 9 membered spiro-heterocycloalkyl, 10 membered spiro-heterocycloalkyl whose ring atoms consist of a nitrogen atom and a carbon atom, 7, 8 or 9 membered fused-heterocycloalkyl, or 5-6 membered monoheterocycloalkyl whose ring atoms contain a silicon atom or a phosphorus atom is optionally substituted by one or more R²³, wherein the 7-8 membered bridged heterocycloalkyl is substituted by one or more R²⁴,
or, R³ is connected to R² or R^{2′}, and together with the carbon atoms connected thereto, forms morpholinyl optionally substituted with one or more R²,
and R², R^{2′} and R³ are not hydrogen at the same time.

23. The compound of formula (I) or the pharmaceutically acceptable salt thereof according to claim 1, wherein R², R² and R³ are each independently selected from the group consisting of hydrogen, amino, aminomethyl, 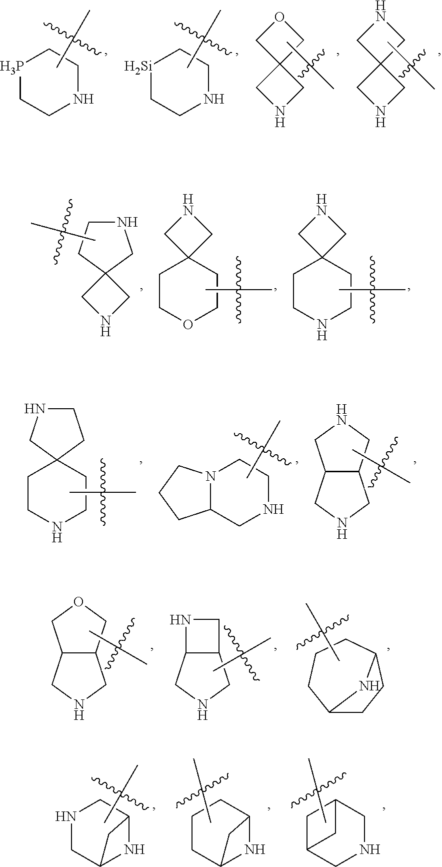
pyrrolidinyl, piperidinyl, piperazinyl, 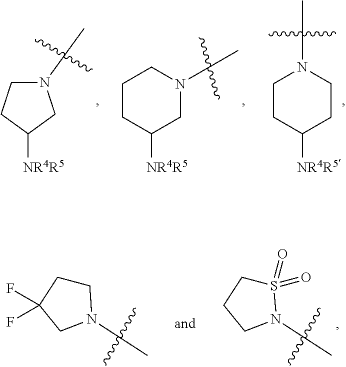
and wherein the amino or aminomethyl is substituted with one or more R²¹, and optionally substituted with one or more R^{21′}, wherein the pyrrolidinyl, piperidinyl or piperazinyl is substituted with one or more R²², and optionally substituted with one or more R^{22′},
wherein the 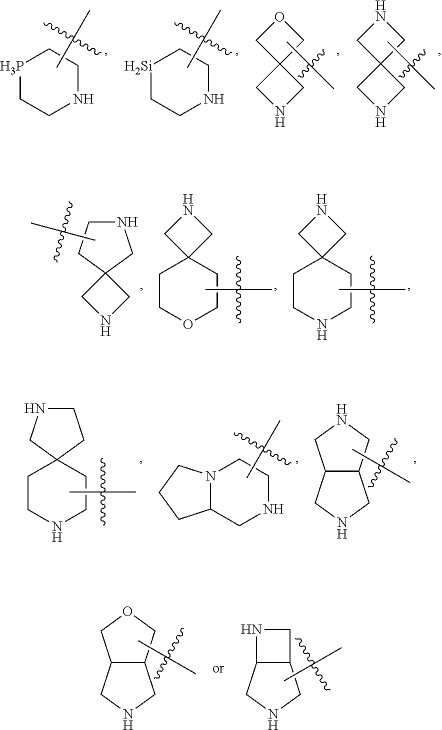
is substituted with one or more R²³, wherein the 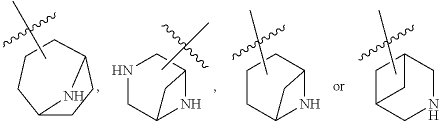
is substituted with one or more R²,
or, R³ is connected to R² or R^{2′}, and together with the carbon atoms connected thereto, forms a 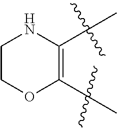 [SMILES: *C1=C(*)OCCN1]
group optionally substituted with one or more R²⁵,
and R², R^{2′} and R³ are not hydrogen at the same time;
or, R², R^{2′} and R³ are each independently selected from the group consisting of hydrogen, amino, aminomethyl, 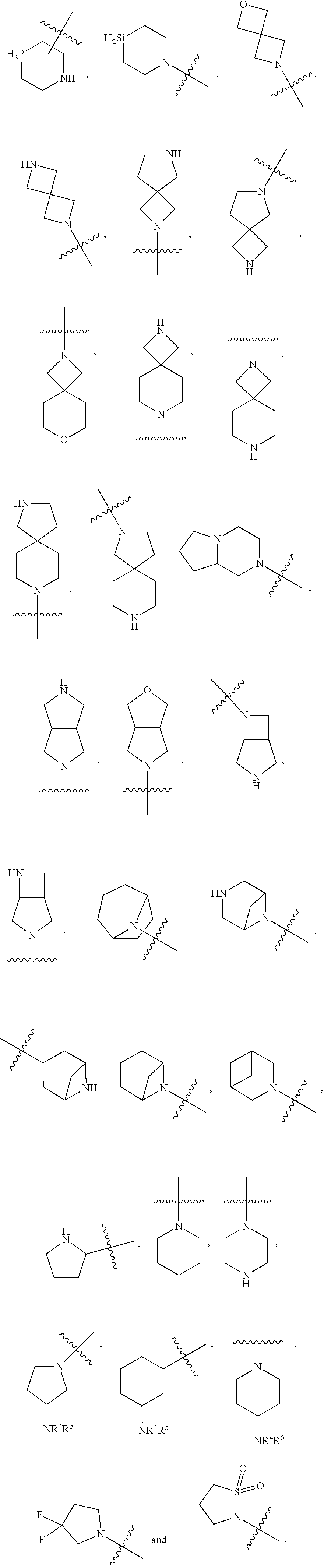
wherein the amino or aminomethyl is substituted with one or more R²¹, and optionally substituted with one or more R^{21′}, wherein the or 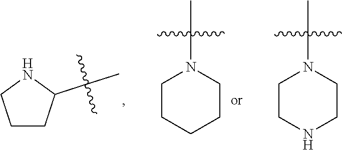 [SMILES: *C1CCCN1 ; *N1CCCCC1 ; *N1CCNCC1]
is substituted with one or more R²², and optionally substituted with one or more R^{22′}, wherein the 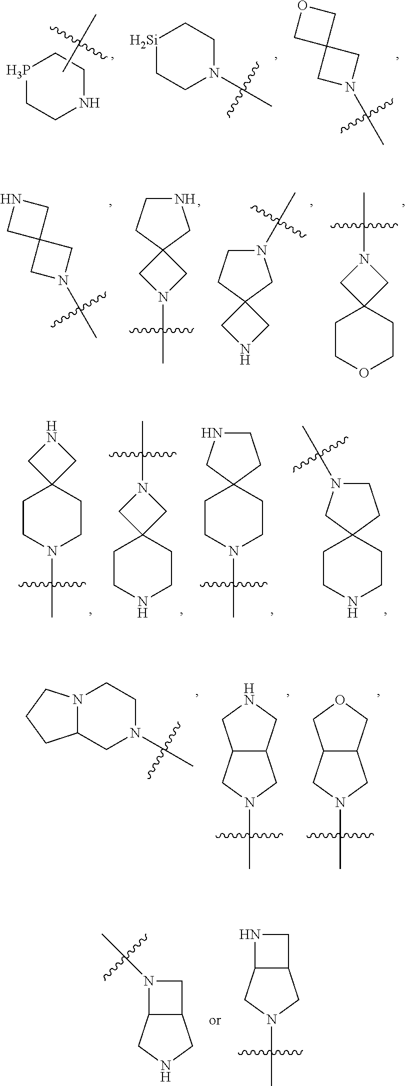
is substituted with one or more R²³,
wherein the 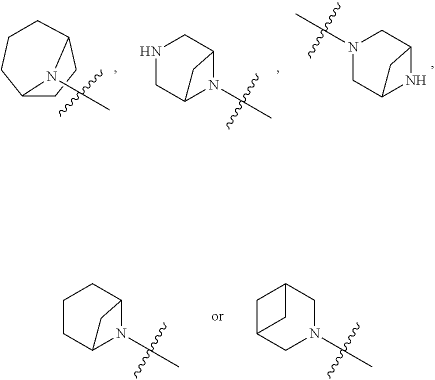 [SMILES: *N1C2CCCC1CC2 ; *N1C2CNCC1C2 ; *N1CC2CC(C1)N2 ; *N1C2CCCC1C2 ; *N1CC2CC(C2)C1]
is substituted with one or more R²⁴,
or, R³ is connected to R² or R^{2′}, and together with the carbon atoms connected thereto, forms a 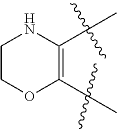 [SMILES: *C1=C(*)OCCN1]
group optionally substituted with one or more R²⁵,
and R², R^{2′} and R³ are not hydrogen at the same time.

24. The compound of formula (I) or the pharmaceutically acceptable salt thereof according to claim 1, wherein R²¹ is selected from the group consisting of C₂-C₄ alkenyl-C(O)—, C₅-C₁₀ cycloalkyl, 4-6 membered heterocyclyl or C₁-C₄ alkylacylamino-C₁-C₄ alkyl-, wherein the C₅-C₁₀ cycloalkyl, 4-6 membered heterocyclyl or C₁-C₄ alkylacylamino-C₁-C₄ alkyl- is optionally substituted with one or more of the following groups selected from the group consisting of: hydroxy, C₁-C₆ alkyl and C₁-C₄ alkylacyl;
or, R²¹ is selected from the group consisting of C₂-C₃ alkenyl-C(O)—, C₁₀ bridged cycloalkyl, 5 membered or 6 membered monoheterocycloalkyl, or C₁-C₃ alkylacylamino-C₁-C₃ alkyl-, wherein the C₁₀ bridged cycloalkyl, 5 membered or 6 membered monoheterocycloalkyl, or C₁-C₃ alkylacylamino-C₁-C₃ alkyl- is optionally substituted with one or more of the following groups selected from the group consisting of: hydroxy, C₁-C₄ alkyl and C₁-C₄ alkylacyl;
or, R²¹ is selected from the group consisting of H₂C═CHC(O)—, 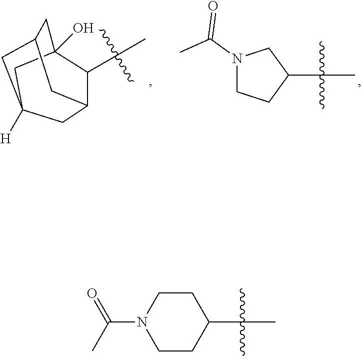
and CH₃C(O)N(CH₃)—CH₃CH₂—;
or, R²¹ is selected from the group consisting of H₂C═CHC(O)—, 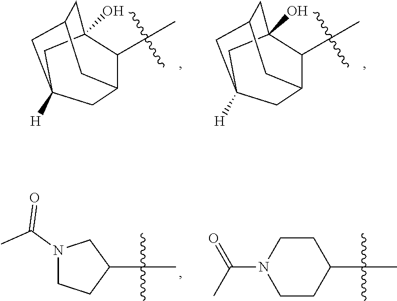
and CH₃C(O)N(CH₃)—CH₃CH₂—.

25. The compound of formula (I) or the pharmaceutically acceptable salt thereof according to claim 1, wherein R²² is selected from the group consisting of C₂-C₃ alkynyl-C(O)— and 4 membered or 5 membered heterocyclyl, wherein the 4 membered or 5 membered heterocyclyl is optionally substituted with one or more [symbol] ;
or, R²² is selected from the group consisting of C₂-C₃ alkynyl-C(O)— and 4 membered or 5 membered heterocycloalkyl, wherein the 4 membered or 5 membered heterocycloalkyl is optionally substituted with one or more [symbol] ;
or, R²² is selected from the group consisting of H₃C≡CC(O)—,

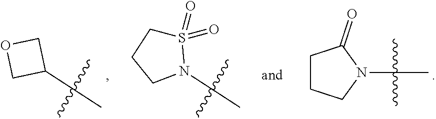 [SMILES: *C1COC1 ; *N1CCCS1(=O)=O ; *N1CCCC1=O]

26. The compound of formula (I) or the pharmaceutically acceptable salt thereof according to claim 1, wherein R²² is selected from the group consisting of C₂-C₃ alkynyl-C(O)—, 4 membered or 5 membered heterocyclyl and 6 membered heterocyclyl whose ring atoms consist of a nitrogen atom and a sulfur atom, wherein the 4 membered or 5 membered heterocyclyl, or 6 membered heterocyclyl whose ring atoms consist of a nitrogen atom and a sulfur atom is optionally substituted with one or more of the following groups selected from the group consisting of: [symbol] , halogen, C₁-C₃ alkyl and C₁-C₆ alkylacyl; R²² is selected from the group consisting of C₂-C₃ alkynyl-C(O)—, 4 membered or 5 membered heterocycloalkyl and 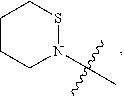 [SMILES: *N1CCCCS1]
wherein the 4 membered or 5 membered heterocycloalkyl or 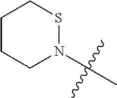 [SMILES: *N1CCCCS1]
is optionally substituted with one or more of the following groups selected from the group consisting of: [symbol] , halogen, C₁-C₃ alkyl and C₁-C₆ alkylacyl; or, R²² is selected from H₃CC≡CC(O)—,

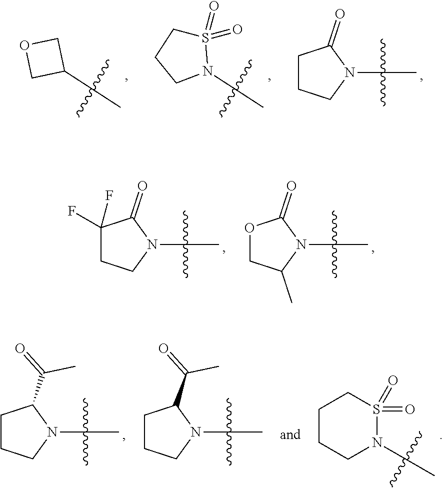

27. The compound of formula (I) or the pharmaceutically acceptable salt thereof according to claim 1, wherein R^{21′} and R^{22′} are each independently selected from the group consisting of deuterium and C₁-C₄ alkyl;
or, R^{21′} and R^{22′} are each independently selected from the group consisting of deuterium and methyl.

28. The compound of formula (I) or the pharmaceutically acceptable salt thereof according to claim 1, wherein R²³ is selected from the group consisting of [symbol] , C₁-C₄ alkyl, C₁-C₄ alkylsulfonyl and C₁-C₄ alkylacyl;
or, R²³ is selected from the group consisting of [symbol] , CH₃—, CH₃S(O)₂— and CH₃C(O)—.

29. The compound of formula (I) or the pharmaceutically acceptable salt thereof according to claim 1, wherein R²⁴ is selected from C₁-C₄ alkylacyl-N(C₁-C₄ alkyl)-;
or, R²⁴ is selected from CH₃C(O)N(CH₃)—.

30. The compound of formula (I) or the pharmaceutically acceptable salt thereof according to claim 1, wherein R²⁵ is selected from C₁-C₄ alkylacyl;
or, R²⁵ is selected from CH₃C(O)—.

31. The compound of formula (I) or the pharmaceutically acceptable salt thereof according to claim 1, wherein R⁴ is selected from the group consisting of C₁-C₄ alkylacyl and C₁-C₄ alkylsulfonyl, and R⁵ is selected from C₁-C₄ alkyl;
or, R⁴ is selected from the group consisting of CH₃C(O)— and CH₃S(O)₂—, and R⁵ is selected from methyl.

32. The compound of formula (I) or the pharmaceutically acceptable salt thereof according to claim 1, wherein R^{5′} is selected from the group consisting of —CD₃ and —CH₃, and when R^{5′} is —CH₃, R¹ or R^{1′} is fluorine.

33. The compound of formula (I) or the pharmaceutically acceptable salt thereof according to claim 1, wherein R², R^{2′} and R³ are each independently selected from the group consisting of hydrogen, 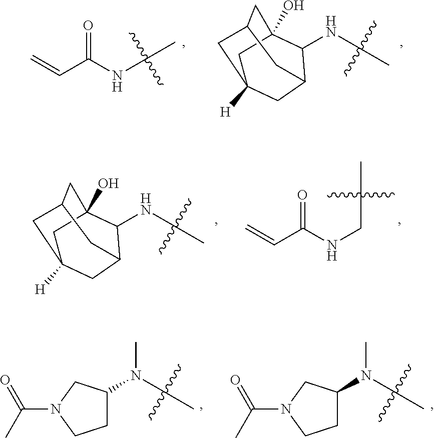 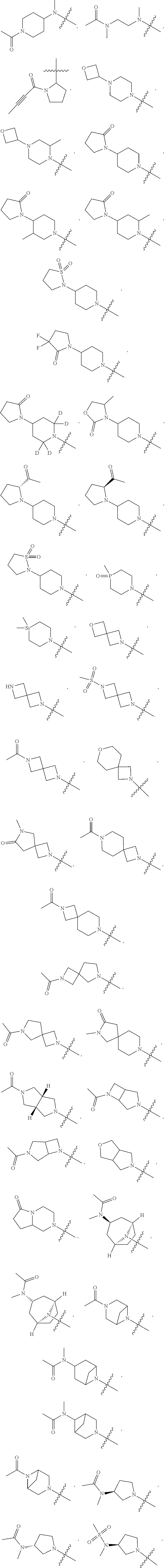 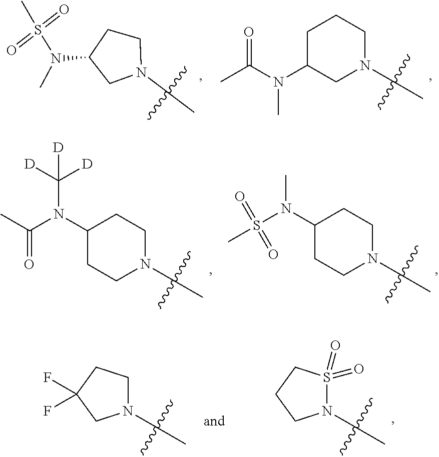 [SMILES: *N1CC[C@@H](N(C)S(C)(=O)=O)C1 ; *N1CCCC(N(C)C(C)=O)C1 ; *N1CCC(N(C(C)=O)C([2H])([2H])[2H])CC1 ; *N1CCC(N(C)S(C)(=O)=O)CC1 ; *N1CCC(F)(F)C1 ; *N1CCCS1(=O)=O]
and or R³ is connected to R² or R^{2′}, such that the structural unit 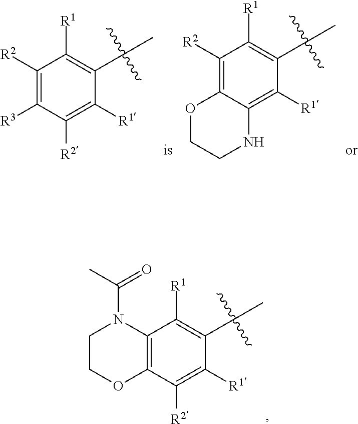
and R², R^{2′} and R³ are not hydrogen at the same time.

34. The compound of formula (I) or the pharmaceutically acceptable salt thereof according to claim 1, selected from:

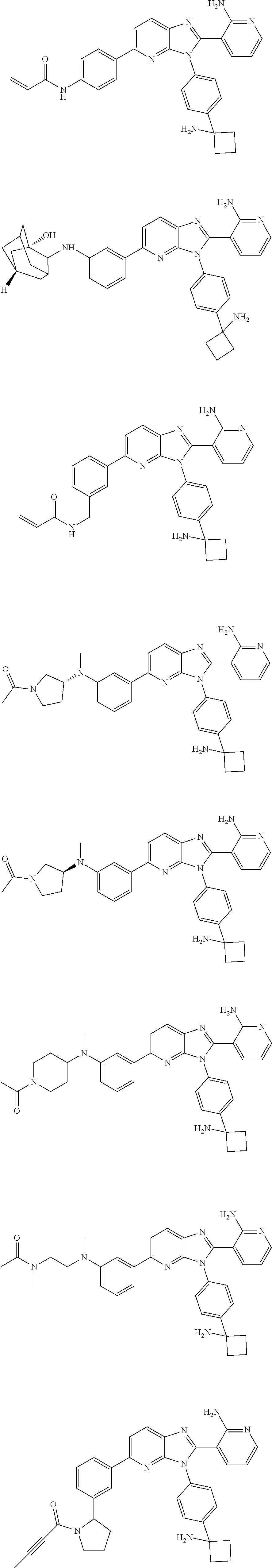

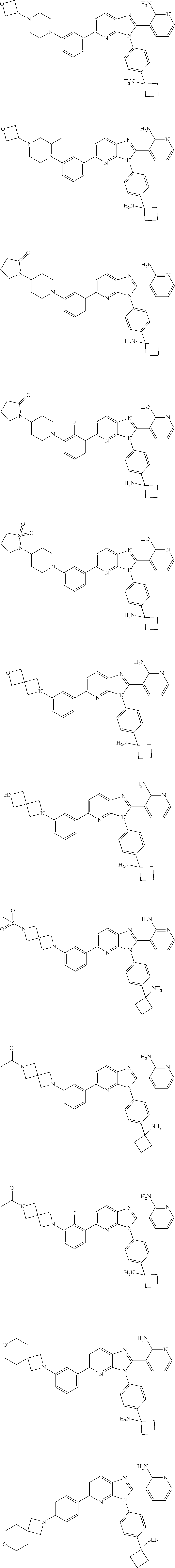

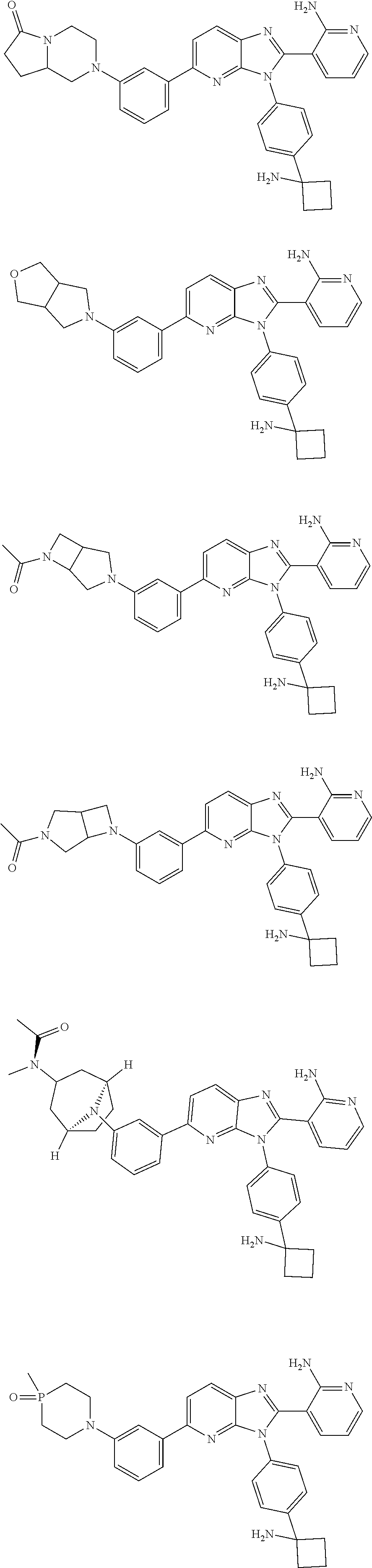

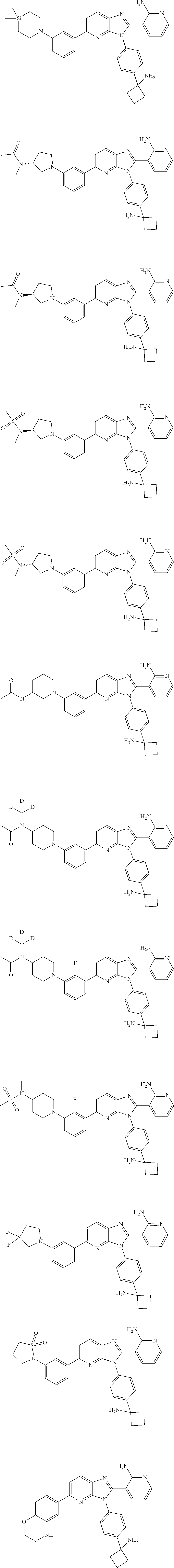

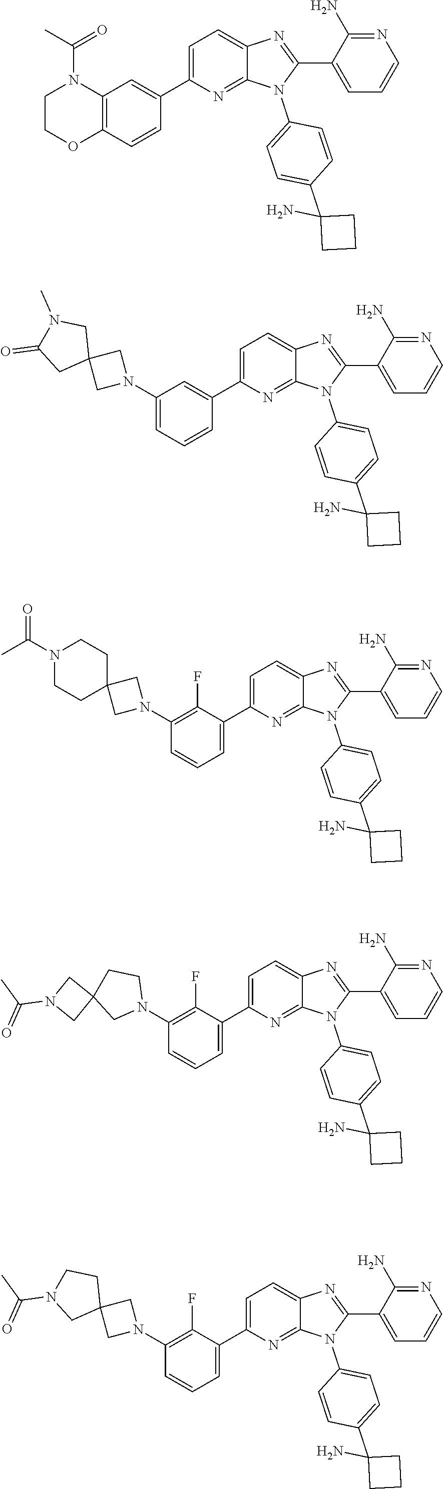 [SMILES: CC(=O)N1CCOc2ccc(-c3ccc4nc(-c5cccnc5N)n(-c5ccc(C6(N)CCC6)cc5)c4n3)cc21 ; CN1CC2(CC1=O)CN(c1cccc(-c3ccc4nc(-c5cccnc5N)n(-c5ccc(C6(N)CCC6)cc5)c4n3)c1)C2 ; CC(=O)N1CCC2(CC1)CN(c1cccc(-c3ccc4nc(-c5cccnc5N)n(-c5ccc(C6(N)CCC6)cc5)c4n3)c1F)C2 ; CC(=O)N1CC2(CCN(c3cccc(-c4ccc5nc(-c6cccnc6N)n(-c6ccc(C7(N)CCC7)cc6)c5n4)c3F)C2)C1 ; CC(=O)N1CCC2(C1)CN(c1cccc(-c3ccc4nc(-c5cccnc5N)n(-c5ccc(C6(N)CCC6)cc5)c4n3)c1F)C2]

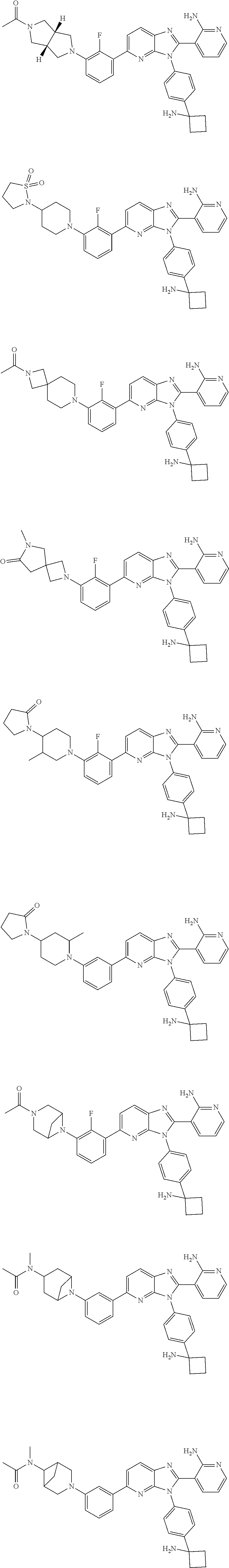

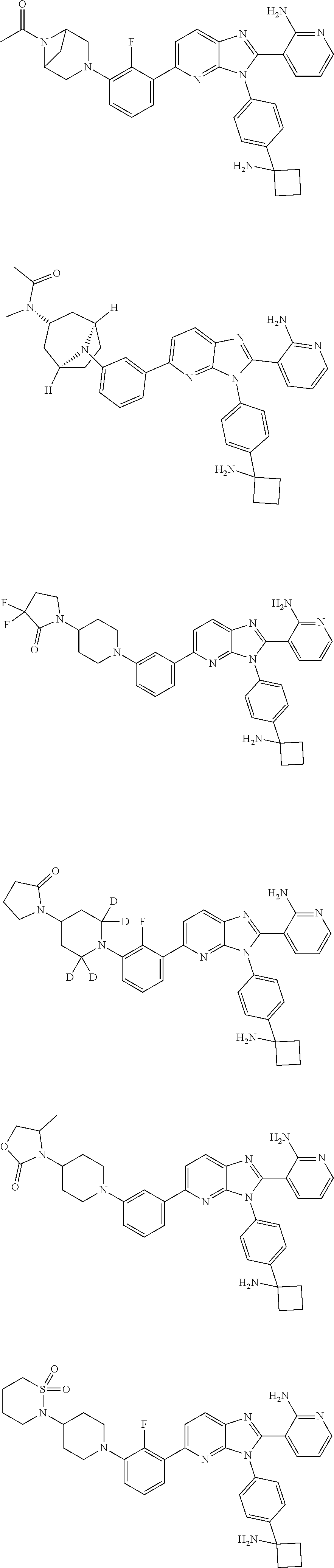

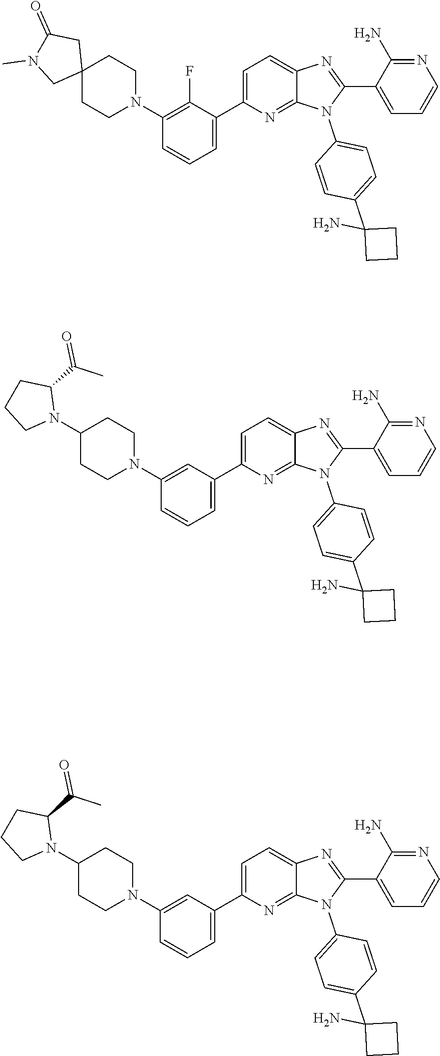 [SMILES: CN1CC2(CCN(c3cccc(-c4ccc5nc(-c6cccnc6N)n(-c6ccc(C7(N)CCC7)cc6)c5n4)c3F)CC2)CC1=O ; CC(=O)[C@H]1CCCN1C1CCN(c2cccc(-c3ccc4nc(-c5cccnc5N)n(-c5ccc(C6(N)CCC6)cc5)c4n3)c2)CC1 ; CC(=O)[C@@H]1CCCN1C1CCN(c2cccc(-c3ccc4nc(-c5cccnc5N)n(-c5ccc(C6(N)CCC6)cc5)c4n3)c2)CC1]

35. A pharmaceutical composition comprising the compound or the pharmaceutically acceptable salt thereof according to claim 1, and a pharmaceutically acceptable excipient.

36. A method for treating a disease mediated by Akt kinase in a mammal, comprising administering to the mammal in need thereof a therapeutically effective amount of the compound or the pharmaceutically acceptable salt thereof according to claim 1.

37. The method according to claim 36, wherein the disease mediated by Akt kinase is selected from cancer.

38. The method according to claim 37, wherein the disease mediated by Akt kinase is selected from the group consisting of prostate cancer and endometrial cancer.
